SEQUENCE LISTING (80 sequences)

SEQ ID NO: 1 - protein, 245 aa, Epstein-barr virus
  source 1..245: note = strain p3hr-1
MMDPNSTSEDVKFTPDPYQVPFVQAFDQATRVYQDLGGPSQAPLPCVLWPVLPEPLPQGQLTAYHVSTAPTGSWFSAPQPAPENAYQAYAAPQLFPVSDITQNQQTNQAGGEAPQPGDNSTVQTAAAVVFACPGANQGQQLADIGVPQPAPVAAPARRTRKPQQPESLEECDSELEIKRYKNRVASRKCRAKFKQLLQHYREVAAAKSSENDRLRLLLKQMCPSLDVDSIIPRTPDVLHEDLLNF

SEQ ID NO: 2 - protein, 42 aa, synthetic construct
  REGION 1..42: note = Derived from Epstein-barr virus (EBV) ZEBRA protein (synthesized)
KRYKNRVASRKSRAKFKQLLQHYREVAAAKSSENDRLRLLLK

SEQ ID NO: 3 - protein, 30 aa, synthetic construct
  REGION 1..30: note = Derived from Epstein-barr virus (EBV) ZEBRA protein (synthesized)
KRYKNRVASRKSRAKFKQLLQHYREVAAAK

SEQ ID NO: 4 - protein, 17 aa, synthetic construct
  REGION 1..17: note = Derived from Epstein-barr virus (EBV) ZEBRA protein (synthesized)
KRYKNRVASRKSRAKFK

SEQ ID NO: 5 - protein, 19 aa, synthetic construct
  REGION 1..19: note = Derived from Epstein-barr virus (EBV) ZEBRA protein (synthesized)
REVAAAKSSENDRLRLLLK

SEQ ID NO: 6 - protein, 35 aa, synthetic construct
  REGION 1..35: note = Derived from Homo sapiens Annexin A2 protein (synthesized)
STVHEILCKLSLEGDHSTPPSAYGSVKPYTNFDAE

SEQ ID NO: 7 - protein, 35 aa, synthetic construct
  REGION 1..35: note = Derived from Homo sapiens Annexin A2 protein (synthesized)
STVHEILSKLSLEGDHSTPPSAYGSVKPYTNFDAE

SEQ ID NO: 8 - protein, 90 aa, synthetic construct
  REGION 1..90: note = Derived from Homo sapiens Fibronectin protein (synthesized)
NIDRPKGLAFTDVDVDSIKIAWESPQGQVSRYRVTYSSPEDGIRELFPAPDGEDDTAELQGLRPGSEYTVSVVALHDDMESQPLIGIQST

SEQ ID NO: 9 - protein, 185 aa, synthetic construct
  REGION 1..185: note = Derived from Homo sapiens High mobility group protein B1 (HMGB1) (synthesized)
MGKGDPKKPRGKMSSYAFFVQTCREEHKKKHPDASVNFSEFSKKCSERWKTMSAKEKGKFEDMAKADKARYEREMKTYIPPKGETKKKFKDPNAPKRPPSAFFLFCSEYRPKIKGEHPGLSIGDVAKKLGEMWNNTAADDKQPYEKKAAKLKEKYEKDIAAYRAKGKPDAAKKGVVKAEKSKKKK

SEQ ID NO: 10 - protein, 314 aa, Homo sapiens
MPLEQRSQHCKPEEGLEARGEALGLVGAQAPATEEQEAASSSSTLVEVTLGEVPAAESPDPPQSPQGASSLPTTMNYPLWSQSYEDSSNQEEEGPSTFPDLESEFQAALSRKVAELVHFLLLKYRAREPVTKAEMLGSVVGNWQYFFPVIFSKAFSSLQLVFGIELMEVDPIGHLYIFATCLGLSYDGLLGDNQIMPKAGLLIIVLAIIAREGDCAPEEKIWEELSVLEVFEGREDSILGDPKKLLTQHFVQENYLEYRQVPGSDPACYEFLWGPRALVETSYVKVLHHMVKISGGPHISYPPLHEWVLREGEE

SEQ ID NO: 11 - protein, 630 aa, Homo sapiens
MALPTARPLLGSCGTPALGSLLFLLFSLGWVQPSRTLAGETGQEAAPLDGVLANPPNISSLSPRQLLGFPCAEVSGLSTERVRELAVALAQKNVKLSTEQLRCLAHRLSEPPEDLDALPLDLLLFLNPDAFSGPQACTRFFSRITKANVDLLPRGAPERQRLLPAALACWGVRGSLLSEADVRALGGLACDLPGRFVAESAEVLLPRLVSCPGPLDQDQQEAARAALQGGGPPYGPPSTWSVSTMDALRGLLPVLGQPIIRSIPQGIVAAWRQRSSRDPSWRQPERTILRPRFRREVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQAPRRPLPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALSGTPCLLGPGPVLTVLALLLASTLA

SEQ ID NO: 12 - protein, 142 aa, Homo sapiens
MGAPTLPPAWQPFLKDHRISTFKNWPFLEGCACTPERMAEAGFIHCPTENEPDLAQCFFCFKELEGWEPDDDPIEEHKKHSSGCAFLSVKKQFEELTLGEFLKLDRERAKNKIAKETNNKKKEFEETAKKVRRAIEQLAAMD

SEQ ID NO: 13 - protein, 180 aa, Homo sapiens
MQAEGRGTGGSTGDADGPGGPGIPDGPGGNAGGPGEAGATGGRGPRGAGAARASGPGGGAPRGPHGGAASGLNGCCRCGARGPESRLLEFYLAMPFATPMEAELARRSLAQDAPPLPVPGVLLKEFTVSGNILTIRLTAADHRQLQLSISSCLQQLSLLMWITQCFLPVFLAQPPSGQRR

SEQ ID NO: 14 - protein, 509 aa, Homo sapiens
MERRRLWGSIQSRYISMSVWTSPRRLVELAGQSLLKDEALAIAALELLPRELFPPLFMAAFDGRHSQTLKAMVQAWPFTCLPLGVLMKGQHLHLETFKAVLDGLDVLLAQEVRPRRWKLQVLDLRKNSHQDFWTVWSGNRASLYSFPEPEAAQPMTKKRKVDGLSTEAEQPFIPVEVLVDLFLKEGACDELFSYLIEKVKRKKNVLRLCCKKLKIFAMPMQDIKMILKMVQLDSIEDLEVTCTWKLPTLAKFSPYLGQMINLRRLLLSHIHASSYISPEKEEQYIAQFTSQFLSLQCLQALYVDSLFFLRGRLDQLLRHVMNPLETLSITNCRLSEGDVMHLSQSPSVSQLSVLSLSGVMLTDVSPEPLQALLERASATLQDLVFDECGITDDQLLALLPSLSHCSQLTTLSFYGNSISISALQSLLQHLIGLSNLTHVLYPVPLESYEDIHGTLHLERLAYLHARLRELLCELGRPSMVWLSANPCPHCGDRTFYDPEPILCPCFMPN

SEQ ID NO: 15 - protein, 193 aa, Homo sapiens
MDGGTLPRSAPPAPPVPVGCAARRRPASPELLRCSRRRRPATAETGGGAAAVARRNERERNRVKLVNLGFQALRQHVPHGGASKKLSKVETLRSAVEYIRALQRLLAEHDAVRNALAGGLRPQAVRPSAPRGPPGTTPVAASPSRASSSPGRGGSSEPGSPRSAYSSDDSGCEGALSPAERELLDFSSWLGGY

SEQ ID NO: 16 - protein, 10 aa, synthetic construct
  REGION 1..10: note = Derived from Homo sapiens Achaete-scute homolog 2 (ASCL2) (synthesized)
SAVEYIRALQ

SEQ ID NO: 17 - protein, 10 aa, synthetic construct
  REGION 1..10: note = Derived from Homo sapiens Achaete-scute homolog 2 (ASCL2) (synthesized)
ERELLDFSSW

SEQ ID NO: 18 - protein, 106 aa, synthetic construct
  REGION 1..106: note = Derived from Homo sapiens Achaete-scute homolog 2 (ASCL2) (synthesized)
AAVARRNERERNRVKLVNLGFQALRQHVPHGGASKKLSKVETLRSAVEYIRALQRLLAEHDAVRNALAGGLRPQAVRPSAPRGPSEGALSPAERELLDFSSWLGGY

SEQ ID NO: 19 - protein, 1255 aa, Homo sapiens
MTPGTQSPFFLLLLLTVLTVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDNRPALGSTAPPVHNVTSASGSASGSASTLVHNGTSARATTTPASKSTPFSIPSHHSDTPTTLASHSTKTDASSTHHSSVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 20 - protein, 9 aa, synthetic construct
  REGION 1..9: note = Derived from Homo sapiens Mucin-1 (MUC-1) (synthesized)
GSTAPPVHN

SEQ ID NO: 21 - protein, 10 aa, synthetic construct
  REGION 1..10: note = Derived from Homo sapiens Mucin-1 (MUC-1) (synthesized)
TAPPAHGVTS

SEQ ID NO: 22 - protein, 10 aa, synthetic construct
  REGION 1..10: note = Derived from Homo sapiens survivin (synthesized)
RISTFKNWPF

SEQ ID NO: 23 - protein, 50 aa, synthetic construct
  REGION 1..50: note = Derived from Homo sapiens survivin (synthesized)
APTLPPAWQPFLKDHRISTFKNWPFLEGSAVKKQFEELTLGEFLKLDRER

SEQ ID NO: 24 - protein, 701 aa, Homo sapiens
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNKLSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVAL

SEQ ID NO: 25 - protein, 120 aa, synthetic construct
  REGION 1..120: note = Derived from Homo sapiens Carcino-embryonic antigen (CEA) (synthesized)
NRTLTLFNVTRNDARAYVSGIQNSVSANRSDPVTLDVLPDSSYLSGANLNLSCHSASPQYSWRINGIPQQHTQVLFIAKITPNNNGTYACFVSNLATGRNNSIVKSITVSASGTSPGLSA

SEQ ID NO: 26 - protein, 10 aa, synthetic construct
  REGION 1..10: note = Derived from Homo sapiens Carcino-embryonic antigen (CEA) (synthesized)
YLSGANLNLS

SEQ ID NO: 27 - protein, 10 aa, synthetic construct
  REGION 1..10: note = Derived from Homo sapiens Carcino-embryonic antigen (CEA) (synthesized)
SWRINGIPQQ

SEQ ID NO: 28 - protein, 567 aa, Homo sapiens
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 29 - protein, 393 aa, Homo sapiens [sequence omitted]

SEQ ID NO: 30 - protein, 189 aa, Homo sapiens [sequence omitted]

SEQ ID NO: 31 - protein, 9 aa, synthetic construct
  REGION 1..9: note = Derived from Homo sapiens Kirsten Ras (Kras) (synthesized)
VVVGAGGVG

SEQ ID NO: 32 - protein, 1046 aa, synthetic construct
  REGION 1..1046: note = Derived from Homo sapiens O-linked N-Acetylglucosamine (GlcNAc) Transferase (OGT) (synthesized)
MASSVGNVADSTEPTKRMLSFQGLAELAHREYQAGDFEAAERHCMQLWRQEPDNTGVLLLLSSIHFQCRRLDRSAHFSTLAIKQNPLLAEAYSNLGNVYKERGQLQEAIEHYRHALRLKPDFIDGYINLAAALVAAGDMEGAVQAYVSALQYNPDLYCVRSDLGNLLKALGRLEEAKACYLKAIETQPNFAVAWSNLGCVFNAQGEIWLAIHHFEKAVTLDPNFLDAYINLGNVLKEARIFDRAVAAYLRALSLSPNHAVVHGNLACVYYEQGLIDLAIDTYRRAIELQPHFPDAYCNLANALKEKGSVAEAEDCYNTALRLCPTHADSLNNLANIKREQGNIEEAVRLYRKALEVFPEFAAAHSNLASVLQQQGKLQEALMHYKEAIRISPTFADAYSNMGNTLKEMQDVQGALQCYTRAIQINPAFADAHSNLASIHKDSGNIPEAIASYRTALKLKPDFPDAYCNLAHCLQIVCDWTDYDERMKKLVSIVADQLEKNRLPSVHPHHSMLYPLSHGFRKAIAERHGNLCLDKINVLHKPPYEHPKDLKLSDGRLRVGYVSSDFGNHPTSHLMQSIPGMHNPDKFEVFCYALSPDDGTNFRVKVMAEANHFIDLSQIPCNGKAADRIHQDGIHILVNMNGYTKGARNELFALRPAPIQAMWLGYPGTSGALFMDYIITDQETSPAEVAEQYSEKLAYMPHTFFIGDHANMFPHLKKKAVIDFKSNGHIYDNRIVLNGIDLKAFLDSLPDVKIVKMKCPDGGDNADSSNTALNMPVIPMNTIAEAVIEMINRGQIQITINGFSISNGLATTQINNKAATGEEVPRTIIVTTRSQYGLPEDAIVYCNFNQLYKIDPSTLQMWANILKRVPNSVLWLLRFPAVGEPNIQQYAQNMGLPQNRIIFSPVAPKEEHVRRGQLADVCLDTPLCNGHTTGMDVLWAGTPMVTMPGETLASRVAASQLTCLGCLELIAKNRQEYEDIAVKLGTDLEYLKKVRGKVWKQRISSPLFNTKQYTMELERLYLQMWEHYAAGNKPDHMIKPVEVTESA

SEQ ID NO: 33 - protein, 434 aa, Homo sapiens
MAEDSGKKKRRKNFEAMFKGILQSGLDNFVINHMLKNNVAGQTSIQTLVPNTDQKSTSVKKDNHKKKTVKMLEYLGKDVLHGVFNYLAKHDVLTLKEEEKKKYYDTKIEDKALILVDSLRKNRVAHQMFTQTLLNMDQKITSVKPLLQIEAGPPESAESTNILKLCPREEFLRLCKKNHDEIYPIKKREDRRRLALIICNTKFDHLPARNGAHYDIVGMKRLLQGLGYTVVDEKNLTARDMESVLRAFAARPEHKSSDSTFLVLMSHGILEGICGTAHKKKKPDVLLYDTIFQIFNNRNCLSLKDKPKVIIVQACRGEKHGELWVRDSPASLALISSQSSENLEADSVCKIHEEKDFIAFCSSTPHNVSWRDRTRGSIFITELITCFQKYSCCCHLMEIFRKVQKSFEVPQAKAQMPTIERATLTRDFYLFPGN

SEQ ID NO: 34 - protein, 520 aa, Homo sapiens
MSSPLASLSKTRKVPLPSEPMNPGRRGIRIYGDEDEVDMLSDGCGSEEKISVPSCYGGIGAPVSRQVPASHDSELMAFMTRKLWDLEQQVKAQTDEILSKDQKIAALEDLVQTLRPHPAEATLQRQEELETMCVQLQRQVREMERFLSDYGLQWVGEPMDQEDSESKTVSEHGERDWMTAKKFWKPGDSLAPPEVDFDRLLASLQDLSELVVEGDTQVTPVPGGARLRTLEPIPLKLYRNGIMMFDGPFQPFYDPSTQRCLRDILDGFFPSELQRLYPNGVPFKVSDLRNQVYLEDGLDPFPGEGRVVGRQLMHKALDRVEEHPGSRMTAEKFLNRZPKFVIRQGEVIDIRGPIRDTLQNCCPLPARIQEIVVETPTLAAERERSQESPNTPAPPLSMLRIKSENGEQAFLLMMQPDNTIGDVRALLAQARVMDASAFEIFSTFPPTLYQDDTLTLQAAGLVPKAALLLRARRAPKSSLKFSPGPCPGPGPGPSPGPGPGPSPGPGPGPSPCPGPSPSPQ

SEQ ID NO: 35 - protein, 380 aa, Homo sapiens
MAFVCLAIGCLYTFLISTTFGCTSSSDTEIKVNPPQDFEIVDPGYLGYLYLQWQPPLSLDHFKECTVEYELKYRNIGSETWKTIITKNLHYKDGFDLNKGIEAKIHTLLPWQCTNGSEVQSSWAETTYWISPQGIPETKVQDMDCVYYNWQYLLCSWKPGIGVLLDTNYNLFYWYEGLDHALQCVDYIKADGQNIGCRFPYLEASDYKDFYICVNGSSENKPIRSSYFTFQLQNIVKPLPPVYLTFTRESSCEIKLKWSIPLGPIPARCFDYEIEIREDDTTLVTATVENETYTLKTTNETRQLCFVVRSKVNIYCSDDGIWSEWSDKQCWEGEDLSKKTLLRFWLPFGFILILVIFVTGLLLRKPNTYPKMIPEFFCDT

SEQ ID NO: 36 - protein, 7 aa, synthetic construct
  REGION 1..7: note = Derived from Homo sapiens IL13Ralpha2 (synthesized)
LPFGFIL

SEQ ID NO: 37 - protein, 579 aa, Homo sapiens
MNKLYIGNLSENAAPSDLESIFKDAKIPVSGPFLVKTGYAFVDCPDESWALKAIEALSGKIELHGKPIEVEHSVPKRQRIRKLQIRNIPPHLQWEVLDSLLVQYGVVESCEQVNTDSETAVVNVTYSSKDQARQALDKLNGFQLENFTLKVAYIPDEMAAQQNPLQQPRGRRGLGQRGSSRQGSPGSVSKQKPCDLPLRLLVPTQFVGAIIGKEGATIRNITKQTQSKIDVHRKENAGAAEKSITILSTPEGTSAACKSILEIMHKEAQDIKFTEEIPLKILAHNNFVGRLIGKEGRNLKKIEQDTDTKITISPLQELTLYNPERTITVKGNVETCAKAEEEIMKKIRESYENDIASMNLQAHLIPGLNLNALGLFPPTSGMPPPTSGPPSAMTPPYPQFEQSETETVHLFIPALSVGAIIGKQGQHIKQLSRFAGASIKIAPAEAPDAKVRMVIITGPPEAQFKAQGRIYGKIKEENFVSPKEEVKLEAHIRVPSFAAGRVIGKGGKTVNELQNLSSAEVVVPRDQTPDENDQVVVKITGHFYACQVAQRKIQEILTQVKQHQQQKALQSGPPQSRRK

SEQ ID NO: 38 - protein, 309 aa, Homo sapiens
MAPKFPDSVEELRAAGNESFRNGQYAEASALYGRALRVLQAQGSSDPEEESVLYSNRAACHLKDGNCRDCIKDCTSALALVPFSIKPLLRRASAYEALEKYPMAYVDYKTVLQIDDNVTSAVEGINRMTRALMDSLGPEWRLKLPSIPLVPVSAQKRWNSLPSENHKEMAKSKSKETTATKNRVPSAGDVEKARVLKEEGNELVKKGNHKKAIEKYSESLLCSNLESATYSNRALCYLVLKQYTEAVKDCTEALKLDGKNVKAFYRRAQAHKALKDYKSSFADISNLLQIEPRNGPAQKLRQEVKQNLH

SEQ ID NO: 39 - protein, 783 aa, Homo sapiens
MSGGHQLQLAALWPWLLMATLQAGFGRTGLVLAAAVESERSAEQKAIIRVIPLKMDPTGKLNLTLEGVFAGVAEITPAEGKLMQSHPLYLCNASDDDNLEPGFISIVKLESPRRAPRPCLSLASKARMAGERGASAVLFDITEDRAAAEQLQQPLGLTWPVVLIWGNDAEKLMEFVYKNQKAHVRIELKEPPAWPDYDVWILMTVVGTIFVIILASVLRIRCRPRHSRPDPLQQRTAWAISQLATRRYQASCRQARGEWPDSGSSCSSAPVCAICLEEFSEGQELRVISCLHEFHRNCVDPWLHQHRTCPLCMFNITEGDSFSQSLGPSRSYQEPGRRLHLIRQHPGHAHYHLPAAYLLGPSRSAVARPPRPGPFLPSQEPGMGPRHHRFPRAAHPRAPGEQQRLAGAQHPYAQGWGLSHLQSTSQHPAACPVPLRRARPPDSSGSGESYCTERSGYLADGPASDSSSGPCHGSSSDSVVNCTDISLQGVHGSSSTFCSSLSSDFDPLVYCSPKGDPQRVDMQPSVTSRPRSLDSVVPTGETQVSSHVHYHRHRHHHYKKRFQWHGRKPGPETGVPQSRPPIPRTQPQPEPPSPDQQVTRSNSAAPSGRLSNPQCPRALPEPAPGPVDASSICPSTSSLFNLQKSSLSARHPQRKRRGGPSEPTPGSRPQDATVHPACQIFPHYTPSVAYPWSPEAHPLICGPPGLDKRLLPETPGPCYSNSQPVWLCLTPRQPLEPHPPGEGPSEWSSDTAEGRPCPYPHCQVLSAQPGSEEELEELCEQAV

SEQ ID NO: 40 - protein, 314 aa, Homo sapiens
MAPPQVLAFGLLLAAATATFAAAQEECVCENYKLAVNCFVNNNRQCQCTSVGAQNTVICSKLAAKCLVMKAEMNGSKLGRRAKPEGALQNNDGLYDPDCDESGLFKAKQCNGTSMCWCVNTAGVRRTDKDTEITCSERVRTYWIIIELKHKAREKPYDSKSLRTALQKEITTRYQLDPKFITSILYENNVITIDLVQNSSQKTQNDVDIADVAYYFEKDVKGESLFHSKKMDLTVNGEQLDLDPGQTLIYYVDEKAPEFSMQGLKAGVIAVIVVVVIAVVAGIVVLVISRKKRMAKYEKAEIKEMGEMHRELNA

SEQ ID NO: 41 - protein, 9 aa, synthetic construct
  REGION 1..9: note = Derived from Homo sapiens EpCAM (synthesized)
GLKAGVIAV

SEQ ID NO: 42 - protein, 1255 aa, Homo sapiens [sequence omitted]

SEQ ID NO: 43 - protein, 449 aa, Homo sapiens
MGSDVRDLNALLPAVPSLGGGGGCALPVSGAAQWAPVLDFAPPGASAYGSLGGPAPPPAPPPPPPPPPHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGVAAGSSSSVKWTEGQSNHSTGYESDNHTTPILCGAQYRIHTHGVFRGIQDVRRVPGVAPTLVRSASETSEKRPFMCAYPGCNKRYFKLSHLQMHSRKHTGEKPYQCDFKDCERRFSRSDQLKRHQRRHTGVKPFQCKTCQRKFSRSDHLKTHTRTHTGKTSEKPFSCRWPSCQKKFARSDELVRHHNMHQRNMTKLQLAL

SEQ ID NO: 44 - protein, 9 aa, synthetic construct
  REGION 1..9: note = Derived from Homo sapiens Melanoma-associated antigen (MAGE) A3 (synthesized)
KVAELVHFL

SEQ ID NO: 45 - protein, 276 aa, synthetic construct
  REGION 1..276: note = Derived from 3 Homo sapiens antigens (synthesized)
NRTLTLFNVTRNDARAYVSGIQNSVSANRSDPVTLDVLPDSSYLSGANLNLSCHSASPQYSWRINGIPQQHTQVLFIAKITPNNNGTYACFVSNLATGRNNSIVKSITVSASGTSPGLSAAPTLPPAWQPFLKDHRISTFKNWPFLEGSAVKKQFEELTLGEFLKLDRERAAVARRNERERNRVKLVNLGFQALRQHVPHGGASKKLSKVETLRSAVEYIRALQRLLAEHDAVRNALAGGLRPQAVRPSAPRGPSEGALSPAERELLDFSSWLGGY

SEQ ID NO: 46 - protein, 498 aa, Lymphocytic choriomeningitis virus
MGQIVTMFEALPHIIDEVINIVIIVLIIITSIKAVYNFATCGILALVSFLFLAGRSCGMYGLNGPDIYKGVYQFKSVEFDMSHLNLTMPNACSANNSHHYISMGSSGLELTFTNDSILNHNFCNLTSAFNKKTFDHTLMSIVSSLHLSIRGNSNHKAVSCDFNNGITIQYNLSFSDPQSAISQCRTFRGRVLDMFRTAFGGKYMRSGWGWAGSDGKTTWCSQTSYQYLIIQNRTWENHCRYAGPFGMSRILFAQEKTKFLTRRLAGTFTWTLSDSSGVENPGGYCLTKWMILAAELKCFGNTAVAKCNVNHDEEFCDMLRLIDYNKAALSKFKQDVESALHVFKTTVNSLISDQLLMRNHLRDLMGVPYCNYSKFWYLEHAKTGETSVPKCWLVTNGSYLNETHFSDQIEQEADNMITEMLRKDYIKRQGSTPLALMDLLMFSTSAYLISIFLHLVKIPTHRHIKGGSCPKPHRLTNKGICSCGAFKVPGVKTIWKRR

SEQ ID NO: 47 - protein, 498 aa, unidentified
  REGION 1..498: note = Mammarenavirus/dandenong virus
MGQLITMFEALPHIIDEVINIVIIVLVIITSIKAVYNFATCGIIALISFCLLAGRSCGLYGVTGPDIYKGLYQFKSVEFNMSQLNLTMPNACSANNSHHYISMGKSGLELTFTNDSIISHNFCNLTDGFKKKTFDHTLMSIVASLHLSIRGNTNYKAVSCDFNNGITIQYNLSFSDAQSAINQCRTFRGRVLDMFRTAFGGKYMRSGYGWKGSDGKTTWCSQTSYQYLIIQNRTWENHCEYAGPFGLSRVLFAQEKTKFLTRRLAGTFTWTLSDSSGTENPGGYCLTKWMLIAAELKCFGNTAVAKCNINHDEEFCDMLRLIDYNKAALKKFKEDVESALHLFKTTVNSLISDQLLMRNHLRDLMGVPYCNYSKFWYLEHVKTGDTSVPKCWLVSNGSYLNETHFSDQIEQEADNMITEMLRKDYIKRQGSTPLALMDLLMFSTSAYLISVFLHLMKIPTHRHIKGGTCPKPHRLTSKGICSCGAFKVPGVKTVWKRR

SEQ ID NO: 48 - protein, 489 aa, unidentified
  REGION 1..489: note = Mammarenavirus/mopeia virus
MGQIVTFFQEVPHILEEVMNIVLMTLSILAILKGIYNVMTCGIIGLITFLFLCGRSCSSIYKDNYEFFSLDLDMSSLNATMPLSCSKNNSHHYIQVGNETGLELTLTNTSIIDHKFCNLSDAHRRNLYDKALMSILTTFHLSIPDFNQYEAMSCDFNGGKISIQYNLSHSNYVDAGNHCGTIANGIMDVFRRMYWSTSLSVASDISGTQCIQTDYKYLIIQNTSWEDHCMFSRPSPMGFLSLLSQRTRNFYISRRLLGLFTWTLSDSEGNDMPGGYCLTRSMLIGLDLKCFGNTAIAKCNQAHDEEFCDMLRLFDFNKQAISKLRSEVQQSINLINKAVNALINDQLVMRNHLRDLMGIPYCNYSKFWYLNDTRTGRTSLPKCWLVTNGSYLNETQFSTEIEQEANNMFTDMLRKEYEKRQSTTPLGLVDLFVFSTSFYLISVFLHLIKIPTHRHIKGKPCPKPHRLNHMAICSCGFYKQPGLPTQWKR

SEQ ID NO: 49 - protein, 422 aa, Vesicular stomatitis virus
MSVTVKRIIDNTVVVPKLPANEDPVEYPADYFRKSKEIPLYINTTKSLSDLRGYVYQGLKSGNVSIIHVNSYLYGALKDIRGKLDKDWSSFGINIGKAGDTIGIFDLVSLKALDGVLPDGVSDASRTSADDKWLPLYLLGLYRVGRTQMPEYRKKLMDGLTNQCKMINEQFEPLVPEGRDIFDVWGNDSNYTKIVAAVDMFFHMFKKHECASFRYGTIVSRFKDCAALATFGHLCKITGMSTEDVTTWILNREVADEMVQMMLPGQEIDKADSYMPYLIDFGLSSKSPYSSVKNPAFHFWGQLTALLLRSTRARNARQPDDIEYTSLTTAGLLYAYAVGSSADLAQQFCVGDNKYTPDDSTGGLTTNAPPQGRDVVEWLGWFEDQNRKPTPDMMQYAKRAVMSLQGLREKTIGKYAKSEFDK

SEQ ID NO: 50 - protein, 265 aa, Vesicular stomatitis virus
MDNLTKVREYLKSYSRLDQAVGEIDEIEAQRAEKSNYELFQEDGVEEHTKPSYFQAADDSDTESEPEIEDNQGLYAPDPEAEQVEGFIQGPLDDYADEEVDVVFTSDWKQPELESDEHGKTLRLTSPEGLSGEQKSQWLSTIKAVVQSAKYWNLAECTFEASGEGVIMKERQITPDVYKVTPVMNTHPSQSEAVSDVWSLSKTSMTFQPKKASLQPLTISLDELFSSRGEFISVGGDGRMSHKEAILLGLRYKKLYNQARVKYSL

SEQ ID NO: 51 - protein, 2109 aa, Vesicular stomatitis virus
MEVHDFETDEFNDFNEDDYATREFLNPDERMTYLNHADYNLNSPLISDDIDNLIRKFNSLPIPSMWDSKNWDGVLEMLTSCQANPIPTSQMHKWMGSWLMSDNHDASQGYSFLHEVDKEAEITFDVVETFIRGWGNKPIEYIKKERWTDSFKILAYLCQKFLDLHKLTLILNAVSEVELLNLARTFKGKVRRSSHGTNICRIRVPSLGPTFISEGWAYFKKLDILMDRNFLLMVKDVIIGRMQTVLSMVCRIDNLFSEQDIFSLLNIYRIGDKIVERQGNFSYDLIKMVEPICNLKLMKLARESRPLVPQFPHFENHIKTSVDEGAKIDRGIRFLHDQIMSVKTVDLTLVIYGSFRHWGHPFIDYYTGLEKLHSQVTMKKDIDVSYAKALASDLARIVLFQQFNDHKKWFVNGDLLPHDHPFKSHVKENTWPTAAQVQDFGDKWHELPLIKCFEIPDLLDPSIIYSDKSHSMNRSEVLKHVRMNPNTPIPSKKVLQTMLDTKATNWKEFLKEIDEKGLDDDDLIIGLKGKERELKLAGRFFSLMSWKLREYFVITEYLIKTHFVPMFKGLTMADDLTAVIKKMLDSSSGQGLKSYEAICIANHIDYEKWNNHQRKLSNGPVFRVMGQFLGYPSLIERTHEFFEKSLIYYNGRPDLMRVHNNTLINSTSQRVCWQGQEGGLEGLRQKGWSILNLLVIQREAKIRNTAVKVLAQGDNQVICTQYKTKKSRNVVELQGALNQMVSNNEKIMTAIKIGTGKLGLLINDDETMQSADYLNYGKIPIFRGVIRGLETKRWSRVTCVTNDQIPTCANIMSSVSTNALTVAHFAENPINAMIQYNYFGTFARLLLMMHDPALRQSLYEVQDKIPGLHSSTFKYAMLYLDPSIGGVSGMSLSRFLIRAFPDPVTESLSFWRFIHVHARSEHLKEMSAVFGNPEIAKFRITHIDKLVEDPTSLNIAMGMSPANLLKTEVKKCLIESRQTIRNQVIKDATIYLYHEEDRLRSFLWSINPLFPRFLSEFKSGTFLGVADGLISLFQNSRTIRNSFKKKYHRELDDLIVRSEVSSLTHLGKLHLRRGSCKMWTCSATHADTLRYKSWGRTVIGTTVPHPLEMLGPQHRKETPCAPCNTSGFNYVSVHCPDGIHDVFSSRGPLPAYLGSKTSESTSILQPWERESKVPLIKRATRLRDAISWFVEPDSKLAMTILSNIHSLTGEEWTKRQHGFKRTGSALHRFSTSRMSHGGFASQSTAALTRLMATTDTMRDLGDQNFDFLFQATLLYAQITTTVARDGWITSCTDHYHIACKSCLRPIEEITLDSSMDYTPPDVSHVLKTWRNGEGSWGQEIKQIYPLEGNWKNLAPAEQSYQVGRCIGFLYGDLAYRKSTHAEDSSLFPLSIQGRIRGRGFLKGLLDGLMRASCCQVIHRRSLAHLKRPANAVYGGLIYLIDKLSVSPPFLSLTRSGPIRDELETIPHKIPTSYPTSNRDMGVIVRNYFKYQCRLIEKGKYRSHYSQLWLFSDVLSIDFIGPFSISTTLLQILYKPFLSGKDKNELRELANLSSLLRSGEGWEDIHVKFFTKDILLCPEEIRHACKFGIAKDNNKDMSYPPWGRESRGTITTIPVYYTTTPYPKMLEMPPRIQNPLLSGIRLGQLPTGAHYKIRSILHGMGIHYRDFLSCGDGSGGMTAALLRENVHSRGIFNSLLELSGSVMRGASPEPPSALETLGGDKSRCVNGETCWEYPSDLCDPRTWDYFLRLKAGLGLQIDLIVMDMEVRDSSTSLKIETNVRNYVHRILDEQGVLIYKTYGTYICESEKNAVTILGPMFKTVDLVQTEFSSSQTSEVYMVCKGLKKLIDEPNPDWSSINESWKNLYAFQSSEQEFARAKKVSTYFTLTGIPSQFIPDPFVNIETMLQIFGVPTGVSHAAALKSSDRPADLLTISLFYMAIISYYNINHIRVGPIPPNPPSDGIAQNVGIAITGISFWLSLMEKDIPLYQQCLAVIQQSFPIRWEAVSVKGGYKQKWSTRGDGLPKDTRISDSLAPIGNWIRSLELVRNQVRLNPFNEILFNQLCRTVDNHLKWSNLRRNTGMIEWINRRISKEDRSILMLKSDLHEENSWRD

SEQ ID NO: 52 - protein, 229 aa, Vesicular stomatitis virus
MSSLKKILGLKGKGKKSKKLGIAPPPYEEDTSMEYAPSAPIDKSYFGVDEMDTYDPNQLRYEKFFFTVKMTVRSNRPFRTYSDVAAAVSHWDHMYIGMAGKRPFYKILAFLGSSNLKATPAVLADQGQPEYHAHCEGRAYLPHRMGKTPPMLNVPEHFRRPFNIGLYKGTIELTMTIYDDESLEAAPMIWDHFNSSKFSDFREKALMFGLIVEKKASGAWVLDSIGHFK

SEQ ID NO: 53 - protein, 498 aa, Lymphocytic choriomeningitis virus; identical to SEQ ID NO: 46

SEQ ID NO: 54 - protein, 265 aa, Vesicular stomatitis virus; identical to SEQ ID NO: 50

SEQ ID NO: 55 - protein, 422 aa, Vesicular stomatitis virus; identical to SEQ ID NO: 49

SEQ ID NO: 56 - protein, 229 aa, Vesicular stomatitis virus; identical to SEQ ID NO: 52

SEQ ID NO: 57 - protein, 2109 aa, Vesicular stomatitis virus; identical to SEQ ID NO: 51

SEQ ID NO: 58 - protein, 498 aa, Vesicular stomatitis virus; identical to SEQ ID NO: 46

SEQ ID NO: 59 - protein, 278 aa, synthetic construct
  REGION 1..278: note = Derived from 3 Homo sapiens antigens (synthesized)
MANRTLTLFNVTRNDARAYVSGIQNSVSANRSDPVTLDVLPDSSYLSGANLNLSCHSASPQYSWRINGIPQQHTQVLFIAKITPNNNGTYACFVSNLATGRNNSIVKSITVSASGTSPGLSAAPTLPPAWQPFLKDHRISTFKNWPFLEGSAVKKQFEELTLGEFLKLDRERAAVARRNERERNRVKLVNLGFQALRQHVPHGGASKKLSKVETLRSAVEYIRALQRLLAEHDAVRNALAGGLRPQAVRPSAPRGPSEGALSPAERELLDFSSWLGGY

SEQ ID NO: 60 - protein, 353 aa, synthetic construct
  REGION 1..353: note = Derived from Homo sapiens and Epstein-barr virus (EBV) (synthesized)
KRYKNRVASRKSRAKFKQLLQHYREVAAAKSSENDRLRLLLKNRTLTLFNVTRNDARAYVSGIQNSVSANRSDPVTLDVLPDSSYLSGANLNLSCHSASPQYSWRINGIPQQHTQVLFIAKITPNNNGTYACFVSNLATGRNNSIVKSITVSASGTSPGLSAAPTLPPAWQPFLKDHRISTFKNWPFLEGSAVKKQFEELTLGEFLKLDRERAAVARRNERERNRVKLVNLGFQALRQHVPHGGASKKLSKVETLRSAVEYIRALQRLLAEHDAVRNALAGGLRPQAVRPSAPRGPSEGALSPAERELLDFSSWLGGYSTVHEILSKLSLEGDHSTPPSAYGSVKPYTNFDAE

SEQ ID NO: 61 - protein, 446 aa, synthetic construct
  REGION 1..446: note = Humanized Mus musculus antibody (synthesized)
EVMLVESGGGLVQPGGSLRLSCTASGFTFSASAMSWVRQAPGKGLEWVAYISGGGGDTYYSSSVKGRFTISRDNAKNSLYLQMNSLRAEDTAVYYCARHSNVNYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 62 - protein, 218 aa, synthetic construct
  REGION 1..218: note = Humanized Mus musculus antibody (synthesized)
EIVLTQSPATLSLSPGERATMSCRASENIDTSGISFMNWYQQKPGQAPKLLIYVASNQGSGIPARFSGSGSGTDFTLTISRLEPEDFAVYYCQQSKEVPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 446 aa, synthetic construct
  REGION 1..446: note = Humanized Mus musculus antibody (synthesized)
EVMLVESGGGLVQPGGSLRLSCTASGFTFSASAMSWVRQAPGKGLEWVAYISGGGGDTYYSSSVKGRFTISRDNAKNSLYLQMNSLRAEDTAVYYCARHSNPNYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 64 - protein, 218 aa, synthetic construct; identical to SEQ ID NO: 62
  REGION 1..218: note = Humanized Mus musculus antibody (synthesized)

SEQ ID NO: 65 - protein, 446 aa, synthetic construct
  REGION 1..446: note = Humanized Mus musculus antibody (synthesized)
EVMLVESGGGLVQPGGSLRLSCTASGFTFSKSAMSWVRQAPGKGLEWVAYISGGGGDTYYSSSVKGRFTISRDNAKNSLYLQMNSLRAEDTAVYYCARHSNVNYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 66 - protein, 218 aa, synthetic construct
  REGION 1..218: note = Humanized Mus musculus antibody (synthesized)
EIVLTQSPATLSLSPGERATMSCRASENIDVSGISFMNWYQQKPGQAPKLLIYVASNQGSGIPARFSGSGSGTDFTLTISRLEPEDFAVYYCQQSKEVPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - protein, 446 aa, synthetic construct; identical to SEQ ID NO: 65
  REGION 1..446: note = Humanized Mus musculus antibody (synthesized)

SEQ ID NO: 68 - protein, 218 aa, synthetic construct; identical to SEQ ID NO: 66
  REGION 1..218: note = Humanized Mus musculus antibody (synthesized)

SEQ ID NO: 69 - protein, 446 aa, synthetic construct; identical to SEQ ID NO: 65
  REGION 1..446: note = Humanized Mus musculus antibody (synthesized)

SEQ ID NO: 70 - protein, 218 aa, synthetic construct; identical to SEQ ID NO: 66
  REGION 1..218: note = Humanized c antibody (synthesized)

SEQ ID NO: 71 - protein, 311 aa, synthetic construct
  REGION 1..311: note = Derived from Homo sapiens Annexin A2 protein and 3 Homo sapiens antigens (synthesized)
NRTLTLFNVTRNDARAYVSGIQNSVSANRSDPVTLDVLPDSSYLSGANLNLSCHSASPQYSWRINGIPQQHTQVLFIAKITPNNNGTYACFVSNLATGRNNSIVKSITVSASGTSPGLSAAPTLPPAWQPFLKDHRISTFKNWPFLEGSAVKKQFEELTLGEFLKLDRERAAVARRNERERNRVKLVNLGFQALRQHVPHGGASKKLSKVETLRSAVEYIRALQRLLAEHDAVRNALAGGLRPQAVRPSAPRGPSEGALSPAERELLDFSSWLGGYSTVHEILSKLSLEGDHSTPPSAYGSVKPYTNFDAE

SEQ ID NO: 72 - protein, 257 aa, Lassa virus
VMNIVLIALSILAVLKGLYNIATCGLIGLVTFFLLCGRSCSSNLYKGVYELQSLDLNMETLNMTMPLSCTKNNSHHYIRVGNETGLELTLTNTSLLDHKFCNLSDAHKKNLYDHALMSIISTFHLSIPNFNQYEAMSCDFNGGKITVQYNLSHSYAGDAARHCGTIANGVLQTFMRMAWGGSYIALDSGHGNWDCIMTSYQYLIIQNTTWEDHCQFSRPSPIGYLSLLSQRTRDIYISRRLLGTFTWTLSDSEGNAT

SEQ ID NO: 73 - protein, 145 aa, synthetic construct
  REGION 1..145: note = Combination of Homo sapiens HMGB1-derived short peptide (Hp91), Epstein-barr virus CPP, and 4 Mus musculus antigens (synthesized)
DPNAPKRPPSAFFLFCSEKRYKNRVASRKSRAKFKQLLQHYREVAAAKSSENDRLRLLLKESLKISQAVHAAHAEINEAGREVVGVGALKVPRNQDWLGVPRFAKFASFEAQGALANIAVDKANLDVEQLESIINFEKLTEWTGS

SEQ ID NO: 74 - protein, 85 aa, synthetic construct
  REGION 1..85: note = Derived from Gallus gallus domesticus (synthesized)
ESLKISQAVHAAHAEINEAGREVVGVGALKVPRNQDWLGVPRFAKFASFEAQGALANIAVDKANLDVEQLESIINFEKLTEWTGS

SEQ ID NO: 75 - protein, 18 aa, synthetic construct
  REGION 1..18: note = Derived from Human papillomavirus (HPV) Mad25 (synthesized)
QAEPDRAHYNIVTFSSKS

SEQ ID NO: 76 - protein, 58 aa, synthetic construct
  REGION 1..58: note = Derived from 2 Mus musculus antigens (synthesized)
NGRVLELFRAAQLANDVVLQIMELSGATRPTGIPVHLELASMTNMELMSSIVHQGNNV

SEQ ID NO: 77 - protein, 85 aa, synthetic construct; identical to SEQ ID NO: 74
  REGION 1..85: note = Derived from 4 Mus musculus antigens (synthesized)

SEQ ID NO: 78 - protein, 18 aa, synthetic construct
  REGION 1..18: note = Derived from 2 Mus musculus antigens (synthesized)
QAEPDRAHYNIVTFCCKC

SEQ ID NO: 79 - protein, 50 aa, synthetic construct
  REGION 1..50: note = Derived from 3 Mus musculus antigens (synthesized)
LFRAAQLANDVVLQIMEHLELASMTNMELMSSIVVISASIIVFNLLELEG

SEQ ID NO: 80 - RNA, 12036 nt, Vesicular stomatitis virus
  source 1..12036: mol_type = genomic RNA
tgcttctgtttgtttggtaataatagtaattttccgagtcctctttgaaattgtcattagttttacagacaatgtcagttctcttagtaactgttgtgtcagcatcaaggttttgaaggacgtttactcctaggtcaccttatgggccgtctaatgaagtcttttagtttcctctaaggagaaatgtagttatgatgtttttcaaacagtctagattctcctatacagatggttccggagtttaggcctttacatagttagtatgtacagttgtcgatgaacatacctcgtaatttcctgtaggccccattcaacctatttctaaccagttcaaagccttatttgtagccctttcgtcccctatgttagccttataaactggaacataggaactttcgggacctgccgcatgaaggtctacctcatagcctacgaaggtcttggtcgcgtctactgtttaccaacggaaacatagatgaaccgaatatgtctcacccgtcttgtgtttacggacttatgtcttttttcgagtacctacccgactgtttagttacgttttactagttacttgtcaaacttggagaacacggtcttccagcactgtaaaaactacacacccctttactgtcattaatgtgtttttaacagcgacgtcacctgtacaagaaggtgtacaagttttttgtacttacacggagcaagtctatgccttgataacaaaggtctaagtttctaacacgacgtaaccgttgtaaacctgtggagacgttttattggccttacagatgtcttctacattgctggacctagaacttggctcttcaacgtctactttaccaggtttactacgaaggtccggttctttaactgttccggctaagtatgtacggaataaactagctgaaacctaacagaagattcagaggtataagaaggcagtttttgggacggaaggtgaagacccccgttaactgtcgagaagacgagtctaggtggtctcgttccttacgggctgtcggactactgtaactcatatgtagagaatgatgtcgtccaaacaacatgcgaatacgtcatcctaggagacggctgaaccgtgttgtcaaaacacaacctctattgtttatgtgaggtctactatcatggcctcctaactgctgattacgtggcggtgttccgtctctacaccagcttaccgagcctaccaaacttctagttttgtcttttggctgaggactatactacgtcatacgcttttctcgtcagtacagtgacgttccggattctctcttctgttaaccgttcatacgattcagtcttaaactgtttactgggatattaagagtctagtggataatatataatacgatgtatactttttttgattgtctatagtacctattagagtgttttcaagcactcatagagttcaggataagagcagacctagtccgccatcctctctatctactctagcttcgtgttgctcgacttttcaggttaatactcaacaaggttctcctacctcaccttctcgtatgattcgggagaataaaagtccgtcgtctactaagactgtgtcttagacttggtctttaacttctgttagttccgaacatacgtggtctaggtcttcgactcgttcaacttccgaaatatgtccccggaaatctactgatacgtctactccttcacctacaacataaatgaagcctgacctttgtcggactcgaacttagactgctcgtacctttctggaatgccaactgtagcggtctcccaaattcacctctcgtctttagggtcaccgaaagctgctaatttcgtcagcacgtttcacggtttatgaccttagaccgtctcacgtgtaaacttcgtagccctcttccccagtaatacttcctcgcggtctattgaggcctacatatattccagtgaggtcactacttgtgtgtaggcagggttagtcttcgtcatagtctacaaaccagagagagtttctgtaggtactgaaaggttgggttctttcgttcagaagtcggagagtggtataggaacctacttaacaagagtagatctcctctcaagtagagacagcctccactgcctgcttacagagtatttctccggtaggacgagccggactctatgtttttcaacatgttagtccgctctcagtttataagagacatctgatactttttttcattgtctatagtgctagattcacaatagggttaggtaagtagtactcaaggaatttcttctaagagccagacttcccctttccattctttagattctttaatccctagcgtggtgggggaatacttctcctgtgatcgtacctcatacgaggctcgcgaggttaactgtttaggataaaacctcaactgctctacctgtggatactaggcttagttaattctatactctttaagaagaaatgtcacttttactgccaatctagattagcaggcaagtcttgtatgagtctacaccgtcggcgacatagggtaaccctagtgtacatgtagccttaccgtccctttgcagggaagatgttttagaaccgaaaaaacccaagaagattagatttccggtgaggtcgccataaccgtctagttccagttggtctcatagtgcgagtgacgcttccgtcccgaataaacggtgtatcctaccccttctggggagggtacgagttacatggtctcgtgaagtcttctggtaagttatatccagaaatgttcccttgctaactcgagtgttactggtagatgctactactcagtgaccttcgtcgaggatactagaccctagtaaagttaagaaggtttaaaagactaaagtctctcttccggaattacaaaccggactaacagctctttttccgtagacctcgcacccaggacctgagatagccggtgaagtttactcgatcagattgaagatcgaagacttgttaggggccaaatgagtcagaggggattaaggtcggagagcttgttgattataggacagaaaagatagggatactttttttgattgtctctagctagacaaatgcgcagtgcctagggggcccgacgtccttaagcggtggtacccggtctagcactggtacaagctccgggacggggtgtagtagctgctccactagttgtagcactagtagcacgagtagtagtagtggtcgtagttccggcacatgttgaagcggtggacgccgtaggaccgggaccactcgaaggacaaggaccggccgtcttcgacgccgtacatgccggacttaccggggctatagatgttcccgcacatggtcaagttctcgcacctcaagctgtactcggtggacttggagtggtacgggttgcggacgtcgcggttgttatcggtggtgatgtagtcgtacccgtcgtcgccggacctcaactggaagtggttgctgtcgtaggacttggtgttgaagacgttggagtggtcgcggaagttgttcttttggaagctggtgtgggagtactcgtagcactcgtcggacgtggactcgtagtctccgttgtcgttggtgttccggcactcgacgctgaagttgttgccgtagtggtaggtcatgttggactcgaagtcgctaggagtctcgcggtagtcggtcacgtcttggaagtctccgtctcacgacctgtacaagtcttggcggaagccgccgttcatgtactcttcgccgaccccgacccggccgtcgctgccgttctggtggaccacgtcggtctggtcgatggtcatggagtagtaggtcttgtcttggaccctcttggtgacgtctatgcggcctggaaagccgtactcgtcttaggacaagcgggtcctcttttggttcaaggagtggtcctctgaccggccgtggaagtggacctgggactcgctgtcgtcgccgcacctcttgggaccgccgatgacggagtggttcacctactaggaccggcggctcgacttcacgaagccgttgtggcggcaccggttcacgttgcacttggtgctgctcctcaagacgctgtacgactctgagtagctgatgttgttccggcgggactcgttcaagttcgtcctgcacctctcgcgggacgtgcacaagttctggtggcacttgtcggagtagtcgctggtcgacgagtactctttggtggactctctggagtacccgcacgggatgacgttgatgtcgttcaagaccatagacctcgtgcggttctggccgctctggtcgcacgggttcacgaccgaccactggttaccgtcgatggacttgctctgggtgaagtcgctggtctagctcgtccttcggctgttgtactagtggctctacgactccttcctgatgtagttctctgtcccgtcgtggggggaccgggagtacctagacgagtacaagtcgtggtcgcggatggagtagtcgtagaaggacgtggaccacttctaggggtgggtgtctgtgtagttcccgccgtcgacggggttcggggtgtctgagtggttgttcccgtagacgtcgacgccgcggaagttccacgggccgcacttttggtagaccttctcctctattcgccggcgatgctggagctgatactttttttgattgtctataggagctgcggtggtaccggttggcctgtgactgggacaagttgcactgggccttactgcggtctcggatgcacaggccgtaggtcttatcgcacaggcggttatcttcgctggggcactgtgacctgcacgacggactatcgtcgatagactcgccgcggttggacttggactcgacggtaagacggagaggtgtcatgtcgaccgcctagttgccttagggagtcgtcgtgtgggtccacgacaaatagcggttctagtggggattgttgttgccgtggatgcggacgaagcacaggttagaccggtggccgtctttgttgtcgtagcacttcaggtagtggcacagacggtcgccgtgtagaggacctaacagacggcgaggatgtgacggaggtcggaccgtcggaaaggacttcctggtgtcttagtcgtggaagttcttgaccgggaaggaccttccgtcgcggcacttcttcgtcaagctccttgactgagacccgctcaaggacttcgacctgtctctttcccggcgacaccggtctgccttgctcgcgctttctttgtctcacttcgaccagttggacccgaaagtccgggactctgtcgtacacggagtaccgcctcggtcgttcttcgactcgttccagctctgtgactcttcgcggcagctcatgtagtctcgggacgtctctgacgaccggcttgtgctgcggcaatctttacgagaccgaccgcctgaatctggagtccgacagtctggaagacgaggatctcctggaagactcccgcgagacagaggacggctttctctcgacgacctgaagtcgagaaccgagccgccgatgactagttgatcggtctaagaagtacaaacctggtttagttgaacactatggtacgagtttctccggagttaatataaactcaaaaattaaaaatactttttttgattgtcgttagtaccttcaggtgctaaaactctggctgctcaagttactaaagttacttctactgatacggtgttctcttaaggacttagggctactcgcgtactgcatgaacttagtacgactaatgttggacttaagaggagattaatcactactataactgttaaattagtcctttaagttaagagaaggttaagggagctacaccctatcattcttgaccctacctcaagaactctacaattgcagtacagttcggttagggtagggttgtagagtctacgtatttacctacccttcaaccaattacagactattagtactacggtcagttcccatatcaaaaaatgtacttcacctgtttctccgtctttattgtaaactgcaccacctctggaagtaggcgccgaccccgttgtttggttaacttatgtagtttttcctttctacctgactgagtaagttttaagagcgaataaacacagttttcaaaaacctgaatgtgttcaactgtaattagaatttacgacagagactccaccttaacgagttgaaccgctcctgaaagtttccgtttcagtcttcttcaagagtaccttgcttgtatacgtcctaatcccaagggtcgaacccaggatgaaaataaagtcttcctacccgaatgaagttctttgaactataagattacctggctttgaaagacaattaccagtttctacactaatatccctcctacgtttgccacgataggtaccatacatcttatctgttggacaagagtctcgttctgtagaagagggaagatttatagatgtcttaacctctattttaacacctctccgtccctttaaaaagaatactgaactaattttaccaccttggctatacgttgaacttcgactactttaatcgttctcttagttccggaaatcagggtgttaagggagtaaaacttttagtatagttctgaagacaactacttccccgtttttaactggctccatattctaaggaggtactagtctattactcacacttttgtcacctagagtgtgaccactaaatacctagcaagtctgtaaccccagtaggaaaatatctaataatgtgacctgatctttttaatgtaagggttcattggtacttctttctataactacacagtatacgttttcgtgaacgttcactaaatcgagcctaacaagataaagttgtcaagttactagtatttttcaccaagcacttacctctgaacgagggagtactagtagggaaattttcagtacaatttcttttatgtaccgggtgtcgacgagttcaagttctaaaacctctatttaccgtacttgaaggcgactaatttacaaaactttatgggctgaatgatctgggtagctattatatgagactgttttcagtaagttacttatccagtctccacaactttgtacaggcttacttaggcttgtgaggatagggatcatttttccacaacgtctgatacaacctgtgtttccgatggttaacctttcttaaagaatttctctaactactcttcccgaatctactactactagattaataaccagaatttcctttcctctcccttgacttcaaccgtccatctaaaaagagggattacagaacctttaacgctcttatgaaacattaatggcttataaactatttctgagtaaagcagggatacaaatttccggactgttaccgcctgctagattgacgtcagtaatttttctacaatctaaggagtaggccggttcctaacttcagtatactccgttaaacgtatcggttagtgtaactaatgctttttaccttattggtggtttccttcaatagtttgccgggtcacaaggctcaatacccggtcaagaatccaataggtaggaattagctctcttgagtacttaaaaaactcttttcagaatatatgatgttaccttctggtctgaactacgcacaagtgttgttgtgtgactagttaagttggagggttgctcaaacaaccgttcctgttctcccacctgaccttccagatgccgtttttcctacctcataggagttagatgaccaataagtttctctccgattttagtctttgtgacgacagtttcagaaccgtgttccactattagttcaataaacgtgtgtcatattttgcttctttagctctttgcaacatcttaatgtcccacgagagttagtttaccaaagattattactcttttaatactgacgttagttttatccctgtcccttcaatcctgaaaactatttactgctactctgatacgttagacgtctaatgaacttaatacctttttatggctaaaaggcacctcactaatctcccaatctctggttctctaccagtgctcactgaacacagtggttactggtttatgggtgaacacgattatattactcgagtcaaaggtgtttacgagagtggcatcgagtaaaacgactcttgggttagttacggtactatgtcatgttaataaaaccctgtaaacgatctgagaacaactactacgtactaggacgagaagcagttagtaacatacttcaagttctattctatggcccgaacgtgtcaagatgaaagtttatgcggtacaacataaacctgggaaggtaacctcctcacagcccgtacagaaacaggtccaaaaactaatctcggaagggtctagggcattgtctttcagagagtaagacctctaagtaggtacatgtacgagcttcactcgtagacttcctctactcacgtcataaacctttggggctctatcggttcaaagcttattgagtgtatctgttcgatcatcttctaggttggagagacttgtagcgatacccttactcaggtcgcttgaacaatttctgactccaattttttacgaattagcttagttctgtttggtagtccttggtccactaattcctacgttggtatataaacatagtacttctcctagccgagtcttcaaagaataccagttatttaggagacaagggatctaaaaattcacttaagtttagtccgtgaaaaaaccctcagcgtctgcccgagtagtcagataaagttttaagagcatgataagccttgaggaaattctttttcatagtatcccttaacctactaaactaacactcctcactccataggagaaactgtgtaaatccctttgaagtaaactcttcccctagtacattttacacctgtacaagtcgatgagtacgactgtgtaattctatgtttaggaccccggcatgtcaataaccctgttgacatggggtaggtaatctttacaacccaggtgttgtagcttttctctgaggaacacgtggtacattgtgtagtcccaagttaatacaaagacacgtaacaggtctgccctaggtactgcagaaatcaagtgcccctggtaacggacgaatagatcccagattttgtagacttagatgtagataaaacgtcggaaccctttccctttcgtttcagggtgactaattttctcgatgtgcagaatctctacgatagagaaccaaacaacttgggctgagatttgatcgttactgatatgaaagattgtaggtgagaaattgtccgcttcttacctggttttccgtcgtacccaagttttcttgtcccagacgggaagtatccaaaagctgtagagcctactcggtaccacccaagcgtagagtctcgtgacgtcgtaactggtccaactaccgttgatgtctgtggtactccctagaccctctagtcttaaagctgaaaaataaggttcgttgcaacgagatacgagtttaatggtggtgacaacgttctctgcctacctagtggtcaacatgtctagtaatagtataacggacattcaggacaaactctgggtatcttctctagtgggacctgagttcatacctgatgtgcgggggtctacatagggtacacgacttctgtacctccttaccccttccaagcacccctgttctctattttgtctagataggaaatcttcccttaaccttcttaaatcgtggacgactcgttaggatagttcagccgtctacatatccaaaagatatacctctgaaccgcatatcttttagatgagtacggctcctgtcaagagataaaggagatagatatgttccagcataatctccagctccaaagaattttcccaacgatctgcctaattactctcgttcaacgacggttcattatgtggcctcttcagaccgagtaaacttctccggccggttgcgtcacatgcctccaaactaaatgaactaactatttaactcacatagtggaggtaaggaaagagaatgatctagtcctggataatctctgcttaatctttgctaaggggtgttctagggttggaggataggctgttcgttggcactatacccccactaacagtctttaatgaagtttatggttacggcagattaacttttcccttttatgtctagtgtaataagtgttaataccaataagagtctacagaataggtatctgaagtaacctggtaagagataaaggtggtgggagaacgtttaggatatgttcggtaaaaatagaccctttctattcttactcaactctctcgaccgtttagaaagaagtaacgattctagtcctctccccacccttctgtatgtacactttaagaagtggttcctgtataataacacaggtctcctttagtctgtacgaacgttcaagccctaacgattcctattattatttctgtactcgatagggggaaccccttcccttaggtctccctgttaatgttgttagggacaaataatatgctggtggggaatgggtttctacgatctctacggaggttcttaggttttaggggacgacaggccttagtccaacccggttaatggttgaccgcgagtaatattttaagcctcatataatgtaccttacccttaggtaatgtccctgaagaactcaacacctctgccgaggcctccctactgacgacgtaatgatgctcttttacacgtatcgtctccttataagttatcagacaatcttaatagtcccagtcagtacgctccgcggagaggactcggggggtcacgggatctttgaaatcctcctctatttagctctacacatttaccactttgtacaacccttataggtagactgaatacactgggttcctgaaccctgataaaggaggctgagtttcgtccgaaccccgaagtttaactaaattaacattacctataccttcaagccctaagaagatgatcggacttttaactctgcttacaatctttaatacacgtggcctaaaacctactcgttcctcaaaattagatgttctgaataccttgtatataaacactctcgcttttcttacgtcattgttaggaaccagggtacaagttctgccagctgaatcaagtttgtcttaaatcatcaagagtttgcagacttcatatataccatacatttccaaacttctttaattagctacttgggttagggctaaccagaaggtagttacttaggacctttttggacatgcgtaaggtcagtagtcttgtccttaaacggtctcgtttcttccaatcatgtatgaaatggaactgtccataagggagggttaagtaaggactaggaaaacatttgtaactctgatacgatgtttataagcctcatgggtgcccacacagagtacgccgacggaattttagtagactatctggacgtctaaataactggtaatcggaaaaaatataccgctaatatagcataatattgtagttagtatagtctcatcctggctatggaggcttggggggtagtctaccttaacgtgttttacacccctagcgatattgaccatattcgaaaaccgactcaaactacctctttctgtaaggtgatatagttgtcacaaatcgtcaataggtcgttagtaagggctaatccaccctccgacaaagtcattttcctcctatgttcgtcttcacctcatgatctccactacccgagggttttctatgggcttaaagtctgaggaaccggggttagcccttgacctagtctagagaccttaaccaggctttggttcaagcagatttaggtaagttactctagaacaagttagtcgatacagcatgtcacctattagtaaactttaccagtttaaacgcttctttgtgtccttactaacttacctagttatctgcttaaagttttcttctggccagatatgactacaacttctcactggatgtgctccttttgagaacctctctaattttttagtactcctctgaggtttgaaattcatactttttttgaaactaggaattctgggagaacaccaaaaataaaaaatagaccaaaacaccagaagca

(19) United States
(12) Patent Application Publication
(10) Pub. No.: US 2025/0312428 A1
(43) Pub. Date: Oct. 9, 2025

(54) Heterologous Prime Boost Vaccine

(71) Applicant: BOEHRINGER INGELHEIM INTERNATIONAL GMBH, Ingelheim Am Rhein (DE)

(72) Inventors: Guido WOLLMANN, Innsbruck (AT); Krishna DAS, Innsbruck (AT); Madiha DEROUAZI, Geneva (CH); Elodie BELNOUE, Geneva (CH); Knut ELBERS, Rum (AT)

(21) Appl. No.: 19/237,140

(22) Filed: Jun. 13, 2025

Related U.S. Application Data
(63) Continuation of application No. 17/474,240, filed on Sep. 14, 2021.

(30) Foreign Application Priority Data
Sep. 14, 2020 (EP) 20195872.5
Nov. 30, 2020 (EP) 20210671.2
Feb. 8, 2021 (EP) 21155814.3
May 27, 2021 (EP) 21176373.5

(51) Int. Cl. A61K 39/00; A61K 39/12; A61P 35/04; C07K 14/47

(52) U.S. Cl. CPC A61K 39/0011; A61K 39/12; A61P 35/04; C07K 14/473; A61K 2039/545; A61K 2039/575

(57) ABSTRACT

The present invention pertains to the provision of a vaccine comprising a first component (K) and a second component (V), wherein the first component (K) comprises a complex in which a cell penetrating peptide, an antigenic domain and a TLR agonist are functionally linked and the second component (V) comprises an oncolytic recombinant vesicular stomatitis virus expressing an antigenic domain. The invention further pertains to the use of the inventive vaccine in the treatment of cancer. The invention also provides a recombinant vesicular stomatitis virus expressing an antigenic domain and its use in cancer vaccines.

### CROSS-REFERENCE TO RELATED PATENT APPLICATIONS

This patent application is a continuation of U.S. patent application Ser. No. 17/474,240 filed 14 Sep. 2021, which claims priority under 35 USC § 119 to European Application No.: 20195872.5 filed 14 Sep. 2020, European Application No.: 20210671.2 filed 30 Nov. 2020, European Application No.: 21155814.3 filed 8 Feb. 2021, and European Application No.: 21176373.5 filed 27 May 2021. The entire contents of each of the above recited patent applications is incorporated herein by reference.

### REFERENCE TO A SEQUENCE LISTING

This application contains references to nucleic acid sequences and/or amino acid sequences which have been submitted concurrently herewith as the sequence listing XML file entitled “19205-000044-US-COA_17_Jun_2025_ST26”, file size 127,074 Bytes (B), created on 17 Jun. 2025. The aforementioned sequence listing is hereby incorporated by reference in its entirety.

### FIELD OF THE INVENTION

The present invention relates to the field of cancer vaccines in particular to heterologous prime boost vaccines which comprise a complex consisting of a cell penetrating peptide, an antigenic domain and a TLR agonist as first component and a recombinant rhabdovirus encoding in its genome an antigenic domain. The invention further relates to the use of the heterologous prime boost vaccine in the treatment of cancer and provides for a recombinant rhabdovirus for use in heterologous prime boost vaccines.

### BACKGROUND OF THE INVENTION

Most of the vaccine strategies under development require multiple immunizations with the same agent such as a viral vector encoding a tumor antigen. More recently, the concept of heterologous prime-boost immunization has been tested in a non-human primate model using a recombinant vaccine virus expressing SVmne gp160 for vaccination and recombinantly expressed gp160 as boost (Science 1992 Jan. 24; 255 (5043): 456-9). This strategy involves the sequential administration of different vaccine platforms which encode same recombinant antigen. Initially the effectiveness of heterologous prime boost was demonstrated in animal studies of infectious diseases, such as malaria and HIV-1. More recently, prime-boost technology is being developed for use in tumor patients: For example, a plasmid DNA expressing truncated human epidermal growth factor receptor 2 (HER2) and granulocyte macrophage colony-stimulation factor (GM-CSF) as a bicistronic message and an adenoviral vector containing the same modified HER2 sequence only was used to treat patients suffering from HER2-expressing breast cancer. (Molecular Therapy—Methods & Clinical Development (2015) 2, 15031)

The principle of heterologous prime-boost technology is to force the immune system to focus its response on a specific recombinant antigen by avoiding an immune response against the antigen carrier or delivery system after sequential administration of the same antigen carrier or delivery system when used in homologous prime-boost regimens. In heterologous prime boost the administration of the first immunogen primes cytotoxic T lymphocytes (CTLs) specific for the recombinant antigen, however, priming also occurs for the antigen carrier or delivery system. By administering an unrelated second antigen carrier or delivery system, such as for example a viral vector during the “boost” phase, the immune system is faced with a large number of new antigens. As the second antigen carrier or delivery system also encodes the recombinant antigen for which primed cells already exist, a strong memory response is raised by the immune system, expanding previously primed CTLs, which are specific for the recombinant antigen.

Various formats of heterologous prime boost vaccination have recently been explored in treatment and prevention of tumors and which are being tested in both pre-clinical testing as well as clinical trials (see for example: Biomedicines 2017, 5, 3).

Therapeutic cancer vaccines which are able to induce tumor specific immune responses are becoming a promising therapeutic approach in oncology. It remains, however, still challenging to overcome self-tolerance and tumor immune evasion in order to induce a robust long-term cellular immune response able to recognize and kill the tumor cells. A number of cancer vaccines targeting multiple tumor specific antigens are now under development to counteract tumor immune evasion and enable induction of a robust cellular immune response.

Other heterologous prime boost approaches employ a combination of immunization using an adenoviral vaccine prior to treatment with an oncolytic vesicular stomatitis virus (VSV) of which both express the same tumor-associated antigen (Bridle, et al. Molecular Therapy 18.8 (2010): 1430-1439). However, this type of heterologous prime boost vaccination requires the production of two recombinant viruses expressing the respective tumor-associated antigens (TAAs), which is technically challenging. Furthermore, adenovirus (Ad)-derived vectors have been shown to successfully elicit strong cellular and humoral immune responses in rodents, non-human primates (NHP) as well as in humans after a single injection (see e.g. Molecular Therapy Vol 10 (4), October 2004, pages 616-629) which can preclude a second administration of the same adenoviral vector due to the strong immune response against the vector in subsequent applications. In addition, pre-existing immunity against adenoviral vectors may hamper clinical use of certain adenoviral serotypes such as hAd5. Another adenoviral-based heterologous prime boost approach utilizes a quadrivalent mutated transgene based on the E6 and E7 proteins of HPV16 and 18 which was cloned into adenoviral and Maraba MG1 viruses (Atherton et al., Cancer Immunol Res 2017 October; 5 (10): 847-859). This approach, however, requires the production of two viruses which is technically challenging.

Thus, there is a need in the art to avoid the technically challenging production of a heterologous prime boost vaccine incorporating two viruses and at the same time to improve the anti-tumor effect of VSV-based heterologous prime boost vaccines by reducing the number of antiviral CD8+ CTLs while at the same time and increasing the number anti-tumor-associated antigen CD8+ CTLs and memory immunity thereby improving the anti-tumoral efficacy of heterologous prime boost vaccines.

### SUMMARY OF THE INVENTION

The present invention addresses the above need by providing a vaccine which comprises two components wherein the first component (K) comprises a complex, which consists of or comprises (i) a cell penetrating peptide; (ii) an antigenic domain, which comprises at least one antigen or antigenic epitope and (iii) at least one TLR peptide agonist, wherein the components (i)-(iii) are covalently linked, and wherein the second component (V) comprises a rhabdovirus, in particular an oncolytic rhabdovirus.

It is to be understood that any embodiment relating to a specific aspect might also be combined with another embodiment also relating to that specific aspect, even in multiple tiers and combinations comprising several embodiments to that specific aspect.

According to a first embodiment the present invention provides a vaccine which comprises two components wherein the first component (K) comprises a complex, which consists of or comprises (i) a cell penetrating peptide; (ii) an antigenic domain, which comprises at least one antigen or antigenic epitope; and (iii) at least one TLR peptide agonist, wherein the components (i)-(iii) are covalently linked, and wherein the second component (V) comprises a rhabdovirus, in particular an oncolytic rhabdovirus.

According to one embodiment, the cell penetrating peptide of the complex of first component (K) of the inventive vaccine comprises an amino acid sequence according to one of SEQ ID NO: 2 (Z13), SEQ ID NO: 3 (Z14), SEQ ID NO: 4 (Z15), or SEQ ID NO: 5 (Z18).

According to one embodiment, the first component of the inventive vaccine comprises more than one TLR peptide agonist, in particular 2, 3, 4, 5, 6, 7, 8, 9, 10 or more TLR peptide agonists, whereby the at least one TLR agonist of the inventive vaccine is preferably a TLR2 peptide agonist and/or a TLR4 peptide agonist.

In one embodiment, the TLR2 agonist of the inventive vaccine comprises or consists of an amino acid sequence according to SEQ ID NO: 6 or 7, the TLR4 agonist comprises an amino acid sequence according to SEQ ID NO: 8, and/or functional sequence variants of SEQ ID NO: 6 or 7 and/or SEQ ID NO:8.

In one embodiment, the TLR2 agonist of the vaccine of the invention is annexin II or an immunomodulatory fragment thereof.

In one embodiment, the TLR2 agonist of the inventive vaccine comprises or consists of an amino acid sequence according to the annexin II coding sequence SEQ ID NO:6 or SEQ ID NO: 7, or fragments and/or functional fragments thereof, or it may comprise or consist of an amino acid sequence according to SEQ ID NO: 9 (High mobility group box 1 protein), or at least one immunomodulatory fragment thereof.

In some embodiments, the TLR2 peptide agonist may comprise or consist of an amino acid sequence according to SEQ ID NO: 9 (High mobility group box 1 protein), or at least one immunomodulatory fragment thereof.

In one embodiment, the TLR4 agonist of the invention consists of or comprises an amino acid sequence according to SEQ ID NO: 8 (EDA).

According to one embodiment, the at least one antigen or antigenic epitope of the antigenic domain of said first component of the inventive vaccine is selected from the group consisting of a peptide, a polypeptide, or a protein, wherein the antigenic domain of the complex of said first component (K) preferably comprises more than one antigen or antigenic epitope, in particular 2, 3, 4, 5, 6, 7, 8, 9, 10 or more antigens or antigenic epitopes, which are preferably positioned consecutively in said complex.

In some embodiments, the at least one antigen or antigenic epitope comprises or consists of at least one tumor or cancer epitope. According to one embodiment, the at least one antigen or antigenic epitope according to the invention comprises or consists of at least one tumor epitope, which preferably is selected from a tumor associated antigen, tumor-specific antigen, or tumor neoantigen.

In one embodiment, the at least one tumor epitope of the antigenic domain of said first component (K) is selected from the group of tumors comprising endocrine tumors, gastrointestinal tumors, genitourinary and gynecologic tumors, breast cancer, head and neck tumors, hematopoietic tumors, skin tumors, thoracic and respiratory tumors. More specifically, the at least one tumor or cancer epitope of the antigenic domain of said first component of the invention may be selected from the group of tumors or cancers of: gastrointestinal tumors comprising anal cancer, appendix cancer, cholangiocarcinoma, carcinoid tumor, gastrointestinal colon cancer, extrahepatic bile duct cancer, gallbladder cancer, gastric (stomach) cancer, gastrointestinal carcinoid tumor, gastrointestinal stromal tumor (GIST), hepatocellular cancer, pancreatic cancer, rectal cancer, colorectal cancer, or metastatic colorectal cancer. Preferably, at least one tumor or cancer epitope of the antigenic domain of said first component (K) is selected from the group of tumor associated antigens, tumor-specific antigens, or tumor neoantigens of colorectal cancer, or metastatic colorectal cancer.

According to one embodiment, the least one tumor or cancer epitope of the antigenic domain of the complex of the first component (K) of the inventive vaccine is an epitope of an antigen selected from the group consisting of EpCAM, HER-2, MUC-1, TOMM34, RNF 43, KOC1, VEGFR, βhCG, survivin, CEA, TGFβR2, p53, KRas, OGT, CASP5, COA-1, MAGE, SART, IL13Ralpha2, ASCL2, NY-ESO-1, MAGE-A3, mesothelin, PRAME, WT1.

According to a preferred embodiment, the least one tumor or cancer epitope of the antigenic domain of the complex of the first component (K) of the inventive vaccine is an epitope of an antigen selected from the group consisting of ASCL2, EpCAM, MUC-1, survivin, CEA, KRas, MAGE-A3 and IL13Ralpha2, preferably the at least one tumor epitope is an epitope of an antigen selected from the group consisting of ASCL2, EpCAM, MUC-1, survivin, CEA, KRas and MAGE-A3, more preferably the at least one tumor epitope is an epitope of an antigen selected from the group consisting of ASCL2, EpCAM, MUC-1, survivin and CEA; even more preferably the at least one tumor epitope is an epitope of an antigen selected from the group consisting of ASCL2, EpCAM, survivin and CEA; still more preferably the at least one tumor epitope is an epitope of an antigen selected from the group consisting of ACSL2, survivin and CEA.

In one embodiment, the antigenic domain of the complex of said first component (K) of the inventive vaccine comprises an epitope of survivin, which preferably comprises a peptide having an amino acid sequence according to SEQ ID NO: 12, or a fragment thereof having a length of at least 10 amino acids, or a functional sequence variant thereof having at least 70%, 75%, 80%, 85%, 90% or 95% sequence identity. More preferably, the antigenic domain of the complex of said first component (K) of the inventive vaccine comprises a peptide having an amino acid sequence according to SEQ ID NO: 22. Even more preferably, the antigenic domain of the complex of said first component (K) of the inventive vaccine comprises an amino acid sequence according to SEQ ID NO: 23 or a functional sequence variant thereof having at least 70%, 75%, 80%, 85%, 90% or 95% sequence identity.

In one embodiment, the antigenic domain of the complex of said first component (K) of the inventive vaccine comprises an epitope of CEA, which preferably comprises a peptide having an amino acid sequence according to SEQ ID NO: 24, or a fragment thereof having a length of at least 10 amino acids, or a functional sequence variant thereof having at least 70%, 75%, 80%, 85%, 90% or 95% sequence identity, more preferably an amino acid sequence according to SEQ ID NO: 26 and or SEQ ID NO: 27, more preferably an amino acid sequence according to SEQ ID NO: 25 or a functional sequence variant thereof having at least 70%, 75%, 80%, 85%, 90% or 95% sequence identity.

In one embodiment, the antigenic domain of the complex of said first component (K) of the inventive vaccine comprises an epitope of ASCL2, which preferably comprises a peptide having an amino acid sequence according to SEQ ID NO: 15, or a fragment thereof having a length of at least 10 amino acids, or a functional sequence variant thereof having at least 70%, 75%, 80%, 85%, 90% or 95% sequence identity, more preferably an amino acid sequence according to SEQ ID NO: 16 and or SEQ ID NO: 17, more preferably an amino acid sequence according to SEQ ID NO: 18 or a functional sequence variant thereof having at least 70%, 75%, 80%, 85%, 90% or 95% sequence identity.

According to a preferred embodiment, the antigenic domain of the vaccine of the invention comprises in N- to C-terminal direction one or more epitopes of CEA or functional sequence variants thereof; one or more epitopes of survivin or functional sequence variants thereof; and one or more epitopes of ASCL2 or functional sequence variants thereof.

According to one preferred embodiment, the antigenic domain of complex of the first component (K) of the vaccine according to the invention comprises in N- to C-terminal direction a peptide having an amino acid sequence according to SEQ ID NO: 24, or a fragment thereof having a length of at least 10 amino acids, or a functional sequence variant thereof, in particular having at least 70%, 75%, 80%, 85%, 90% or 95% sequence identity; a peptide having an amino acid sequence according to SEQ ID NO: 12, or a fragment thereof having a length of at least 10 amino acids, or a functional sequence variant thereof, in particular having at least 70%, 75%, 80%, 85%, 90% or 95% sequence identity; and a peptide having an amino acid sequence according to SEQ ID NO: 15, or a fragment thereof having a length of at least 10 amino acids, or a functional sequence variant thereof, in particular having at least 70%, 75%, 80%, 85%, 90% or 95% sequence identity. Preferably, the peptide consisting of the amino acid according to SEQ ID NO: 24 is directly linked to the N-terminus of the peptide consisting of the amino acid sequence according to SEQ ID NO: 12, which is directly linked to the N-terminus of the peptide consisting of the amino acid sequence according to SEQ ID NO: 15.

According to one preferred embodiment, the antigenic domain of complex of the first component (K) of the vaccine of the invention comprises a peptide consisting of an amino acid sequence according to SEQ ID NO: 25 or a functional sequence variant thereof, in particular having at least 70%, 75%, 80%, 85%, 90% or 95% sequence identity, a peptide consisting of an amino acid sequence according to SEQ ID NO: 23 or a functional sequence variant thereof, in particular having at least 70%, 75%, 80%, 85%, 90% or 95% sequence identity, and a peptide consisting of an amino acid sequence according to SEQ ID NO: 18 or a functional sequence variant thereof, in particular having at least 70%, 75%, 80%, 85%, 90% or 95% sequence identity, preferably wherein the peptides are linked as disclosed above.

According to a more preferred embodiment, the antigenic domain of the complex of the first component (K) of the vaccine of the invention comprises a peptide consisting of an amino acid sequence according to SEQ ID NO: 45 or a functional sequence variant thereof, in particular having at least 70%, 75%, 80%, 85%, 90% or 95% sequence identity.

In a preferred embodiment of the invention, the complex of the first component (K) comprises in N- to C-terminal direction a cell penetrating peptide, an antigenic domain and a TLR agonist wherein the complex comprises the amino acid sequence according to SEQ ID NO: 60 or a functional sequence variant thereof having at least 70%, 75%, 80%, 85%, 90% or 95% sequence identity.

In one embodiment, the second component (V) of the vaccine of the invention comprises a recombinant rhabdovirus, preferably a recombinant oncolytic rhabdovirus selected from the genus of vesiculoviridae, preferably a vesiculovirus, more preferably a vesicular stomatitis virus.

In one embodiment, the recombinant vesiculovirus, in particular the oncolytic recombinant vesiculovirus, of the invention as disclosed above is selected from the group comprising: Vesicular stomatitis alagoas virus (VSAV), Carajás virus (CJSV), Chandipura virus (CHPV), Cocal virus (COCV), Vesicular stomatitis Indiana virus (VSIV), Isfahan virus (ISFV), Maraba virus (MARAV), Vesicular stomatitis New Jersey virus (VSNJV), or Piry virus (PIRYV), preferably the recombinant vesiculovirus, in particular the oncolytic recombinant vesiculovirus, of the invention is a vesicular stomatitis virus, more preferably the recombinant vesiculovirus, in particular the oncolytic recombinant vesiculovirus, of the invention is one of Vesicular stomatitis Indiana virus (VSIV) or Vesicular stomatitis New Jersey virus (VSNJV).

In one embodiment, the recombinant vesicular stomatitis virus, preferably the oncolytic recombinant vesicular stomatitis virus, such as the Vesicular stomatitis Indiana virus (VSIV) or Vesicular stomatitis New Jersey virus (VSNJV), of the invention lacks a (functional) gene coding for glycoprotein G, and/or lacks a (functional) glycoprotein G, which may be replaced by the gene coding for the glycoprotein GP of another virus, and/or the glycoprotein G is replaced by the glycoprotein GP of another virus. In some embodiments, the gene coding for glycoprotein G is replaced by the gene coding for the glycoprotein GP of an arenavirus; and/or the glycoprotein G is replaced by the glycoprotein GP of an arenavirus. In other embodiments, the gene coding for glycoprotein G is replaced by the gene coding for the glycoprotein GP of Dandenong virus or Mopeia virus; and/or the glycoprotein G is replaced by the glycoprotein GP of Dandenong virus or Mopeia virus. Preferably, the gene coding for glycoprotein G is replaced by the gene coding for the glycoprotein GP of Lymphocyte choriomeningitis virus (LCMV), and/or the glycoprotein G is replaced by the glycoprotein GP of LCMV.

According to a preferred embodiment, the recombinant vesicular stomatitis virus, preferably the oncolytic recombinant vesicular stomatitis virus, such as the Vesicular stomatitis Indiana virus (VSIV) or Vesicular stomatitis New Jersey virus (VSNJV), according to the invention as disclosed above comprises the gene coding for the glycoprotein GP of LCMV and/or the glycoprotein GP of LCMV, wherein the glycoprotein GP of LCMV comprises or consists of the amino acid sequence according to SEQ ID NO: 46, or a sequence that is at least 80%, 85%, 90%, 95% identical thereto. In addition, the inventive recombinant vesicular stomatitis virus of the second component (V) preferably encodes in its genome a vesicular stomatitis virus nucleoprotein (N), large protein (L), phosphoprotein (P), matrix protein (M), glycoprotein (G) and at least one antigen or antigenic epitope of the antigenic domain of the complex of the first component (K) as disclosed above.

In some embodiments, the recombinant vesicular stomatitis virus, preferably the oncolytic recombinant vesicular stomatitis virus, of the second component (V) encodes in its genome at least one antigen or antigenic epitope of the complex of the first component (K) as described herein, wherein the gene coding for the glycoprotein G of the vesicular stomatitis virus is replaced by the gene coding for the glycoprotein GP of lymphocyte choriomeningitis virus (LCMV), and/or the glycoprotein G of the vesicular stomatitis virus is replaced by the glycoprotein GP of LCMV. In some embodiments, the recombinant vesicular stomatitis virus, preferably the oncolytic recombinant vesicular stomatitis virus, of the second component (V) encodes in its genome a vesicular stomatitis virus nucleoprotein (N), large protein (L), phosphoprotein (P), matrix protein (M), glycoprotein (G) and at least one antigen or antigenic epitope of the complex of the first component (K) as described herein, wherein the gene coding for the glycoprotein G of the vesicular stomatitis virus is replaced by the gene coding for the glycoprotein GP of lymphocyte choriomeningitis virus (LCMV), and/or the glycoprotein G is replaced by the glycoprotein GP of LCMV.

In some embodiments, the recombinant vesicular stomatitis virus, preferably the oncolytic recombinant vesicular stomatitis virus, of the second component (V) encodes in its genome a second antigenic domain consisting of the amino acid sequence of the antigenic domain of the first component (K), in particular as described herein.

According to one embodiment, the inventive recombinant vesicular stomatitis virus, preferably the oncolytic recombinant vesicular stomatitis virus, of the second component (V) of the vaccine of the invention encodes in its genome the second antigenic domain, which comprises at least one antigen or antigenic epitope selected from the group comprising CEA (SEQ ID NO: 24), Survivin (SEQ ID NO: 12), ASCL2 (SEQ ID NO: 15), MUC-1 (SEQ ID NO: 19), EpCAM (SEQ ID NO: 40), KRas (SEQ ID NO: 30), and MAGE-A3 (SEQ ID NO: 10). Preferably, the (second) antigenic domain (encoded in the genome) of the recombinant vesicular stomatitis virus, preferably the oncolytic recombinant vesicular stomatitis virus, of the invention as disclosed above comprises at least one antigen or antigenic epitope of CEA (SEQ ID NO: 24). It is also preferred that the (second) antigenic domain (encoded in the genome) of the recombinant vesicular stomatitis virus, preferably the oncolytic recombinant vesicular stomatitis virus, of the invention as disclosed above comprises at least one antigen or antigenic epitope of survivin (SEQ ID NO: 12). It is also preferred that the (second) antigenic domain (encoded in the genome) of the recombinant vesicular stomatitis virus, preferably the oncolytic recombinant vesicular stomatitis virus, of the invention as disclosed above comprises at least one antigen or antigenic epitope of ASCL2 (SEQ ID NO: 15).

According to a preferred embodiment, the recombinant vesicular stomatitis virus, preferably the oncolytic recombinant vesicular stomatitis virus, of the second component (V) of the invention encodes in its genome an antigenic domain comprising in N- to C-terminal direction one or more epitopes of CEA or functional sequence variants thereof, one or more epitopes of survivin or functional sequence variants thereof and one or more epitopes of ASCL2 or functional sequence variants thereof. It is preferred that the antigenic domain encoded in the genome of the recombinant vesicular stomatitis virus, preferably the oncolytic recombinant vesicular stomatitis virus, of the second component (V) according to the invention as disclosed herein comprises the amino acid sequence consisting of SEQ ID NO: 45.

According to one embodiment, the vaccine of the invention as disclosed above is for use in the treatment and/or prevention (or prophylaxis) of a tumor or cancer in a patient in need thereof. Accordingly, the first component (K) and second component (V) of the inventive vaccine as disclosed herein are preferably administered at least once, preferably in the order (K)-(V), more preferably in the order K-V-K. It is preferred that the first component (K) and second component (V) of the inventive vaccine are administered sequentially between 14 days to about 30 days from each other. In order to achieve a long lasting T-cell memory against the antigens of the antigenic domain as disclosed herein, the first component (K) of the invention may be administered repeatedly e.g. 14 days, 21 days, 60 days, 180 days following the last administration of the first component (K) of the inventive vaccine as disclosed herein, e.g. in the order K-V-K-K.

The vaccine according to the present invention may be used (in medicine) in combination with a therapeutically active agent, such as a chemotherapeutic agent, immunotherapeutic agent (such as an immune checkpoint inhibitor), or targeted drug. In one embodiment, the inventive vaccine as disclosed above is administered in combination with a therapeutically active agent, such as a chemotherapeutic agent, an immune checkpoint inhibitor, immunotherapeutic agent, or targeted drug. The immune checkpoint inhibitor for use (in medicine) in combination with the inventive vaccine is preferably an inhibitor of the PD-1/PD-L1 pathway, whereby the PD-1/PD-L1 pathway inhibitor may e.g. administered concomitantly, sequentially or alternately with first component (K) and/or second component (V) of the vaccine.

In one embodiment, the present invention provides for a method of increasing tumor infiltration with tumor antigen-specific T-cells, whereby the method comprises administering to a mammal, preferably a human, the vaccine according to the invention as disclosed above. In particular, the present invention provides a method of increasing the infiltration of a tumor with tumor antigen-specific T-cells in a patient, the method comprising administering to a patient (afflicted with a tumor or cancer) the vaccine according to the present invention.

In one embodiment, the present invention further provides a recombinant vesicular stomatitis virus, preferably an oncolytic recombinant vesicular stomatitis virus, as defined herein encoding in its genome a second antigenic domain which comprises at least one, two, three, or preferably all of the antigens or antigenic epitopes of the antigenic domain of the complex of the first component (K). The present invention further pertains to the use of the recombinant vesicular stomatitis virus, preferably the oncolytic recombinant vesicular stomatitis virus, as defined herein in modulating a cellular cytotoxic immune response against a tumor in a mammal as well as its use in the vaccine of the invention.

In one embodiment, the present invention provides for a method of treating a patient in need thereof afflicted with a tumor, whereby the method comprises administering to said patient the vaccine of the invention as disclosed herein. As disclosed herein, the method of treatment e.g. also comprises administering the vaccine of the invention and at least one further pharmaceutically active agent, such as e.g., an immune checkpoint inhibitor and/or chemotherapy.

In one embodiment, the present invention provides for a kit for use in vaccination for treating, preventing and/or stabilizing colorectal cancer, comprising the vaccine as disclosed herein and further pharmaceutically active agents as disclosed herein for use in the prevention and/or treatment of colorectal cancer.

According to a further embodiment, the present invention provides for a vesicular stomatitis virus, wherein the RNA genome of the vesicular stomatitis virus comprises or consists of an RNA sequence identical or at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 80. Accordingly, the rhabdovirus, preferably the oncolytic rhabdovirus, of the second component (V) of the vaccine of the invention may be a vesicular stomatitis virus, wherein the RNA genome of the vesicular stomatitis virus comprises or consists of an RNA sequence identical or at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 80.

In a preferred embodiment, the present invention provides for a vesicular stomatitis virus, wherein the RNA genome of the vesicular stomatitis virus comprises or consists of an RNA sequence identical or at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 80, wherein the vesicular stomatitis virus encodes in its genome a phosphoprotein (P) comprising the amino acid consisting of SEQ ID NO: 50, a nucleoprotein (N) comprising the amino acid sequence consisting of SEQ ID NO: 49, a matrix protein (M) comprising the amino acid sequence consisting of SEQ ID NO: 52, a large protein (L) comprising the amino acid sequence consisting of SEQ ID NO: 51, a glycoprotein (GP) comprising the amino acid sequence consisting of SEQ ID NO: 53, and an antigenic domain which comprises the amino acid sequence consisting of SEQ ID NO: 45 or SEQ ID NO: 59. Accordingly, the rhabdovirus, preferably the oncolytic rhabdovirus, of the second component (V) of the vaccine of the invention may be a vesicular stomatitis virus, wherein the RNA genome of the vesicular stomatitis virus comprises or consists of an RNA sequence identical or at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 80, wherein the vesicular stomatitis virus encodes in its genome a phosphoprotein (P) comprising the amino acid consisting of SEQ ID NO: 50, a nucleoprotein (N) comprising the amino acid sequence consisting of SEQ ID NO: 49, a matrix protein (M) comprising the amino acid sequence consisting of SEQ ID NO: 52, a large protein (L) comprising the amino acid sequence consisting of SEQ ID NO: 51, a glycoprotein (GP) comprising the amino acid sequence consisting of SEQ ID NO: 53, and an antigenic domain which comprises the amino acid sequence consisting of SEQ ID NO: 45 or SEQ ID NO: 59.

In a further preferred embodiment, the present invention provides for a vaccine which comprises two components wherein the first component (K) comprises a complex, which consists of or comprises (i) a cell penetrating peptide; (ii) an antigenic domain, which comprises at least one antigen or antigenic epitope; and (iii) at least one TLR peptide agonist, wherein the components (i)-(iii) are covalently linked, and wherein the second component (V) comprises a rhabdovirus, preferably an oncolytic rhabdovirus, wherein the rhabdovirus, preferably the oncolytic rhabdovirus, of the second component (V) is a vesicular stomatitis virus, wherein the RNA genome of the vesicular stomatitis virus comprises or consists of an RNA sequence identical or at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 80.

In a more preferred embodiment, the present invention provides for a vaccine which comprises two components wherein the first component (K) comprises a complex, which consists of or comprises a cell penetrating peptide; an antigenic domain, which comprises at least one antigen or antigenic epitope; and at least one TLR peptide agonist, and wherein the second component (V) comprises a rhabdovirus, preferably an oncolytic rhabdovirus, wherein the rhabdovirus, preferably the oncolytic rhabdovirus, of the second component (V) is a vesicular stomatitis virus, wherein the RNA genome of the vesicular stomatitis virus comprises or consists of an RNA sequence identical or at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 80, wherein the vesicular stomatitis virus encodes in its genome a phosphoprotein (P) comprising the amino acid consisting of SEQ ID NO: 50, a nucleoprotein (N) comprising the amino acid sequence consisting of SEQ ID NO: 49, a matrix protein (M) comprising the amino acid sequence consisting of SEQ ID NO: 52, a large protein (L) comprising the amino acid sequence consisting of SEQ ID NO: 51, a glycoprotein (GP) comprising the amino acid sequence consisting of SEQ ID NO: 53, and an antigenic domain which comprises the amino acid sequence consisting of SEQ ID NO: 45 or SEQ ID NO: 59.

In a further aspect, the present invention provides for a polypeptide comprising or consisting of an amino acid sequence of SEQ ID NO: 60 for use in medicine, in particular for use in an immunization regimen, in combination with a vesicular stomatitis virus, wherein the RNA genome of the vesicular stomatitis virus comprises or consists of an RNA sequence identical or at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 80. In a preferred embodiment, the present invention provides for a polypeptide comprising or consisting of an amino acid sequence of SEQ ID NO: 60 for use in medicine, in particular for use in an immunization regimen, in combination with a vesicular stomatitis virus, wherein the RNA genome of the vesicular stomatitis virus comprises or consists of an RNA sequence identical or at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 80, wherein the vesicular stomatitis virus encodes in its genome a phosphoprotein (P) comprising the amino acid consisting of SEQ ID NO: 50, a nucleoprotein (N) comprising the amino acid sequence consisting of SEQ ID NO: 49, a matrix protein (M) comprising the amino acid sequence consisting of SEQ ID NO: 52, a large protein (L) comprising the amino acid sequence consisting of SEQ ID NO: 51, a glycoprotein (GP) comprising the amino acid sequence consisting of SEQ ID NO: 53, and an antigenic domain which comprises the amino acid sequence consisting of SEQ ID NO: 45 or SEQ ID NO: 59.

In another related aspect, the present invention provides for a vesicular stomatitis virus, wherein the RNA genome of the vesicular stomatitis virus comprises or consists of an RNA sequence identical or at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 80 for use in medicine, in particular for use in an immunization regimen, in combination with a polypeptide comprising or consisting of an amino acid sequence of SEQ ID NO: 60.

In a preferred embodiment, the present invention provides for a vesicular stomatitis virus, wherein the RNA genome of the vesicular stomatitis virus comprises or consists of an RNA sequence identical or at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 80 for use in medicine, in particular for use in an immunization regimen, in combination with a polypeptide comprising or consisting of an amino acid sequence of SEQ ID NO: 60, wherein the vesicular stomatitis virus encodes in its genome a phosphoprotein (P) comprising the amino acid consisting of SEQ ID NO: 50, a nucleoprotein (N) comprising the amino acid sequence consisting of SEQ ID NO: 49, a matrix protein (M) comprising the amino acid sequence consisting of SEQ ID NO: 52, a large protein (L) comprising the amino acid sequence consisting of SEQ ID NO: 51, a glycoprotein (GP) comprising the amino acid sequence consisting of SEQ ID NO: 53, and an antigenic domain which comprises the amino acid sequence consisting of SEQ ID NO: 45 or SEQ ID NO: 59.

In yet another related aspect, the present invention provides for a kit of parts comprising a polypeptide and a vesicular stomatitis virus, wherein the polypeptide comprises or consists of an amino acid sequence of SEQ ID NO: 60, and wherein the RNA genome of the vesicular stomatitis virus comprises or consists of an RNA sequence identical or at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 80, preferably further comprising an immune checkpoint inhibitor of the PD-1/PD-L1 pathway, preferably selected from the group consisting of pembrolizumab; nivolumab; pidilizumab; cemiplimab; PDR-001; atezolizumab; avelumab; durvalumab, an antibody comprising a heavy chain comprising the amino acid sequence of SEQ ID NO:61 and a light chain comprising the amino acid sequence of SEQ ID NO:62; an antibody comprising a heavy chain comprising the amino acid sequence of SEQ ID NO:63 and a light chain comprising the amino acid sequence of SEQ ID NO:64; an antibody comprising a heavy chain comprising the amino acid sequence of SEQ ID NO:65 and a light chain comprising the amino acid sequence of SEQ ID NO:66; an antibody comprising a heavy chain comprising the amino acid sequence of SEQ ID NO:67 and a light chain comprising the amino acid sequence of SEQ ID NO:68; and an antibody comprising a heavy chain comprising the amino acid sequence of SEQ ID NO:69 and a light chain comprising the amino acid sequence of SEQ ID NO:70.

In a preferred embodiment, the present invention provides for a kit of parts comprising a polypeptide and a vesicular stomatitis virus, wherein the polypeptide comprises or consists of an amino acid sequence of SEQ ID NO: 60, and wherein the RNA genome of the vesicular stomatitis virus comprises or consists of an RNA sequence identical or at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 80, wherein the vesicular stomatitis virus encodes in its genome a phosphoprotein (P) comprising the amino acid consisting of SEQ ID NO: 50, a nucleoprotein (N) comprising the amino acid sequence consisting of SEQ ID NO: 49, a matrix protein (M) comprising the amino acid sequence consisting of SEQ ID NO: 52, a large protein (L) comprising the amino acid sequence consisting of SEQ ID NO: 51, a glycoprotein (GP) comprising the amino acid sequence consisting of SEQ ID NO: 53, and an antigenic domain which comprises the amino acid sequence consisting of SEQ ID NO: 45 or SEQ ID NO: 59.

### BRIEF DESCRIPTION OF THE DRAWINGS

FIG. 1: A) Cartoon of the genomic organization of a vesicular stomatitis virus of the inventive vaccine in which the glycoprotein G was replaced by the glycoprotein GP of LCMV expressing one or more tumor antigens as an additional transgene. (B) Cartoon of the first component (K) of the vaccine comprising a cell penetrating peptide (CPP), a multi-antigenic domain (Mad) and a TLR agonist (TLRag).

FIG. 2: Heterologous prime-boost vaccination using the first component (K) and the second component (V, “VSV-GP-TAA”) according to the invention is superior to homologous vaccination with either vaccine platform. (A-C) Non-tumor bearing C57BL/6 mice were immunized against (A) ovalbumin (Ova, model antigen), (B) Adpgk and Reps1 (neoantigen, MC38 tumor model, Mad24; Mad24 contains two class I neoepitopes, i.e. adpgk and reps1 (described in Yadav et al., 2014) or (C) E7 (viral antigen, Tc1 tumor model) with either 2 nmol of the first component (K) given s.c. or 10⁷ TCID₅₀ VSV-GP-TAA (second component (V)) targeting the respective tumor-antigen administered (A) i.m. or (B, C) i.v on days indicated by arrows. The fraction of CD8⁺ T cells specific for (A) Ova, (B) Adppgk and (C) E7 in peripheral blood of immunized mice was measured 7 days after each vaccination using MHC-multimers.

FIG. 3: Heterologous prime-boost vaccination using the first component (K) and the second component (V, “VSV-GP-TAA”) according to the invention is superior to homologous vaccination with either vaccine platform. Mice were immunized with 2 nmol of a first component (K) encoding OVA as antigen in the antigenic domain, i.e. Mad5 (SEQ ID NO: 74) of the vaccine of the invention, (s.c.) on day 0 and 14 and 1×10⁷ TCID₅₀ VSV-GP-Ova was administered as a second component (V) either i.m. or i.v. on day 7 as indicated by arrows. (A) The frequency of Ova reactive CTLs among CD8⁺ T cells in circulation was assessed on day 4, 14, 21, 44, 72, 98 and 134 post prime. The number of Ova reactive CTLs in the (B) spleen and (C) bone marrow of the immunized mice was measured 19 weeks post prime. The number of Ova specific cytotoxic T cells with effector (KLRG1⁺) and memory precursor (CD127⁺) phenotype was estimated in (D) peripheral blood, (E) spleen and (F) bone marrow 134 days after first vaccination. The Mad5 multi-antigen construct contains four mouse specific antigenic epitopes: a class I and a class II epitopes derived from the ovalbumin (OVA257-264 and OVA323-339 respectively), a class I epitope derived from the self-antigen glycoprotein 100 (gp 10025-33) and a class II epitope from the alpha-chain of the I-E class II molecule (Ea52-68).

FIG. 4: Priming with the first component (K) improves functionality of peripheral antigen specific CTLs. (A) Schematic of experimental plan. Mice with palpable TC-1 tumors were vaccinated either with 2 nmol of the first component (K) (antigenic domain comprising Mad25, administered s.c.) on day 7 and 1×10⁷ TCID₅₀ of the second component (V) (VSV-HPV administered i.v.)) on day 14, or twice with 2 nmol of the first component (K) on days 7 and 14, or twice with the second component (V) on days 7 and 14. Spleens were harvested on day 21 post implantation of the TC-1 tumors for analysis of CD8 lymphocytes. The VSV-HPV contains the full-length genes encoding the three different antigens E2, E6* and E7* (comprising the same antigenic domain of Mad25), which are derived from HPV16. The original E6 and E7 sequences were mutated to carry point mutations that abrogate their oncogenicity. (B) Cytokine production by ex vivo restimulated HPV-specific CD8 T cells measured by intracellular flow cytometry staining. (C) Granzyme B expression by splenic CD8 T cells was measured by flow cytometry. Granzyme B is a degranulation marker.

FIG. 5: Priming with the first component (K) of the vaccine improves functionality of intratumoral antigen specific CTLs. (A) Schematic of experimental plan. Mice with palpable TC-1 tumors were vaccinated with either 2 nmol of the first component (K) of the vaccine (antigenic domain: Mad25, s.c.) on day 7 and 1×10⁷ TCID₅₀ of the second component (V) (VSV-HPV comprising the same antigenic domain Mad25, administered i.v.) on day 14 after TC-1 tumor implantation, or twice with the second component (V) (VSV-HPV, administered i.v.) on days 7 and 14 after TC-1 tumor implantation. Tumors were harvested on day 21 post implantation for analysis of tumor-infiltrating lymphocytes (TILs). The VSV-HPV contains the full-length genes encoding the three different antigens E2, E6* and E7* (comprising the same antigenic domain of Mad25), which are derived from HPV16. The original E6 and E7 sequences were mutated to carry point mutations that abrogate their oncogenicity. (B) Frequency of activation and exhaustion marker expression (PD1, Tim3, KLRG1) by HPV-specific CD8 T cells was measured by flow cytometry. (C) Cytokine production by ex vivo restimulated HPV-specific CD8 T cells was measured by intracellular flow cytometry staining.

FIG. 6: Remodeling of the immunosuppressive tumor microenvironment (TME) after heterologous vaccination. Mice bearing palpable Tc1 tumors were immunized with 2 nmol of a first component (K) (antigenic domain: Mad25 comprising amino acid sequence according to SEQ ID NO: 75, administered s.c.) and the second component (V) (VSV-GP-HPV, comprising the antigenic domain Mad25 (SEQ ID NO: 75), administered i.v) on day 7 and 14, respectively. Tumors were harvested on day 21 and tumor infiltrating immune cells were characterized by flow cytometry. (A) The proportion of various immune cell subsets among all leukocytes and (B) the fraction of different dendritic cell (DC) subsets within all DCs is shown.

FIG. 7: Therapeutic effect of KVKK vaccine in syngeneic tumor models expressing ovalbumin. C57BL/6 mice were injected with 3×10⁵ EG.7 cells. Mice were treated with 2 nmol of a first component (K) comprising the antigenic domain Mad39 comprising the amino acid sequence according to SEQ ID NO: 77 (given s.c., dotted lines), 1×10⁷ TCID₅₀ VSV-GP-OVA (comprising the antigenic domain Mad39 and the full-length gene encoding Ovalbumin in its genome) given i.v. (dotted lines) or 200 μg αPD-1 antibody given i.v. Blood was drawn 7 days after vaccination for tetramer analysis. Administration of either the first component (K) or the second component (V) (VSV-GP-OVA) was performed on days 5, 12, 19 and 26 post tumor implantation. Administration of the αPD-1 antibody was performed on days 7, 11, 15, 19, 23 and 27 post tumor implantation. Controls were performed with mock treatment and αPD-1 antibody only. Four different treatment regimens were tested: VVVV, KKKK, KVKK, and KVKK+αPD-1. (A) Tumor growth and (B) survival after treatment is depicted. The number of complete responders (grey), i.e. tumor-free mice, among total mice (black) is indicated in parentheses beside the tumor growth curves for every treatment group. (C) Frequency of Ova-specific CTLs and (D) proportion of PD-1 positive among Ova tetramer positive cells in peripheral blood is shown. (E) Correlation between the magnitude of Ova response (day26) and tumor sizes (day25) is depicted for three different treatment groups.

FIG. 8: Efficacy of therapeutic cancer vaccination using the first component (K) and the second component (V) of the vaccine (VSV-GP-TAA) in syngeneic tumor models targeting neoepitopes. C57BL/6 mice were injected with 2×10⁵ MC-38 cells subcutaneously in the right flank. Mice were vaccinated against Adpgk and Reps1 (MC-38 neo-epitopes, antigenic domain Mad24, SEQ ID NO: 76) using 2 nmol of the first component (K) comprising Mad24 administered s.c. or 1×10⁷ TCID₅₀ of the second component (V) (VSV-GP-TAA, comprising Mad24, administered i.v.) on days indicated (dotted lines). Additionally, mice received 200 μg of αPD-L1 antibody i.p. on days indicated (dotted lines). Administration of either the first component (K) or the second component (V) (VSV-GP-TAA) was performed on days 3, 10, 17 and 24 post MC-38 cells injection. Administration of the αPD-1 antibody was performed on days 6, 10, 13, 17, 20, 24 and 27 post MC-38 cells injection. Controls were performed with mock treatment and αPD-1 antibody only. Four different treatment regimens were tested: VVVV, KKKK, KVKK, and KVKK+αPD-1. (A) Tumor growth curve and (B) survival of the animals is depicted. The number of complete responders (grey), i.e. tumor-free mice, among total mice (black) is indicated in parentheses beside the tumor growth curves for every treatment group. (C) The frequency of circulating Adpgk-specific CD8 T cells was assessed by flow cytometry 7 days after each vaccination.

FIG. 9: Efficacy of therapeutic cancer vaccination using a first component (K) and the second component (V) VSV-GP-TAA in syngeneic tumor models targeting an oncoviral antigen. As a proof-of-concept C57BL/6 mice were injected with 1.5×10⁵ Tc-1 cells subcutaneously in the right flank. Mice were vaccinated against E7 (an HPV-derived oncoprotein expressed in TC-1 cells) using 2 nmol of a first component (K) comprising the antigenic domain Mad25 (SEQ ID NO: 75) s.c. and 1×10⁷ TCID₅₀ VSV-GP-TAA i.v. on days indicated (red dotted lines). Additionally, mice received 200 μg of αPD-1 antibody i.v. on days indicated (black dotted lines). Administration of either the first component (K) or the second component (V) (VSV-GP-TAA) was performed on days 7, 14, 28 and 49 post TC-1 cells injection. Administration of the αPD-1 antibody was performed on days 7, 14 and 28 post TC-1 cells injection. Controls were performed with mock treatment and αPD-1 antibody only. Four different treatment regimens were tested: VVVV, KKKK, KVKK, and KVKK+αPD-1. (A) Tumor growth curves and (B) survival of the animals is depicted. (C) The frequency of circulating HPV-E7-specific CD8 T cells was assessed by flow cytometry 7 days after each vaccination. (D) Correlation between proportion of antigen specific CTLs and tumor size is shown. The number of complete responders (grey) among total mice (black) is indicated in parentheses beside the tumor growth curves for every treatment group.

FIG. 10: Generation of long lasting immune memory in vaccinated mice. The presence of circulating tumor-specific CTLs against vaccinated antigens was assessed in long-term survivors which had rejected subcutaneous tumors after therapeutic vaccination. The frequency of (A) Ova specific, (B) Adpgk specific and (C) E7 specific CD8⁺ T cells in peripheral blood of mice which rejected (A) EG.7, (B) MC38 and (C) Tc1 tumors is depicted.

FIG. 11: Tumor rechallenge protection in vaccinated mice. Surviving mice from different treatment groups mice were rechallenged with (A) EG.7 or (B) Tc1 cells on the contralateral flank and tumor growth is depicted. Panel B shows data combined from 3 independent experiments. Age-matched littermates (control) were included. The number of tumor-free mice (grey) among total mice (black) is indicated in parentheses beside the tumor growth curves for every tumor growth curve.

FIG. 12: Component K promotes formation of memory T cells in vaccinated mice. (A-C) Non-tumor bearing mice were immunized with 2 nmol of a first component (K) comprising the antigenic domain Mad5 (SEQ ID NO: 74) s.c. or 1×10⁷ TCID₅₀ VSV-GP-Ova (comprising the antigenic domain Mad5 and the full-length gene encoding Ovalbumin) i.m. on days 0, 14 and 28 (indicated by arrows). The proportion of CD127⁻KLRG-1⁻ early effector cells (EECs), KLRG-1⁺ short-lived effector cells (SLECs) and CD127⁺ memory precursor effector cells (MPECs) among Ova-specific CD8⁺ T cells was measured in the peripheral blood 7 days post each immunization and is depicted for (A) homologous vaccination (KKK), (B) homologous VSV-GP-Ova vaccination (VVV) and (C) heterologous prime boost vaccination (KVK).

FIG. 13: Immunogenicity of the first component (K) and second component (V) in heterologous prime-boost. (A) Frequencies of circulating CEA-specific CD8 T cells following 3 vaccinations with either first component (K) (SEQ ID NO: 60, “ATP128”, VSV-GP-empty virus ((VSVØ), VSV-GP-Mad128 (SEQ ID NO: 80) which is a VSV-GP encoding an antigenic domain according to SEQ ID NO: 45, VSV-GP-Mad128Anaxa which is a VSV-GP encoding an amino acid sequence comprising SEQ ID NO: 71 which comprises an antigenic domain comprising SEQ ID NO: 45 and an immunomodulatory fragment of annexin II (SEQ ID NO: 7) or VSV-GP-ATP128 which is a VSV-GP encoding in its genome the complex comprising the amino acid sequence according to SEQ ID NO: 60, assessed by dextramer staining on blood cells (3-5 mice/group) in tumor-free mice. (B) The amount of KLRG1+PD-1+ activated cells among CEA-specific circulating CD8 T cells was quantified. *, p<0.05; **, p<0.01.

FIG. 14: CEA-specific immune response in the periphery. IFNγ-producing CEA-specific T cells were quantified by an Elispot assay (A), and by intracellular staining for cytokine-producing CD8 T cells (B), in the spleen 1 week after the 3rd vaccination. K is ATP128 (SEQ ID NO: 60). VSV-GP-empty virus ((VSVØ), VSV-GP-Mad128 (SEQ ID NO: 80), VSV-GP-Mad128Anaxa, and VSV-GP-ATP128 are identical to the components presented in FIG. 13. *, p<0.05; **, p<0.01; ***, p<0.001.

FIG. 15: Frequencies of Granzyme B positive circulating CEA-specific CD8 T cells. Tumor-free mice received 3 vaccinations with either the first component K (“ATP128”, SEQ ID NO: 60), VSV-GP-empty virus, VSV-GP-Mad128 (Mad128 comprises the antigenic cargo comprising the amino acid according to SEQ ID NO: 45), VSV-GP-Mad128Anaxa (antigenic domain comprising the amino acid sequence according to SEQ ID NO: 71) or VSV-GP-ATP128 (VSV-GP encoding in its genome the first component K, comprising the amino acid sequence according to SEQ ID NO: 60). Frequencies of Granzyme B (GzB) positive circulating CEA-specific CD8 T cells was assessed by dextramer staining on blood cells (5 mice per group were tested). *, p<0.05; **, p<0.01.

FIG. 16: Priming with the first component (K) improves functionality of peripheral HPV-specific T cells. C57BL/6J mice were injected s.c. with TC-1 cells on day 0 and vaccinated with the first component K-(antigenic domain comprising Mad25, administered s.c.) or the second component (V) (VSV-HPV, administered i.v.) on day 7 and 14. Blood, spleen and tumors were harvested on day 21 for flow cytometric analysis. 4-5 mice analyzed per group. (A) Frequency and (B) number of HPV-E7-specific CD8+ T cells were measured by flow cytometry in blood. Mann-Whitney test (*, p<0.05). (C) Proportions of peripheral HPV-E7-specific CD8+ T cells expressing activation and exhaustion markers are depicted. 2-way ANOVA with Sidak's multiple comparison (***, p<0.001; ****, p<0.0001).

FIG. 17: Priming with the first component (K) improves functionality of intratumoral HPV-specific T cells. (A-J) C57BL/6J mice were injected s.c. with TC-1 cells on day 0 and vaccinated with the first component K-(antigenic domain comprising Mad25, administered s.c.) or the second component (V) (VSV-HPV, administered i.v.) on day 7 and 14. Blood, spleen and tumors were harvested on day 21 for flow cytometric analysis. 4-5 mice analyzed per group. (A) Frequency and (B) number of HPV-E7-specific CD8+ T cells were measured by flow cytometry in tumors. Mann-Whitney test (*, p<0.05).

FIG. 18: Gene signatures after heterologous vaccination indicate strong immune activation in treated tumors. (A-I) C57BL/6J mice bearing TC-1 tumors were immunized as in Examples 7 and 8, or left untreated (mock). Tumors were harvested on day 23 post tumor implantation for transcriptome analysis using NanoString® technology. (A, B) Gene expression in TC-1 tumors from each vaccination group was normalized to mock tumors and the proportion of (A) significantly upregulated (fold change [FC]>2 and p<0.05) and (B) significantly downregulated (negative reciprocal of FC<−2 and p value <0.05) genes is displayed. (C, D) Venn diagrams depict the total number of significantly (C) upregulated and (D) downregulated genes after different vaccine regimens and the overlap between each gene set. (E-I) Heatmaps display relative gene expression as z-scores (scaled to each gene) and hierarchichal clustering (Euclidean distance, average linkage) was applied to sample data with each column representing one individual tumor. Expression of typical genes associated with (E) cytotoxic T cells, (F) cytokines, (G) dendritic cells, (H) chemokines and (I) antigen presentation is shown. 7-10 mice analyzed for each treatment group, p values were calculated using two-tailed t test and false discovery rate (FDR) adjusted p values calculated using Benjamini-Yekutieli procedure are reported.

FIG. 19: Genes uniquely upregulated after KV vaccination. List of genes uniquely upregulated in TC-1 tumors after heterologous KV vaccination. Genes with fold change >2.0 (normalized to mock treated tumors) and FDR adjusted p value <0.05 as shown in FIG. 18C.

FIG. 20: Remodelling of immunosuppressive tumor microenvironment (TME) after heterologous prime-boost vaccination. Mice bearing palpable TC-1 tumors were immunized as in Examples 7 and 8. (A) Cytokine and chemokine levels in plasma were quantified on day 15 and are shown as mean±SEM. One-way ANOVA with Tukey's multiple comparison (*p<0.05, ** p<0.01; *** p<0.001, **** p<0.0001). Dotted lines indicate the limit of quantification. (B-C) Tumors were harvested on day 21 and tumor-infiltrating leukocytes were characterized by flow cytometry (2-3 mice analyzed per group). Total CD45+ leukocytes (mean) is shown. (C) Representative immunohistochemistry images show T cell infiltration (CD8) in TC-1 tumors day 23 post tumor implantation after different vaccinations. Lower panel shows higher magnification view of boxed area from upper panel. Scale bars: 500 μm upper row, 50 μm lower row.

FIG. 21: Role of prime with the first component (K). Mice were injected with 1×10⁵ TC-1 cells s.c. and were immunized with 2 nmole the first component (K)-(antigenic domain comprising Mad25) s.c. 7 days later or with 1×10⁷ TCID₅₀ VSV-GP-HPV i.v. 14 days post tumor implantation essentially as described above for the TC-1 model. Additional doses of K and V were administered as indicated by dotted lines and tumor growth was monitored (n=7). (A) Tumor growth (mean±SEM), (B) survival and (C) individual tumor growth is depicted. Log-rank test was performed (*** p<0.001).

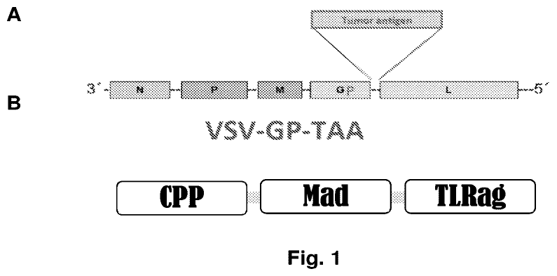

### DETAILED DESCRIPTION OF THE INVENTION

Although the present invention is described in detail below, it is to be understood that this invention is not limited to the particular methodologies, protocols and reagents described herein as these may vary. It is also to be understood that the terminology used herein is not intended to limit the scope of the present invention which will be limited only by the appended claims. Unless defined otherwise, all technical and scientific terms used herein have the same meanings as commonly understood by one of ordinary skill in the art.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

Throughout this specification and the claims which follow, unless the context requires otherwise, the term “comprise”, and variations such as “comprises” and “comprising”, will be understood to imply the inclusion of a stated member, integer or step but not the exclusion of any other non-stated member, integer or step. The term “consist of” is a particular embodiment of the term “comprise”, wherein any other non-stated member, integer or step is excluded. In the context of the present invention, the term “comprise” encompasses the term “consist of”. The term “comprising” thus encompasses “including” as well as “consisting” e.g., a composition “comprising” X may consist exclusively of X or may include something additional e.g., X+Y.

The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. No language in the specification should be construed as indicating any non-claimed element essential to the practice of the invention. The word “substantially” does not exclude “completely” e.g., a composition which is “substantially free” from Y may be completely free from Y. Where necessary, the word “substantially” may be omitted from the definition of the invention.

The term “about” in relation to a numerical value x as used herein means x±10%.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will supersede any other definition. In addition, the materials, methods, and examples are illustrative only and not intended to be limiting.

In a first aspect the present invention provides a vaccine which comprises a first component (K) and a second component (V), wherein the first component (K) comprises a complex, whereby said complex consists of or comprises:
(i) a cell penetrating peptide;
(ii) an antigenic domain, comprising at least one antigen or antigenic epitope; and
(iii) at least one TLR peptide agonist,
wherein the components i)-iii) are covalently linked, and
wherein the second component (V) comprises a rhabdovirus, preferably an oncolytic rhabdovirus.

In particular, the rhabdovirus, preferably the oncolytic rhabdovirus, of the second component (V) may encode the antigenic domain, or at least one antigen (fragment) or an antigenic epitope thereof, of the complex of the first component (K). In other words, at least one corresponding antigen (fragment) or epitope may be (1) comprised in the complex of the first component (K) and (2) encoded by (e.g., in the genome of) the rhabdovirus, preferably the oncolytic rhabdovirus, of the second component (V). Further details regarding the corresponding antigens of the first and second component are described below.

It was surprisingly found that a vaccine according to the present invention results in a (i) stimulation of multi-epitopic cytotoxic T cell-mediated immunity against the epitopes of the antigenic domain, (ii) induction of Tₕ cells, (iii) promotion of immunological memory, (iv) a shift of the ratio of anti-vector to anti-target T cell responses and at the same time overcomes the technical challenges associated with the production of multiple viruses.

Preferably, the complex of the first component (K) of the vaccine of the invention is a polypeptide or a protein, in particular a recombinant polypeptide or a recombinant protein, preferably a recombinant fusion protein or a recombinant fusion polypeptide. The term “recombinant” as used herein means that the polypeptide or the protein does not occur naturally. Accordingly, the complex of the first component (K) (for use) according to the present invention is a recombinant polypeptide or a recombinant protein and typically comprises components (i) to (iii), wherein the components (i) to (iii) are of different origins, i.e. do not naturally occur in this combination.

As used herein, the term “vaccine” refers to any compound/agent or combinations thereof, capable of inducing/eliciting an immune response in a host and which permits to treat and/or prevent an infection and/or a disease. A vaccine according to the invention affects the course of the disease by causing an effect on cells of the adaptive immune response, namely, B cells and/or T cells. The effect of vaccines can include, for example, induction of cell-mediated immunity or alteration of the response of the T cell to its antigen. The vaccine of the invention can e.g. be used for therapeutic administration or prophylactic administration.

The term “heterologous prime boost” according to the invention refers to the administration of two different (“heterologous”) vectors or delivery systems which each comprise at least one common antigen or antigenic epitope against which it is desired to raise an immune response. In particular, one of the different (“heterologous”) vectors or delivery systems is administered first (to “prime” the immune response), while the other is administered later (“boost”), e.g. 1, 2, 3, 4, 5, 6, 7, 8, 9, 10, 11, 12, 13, 14, 15, 16, 17, 18, 19, 20 or more days or weeks after the “prime” administration. For example, the first component (K) comprises a complex comprising at least one antigen or antigenic epitope (antigenic domain) as defined herein and the recombinant rhabdovirus, preferably the oncolytic recombinant rhabdovirus, of the second component (V) encodes/expresses at least one antigen or antigenic epitope which is identical in sequence to a corresponding antigen or antigenic epitope in the complex of the first component (K). Accordingly, the at least one antigen or antigenic epitope comprised in the antigenic domain encoded in the genome of the recombinant rhabdovirus, preferably the oncolytic recombinant rhabdovirus, of the invention may comprise the identical sequence as the corresponding antigenic domain comprised in the complex of the first component (K), or e.g. it may comprise the identical sequence for at least one antigen or antigenic epitope as comprised in the antigenic domain of the complex of the first component (K) according to the invention. The antigenic domain of the complex of the first component (K) of the invention and the antigenic domain as encoded in the genome by the recombinant rhabdovirus, preferably the oncolytic recombinant rhabdovirus as disclosed herein may e.g. be overlapping. The term “overlapping” as used according to the invention refers e.g. to two amino acid sequences which each have a continuous sequence element that is identical to a respective sequence in the other sequence and such overlapping sequence comprises at least one antigen or antigenic epitope as defined herein. For example, the amino acid sequences of the complex of the first component (K) and the antigenic domain as encoded in the genome of the recombinant rhabdovirus, or the oncolytic recombinant rhabdovirus as disclosed herein may be identical in a continuous sequence element from about 10, 15, 20, 25 to about 30, 35, 40, 45, 50, or from about 10 amino acids to about 15, 20, 25 amino acids in length, whereby the identical sequence element comprises at least one antigen or antigenic epitope. It is to be understood that N- and/or C-terminally to the identical sequence the amino acid sequence of the antigenic domain of the complex of the first component (K) according to the invention and that of the antigenic domain as encoded in the genome of the second component (V) may be different, e.g. they may comprise different antigens or antigenic epitopes.

The term “vaccine” as used in the present invention also implies that the vaccine of the invention as disclosed herein is in particular for use in medicine, in particular for use in the treatment of a disease, e.g. for use in the treatment or prevention of cancer, neoplastic disease, or tumors and/or cancer. For example, (the vaccine comprising) the first component (K) and/or the second component (V) according to the invention are for use in medicine, in particular for use in the treatment or prevention of cancer, neoplastic disease, or tumors.

It is to be understood that the vaccine according to the invention is not restricted to a single composition, but that it comprises at least two distinct components, which may e.g. be provided in separate packaging units. Accordingly, the vaccine of the invention is a combination comprising two distinct components, the first component K as defined herein and a second component (V) as defined herein. In particular, due to the distinct time points of administration in a heterologous prime boost vaccination scheme, the first component (K) and the second component (V) are preferably comprised in distinct compositions and/or separate packaging units. Accordingly, the vaccine as described herein may be provided as kit, e.g. as described herein.

In the context of the present invention, i.e. throughout the present application, the terms “peptide”, “polypeptide”, “protein” and variations of these terms refer to peptide, oligopeptide, oligomer or protein including fusion protein, respectively, comprising at least two amino acids joined to each other preferably by a normal peptide bond, or, alternatively, by a modified peptide bond, such as for example in the cases of isosteric peptides. A peptide, polypeptide or protein can be composed of L-amino acids and/or D-amino acids. Preferably, a peptide, polypeptide or protein is either (entirely) composed of L-amino acids or (entirely) of D-amino acids, thereby forming “retro-inverso peptide sequences”. The term “retro-inverso (peptide) sequences” refers to an isomer of a linear peptide sequence in which the direction of the sequence is reversed and the chirality of each amino acid residue is inverted (see e.g. Jameson et al., Nature, 368, 744-746 (1994); Brady et al., Nature, 368, 692-693 (1994)). In particular, the terms “peptide”, “polypeptide”, or “protein” also include “peptidomimetics” which are defined as peptide analogs containing non-peptidic structural elements, which peptides are capable of mimicking or antagonizing the biological action(s) of a natural parent peptide. A peptidomimetic lacks classical peptide characteristics such as enzymatically scissile peptide bonds. For example, the use of peptidomimetics in the complex of the first component if the inventive vaccine may be particularly useful or desirable if the antigenic domain comprises epitopes that require a particular shape or secondary structure on the peptide for immunogenicity, or to provide stability against rapid enzymatic degradation of the antigenic domain.

For example, in one embodiment, the first component (K) of the invention may consist of a peptide, polypeptide or protein which comprises amino acids other than the 20 amino acids defined by the genetic code in addition to these amino acids, or it can be composed of amino acids other than the 20 amino acids defined by the genetic code. In particular, a peptide, polypeptide or protein in the context of the present invention can equally be composed of amino acids modified by natural processes, such as post-translational maturation processes or by chemical processes, which are well known to a person skilled in the art. Such modifications are fully detailed in the literature. These modifications can appear anywhere in the polypeptide: in the peptide skeleton, in the amino acid chain or even at the carboxy- or amino-terminal ends. In particular, a peptide or polypeptide can be branched following an ubiquitination or be cyclic with or without branching. This type of modification can be the result of natural or synthetic post-translational processes that are well known to a person skilled in the art.

The terms “peptide”, “polypeptide”, “protein” in the context of the present invention in particular also include modified peptides, polypeptides and proteins. For example, peptide, polypeptide or protein modifications can include acetylation, acylation, ADP-ribosylation, amidation, covalent fixation of a nucleotide or of a nucleotide derivative, covalent fixation of a lipid or of a lipidic derivative, the covalent fixation of a phosphatidylinositol, covalent or non-covalent cross-linking, cyclization, disulfide bond formation, demethylation, glycosylation including pegylation, hydroxylation, iodization, methylation, myristoylation, oxidation, proteolytic processes, phosphorylation, prenylation, racemization, seneloylation, sulfatation, amino acid addition such as arginylation or ubiquitination. Such modifications are fully detailed in the literature (Proteins Structure and Molecular Properties (1993) 2nd Ed., T. E. Creighton, New York; Post-translational Covalent Modifications of Proteins (1983) B. C. Johnson, Ed., Academic Press, New York; Seifter et al. (1990) Analysis for protein modifications and nonprotein cofactors, Meth. Enzymol. 182:626-646 and Rattan et al., (1992) Protein Synthesis: Post-translational Modifications and Aging, Ann NY Acad Sci, 663:48-62). Accordingly, the terms “peptide”, “polypeptide”, “protein” may e.g. also include lipopeptides, lipoproteins, glycopeptides, glycoproteins and the like.

In some embodiments, the complex of the first component (K) is a peptide, polypeptide or protein. In a particularly preferred embodiment, the complex (K) of the inventive vaccine is a “classical” peptide, polypeptide or protein, whereby a “classical” peptide, polypeptide or protein is typically composed of amino acids selected from the 20 amino acids defined by the genetic code, linked to each other by a peptide bond.

According to one embodiment, the complex (K) of the vaccine of the invention is a polypeptide or protein comprising at least 20, at least 40, at least 50, at least 60, at least 70, preferably at least 80, at least 90, more preferably at least 100, at least 110, even more preferably at least 120, at least 130, particularly preferably at least 140, or most preferably at least 150 amino acid residues.

According to a preferred embodiment, the complex of the first component (K) of the vaccine according to the invention is a recombinant peptide, polypeptide or protein. 1) a polypeptide of semisynthetic or synthetic origin resulting from the expression of a combination of DNA molecules of different origin that are joined using recombinant DNA technologies; (2) a polypeptide of semisynthetic or synthetic origin that, by virtue of its origin or manipulation, is not associated with all or a portion of a protein with which it is associated in nature; (3) a polypeptide of semisynthetic or synthetic origin that is linked to a polypeptide other than that to which it is linked in nature; or (4) a polypeptide of semisynthetic or synthetic origin that does not occur in nature. Recombinant polypeptides such as e.g. complex (K) according to the invention may be produced by any method known in the art, such as e.g. prokaryotic and eukaryotic expression systems using well established protocols (see e.g. LaVallie, Current Protocols in Protein Science (1995) 5.1.1-5.1.8; Chen et al., Current Protocols in Protein Science (1998) 5.10.1-5.10.41), or e.g. by solid phase synthesis (see e.g. Nat Protoc. 2007; 2 (12): 3247-56).

According to one embodiment the complex of the first component (K) of the vaccine of the invention comprises a cell penetrating peptide (“CPP”). The term “cell penetrating peptide” (“CPP”) is generally used to designate short peptides that are able to transport different types of cargo molecules across plasma membrane, and, thus, facilitate cellular uptake of various molecular cargoes (from nanosize particles to small chemical molecules and large fragments of DNA). “Cellular internalization” of the cargo molecule linked to the cell penetrating peptide generally means transport of the cargo molecule across the plasma membrane and thus entry of the cargo molecule into the cell. Depending on the particular case, the cargo molecule can, then, be released in the cytoplasm, directed to an intracellular organelle, or further presented at the cell surface. Cell penetrating ability, or internalization, of the cell penetrating peptide or complex comprising said cell penetrating peptide, according to the invention can be checked by standard methods known to one skilled in the art, including flow cytometry or fluorescence microscopy of live and fixed cells, immunocytochemistry of cells transduced with said peptide or complex, and Western blot.

Cell penetrating peptides typically have an amino acid composition that either contains a high relative abundance of positively charged amino acids such as lysine or arginine or have a sequence that contains an alternating pattern of polar/charged amino acids and non-polar, hydrophobic amino acids. These two types of structures are referred to as polycationic or amphipathic, respectively. Cell-Penetrating peptides are of different sizes, amino acid sequences, and charges but all CPPs have a common characteristic that is the ability to translocate the plasma membrane and facilitate the delivery of various molecular cargoes to the cytoplasm or to an organelle of a cell. At present, the theories of CPP translocation distinguish three main entry mechanisms: direct penetration in the membrane, endocytosis-mediated entry, and translocation through the formation of a transitory structure. CPP transduction is an area of ongoing research. Cell-penetrating peptides have found numerous applications in medicine as drug delivery agents in the treatment of different diseases including cancer and virus inhibitors, as well as contrast agents for cell labeling and imaging.

Typically, cell penetrating peptides (CPPs) are peptides of 8 to 50 residues that have the ability to cross the cell membrane and enter into most cell types. Alternatively, they are also called protein transduction domain (PTDs) reflecting their origin as occurring in natural proteins. Frankel and Pabo simultaneously to Green and Lowenstein described the ability of the trans-activating transcriptional activator from the human immunodeficiency virus 1 (HIV-TAT) to penetrate into cells (Frankel, A. D. and C. O. Pabo, Cellular uptake of the tat protein from human immunodeficiency virus. Cell, 1988. 55 (6): p. 1189-93). In 1991, transduction into neural cells of the Antennapedia homeodomain (DNA-binding domain) from Drosophila melanogaster was described (Joliot, A., et al., Antennapedia homeobox peptide regulates neural morphogenesis. Proc Natl Acad Sci U SA, 1991. 88 (5): p. 1864-8). In 1994, the first 16-mer peptide CPP called Penetratin, having the amino acid sequence was characterized from the third helix of the homeodomain of Antennapedia (Derossi, D., et al., The third helix of the Antennapedia homeodomain translocates through biological membranes. J Biol Chem, 1994. 269 (14): p. 10444-50), followed in 1998 by the identification of the minimal domain of TAT, having the amino acid sequence required for protein transduction (Vives, E., P. Brodin, and B. Lebleu, A truncated HIV-1 Tat protein basic domain rapidly translocates through the plasma membrane and accumulates in the cell nucleus. J Biol Chem, 1997. 272 (25): p. 16010-7). Over the past two decades, a multitude of peptides have been described from different origins including viral proteins, e.g. VP22 (Elliott, G. and P. O'Hare, Intercellular trafficking and protein delivery by a herpesvirus structural protein. Cell, 1997. 88 (2): p. 223-33) and ZEBRA (Rothe, R., et al., Characterization of the cell-penetrating properties of the Epstein-Barr virus ZEBRA trans-activator. J Biol Chem, 2010. 285 (26): p. 20224-33), or from venoms, e.g. melittin (Dempsey, C. E., The actions of melittin on membranes. Biochim Biophys Acta, 1990. 1031 (2): p. 143-61), mastoporan (Konno, K., et al., Structure and biological activities of eumenine mastoparan-AF (EMP-AF), a new mast cell degranulating peptide in the venom of the solitary wasp (Anterhynchium flavomarginatum micado). Toxicon, 2000. 38 (11): p. 1505-15), maurocalcin (Esteve, E., et al., Transduction of the scorpion toxin maurocalcine into cells. Evidence that the toxin crosses the plasma membrane. J Biol Chem, 2005. 280 (13): p. 12833-9), crotamine (Nascimento, F. D., et al., Crotamine mediates gene delivery into cells through the binding to heparan sulfate proteoglycans. J Biol Chem, 2007. 282 (29): p. 21349-60) or buforin (Kobayashi, S., et al., Membrane translocation mechanism of the antimicrobial peptide buforin 2. Biochemistry, 2004. 43 (49): p. 15610-6). Synthetic CPPs were also designed including the poly-arginine (R8, R9, R10 and R12) (Futaki, S., et al., Arginine-rich peptides. An abundant source of membrane-permeable peptides having potential as carriers for intracellular protein delivery. J Biol Chem, 2001. 276 (8): p. 5836-40) or transportan (Pooga, M., et al., Cell penetration by transportan. FASEB J, 1998. 12 (1): p. 67-77). Any of the CPPs as disclosed may e.g. be used as cell penetrating peptide in the complex of the invention.

The use of CPPs according to the invention allows for efficient delivery, i.e. transport and loading, in particular of at least one antigen or antigenic epitope, into the antigen presenting cells (APCs), in particular into the dendritic cells (DCs) and thus to the dendritic cells' antigen processing machinery.

Preferably, the CPPs for use according to the invention are derived from the “ZEBRA” protein of the Epstein-Barr virus (EBV). “ZEBRA” (also known as Zta, Z, EB1, or BZLF1) generally refers to the basic-leucine zipper (bZIP) transcriptional activator of the Epstein-Barr virus (EBV). The minimal domain of ZEBRA, which exhibits cell penetrating properties, has been identified as spanning from residue 170 to residue 220 of ZEBRA. The amino acid sequence of ZEBRA is disclosed under NCBI accession number YP_401673 and comprises 245 amino acids represented in SEQ ID NO: 1

SEQ ID NO: 1
MMDPNSTSEDVKFTPDPYQVPFVQAFDQATRVYQD
LGGPSQAPLPCVLWPVLPEPLPQGQLTAYHVSTAP
TGSWFSAPQPAPENAYQAYAAPQLFPVSDITQNQQ
TNQAGGEAPQPGDNSTVQTAAAVVFACPGANQGQQ
LADIGVPQPAPVAAPARRTRKPQQPESLEECDSEL
EIKRYKNRVASRKCRAKFKQLLQHYREVAAAKSSE
NDRLRLLLKQMCPSLDVDSIIPRTPDVLHEDLLNF

CPPs derived from the viral protein ZEBRA have been described to transduce protein cargoes across biological membranes by both (i) direct translocation and (ii) lipid raft-mediated endocytosis (Rothe R, Liguori L, Villegas-Mendez A, Marques B, Grunwald D, Drouet E, et al. Characterization of the cell-penetrating properties of the Epstein-Barr virus ZEBRA trans-activator. The Journal of biological chemistry 2010; 285 (26): 20224-33). It is assumed that these two mechanisms of entry promote both MHC class I and as well as MHC class II restricted presentation of cargo antigens to CD8⁺ and CD4⁺ T cells, respectively. Accordingly, ZEBRA-derived CPPs can deliver multi-epitopic peptides, such as the complex (K) of the present invention comprising a antigenic domain (MAD) to dendritic cells (DCs), and subsequently to promote CTL and Tₕ cell activation and anti-tumor function. Thus, CPPs can thus efficiently deliver the complex for use according to the present invention to antigen presenting cells (APCs) and lead to multi-epitopic MHC class I and II restricted presentation. For example, ZEBRA-derived CPPs as disclosed in US 2018/0133327 are preferred for use in the complex (K) of the invention, more preferably the CPP of the first complex (K) of the invention is selected Z13, Z14, Z15 or Z18 as disclosed in US 2018/0133327, whereby the CPPs Z13, Z14, Z15, Z18 comprises or consists of an amino acid according to SEQ ID NO: 2

SEQ ID NO: 3
(KRYKNRVASRKSRAKFKQLLQHYREVAAAKSSENDRLRLLLK, Z13),
SEQ ID NO: 4
(KRYKNRVASRKSRAKFKQLLQHYREVAAAK, Z14),
(KRYKNRVASRKSRAKFK, Z15),
or
SEQ ID NO: 5
(REVAAAKSS END RLRLLLK, Z18).

CPPs that e.g. may be used with the complex (K) of the invention can also include sequence variants of any of the sequences disclosed above, which share at least 70%, 75%, 80%, 85%, 90%, 95% sequence identity with the respective sequence. Sequence variants may e.g. also include fragments of any of the sequences as disclosed above, whereby the term “fragment” refers to truncations of the sequences as disclosed above, i.e. an amino acid sequence, which is N-terminally, C-terminally and/or intrasequentially truncated compared to the amino acid sequence of the native sequences as disclosed above.

The term “sequence variant” as used in the context of the present invention refers to any alteration in a respective sequence in comparison to the corresponding reference sequence. The term “sequence variant” includes nucleotide sequence variants and amino acid sequence variants, preferably amino acid variants. Preferably, a reference sequence is any of the sequences disclosed herein, such as the CPP sequences disclosed above, or the sequences as listed in the “Table of Sequences and SEQ ID Numbers” and in the Sequence listing, respectively, i.e. SEQ ID NO: 1 to SEQ ID NO: 80. Preferably, a sequence variant shares, in particular over the entire length of the sequence, at least 70%, at least 75%, preferably at least 80%, more preferably at least 85%, even more preferably at least 90%, particularly preferably at least 95%, most preferably at least 99% sequence identity with a reference sequence, whereby sequence identity is calculated as described below. In general, for all variant sequences described herein, the higher the %-identity to the respective reference sequence, the more preferred is the sequence variant. In particular, a sequence variant preserves the specific function of the reference sequence.

Sequence identity according to the invention may e.g. be determined over the whole length of each of the sequences being compared to a respective reference sequence (so-called “global alignment”), that is particularly suitable for sequences of the same or similar length, or over shorter, defined lengths (so-called “local alignment”), that is more suitable for sequences of unequal length. In the above context, an amino acid sequence having a “sequence identity” of at least, for example, 95% to a query amino acid sequence, is intended to mean that the sequence of the subject amino acid sequence is identical to the query sequence except that the subject amino acid sequence may include up to five amino acid alterations per each 100 amino acids of the query amino acid sequence. In other words, to obtain an amino acid sequence having a sequence of at least 95% identity to a query amino acid sequence, up to 5% (5 of 100) of the amino acid residues in the subject sequence may be inserted or substituted with another amino acid or deleted. Methods for comparing the identity and homology of two or more sequences are well known in the art. The percentage to which two sequences are identical can for example be determined by using a mathematical algorithm. A preferred, but not limiting, example of a mathematical algorithm which can be used is the algorithm of Karlin et al. (1993), PNAS USA, 90:5873-5877. Such an algorithm is integrated in the BLAST family of programs (see also Altschul et al., 1990, J. Mol. Biol. 215, 403-410 or Altschul et al. (1997), Nucleic Acids Res, 25:3389-3402;), accessible through the home page of the NCBI at world wide web site ncbi.nlm.nih.gov) and FASTA (Pearson (1990), Methods Enzymol. 83, 63-98; Pearson and Lipman (1988), Proc. Natl. Acad. Sci. U.S.A 85, 2444-2448.). Sequences which are identical to other sequences to a certain extent can be identified by these programs. Furthermore, programs available in the Wisconsin Sequence Analysis Package (Devereux et al, 1984, Nucleic Acids Res., 387-395; Womble Methods Mol Biol. 2000; 132:3-22), for example the programs BESTFIT and GAP, may be used to determine the % identity between two polypeptide sequences. BESTFIT uses the “local homology” algorithm of (Smith and Waterman (1981), J. Mol. Biol. 147, 195-197.) and finds the best single region of similarity between two sequences. For example “gapped BLAST” may be utilized as described in Altschul et al., 1997, Nucleic Acids Res. 25:3389-3402. Alternatively, PSI-Blast can be used to perform an iterated search which detects distant relationships between molecules. When using any of the above BLAST, Gapped BLAST programs, the default parameters of the respective programs (e.g., XBLAST and NBLAST) may be used. Another preferred, non-limiting example of a mathematical algorithm utilized for the comparison of sequences as disclosed herein is the algorithm of Myers and Miller, CABIOS (1989). Such an algorithm is incorporated into the ALIGN program (version 2.0) which is part of the GCG sequence alignment software package. The ALIGN program may e.g. be used for comparing amino acid sequences using a PAM120 weight residue table, a gap length penalty of 12, and a gap penalty of 4. Additional algorithms for sequence analysis are known in the art and include ADVANCE and ADAM as described in Torellis and Robotti, 1994, Comput. Appl. Biosci. 10:3-5. Alternatively, protein sequence alignment may be carried out using the CLUSTAL W algorithm, as described by Higgins et al., 1996, Methods Enzymol. 266:383-402.

Amino acid substitutions of amino acid sequences as disclosed in the present invention may be “conservative” or “non-conservative” amino acid substitutions. The term “conservative substitutions” as used in the present invention are for example substituting a basic amino acid residue (Lys, Arg, His) for another basic amino acid residue (Lys, Arg, His), substituting an aliphatic amino acid residue (Gly, Ala, Val, Leu, lie) for another aliphatic amino acid residue, substituting an aromatic amino acid residue (Phe, Tyr, Trp) for another aromatic amino acid residue, substituting threonine by serine or leucine by isoleucine.

Substitutions of one or more L-amino acids with one or more D-amino acids are to be considered as conservative substitutions in the context of the present invention. Exemplary amino acid substitutions are presented in Table 1 below:

TABLE 1

| Original residues | Examples of substitutions |
|---|---|
| Ala (A) | Val, Leu, Ile, Gly |
| Arg (R) | His, Lys |
| Asn (N) | Gln |
| Asp (D) | Glu |
| Cys (C) | Ser |
| Gln (Q) | Asn |
| Glu (E) | Asp |
| Gly (G) | Pro, Ala |
| His (H) | Lys, Arg |
| Ile (I) | Leu, Val, Met, Ala, Phe |
| Leu (L) | Ile, Val, Met, Ala, Phe |
| Lys (K) | Arg, His |
| Met (M) | Leu, Ile, Phe |
| Phe (F) | Leu, Val, Ile, Tyr, Trp, Met |
| Pro (P) | Ala, Gly |
| Ser (S) | Thr |
| Thr (T) | Ser |
| Trp (W) | Tyr, Phe |
| Tyr (Y) | Trp, Phe |
| Original residues | Examples of substitutions |
| Val (V) | Ile, Met, Leu, Phe, Ala |

The term “non-conservative substitutions” as used in the present invention refers to substitution of an amino acid in the polypeptide with an amino acid with significantly differing side chain properties. Non-conservative substitutions may use amino acids between, rather than within, the defined groups and affects (a) the structure of the peptide backbone in the area of the substitution (e.g., proline for glycine) (b) the charge or hydrophobicity, or (c) the bulk of the side chain. By way of example and not limitation, an exemplary non-conservative substitution can be an acidic amino acid substituted with a basic or aliphatic amino acid; an aromatic amino acid substituted with a small amino acid; and a hydrophilic amino acid substituted with a hydrophobic amino acid. In the context of the present invention conservative amino acid substitutions are preferred.

In the context of the present invention, the term “MHC class I” designates one of the two primary classes of the Major Histocompatibility Complex molecules. The MHC class I (also noted “MHC I”) molecules are found on every nucleated cell of the body. The function of MHC class I is to display an epitope to cytotoxic cells (CTLs). In humans, MHC class I molecules consist of two polypeptide chains, α- and β2-microglobulin (b2m). Only the α chain is polymorphic and encoded by a HLA gene, while the b2m subunit is not polymorphic and encoded by the Beta-2 microglobulin gene. In the context of the present invention, the term “MHC class II” designates the other primary class of the Major Histocompatibility Complex molecules. The MHC class II (also noted “MHC II”) molecules are found only on a few specialized cell types, including macrophages, dendritic cells and B cells, all of which are dedicated antigen-presenting cells (APCs).

In one embodiment, the complex of the first component (K) of the vaccine according to the invention comprises more than one TLR peptide agonist, in particular 2, 3, 4, 5, 6, 7, 8, 9, 10 or more TLR peptide agonists.

The TLR peptide agonist as comprised in the complex of the first component (K) of the invention (or e.g. for use according to the invention) allows an increased targeting of the first component of the vaccine towards dendritic cells along with self-adjuvanticity. Physical linkage of a TLR peptide agonist to the CPP and the at least one antigen or antigenic epitope according to the present invention in the complex for use according to the present invention provides an enhanced immune response by simultaneous stimulation of antigen presenting cells, in particular dendritic cells, that internalize, process and display antigen(s).

As used in the context of the present invention, in particular in the context of the first component (K) according to the invention, a “TLR peptide agonist” is an agonist of a Toll-like receptor (TLR), i.e. it binds to a TLR and activates the TLR, in particular to produce a biological response. Moreover, the TLR peptide agonist is a peptide, a polypeptide or a protein as defined above. Preferably, the TLR peptide agonist comprises from 10 to about 150, 160, 170, 180, 190 amino acids, more preferably from 15 to 130 amino acids, even more preferably from 20 to 120 amino acids, particularly preferably from 25 to 110 amino acids, and most preferably from 30 to 100 amino acids.

Toll like receptors (TLRs) are transmembrane proteins that are characterized by extracellular, transmembrane, and cytosolic domains. The extracellular domains containing leucine-rich repeats (LRRs) with horseshoe-like shapes are involved in recognition of common molecular patterns derived from diverse microbes. Toll like receptors include TLRs1-10. Compounds capable of activating TLR receptors and modifications and derivatives thereof are well documented in the art. TLR1 may be activated by bacterial lipoproteins and acetylated forms thereof, TLR2 may in addition be activated by Gram positive bacterial glycolipids, LPS, LP A, LTA, fimbriae, outer membrane proteins, heat shock proteins from bacteria or from the host, and Mycobacterial lipoarabinomannans. TLR3 may be activated by dsRNA, in particular of viral origin, or by the chemical compound poly (LC). TLR4 may be activated by Gram negative LPS, LTA, Heat shock proteins from the host or from bacterial origin, viral coat or envelope proteins, taxol or derivatives thereof, hyaluronan containing oligosaccharides and fibronectins. TLR5 may be activated with bacterial flagellae or flagellin. TLR6 may be activated by mycobacterial lipoproteins and group B streptococcus heat labile soluble factor (GBS-F) or staphylococcus modulins. TLR7 may be activated by imidazoquinolines. TLR9 may be activated by unmethylated CpG DNA or chromatin-IgG complexes (see e.g. De Nardo, Cytokine 74 (2015) 181-189).

Preferably, the TLR peptide agonist comprised by the complex for use according to the present invention is an agonist of TLR1, 2, 4, 5, 6, and/or 10. TLRs are expressed either on the cell surface (TLR1, 2, 4, 5, 6, and 10) or on membranes of intracellular organelles, such as endosomes (TLR3, 4, 7, 8, and 9). The natural ligands for the endosomal receptors turned out to be nucleic acid-based molecules (except for TLR4). The cell surface-expressed TLR1, 2, 4, 5, 6, and 10 recognize molecular patterns of extracellular microbes (Monie, T. P., Bryant, C. E., et al. 2009: Activating immunity: Lessons from the TLRs and NLRs. Trends Biochem. Sci. 34 (11), 553-561). TLRs are expressed on several cell types but virtually all TLRs are expressed on DCs allowing these specialized cells to sense all possible pathogens and danger signals.

However, TLR2, 4, and 5 are constitutively expressed at the surface of DCs. Accordingly, the TLR peptide agonist comprised by the complex of the first component (K) of the vaccine according to the present invention is more preferably a peptide agonist of TLR2, TLR4 and/or TLR5. Even more preferably, the TLR peptide agonist is a TLR2 peptide agonist and/or a TLR4 peptide agonist. Particularly preferably, the TLR peptide agonist is a TLR4 peptide agonist, or alternatively is both, a TLR2 and a TLR4 agonist. TLR2 can detect a wide variety of ligands derived from bacteria, viruses, parasites, and fungi. The ligand specificity is often determined by the interaction of TLR2 with other TLRs, such as TLR1, 6, or 10, or non-TLR molecules, such as dectin-1, CD14, or CD36. The formation of a heterodimer with TLR1 enables TLR2 to identify triacyl lipoproteins or lipopeptides from (myco)bacterial origin, such as Pam3CSK4 and peptidoglycan (PGA; Gay, N. J., and Gangloff, M. (2007): Structure and function of Toll receptors and their ligands. Annu. Rev. Biochem. 76, 141-165; Spohn, R., Buwitt-Beckmann, U., et al. (2004): Synthetic lipopeptide adjuvants and Toll-like receptor 2-Structure-activity relationships. Vaccine 22(19), 2494-2499). Heterodimerization of TLR2 and 6 enables the detection of diacyl lipopeptides and zymosan. Lipopolysaccharide (LPS) and its derivatives are ligands for TLR4 and flagellin for TLR5 (Bryant, C. E., Spring, D. R., et al. (2010). The molecular basis of the host response to lipopolysaccharide. Nat. Rev. Microbiol. 8(1), 8-14).

TLR2 interacts with a broad and structurally diverse range of ligands, including molecules expressed by microbes and fungi. Multiple TLR2 agonists have been identified, including natural and synthetic lipopeptides (e.g. Mycoplasma fermentas macrophage-activating lipopeptide (MALP-2)), peptidoglycans (PG such as those from S. aureus), lipopolysaccharides from various bacterial strains (LPS), polysaccharides (e.g. zymosan), glycosylphosphatidyl-inositol-anchored structures from gram positive bacteria (e.g. lipoteichoic acid (LTA) and lipo-arabinomannan from mycobacteria and lipomannas from M. tuberculosis). Certain viral determinants may also trigger via TLR2 (Barbalat R, Lau L, Locksley R M, Barton G M. Toll-like receptor 2 on inflammatory monocytes induces type I interferon in response to viral but not bacterial ligands. Nat Immunol. 2009: 10(11):1200-7). Bacterial lipopeptides are structural components of cell walls. They consist of an acylated s-glycerylcysteine moiety to which a peptide can be conjugated via the cysteine residue. Examples of TLR2 agonists, which are bacterial lipopeptides, include MALP-2 and it's synthetic analogue di-palmitoyl-S-glyceryl cysteine (Pam2Cys) or tri-palmitoyl-S-glyceryl cysteine (Pam₃Cys).

In addition, high-mobility group box 1 protein (HMGB1) and peptide fragments thereof are assumed to act as an enhancer of TLR2-mediated inflammatory activities (see e.g. Aucott et al. Molecular Medicine (2018) 24:19). HMGB1-derived peptides which may e.g. be used as enhancers of TLR2-mediated signaling comprise those e.g. disclosed in WO2006/083301, or e.g. Δ30 HMGB1 and which can act as an enhancer of TLR2-mediated inflammatory activities in combination with TLR2/TLR4 peptide agonists. Accordingly, in one embodiment the inventive complex of the first component (K) of the vaccine may e.g. comprise as part of the TLR agonist Δ30 HMGB1 or any immunomodulatory fragment thereof, such as those disclosed in WO2006/083301 A1 in combination with a TLR2/TLR4 peptide agonist, such as e.g. ANAXA (SEQ ID NO:6) or sequence variants thereof such as SEQ ID NO: 7. Accordingly, the inventive complex of the first component (K) may comprise in addition to the TLR peptide agonists disclosed above Δ30 HMGB1 (SEQ ID NO: 8), or any immunomodulatory fragment thereof, or any of the peptides Hp-1-HP-166 as disclosed in WO2006/083301 A1, preferably Hp-31. Hp-46, Hp-106. For example, the complex of the first component (K) of the invention may comprise at least the TLR peptide agonists EDA (SEQ ID NO: 8) and Δ30 HMGB1 (SEQ ID NO: 9), or EDA (SEQ ID NO: 8) and Hp-31, or Hp-46, or Hp-106, preferably the complex of the first component (K) of the invention comprises at least the TLR peptide agonists ANAXA (SEQ ID NO: 6) and A30 HMGB1 (SEQ ID NO: 9), or ANAXA (SEQ ID NO: 6) and Hp-31, or Hp-46, or Hp-106, or ANAXA sequence variant (SEQ ID NO: 7) and Hp-31, or Hp-46, or Hp-106. The use of any such combination may e.g. be advantageous if a stronger self-adjuvancy of the complex of the first component (K) of the vaccine of the invention is desired.

A diversity of ligands interact with TLR4, including Monophosphoryl Lipid A from Salmonella minnesota R595 (MPLA), lipopolysaccharides (LPS), mannans (Candida albicans), glycoinositolphospholipids (Trypanosoma), viral envelope proteins (RSV and MMTV) and endogenous antigens including fibrinogen and heat-shock proteins. Such agonists of TLR4 are for example described in Akira S, Uematsu S, Takeuchi O. Pathogen recognition and innate immunity. Cell. February 24; 2006: 124(4):783-801 or in Kumar H, Kawai T, Akira S. Toll-like receptors and innate immunity. Biochem Biophys Res Commun. October 30; 2009 388(4):621-5. LPS, which is found in the outer membrane of gram negative bacteria, is the most widely studied of the TLR4 ligands. Suitable LPS-derived TLR4 agonist peptides are described for example in WO 2013/120073 (A1). While TLR peptide agonists are preferred for the complex of the first component (K) according to the invention, non-peptide TLR agonists such as LPS may be used and covalently conjugated to the complex. For example, conjugation may e.g. be performed between the carbonyl group of the reducing terminal of 3-deoxy-D-manno-oct-2-ulosonic acid (Kdo) exposed after acid hydrolyses of LPS and the aminooxy group of a bifunctional linker bound to the protein (see e.g. Methods Mol Biol. 2011; 751:317-27).

In some embodiments, the TLR peptide agonist is a fragment of a (naturally occurring) protein, or a variant thereof, which shares at least 70% or at least 75%, preferably at least 80% or at least 85%, more preferably at least 90% or at least 95%, even more preferably at least 97% or at least 98%, particularly preferably at least 99% sequence identity. Such fragments may have a minimum length of at least 20 or 25, preferably at least 30 or 35, more preferably at least 40 or 50, even more preferably 60 or 70, still more preferably at least 80 or 90, such as at least 100, amino acids. In particular, the fragment exhibits TLR agonist functionality. The fragment of the protein may advantageously be selected such that it provides the “TLR agonist domain” of the protein, but preferably does not include any other domain (other than the TLR agonist domain) of the protein. Therefore, in some embodiments, the TLR agonist does not comprise another immunological active domain (other than the TLR agonist domain), more preferably the TLR agonist does not comprise another biological active domain (other than the TLR agonist domain). For example, in some embodiments, the TLR agonist is not flagellin (which includes further domains in addition to the TLR agonist functionality). However, in some embodiments, the TLR agonist may be a fragment of flagellin including the TLR agonist domain of flagellin (but no other domain of flagellin).

Another suitable TLR peptide agonist comprises or consists of Hp91, or a fragment or variant thereof as described herein. Hp91 is a TLR4-agonist, as described, e.g., in U.S. Pat. No. 9,539,321 B2 and has the following amino acid sequence:

[SEQ ID NO: 53]
DPNAPKRPPSAFFLFCSEKRYKNRVASRKSRAKFK
QLLQHYREVAAAKSSENDRLRLLLKESLKISQAVH
AAHAEINEAGREVVGVGALKVPRNQDWLGVPRFAK
FASFEAQGALANIAVDKANLDVEQLESIINFEKLT
EWTGS

TLR5 is triggered by a region of the flagellin molecule expressed by nearly all motile bacteria. Thus, flagellin, or peptides or proteins derived from flagellin and/or variants or fragments of flagellin are also suitable as TLR peptide agonists comprised by the complex of the first component (K) (for use) according to the present invention.

In some embodiments, it is preferred that the TLR peptide agonist according to the invention is not flagellin and/or any unmodified fragment or fragments thereof. For example, entolimod (CBLB502) may be used as a TLR5 peptide agonist in the complex of the first component (K) according to the invention.

Examples of TLR peptide agonists for use according to the invention thus include the TLR2 lipopeptide agonists MALP-2, Pam₂Cys and Pam₃Cys or modifications thereof, different forms of the TLR4 agonist LPS, e.g. N. meningitidis wild-type L3-LPS and mutant penta-acylated LpxL1-LPS, and the TLR5 agonist flagellin.

However, it is preferred that the TLR peptide agonist comprised by the complex of the first component of the vaccine (for use) according to the present invention is neither a lipopeptide nor a lipoprotein, neither a glycopeptide nor a glycoprotein, more preferably, the TLR peptide agonist comprised by the complex of the first component of the vaccine (for use) according to the present invention is a classical peptide, polypeptide or protein as defined herein.

A preferred TLR2/4 peptide agonist is annexin II or an immunomodulatory fragment thereof, which is described in detail in WO 2012/048190 A1 and U.S. patent application Ser. No. 13/033,1546, in particular a TLR2 peptide agonist comprising an amino acid sequence according to the annexin II coding sequence SEQ ID NO: 4 or SEQ ID NO: 7 of WO 2012/048190 A1 or fragments or variants thereof are preferred.

Thereby, a TLR2/4 peptide agonist comprising or consisting of an amino acid sequence according to SEQ ID NO: 6 or a sequence variant thereof as described above is particularly preferred as component (iii), i.e. as the at least one TLR peptide agonist, comprised by the complex of the first component (K) (for use) according to the present invention.

[SEQ ID NO: 6]
STVHEILCKLSLEGDHSTPPSAYGSVKPYTNFDAE,
TLR2/4 peptide agonist Anaxa

A particularly preferred functional sequence variant of the TLR peptide agonist according to SEQ ID NO: 6 is the TLR peptide agonist according to SEQ ID NO: 7:

[SEQ ID NO: 7]
STVHEILSKLSLEGDHSTPPSAYGSVKPYTNFDAE

Accordingly, a TLR2/4 peptide agonist comprising or consisting of an amino acid sequence according to SEQ ID NO: 7 or a sequence variant thereof as described above is particularly preferred as component (iii) of the complex of the first component (K) of the vaccine/kit of the invention, i.e. as the at least one TLR peptide agonist, comprised by the complex. In other words, the TLR peptide agonist in the complex of the first component (K) most preferably comprises or consists of a peptide having an amino acid sequence according to SEQ ID NO: 7, or a functional sequence variant thereof having at least 70% sequence identity (preferably at least 75%, more preferably at least 80%, even preferably at least 85%, still more preferably at least 90%, particularly preferably at least 95%, most preferably at least 99% sequence identity).

Regarding TLR4, TLR peptides agonists are particularly preferred, which in particular correspond to motifs that bind to TLR4, in particular (i) peptides mimicking the natural LPS ligand (RS01: Gln-Glu-Ile-Asn-Ser-Ser-Tyr and RS09: Ala-Pro-Pro-His-Ala-Leu-Ser) and (ii) Fibronectin derived peptides. The cellular glycoprotein Fibronectin (FN) has multiple isoforms generated from a single gene by alternative splicing of three exons. One of these isoforms is the extra domain A (EDA), which interacts with TLR4.

Further suitable TLR peptide agonists comprise a fibronectin EDA domain or a fragment or variant thereof. Such suitable fibronectin EDA domains or a fragments or variants thereof are disclosed in EP 1 913 954 B1, EP 2 476 440 A1, US 2009/0220532 A1, and WO 2011/101332 A1. Thereby, a TLR4 peptide agonist comprising or consisting of an amino acid sequence according to SEQ ID NO: 8 or a sequence variant thereof as described above is particularly preferred as component (iii), i.e. as the at least one TLR peptide agonist, comprised by the complex of the first component (K) (for use) according to the present invention.

[SEQ ID NO: 8]
NIDRPKGLAFTDVDVDSIKIAWESPQGQVSRYRVTYSS
PEDGIRELFPAPDGEDDTAELQGLRPGSEYTVSVVALH
DDMESQPLIGIQST
(TLR4 peptide agonist EDA)

The complex of the first component (K) of the vaccine of the invention comprises at least one TLR peptide agonist, preferably the complex of the first component (K) according to the present invention comprises more than one TLR peptide agonist, in particular 2, 3, 4, 5, 6, 7, 8, 9, 10 or more TLR peptide agonists, more preferably the complex of the first component (K) according to the present invention comprises (at least) two or three TLR peptide agonists, even more preferably the complex of the first component (K) (for use) according to the present invention comprises (at least) four or five TLR peptide agonists. If more than one TLR peptide agonist is comprised by the complex of the first component (K) (for use) according to the present invention it is understood that said TLR peptide agonist is in particular also covalently linked in the complex for use according to the present invention, e.g. to another TLR peptide agonist and/or to a component (i), i.e. a cell penetrating peptide, and/or to a component (ii), i.e. an antigen or antigenic epitope.

The TLR peptide agonists comprised by the complex of the first component (K) according to the present invention may e.g. be the same or different. Preferably, the TLR peptide agonists comprised by the complex of the first component (K) according to the present invention are different from each other.

In a particularly preferred embodiment, the complex of the first component (K) according to the present invention comprises one single TLR peptide agonist, e.g. one single TLR agonist selected from those as disclosed above. In a particularly preferred embodiment, the complex of the first component (K) according to the present invention comprises one single TLR peptide agonist and no further component having TLR agonist properties except the one single TLR peptide agonist as disclosed above.

In one embodiment, the antigenic domain of the complex of the first component (K) of the vaccine according to the invention comprises at least one antigen or antigenic epitope. As used herein, an “antigen” is any structural substance which serves as a target for the receptors of an adaptive immune response, in particular as a target for antibodies, T cell receptors, and/or B cell receptors. An “epitope”, also known as “antigenic determinant”, is the part (or fragment) of an antigen that is recognized by the immune system, in particular by antibodies, T cell receptors, and/or B cell receptors and prompts an immune response. Thus, one antigen has at least one epitope, i.e. a single antigen may have or comprise more than one, e.g. one or more epitopes. In the context of the present invention, the term “epitope” is mainly used to designate T cell epitopes, which are presented on the surface of an antigen-presenting cell, where they are bound to Major Histocompatibility Complex (MHC). T cell epitopes presented by MHC class I molecules are typically, but not exclusively, peptides between 8 and 11 amino acids in length, whereas MHC class ∥ molecules present longer peptides, generally, but not exclusively, between 12 and 25 amino acids in length.

Preferably, the at least one antigen or antigenic epitope comprised in the antigenic domain of the complex of the first component (K) of the invention is selected from the group consisting of a peptide, a polypeptide, or a protein. It is to be understood that the at least one antigen or antigenic epitope according to the invention can comprise for example at least one, i.e. one or more, peptides, polypeptides or proteins linked together.

According to one embodiment, the antigenic domain of the complex of the first component (K) of the invention comprises more than one antigen or antigenic epitope, e.g. the complex of the first component (K) of the invention may comprise in particular 2, 3, 4, 5, 6, 7, 8, 9, 10 or more antigens or antigenic epitopes. Preferably, the one or more antigen or antigenic epitopes comprised in the complex of the first component (K) of the invention are positioned consecutively (or overlapping), in particular the more than one antigen or antigenic epitope, in particular 2, 3, 4, 5, 6, 7, 8, 9, 10 or more antigens or antigenic epitopes are positioned consecutively in the antigenic domain of the first component. This means in particular that all antigens and/or antigenic epitopes comprised by the complex of the first component (K) are functionally directly linked to each other without any intervening sequences. For example, no CPP, or TLRag of the invention is positioned within the amino acid sequences of the more than one antigen or antigenic epitope. It is preferred that the elements (i)-(iii) of the complex of the first component (K) of the invention are positioned in the following N-terminal to C-terminal order:
(i) CPP—(ii) antigenic domain—(iii) TLRag.

Alternatively, the elements (i)-(iii) of the complex of the first component (K) of the invention may also be linked in the order (i)-(iii)-(ii), or (ii)-(i)-(iii), or e.g. antigens and/or antigenic epitopes which may be positioned consecutively in such a way and linked to each other by a spacer or linker which is not one of the elements (i)-(iii) as disclosed above, i.e. a cell penetrating peptide, nor component c), i.e. a TLR peptide agonist. However, a direct linkage of the elements of the antigenic domain of the invention in the order (i)-(ii)-(iii) is preferred.

Alternatively, however, the various antigens and/or antigenic epitopes as disclosed herein may also be positioned in any other way in the complex of the first component (K) (for use) according to the present invention, for example with element (i) and/or component (iii) positioned in between two or more antigens and/or antigenic epitopes, or e.g. one or more antigens and/or antigenic epitopes positioned at the respective other end of element (i) and/or element (iii) of the complex of the invention. The term “element” or “elements” as used herein refers to the functional or structural elements of the complex of the first component (K) of the vaccine of the invention. Accordingly, the CPP, the antigenic domain and the TLRag may each be referred to as elements of the complex of the first component (K) of the invention.

In one preferred embodiment the least one antigen or antigenic epitope comprised in the complex of the first component of the invention is at least one CD4+ epitope and/or at least one CD8+ epitope, e.g. the first component (K) (for use) according to the present invention comprises at least one antigen or antigenic epitope, which is at least one CD4⁺ epitope and/or at least one CD8⁺ epitope. The terms “CD4⁺ epitope” or “CD4⁺-restricted epitope”, as used herein, designate an epitope recognized by a CD4⁺ T cell, said epitope in particular consisting of an antigen fragment lying in the groove of a MHC class II molecule. A single CD4⁺ epitope comprised in the first component (K) (for use) according to the present invention preferably consists of about 6, 7, 8, 9, 10, 11, 12 to about 25, 30, 40, 50, 60, 75, 100 amino acids.

The terms “CD8⁺ epitope” or “CD8⁺-restricted epitope”, as used in the context of the present invention, designate an epitope recognized by a CD8⁺ T cell, said epitope in particular consisting of an antigen fragment lying in the groove of a MHC class I molecule. A single CD8⁺ epitope comprised in the complex for use according to the present invention preferably consists of about 8-11 amino acids, or e.g. of about 8-15 amino acids, or of about 6, 7, 8, 9, 10, 11, 12, 13, 14, 15 to about 50, 60, 70, 80, 90, 100 amino acids.

Preferably, the at least one antigen of the invention can comprise or the at least one antigenic epitope can consist of a CD4⁺ epitope and/or a CD8⁺ epitope corresponding to antigenic determinant(s) of a cancer/tumor-associated antigen, a cancer/tumor-specific antigen, or an antigenic protein from a pathogen. More preferably, the at least one antigen can comprise or the at least one antigenic epitope can consist of a CD4⁺ epitope and/or a CD8⁺ epitope corresponding to antigenic determinant(s) of a cancer/tumor-associated antigen or a cancer/tumor-specific antigen. Most preferably, the at least one antigen can comprise or the at least one antigenic epitope can consist of a CD4⁺ epitope and/or a CD8⁺ epitope corresponding to antigenic determinant(s) of a tumor-associated antigen or a tumor-specific antigen. Throughout this present invention the term “cancer epitope” may be synonymously used with the term “tumor epitope”. The tumor or cancer types as disclosed herein may be benign, pre-malignant, or malignant, metastatic or non-metastatic.

It is also preferred that the complex of the first component (K) (for use) according to the present invention comprises at least two antigens or antigenic epitopes, wherein at least one antigen or antigenic epitope comprises or consists a CD4⁺ epitope and at least one antigen or antigenic epitope comprises or consists a CD8⁺ epitope. It is now established that Tₕ cells (CD4⁺) play a central role in the anti-tumor immune response both in DC licensing and in the recruitment and maintenance of CTLs (CD8⁺) at the tumor site. Therefore, a complex of the first component (K) (for use) according to the present invention comprising at least two antigens or antigenic epitopes, wherein at least one antigen or antigenic epitope comprises or consists of a CD4⁺ epitope and at least one antigen or antigenic epitope comprises or consists a CD8⁺ epitope, provides an integrated immune response allowing simultaneous priming of CTLs and Tₕ cells and is thus preferable to immunity against only one CD8⁺ epitope or only one CD4⁺ epitope. For example, the complex of the first component (K) (for use) according to the present invention may preferably comprise an Ealpha-CD4⁺ epitope and a gp100-CD8⁺ epitope.

Preferably, the complex of the first component (K) (for use) according to the present invention comprises at least two antigens or antigenic epitopes, wherein the at least two antigens or antigenic epitopes comprise or consist of at least two, e.g. 2, 3, 4, 5, 6, 7, 8, 9, or more, CD4⁺ epitopes and/or at least two, e.g. 2, 3, 4, 5, 6, 7, 8, 9, or more, CD8⁺ epitopes. Thereby, the at least two antigens or antigenic epitopes are preferably different antigens or antigenic epitopes, more preferably the at least two antigens or antigenic epitopes are different from each other but relating to the same kind of tumor. Use of a antigenic domain in the vaccine of the invention will (i) avoid outgrowth of antigen-loss variants, (ii) target different tumor cells within a heterogeneous tumor mass and (iii) circumvent patient-to-patient tumor variability, which may e.g. caused by different TAAs expressed by the tumor to be treated, or e.g. which may be caused by a different expression levels of the respective TAAs on the tumor to be treated. Thus, the complex of the first component (K) (for use) according to the present invention particularly preferably comprises at least four antigens or antigenic epitopes, in particular with at least two CD8⁺ epitopes and at least two CD4⁺ epitopes. Such a complex for use according to the present invention induces multi-epitopic CD8 CTLs and CD4 Tₕ cells to function synergistically with the second component (V) of the vaccine of the invention to counter tumor cells and promote efficient anti-tumor immunity. Tₕ cells are also involved in the maintenance of long-lasting cellular immunity that was monitored after vaccination. Such a complex of the vaccine (for use) according to the present invention acts synergistically with the second component (V) of the vaccine and induces polyclonal, multi-epitopic immune responses and poly-functional CD8⁺ and CD4⁺ T cells, and thus efficacious anti-tumor activity, in particular when the complex of the first component (K) (for use) according to the invention is administered prior to the administration of the second component (V).

Preferably, the complex of the first component (K) (for use) according to the present invention comprises at least two antigens or antigenic epitopes, more preferably the complex of the first component (K) (for use) according to the present invention comprises at least three antigens or antigenic epitopes, even more preferably the complex of the first component (K) (for use) according to the present invention comprises at least four antigens or antigenic epitopes, particularly preferably the complex of the first component (K) (for use) according to the present invention comprises at least five antigens or antigenic epitopes and most preferably the complex of the first component (K) (for use) according to the present invention comprises at least six antigens or antigenic epitopes. The antigens or antigenic epitopes comprised by the complex of the first component (K) (for use) according to the present invention may be the same or different, preferably the antigens or antigenic epitopes comprised by the complex of the first component (K) (for use) according to the present invention are different from each other. Preferably, the complex of the first component (K) (for use) according to the present invention comprises at least one CD4⁺ epitope and at least one CD8⁺ epitope.

Preferably, the complex of the first component (K) of the vaccine for use according to the present invention comprises more than one CD4⁺ epitope, e.g. two or more CD4⁺ epitopes from the same antigen or from different antigens, and preferably no CD8⁺ epitope. It is also preferred that the complex of the first component (K) of the vaccine for use according to the present invention comprises more than one CD8⁺ epitope, e.g. two or more CD8⁺ epitopes from the same antigen or from different antigens, and preferably no CD4⁺ epitope. Most preferably, however, the complex of the first component (K) of the vaccine for use according to the present invention comprises (i) at least one CD4⁺ epitope, e.g. two or more CD4⁺ epitopes from the same antigen or from different antigens, and (ii) at least one CD8⁺ epitope, e.g. two or more CD8⁺ epitopes from the same antigen or from different antigens.

For example, in one embodiment the antigenic domain of the complex of first component (K) according to the invention comprises at least one antigen or antigenic epitope which comprises or consists of at least one tumor epitope. As used in the present invention, a tumor epitope or tumor antigen is a peptide antigen that is produced in tumor cells. Many tumor antigens have been identified in humans as well as mice, for example, various abnormal products of Kras and p53 are found in a variety of tumors.

For example, in one embodiment the antigenic domain of the complex of the first component (K) may comprise at least one antigen or antigenic epitope which comprises or consists of tumor-associated antigen or a tumor-specific antigen. The term “tumor-associated antigen” (TAA) as used in the present invention refers to a protein or polypeptide antigen that is expressed by a tumor cell. For example, a TAA may be one or more surface proteins or polypeptides, nuclear proteins or glycoproteins, or fragments thereof, expressed by a tumor cell. For example, human tumor-associated antigens include differentiation antigens (such as melanocyte differentiation antigens), mutational antigens (such as p53), overexpressed cellular antigens (such as HER2), viral antigens (such as human papillomavirus proteins), and cancer/testis (CT) antigens that are expressed in germ cells of the testis and ovary but are silent in normal somatic cells (such as MAGE and NY-ESO-1). Many TAAs are not cancer- or tumor-specific and may also be found on normal tissues.

The term “tumor-specific antigens” (TSAs) as used in the present invention refers to a repertoire of peptides that is displayed on the tumor cell surface and can be specifically recognized by neoantigen-specific T cell receptors (TCRs) in the context of major histocompatibility complexes (MHCs) molecules. TSAs may also be referred to as which are also referred to as “tumor neoantigens” in the context of this invention. From an immunological perspective, tumor neoantigen is the truly foreign protein and entirely absent from normal human organs/tissues. For most human tumors without a viral etiology, tumor neoantigens can e.g. derive from a variety of nonsynonymous genetic alterations including single-nucleotide variants (SNVs), insertions and deletions (indel), gene fusions, frameshift mutations, and structural variants (SVs). The term “tumor-specific antigens” (TSAs) as used according to the invention also includes oncoviral antigens, such as e.g. antigens of human papilloma virus, or Merkel cell polyomavirus (MCPyV). Typically, oncoviral antigens are only found expressed on cells infected with the the respective virus. Tumor-neoantigens may be identified using in silico prediction tools known in the art as disclosed in Trends in Molecular Medicine, November 2019, Pages 980-992.

Preferably, the at least one tumor epitope, or the at least one TAA, or the at least one TSA of the antigenic domain of the invention is selected from the group of tumors or cancers comprising endocrine tumors, gastrointestinal tumors, genitourinary and gynecologic tumors, head and neck tumors, hematopoietic tumors, skin tumors, thoracic and respiratory tumors. More specifically, the at least one tumor epitope, or the at least one TAA, or the at least one TSA of the antigenic domain of the invention is selected from the group of tumors and/or cancers comprising breast cancer, including triple-negative breast cancer, biliary tract cancer; bladder cancer; brain cancer including glioblastomas and medulloblastomas; cervical cancer; choriocarcinoma; colon cancer; endometrial cancer; esophageal cancer; gastric cancer; gastrointestinal stromal tumor (GIST), appendix cancer, cholangiocarcinoma, carcinoid tumor, gastrointestinal colon cancer, extrahepatic bile duct cancer, gallbladder cancer, gastric (stomach) cancer, gastrointestinal carcinoid tumor, colorectal cancer, or metastatic colorectal cancer, hematological neoplasms including acute lymphocytic and myelogenous leukemia; T-cell acute lymphoblastic leukemia/lymphoma; hairy cell leukemia; chronic myelogenous leukemia, multiple myeloma; AIDS-associated leukemias and adult T-cell leukemia lymphoma; intraepithelial neoplasms including Bowen's disease and Paget's disease; liver cancer; lung cancer, including non-small cell lung cancer, lymphomas including Hodgkin's disease and lymphocytic lymphomas; neuroblastomas; glioblastoma, oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, and osteosarcoma; skin cancer including melanoma, Merkel cell carcinoma, Kaposi's sarcoma, basal cell carcinoma, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; and renal cancer including adenocarcinoma and Wilms tumor.

Specifically preferably, the at least one tumor epitope, or the at least one TAA, or the at least one TSA of the antigenic domain of the complex of the first component (K) of the invention is selected from the group of tumors or cancers of colorectal cancer, metastatic colorectal cancer, pancreatic cancer, or breast cancer, including triple-negative breast cancer (TNBC). The term “triple negative breast cancer” as used herein refers to breast cancer that lacks the expression of estrogen receptor (ER), progesterone receptor (PgR) and HER2, all of which are molecular targets of therapeutic agents. TNBC accounts for 10-20% of invasive breast cancer cases and encompasses more than one molecular subtype. Typically, patients afflicted with TNBC have a relatively poorer outcome compared with those with other breast cancer subtypes owing to an inherently aggressive clinical behavior and a lack of recognized molecular targets for therapy. Triple negative breast cancer is a phenotype and its major components in molecular assays are the basal-like tumors, normal breast-like tumors and the more recently recognized, the uncommon but intriguing, claudin-low molecular subtypes and also includes BRCA1-deficient subtypes. TAAs that are expressed by TNBC comprise for example MAGE-A3, MUC-1, PRAME, ASCL2, and NY-ESO-1.

The term “pancreas cancer” or “pancreatic cancer” as used herein relates to cancer which is derived from pancreatic cells. Preferably, pancreatic cancer as used herein refers to pancreatic adenocarcinoma, including pancreatic ductal adenocarcinoma and ist morphological variants, e.g. adenosquamous carcinoma, colloid/mucinous carcinoma, undifferentiated/anaplastic carcinoma, signet ring cell carcinoma, medullary carcinoma, hepatoid carcinoma. Pancreatic adenocarcinoma is a lethal condition with poor outcomes and an increasing incidence. Pancreatic cancer is typically a disease of the elderly. It is extremely rare for patients to be diagnosed before the age of 30, and 90% of newly diagnosed patients are aged over 55 years of age, with the majority in their 7th and 8th decade of life, with a higher incidence in males compared to females. Pancreatic cancer is characterized by the expression of tumor-associated antigens comprising mesothelin, survivin, and NY-ESO-1.

As used herein, colorectal cancer (CRC, also known as “bowel cancer”) is a cancer that comprises colon cancers and rectal cancers (CC). Both individual cancers have many features in common, but the cancer starting point. According to Siegel, R., C. Desantis, and A. Jemal, Colorectal cancer statistics, 2014. CA Cancer J Clin, 2014. 64(2): p. 104-17, in the United States between 2006 and 2010, the incidence by tumor site is slightly more important in the proximal colon (first and middle parts of the colon). With about 19 cases on 100,000 people, it represents 42% of the cases. It is followed by the rectal cancer, with 28% of the cases and the distal colon (bottom part of the colon) with an incidence of 10 cases on 100,000 people. Anatomically, the term “colorectal cancer” includes (i) cancers of colon, such as cancers of cecum (including cancers the ileocecal valve), appendix, ascending colon, hepatic flexure, transverse colon, splenic flexure, descending colon, sigmoid colon (including cancers of sigmoid (flexure)) as well as cancers of overlapping sites of colon; (ii) cancers of recto-sigmoid junction, such as cancers of colon and rectum and cancers of rectosigmoid; and (iii) cancers of rectum, such as cancers of rectal ampulla.

Preferably, the colorectal cancer is a cancer of colon, such as a cancer of cecum (including cancer the ileocecal valve), cancer of appendix, cancer of ascending colon, cancer of hepatic flexure, cancer of transverse colon, cancer of splenic flexure, cancer of descending colon, cancer of sigmoid colon (including cancers of sigmoid (flexure)) or a combination thereof.

It is also preferred that the colorectal cancer is a cancer of rectosigmoid junction, such as (i) a cancer of colon and rectum or (ii) a cancer of rectosigmoid. Furthermore, it is also preferred that the colorectal cancer is a cancer of rectum, such as a cancer of rectal ampulla.

Colorectal cancer comprises different cell types such as e.g. the cell type, colorectal cancers include colorectal adenocarcinoma, colorectal stromal tumors, primary colorectal lymphoma, colorectal leiomyosarcoma, colorectal melanoma, colorectal squamous cell carcinoma and colorectal carcinoid tumors, such as, for example, carcinoid tumors of cecum, appendix, ascending colon, transverse colon, descending colon, sigmoid colon and/or rectum. Thus, preferred types of colorectal cancers include colorectal adenocarcinoma, colorectal stromal tumors, primary colorectal lymphoma, colorectal leiomyosarcoma, colorectal melanoma, colorectal squamous cell carcinoma and colorectal carcinoid tumors, such as, for example, carcinoid tumors of cecum, appendix, ascending colon, transverse colon, descending coloncom, sigmoid colon and/or rectum. More preferably, the colorectal cancer is a colorectal adenocarcinoma or a colorectal carcinoid carcinoma. Even more preferably, the colorectal cancer is a colorectal adenocarcinoma. Accordingly, the at least one tumor or cancer epitope of the antigenic domain of the complex of the first component (K) according to the invention can be selected from any of the colorectal cancer cell types disclosed above.

Since colorectal cancer expresses different TAAs, or TSAs depending on the staging of the tumor according to the TMN staging system, the at least one tumor or cancer epitope of the antigenic domain of the antigenic domain of the complex of the first component (K) of the vaccine of the invention preferably includes TAAs, or TSAs of for example the following stages for primary tumors (“T” stages): TX—Primary tumour cannot be assessed, TO—No evidence of primary tumour, Ta—Non-invasive papillary carcinoma, Tis—Carcinoma in situ: intraepithelial or invasion of lamina propria, T1—Tumour invades submucosa, T2—Tumour invades muscularis propria, T3—Tumour invades through the muscularis propria into the pericolorectal tissues, T4a—Tumour penetrates to the surface of the visceral peritoneum and T4b—Tumour directly invades or is adherent to other organs or structures; following stages for lymph nodes (“N” stages): NX—Regional lymph nodes cannot be assessed, NO—No regional lymph node metastasis, N1—Metastasis in 1-3 regional lymph nodes with N1a—Metastasis in 1 regional lymph node, N1b—Metastasis in 2-3 regional lymph nodes and N1c—Tumor deposit(s) in the subserosa, mesentery, or nonperitonealized pericolic or perirectal tissues without regional nodal metastasis, N2—Metastasis in 4 or more lymph nodes with N2a—Metastasis in 4-6 regional lymph nodes and N2b—Metastasis in 7 or more regional lymph nodes; and the following stages for distant metastasis (“M” stages): M0—No distant metastasis and M1-Distant metastasis with M1a—Metastasis confined to 1 organ or site (eg, liver, lung, ovary, nonregional node) and M1b—Metastases in more than 1 organ/site or the peritoneum. The stages can be integrated into the following numerical staging of colorectal cancer: Stage 0: Tis, NO, M0; Stage I: T1, NO, M0 or T2, NO, M0; Stage IIA: T3, NO, M0; Stage IIB: T4a, NO, M0; Stage IIC: T4b, NO, M0; Stage IlIlA: T1-T2, N1/N1c, M0 or T1, N2a, M0; Stage IllB: T3-T4a, N1/N1c, M0 or T2-T3, N2a, M0 or T1-T2, N2b, M0; Stage IllC: T4a, N2a, M0 or T3-T4a, N2b, M0 or T4b, N1-N2, M0; Stage IVA: any T, any N, M1a and Stage IVB: any T, any N, M1b. Briefly, in Stage 0, the cancer has not grown beyond the inner layer of the colon or rectum; in Stage I the cancer has spread from the mucosa to the muscle layer; in Stage II the cancer has spread through the muscle layer to the serosa nearby organs; in Stage III the cancer has spread to nearby lymph node(s) or cancer cells have spread to tissues near the lymph nodes; and in Stage IV the cancer has spread through the blood and lymph nodes to other parts of the body.

Various tumor associated antigens of the above colorectal cancer cell types and stages have been reported and comprise e.g. CEA, MAGE, MUC1, survivin, WT1, RNF43, TOMM34, VEGFR-1, VEGFR-2, KOC1, ART4, KRas, EpCAM, HER-2, COA-1 SAP, TGF-βRII, p53, ASCL2, IL13Ralpha2, ASCL2, NY-ESO-1, MAGE-A3, PRAME and SART 1-3 (see e.g. World J Gastroenterol 2018 December 28; 24(48): 5418-5432). Accordingly, the at least one tumor epitope of the antigenic domain of the complex of the first component (K) of the vaccine according to the invention is an epitope of an antigen selected from the group consisting of EpCAM, HER-2, MUC-1, TOMM34, RNF 43, KOC1, VEGFR, βhCG, survivin, CEA, ASCL2, TGFβR2, p53, KRas, OGT, mesothelin, CASP5, COA-1, MAGE, SART, IL13Ralpha2, ASCL2, NY-ESO-1, MAGE-A3, PRAME.

### Melanoma-Associated Antigen (MAGE)

The mammalian members of the MAGE (melanoma-associated antigen) gene family were originally described as completely silent in normal adult tissues, with the exception of male germ cells and, for some of them, placenta. By contrast, these genes were expressed in various kinds of tumors. Therefore, the complex for use according to the present invention preferably comprises an antigen of the MAGE-family (a “MAGE” antigen) or an epitope thereof. Of the MAGE family, in particular MAGE-A3 and MAGE-D4 are preferred, and MAGE-A3 is particularly preferred. The normal function of MAGE-A3 in healthy cells is unknown. MAGE-A3 which may e.g. also be referred to as Cancer/Testis Antigen 1.3, is a tumor-specific protein, and has been identified on many tumors. The amino acid sequence of MAGE-A3 is shown in the following:

[SEQ ID NO: 10]
MPLEQRSQHCKPEEGLEARGEALGLVGAQAPATEE
QEAASSSSTLVEVTLGEVPAAESPDPPQSPQGASS
LPTTMNYPLWSQSYEDSSNQEEEGPSTFPDLESEF
QAALSRKVAELVHFLLLKYRAREPVTKAEMLGSVV
GNWQYFFPVIFSKAFSSLQLVFGIELMEVDPIGHL
YIFATCLGLSYDGLLGDNQIMPKAGLLIIVLAIIA
REGDCAPEEKIWEELSVLEVFEGREDSILGDPKKL
LTQHFVQENYLEYRQVPGSDPACYEFLWGPRALVE
TSYVKVLHHMVKISGGPHISYPPLHEWVLREGEE

Accordingly, a preferred complex of the first component (K) of the vaccine for use according to the present invention comprises at least one antigen of the amino acid sequence according to SEQ ID NO: 10.

### Mesothelin

Mesothelin, which was initially identified in ovarian cancer as a protein reacting with an antibody termed “mAb K1” is a tumor antigen that is highly expressed in many human cancers, including malignant mesothelioma and pancreatic, ovarian, and lung adenocarcinomas. The amino acid sequence of mesothelin according to UniProtKB Q13421 is shown below:

[SEQ ID NO: 11]
MALPTARPLLGSCGTPALGSLLFLLFSLGWVQPSRT
LAGETGQEAAPLDGVLANPPNISSLSPRQLLGFPC
AEVSGLSTERVRELAVALAQKNVKLSTEQLRCLAH
RLSEPPEDLDALPLDLLLFLNPDAFSGPQACTRFF
SRITKANVDLLPRGAPERQRLLPAALACWGVRGSL
LSEADVRALGGLACDLPGRFVAESAEVLLPRLVSC
PGPLDQDQQEAARAALQGGGPPYGPPSTWSVSTMD
ALRGLLPVLGQPIIRSIPQGIVAAWRQRSSRDPSW
RQPERTILRPRFRREVEKTACPSGKKAREIDESLI
FYKKWELEACVDAALLATQMDRVNAIPFTYEQLDV
LKHKLDELYPQGYPESVIQHLGYLFLKMSPEDIRK
WNVTSLETLKALLEVNKGHEMSPQAPRRPLPQVAT
LIDRFVKGRGQLDKDTLDTLTAFYPGYLCSLSPEE
LSSVPPSSIWAVRPQDLDTCDPRQLDVLYPKARLA
FQNMNGSEYFVKIQSFLGGAPTEDLKALSQQNVSM
DLATFMKLRTDAVLPLTVAEVQKLLGPHVEGLKAE
ERHRPVRDWILRQRQDDLDTLGLGLQGGIPNGYLV
LDLSMQEALSGTPCLLGPGPVLTVLALLLASTLA

Accordingly, a preferred complex of the first component (K) of the vaccine for use according to the present invention comprises at least one antigen of the amino acid sequence according to SEQ ID NO: 11.

### Survivin

In some embodiments, the antigenic domain of the complex (of the first component) comprises at least one epitope of survivin. Survivin, also called baculoviral inhibitor of apoptosis repeat-containing 5 or BIRC5 (UniProtKB 015392), is a member of the inhibitor of apoptosis (IAP) family. The survivin protein functions to inhibit caspase activation, thereby leading to negative regulation of apoptosis or programmed cell death. The amino acid sequence of survivin is shown in the following:

[SEQ ID NO: 12]
MGAPTLPPAWQPFLKDHRISTFKNWPFLEGCACTP
ERMAEAGFIHCPTENEPDLAQCFFCFKELEGWEPD
DDPIEEHKKHSSGCAFLSVKKQFEELTLGEFLKLD
RERAKNKIAKETNNKKKEFEETAKKVRRAIEQLAA
MD

Accordingly, a preferred complex of the first component (K) (for use) according to the present invention comprises an amino acid sequence according to SEQ ID NO: 12 or a fragment or a variant thereof as described herein. In particular, it is preferred that the antigenic domain of said first component (K) comprises a peptide consisting of the amino acid sequence according to SEQ ID NO: 12, or a fragment thereof having a length of at least 10 amino acids, or a functional sequence variant thereof having at least 70%, 75%, 80%, 85%, 90% or 95% sequence identity.

Several epitopes of survivin are known to the skilled person. A preferred survivin epitope, which is preferably comprised by the complex of the first component (K) (for use) according to the present invention, includes the following epitope (the epitope sequence shown in the following is a fragment of the above survivin sequence and is, thus, shown in the above survivin sequence underlined; the following epitope sequence may refer to one epitope or more than one (overlapping) epitopes):

[SEQ ID NO: 22]
RISTFKNWPF

Accordingly, a preferred complex of the first component (K) (for use) according to the present invention comprises an amino acid sequence according to SEQ ID NO: 22.

Accordingly, it is preferred that the complex of the first component (K) (for use) according to the present invention comprises an epitope of survivin. More preferably, the complex of the first component (K) comprises a peptide having an amino acid sequence according to SEQ ID NO: 12, or a fragment thereof having a length of at least 10 amino acids (preferably at least 15 amino acids, more preferably at least 20 amino acids, even more preferably at least 25 amino acids and most preferably at least 30 amino acids), or a functional sequence variant thereof having at least 70% sequence identity (preferably at least 75%, more preferably at least 80%, even preferably at least 85%, still more preferably at least 90%, particularly preferably at least 95%, most preferably at least 99% sequence identity). Even more preferably, the complex of the first component (K) comprises a peptide having an amino acid sequence according to SEQ ID NO: 22. Most preferably, the complex comprises a peptide having an amino acid sequence according to SEQ ID NO: 23 or a functional sequence variant thereof having at least 70% sequence identity (preferably at least 75%, more preferably at least 80%, even preferably at least 85%, still more preferably at least 90%, particularly preferably at least 95%, most preferably at least 99% sequence identity).

### NY-ESO-1

NY-ESO-1 (also referred to as “Cancer/testis antigen 1”, or “New York esophageal squamous cell carcinoma 1”, UniProtKB P78358) is a well-known cancer-testis antigen (CTAs) with re-expression in numerous cancer types. NY-ESO-1 elicits spontaneous humoral and cellular immune responses and is characterized by a restricted expression pattern, render it a good candidate target for cancer immunotherapy. NY-ESO-1-specific immune responses have been observed in various cancer types. The amino acid sequence of NY-ESO-1 is shown in the following:

[SEQ ID NO: 13]
MQAEGRGTGGSTGDADGPGGPCIPDGPGGNAGGPG
EAGATGGRGPRGAGAARASGPGGGAPRGPHGGAAS
GLNGCCRCGARGPESRLLEFYLAMPFATPMEAELA
RRSLAQDAPPLPVPGVLLKEFTVSGNILTIRLTAA
DHRQLQLSISSCLQQLSLLMWITQCFLPVFLAQPP
SGQRR

In a preferred embodiment, the at least one tumor epitope of the antigenic domain of complex of the first component (K) of the vaccine according to the invention as disclosed above is an epitope of an antigen selected from the group consisting of mesothelin, survivin, and NY-ESO-1. For example, the at least one tumor epitope of the antigenic domain of the first component (K) of the vaccine according to the invention is an epitope selected from mesothelin, survivin, or mesothelin and NY-ESO-1, or survivin and NY-ESO-1. According to one embodiment, the at least one tumor epitope of the antigenic domain of the first component (K) of the vaccine according to the invention as disclosed above comprises an epitope of the antigen mesothelin, or NY-ESO-1, or survivin, or a fragment thereof, or a sequence variant of the tumor antigen, or fragment thereof. The term “fragment” as used throughout the instant invention comprises at least 10 consecutive amino acids of the antigen, preferably at least 15 consecutive amino acids of the antigen, more preferably at least 20 consecutive amino acids of the antigen, even more preferably at least 25 consecutive amino acids of the antigen and most preferably at least 30 consecutive amino acids of the antigen. A “sequence variant” is as defined above, namely a sequence variant has an (amino acid) sequence which is at least 70%, preferably at least 75%, more preferably at least 80%, even preferably at least 85%, still more preferably at least 90%, particularly preferably at least 95%, most preferably at least 99% identical to the reference sequence. A “functional” sequence variant means in the context of an antigen, antigen fragment, or epitope, that the function of the epitope(s), e.g. comprised by the antigen (fragment), is not impaired or abolished, i.e. that it is immunogenic, preferably has the same immunogenicity as the epitope comprised in the full length antigen. Preferably, however, the amino acid sequence of the epitope(s), e.g. comprised by the cancer/tumor antigen (fragment) as described herein, is not mutated and, thus, identical to the reference epitope sequence. For example, a vaccine according to the invention as disclosed above comprises an antigenic domain which comprises at least one, e.g. one, two, three, four, five, six, seven, eight, nine, ten or more epitopes selected from at least one, two or all of the antigens as disclosed above, e.g. mesothelin, survivin, and NY-ESO-1, may be particularly useful in the context of pancreatic cancer.

### PRAME

PRAME (Melanoma antigen preferentially expressed in tumors, UniProtKB P78395) otherwise known as cancer testis antigen 130 (CT130), MAPE (melanoma antigen preferentially expressed in tumors) and OIP4 (OPA-interacting protein 4) is a member of the cancer testis antigen (CTA) family. PRAME expression in normal somatic tissues is epigenetically restricted to adult germ cells with low expression in the testis, epididymis, endometrium, ovaries and adrenal glands. Similar to the CTA member NY-ESO-1, PRAME was identified as an immunogenic tumor-associated antigen in melanoma, and since its discovery its expression has been demonstrated in a variety of solid and hematological malignancies including triple negative breast cancer. The amino acid sequence of PRAME is shown below:

[SEQ ID NO: 14]
MERRRLWGSIQSRYISMSVWTSPRRLVELAGQSLL
KDEALAIAALELLPRELFPPLFMAAFDGRHSQTLK
AMVQAWPFTCLPLGVLMKGQHLHLETFKAVLDGLD
VLLAQEVRPRRWKLQVLDLRKNSHQDFWTVWSGNR
ASLYSFPEPEAAQPMTKKRKVDGLSTEAEQPFIPV
EVLVDLFLKEGACDELFSYLIEKVKRKKNVLRLCC
KKLKIFAMPMQDIKMILKMVQLDSIEDLEVTCTWK
LPTLAKFSPYLGQMINLRRLLLSHIHASSYISPEK
EEQYIAQFTSQFLSLQCLQALYVDSLFFLRGRLDQ
LLRHVMNPLETLSITNCRLSEGDVMHLSQSPSVSQ
LSVLSLSGVMLTDVSPEPLQALLERASATLQDLVF
DECGITDDQLLALLPSLSHCSQLTTLSFYGNSISI
SALQSLLQHLIGLSNLTHVLYPVPLESYEDIHGTL
HLERLAYLHARLRELLCELGRPSMVWLSANPCPHC
GDRTFYDPEPILCPCFMPN

### ASCL2 (Achaete-Scute Homoloq 2)

In some embodiments, the antigenic domain of said first component (K) comprises at least one epitope of ASCL2. ASCL2 is a basic helix-loop-helix transcription factor essential for the maintenance of proliferating trophoblasts during placental development. ASCL2 was found to be a putative regulator of proliferation that is overexpressed in intestinal neoplasia. The amino acid sequence of ASCL2 is shown in the following:

[SEQ ID NO: 15]
MDGGTLPRSAPPAPPVPVGCAARRRPASPELLRCS
RRRRPATAETGGGAAAVARRNERERNRVKLVNLGF
QALRQHVPHGGASKKLSKVETLRSAVEYIRALQRL
LAEHDAVRNALAGGLRPQAVRPSAPRGPPGTTPVA
ASPSRASSSPGRGGSSEPGSPRSAYSSDDSGCEGA
LSPAERELLDFSSWLGGY

Accordingly, the antigenic domain of said first component (K) preferably comprises a peptide having an amino acid sequence according to SEQ ID NO: 15, or a fragment thereof having a length of at least 10 amino acids, or a functional sequence variant thereof having at least 70%, 75%, 80%, 85%, 90% or 95% sequence identity. More preferably, the antigenic domain of said first component (K) comprises a peptide consisting of an amino acid sequence according to SEQ ID NO: 18 or a functional sequence variant thereof having at least 70%, 75%, 80%, 85%, 90% or 95% sequence identity.

Several epitopes of ASCL2 are known to the skilled person. Preferred ASCL2 epitopes, which are preferably comprised by the complex for use according to the present invention, include the following epitopes (the epitope sequences shown in the following are fragments of the above ASCL2 sequence and are, thus, shown in the above ASCL2 sequence underlined; each of the following epitope sequences may refer to one epitope or more than one (overlapping) epitopes):

[SEQ ID NO: 16]
SAVEYIRALQ
[SEQ ID NO: 17]
ERELLDFSSW

Accordingly, a preferred complex of the first component (K) (for use) according to the present invention comprises an amino acid sequence according to SEQ ID NO: 16 and/or an amino acid sequence according to SEQ ID NO: 17. In other words, the antigenic domain preferably comprises a peptide consisting of an amino acid sequence according to SEQ ID NO: 16 and/or a peptide having an amino acid sequence according to SEQ ID NO: 17.

Accordingly, it is preferred that the complex of the first component (K) for use according to the present invention comprises an epitope of ASCL2. More preferably, the complex of the first component (K) comprises a peptide having an amino acid sequence according to SEQ ID NO: 15, or a fragment thereof having a length of at least 10 amino acids (preferably at least 15 amino acids, more preferably at least 20 amino acids, even more preferably at least 25 amino acids and most preferably at least 30 amino acids), or a functional sequence variant thereof having at least 70% sequence identity (preferably at least 75%, more preferably at least 80%, even preferably at least 85%, still more preferably at least 90%, particularly preferably at least 95%, most preferably at least 99% sequence identity). Even more preferably, the complex of the first component (K) comprises a peptide having an amino acid sequence according to SEQ ID NO: 16 and/or a peptide having an amino acid sequence according to SEQ ID NO: 17. Most preferably, the complex of the first component (K) comprises a peptide having an amino acid sequence according to SEQ ID NO: 18 or a functional sequence variant thereof having at least 70% sequence identity (preferably at least 75%, more preferably at least 80%, even preferably at least 85%, still more preferably at least 90%, particularly preferably at least 95%, most preferably at least 99% sequence identity).

### Mucin-1 (MUC-1)

MUC-1 (UniProtKB P15941) is a human epithelial mucin, acting on cell adhesion. The amino acid sequence of MUC-1 is shown in the following:

[SEQ ID NO: 19]
MTPGTQSPFFLLLLLTVLTVVTGSGHASSTPGGEK
ETSATQRSSVPSSTEKNAVSMTSSVLSSHSPGSSS
TTQGQDVTLAPATEPASGSAATWGQDVTSVPVT
RPALGSTTPPAHDVTSAPDNKPAPGSTAPPAHGVT
SAPDTRPAPGSTAPPAHGVTSAPDTRPAPGSTAPP
AHGVTSAPDTRPAPGSTAPPAHGVTSAPDTRPAPG
STAPPAHGVTSAPDTRPAPGSTAPPAHGVTSAPDT
RPAPGSTAPPAHGVTSAPDTRPAPGSTAPPAHGVT
SAPDTRPAPGSTAPPAHGVTSAPDTRPAPGSTAPP
AHGVTSAPDTRPAPGSTAPPAHGVTSAPDTRPAPG
STAPPAHGVTSAPDTRPAPGSTAPPAHGVTSAPDT
RPAPGSTAPPAHGVTSAPDTRPAPGSTAPPAHGVT
SAPDTRPAPGSTAPPAHGVTSAPDTRPAPGSTAPP
AHGVTSAPDTRPAPGSTAPPAHGVTSAPDTRPAPG
STAPPAHGVTSAPDTRPAPGSTAPPAHGVTSAPDT
RPAPGSTAPPAHGVTSAPDTRPAPGSTAPPAHGVT
SAPDTRPAPGSTAPPAHGVTSAPDTRPAPGSTAPP
AHGVTSAPDTRPAPGSTAPPAHGVTSAPDTRPAPG
STAPPAHGVTSAPDTRPAPGSTAPPAHGVTSAPDT
RPAPGSTAPPAHGVTSAPDTRPAPGSTAPPAHGVT
SAPDTRPAPGSTAPPAHGVTSAPDTRPAPGSTAPP
AHGVTSAPDTRPAPGSTAPPAHGVTSAPDTRPAPG
STAPPAHGVTSAPDTRPAPGSTAPPAHGVTSAPDT
RPAPGSTAPPAHGVTSAPDTRPAPGSTAPPAHGVT
SAPDTRPAPGSTAPPAHGVTSAPDTRPAPGSTAPP
AHGVTSAPDTRPAPGSTAPPAHGVTSAPDTRPAPG
STAPPAHGVTSAPDTRPAPGSTAPPAHGVTSAPDN
RPALGSTAPPVHNVTSASGSASGSASTLVHNGTSA
RATTTPASKSTPFSIPSHHSDTPTTLASHSTKTDA
SSTHHSSVPPLTSSNHSTSPQLSTGVSFFFLSFHI
SNLQFNSSLEDPSTDYYQELQRDISEMFLQIYKQG
GFLGLSNIKFRPGSVVVQLTLAFREGTINVHDVET
QFNQYKTEAASRYNLTISDVSVSDVPFPFSAQSGA
GVPGWGIALLVLVCVLVALAIVYLIALAVCQCRRK
NYGQLDIFPARDTYHPMSEYPTYHTHGRYVPPSST
DRSPYEKVSAGNGGSSLSYTNPAVAATSANL

Accordingly, a preferred complex of the first component (K) for use according to the present invention comprises an amino acid sequence according to SEQ ID NO: 19 or a fragment or a variant thereof as described herein.

Several epitopes of MUC-1 are known to the skilled person. Preferred MUC-1 epitopes, which are preferably comprised by the complex of the first component (K) for use according to the present invention, include the following epitopes (the epitope sequences shown in the following are fragments of the above MUC-1 sequence and are, thus, shown in the above MUC-1 sequence underlined; each of the following epitope sequences may refer to one epitope or more than one (overlapping) epitopes):

[SEQ ID NO: 20]
GSTAPPVHN
[SEQ ID NO: 21]
TAPPAHGVTS

Accordingly, a preferred complex of the first component (K) (for use) according to the present invention comprises an amino acid sequence according to SEQ ID NO: 20 and/or an amino acid sequence according to SEQ ID NO: 21.

### Carcino-Embryonic Antigen (CEA)

In some embodiments, the antigenic domain of said first component (K) pr at les one epitope of CEA. CEA is an intracellular adhesion glycoprotein. CEA is normally produced in gastrointestinal tissue during fetal development, but the production stops before birth.

Therefore, CEA is usually present only at very low levels in the blood of healthy adults. The amino acid sequence of CEA is shown in the following:

[SEQ ID NO: 24]
MESPSAPPHRWCIPWQRLLLTASLLTFWNPPTTAK
LTIESTPFNVAEGKEVLLLVHNLPQHLFGYSWYKG
ERVDGNRQIIGYVIGTQQATPGPAYSGREIIYPNA
SLLIQNIIQNDTGFYTLHVIKSDLVNEEATGQFRV
YPELPKPSISSNNSKPVEDKDAVAFTCEPETQDAT
YLWWVNNQSLPVSPRLQLSNGNRTLTLFNVTRNDT
ASYKCETQNPVSARRSDSVILNVLYGPDAPTISPL
NTSYRSGENLNLSCHAASNPPAQYSWFVNGTFQQS
TQELFIPNITVNNSGSYTCQAHNSDTGLNRTTVTT
ITVYAEPPKPFITSNNSNPVEDEDAVALTCEPEIQ
NTTYLWWVNNQSLPVSPRLQLSNDNRTLTLLSVTR
NDVGPYECGIQNKLSVDHSDPVILNVLYGPDDPTI
SPSYTYYRPGVNLSLSCHAASNPPAQYSWLIDGNI
QQHTQELFISNITEKNSGLYTCQANNSASGHSRTT
VKTITVSAELPKPSISSNNSKPVEDKDAVAFTCEP
EAQNTTYLVWWNGQSLPVSPRLQLSNGNRTLTLFN
VTRNDARAYVCGIQNSVSANRSDPVTLDVLYGPDT
PIISPPDSSYLSGANLNLSCHSASNPSPQYSWRIN
GIPQQHTQVLFIAKITPNNNGTYACFVSNLATGRN
NSIVKSITVSASGTSPGLSAGATVGIMIGVLVGVA
L

Accordingly, a preferred complex of the first component (K) (for use) according to the present invention comprises an amino acid sequence according to SEQ ID NO: 24 or a fragment or a variant thereof as described herein. Preferably, the antigenic domain of said first component (K) comprises a peptide having an amino acid sequence according to SEQ ID NO: 24, or a fragment thereof having a length of at least 10 amino acids, or a functional sequence variant thereof having at least 70%, 75%, 80%, 85%, 90% or 95% sequence identity. More preferably, the antigenic domain comprises a peptide having an amino acid sequence according to SEQ ID NO: 25 or a functional sequence variant thereof having at least 70%, 75%, 80%, 85%, 90% or 95% sequence identity.

Several epitopes of CEA are known to the skilled person. Preferred CEA epitopes, which are preferably comprised by the complex of the first component (K) (for use) according to the present invention, include the following epitopes (the epitope sequences shown in the following are fragments of the above CEA sequence and are, thus, shown in the above CEA sequence underlined; each of the following epitope sequences may refer to one epitope or more than one (overlapping) epitopes):

[SEQ ID NO: 26]
YLSGANLNLS
[SEQ ID NO: 27]
SWRINGIPQQ

Accordingly, a preferred complex of the first component (K) (for use) according to the present invention comprises an amino acid sequence according to SEQ ID NO: 26 and/or an amino acid sequence according to SEQ ID NO: 27. In other words, the antigenic domain of said first component (K) preferably comprises a peptide having an amino acid sequence according to SEQ ID NO: 26 and/or a peptide having an amino acid sequence according to SEQ ID NO: 27.

Accordingly, it is preferred that the complex of the first component (K) for use according to the present invention comprises an epitope of CEA. More preferably, the complex comprises a peptide having an amino acid sequence according to SEQ ID NO: 24, or a fragment thereof having a length of at least 10 amino acids (preferably at least 15 amino acids, more preferably at least 20 amino acids, even more preferably at least 25 amino acids and most preferably at least 30 amino acids), or a functional sequence variant thereof having at least 70% sequence identity (preferably at least 75%, more preferably at least 80%, even preferably at least 85%, still more preferably at least 90%, particularly preferably at least 95%, most preferably at least 99% sequence identity). Even more preferably, the complex of the first component (K) comprises a peptide having an amino acid sequence according to SEQ ID NO: 26 and/or a peptide having an amino acid sequence according to SEQ ID NO: 27. Most preferably, the complex of the first component (K) comprises a peptide having an amino acid sequence according to SEQ ID NO: 25 or a functional sequence variant thereof having at least 70% sequence identity (preferably at least 75%, more preferably at least 80%, even preferably at least 85%, still more preferably at least 90%, particularly preferably at least 95%, most preferably at least 99% sequence identity).

### Transforming Growth Factor Beta Receptor 2 (TGFβR2)

TGFβ receptors are single pass serine/threonine kinase receptors. They exist in several different isoforms. TGFβR2 (UniProtKB P37137) is a transmembrane protein that has a protein kinase domain, forms a heterodimeric complex with another receptor protein, and binds TGF-beta. This receptor/ligand complex phosphorylates proteins, which then enter the nucleus and regulate the transcription of a subset of genes related to cell proliferation.

[SEQ ID NO: 28]
MGRGLLRGLWPLHIVLWTRIASTIPPHVQKSVNNDM
IVTDNNGAVKFPQLCKFCDVRFSTCDNQKSCMSNC
SITSICEKPQEVCVAVWRKNDENITLETVCHDPKL
PYHDFILEDAASPKCIMKEKKKPGETFFMCSCSSD
ECNDNIIFSEEYNTSNPDLLLVIFQVTGISLLPPL
GVAISVIIIFYCYRVNRQQKLSSTWETGKTRKLME
FSEHCAIILEDDRSDISSTCANNINHNTELLPIEL
DTLVGKGRFAEVYKAKLKQNTSEQFETVAVKIFPY
EEYASWKTEKDIFSDINLKHENILQFLTAEERKTE
LGKQYWLITAFHAKGNLQEYLTRHVISWEDLRKLG
SSLARGIAHLHSDHTPCGRPKMPIVHRDLKSSNIL
VKNDLTCCLCDFGLSLRLDPTLSVDDLANSGQVGT
ARYMAPEVLESRMNLENVESFKQTDVYSMALVLWE
MTSRCNAVGEVKDYEPPFGSKVREHPCVESMKDNV
LRDRGRPEIPSFWLNHQGIQMVCETLTECWDHDPE
ARLTAQCVAERFSELEHLDRLSGRSCSEEKIPEDG
SLNTTK

Accordingly, a preferred complex of the first component (K) of the first component of the vaccine for use according to the present invention comprises an amino acid sequence according to SEQ ID NO: 28 or a fragment or a variant thereof as described herein, comprising at least one antigen for use in the antigenic domain of the invention.

### P53

P53 (UniProtKB P04637) is a tumor suppressor protein having a role in preventing genome mutation. P53 has many mechanisms of anticancer function and plays a role in apoptosis, genomic stability, and inhibition of angiogenesis. In its anti-cancer role, p53 works through several mechanisms: it an activate DNA repair proteins when DNA has sustained damage; it can arrest growth by holding the cell cycle at the G1/S regulation point on DNA damage recognition; and it can initiate apoptosis.

[SEQ ID NO: 29]
MEEPQSDPSVEPPLSQETFSDLWKLLPENN
VLSPLPSQAMDDLMLSPDDIEQWFTEDPGP
DEAPRMPEAAPPVAPAPAAPTPAAPAPAPS
WPLSSSVPSQKTYQGSYGFRLGFLHSGTAK
SVTCTYSPALNKMFCQLAKTCPVQLWVDS
TPPPGTRVRAMAIYKQSQHMTEVVRRCPHH
ERCSDSDGLAPPQHLIRVEGNLRVEYLDDR
NTFRHSVVVPYEPPEVGSDCTTIHYNYMCN
SSCMGGMNRRPILTIITLEDSSGNLLGRNS
FEVRVCACPGRDRRTEEENLRKKGEPHHEL
PPGSTKRALPNNTSSSPQPKKKPLDGEYFT
LQIRGRERFEMFRELNEALELKDAQAGKEP
GGSRAHSSHLKSKKGQSTSRHKKLMFKTEG
PDSD

Accordingly, a preferred complex of the first component (K) of the vaccine for use according to the present invention comprises an amino acid sequence according to SEQ ID NO: 29 or a fragment or a variant thereof as described herein, comprising at least one antigen for use in the antigenic domain of the invention.

### Kirsten Ras (KRas)

GTPase KRas also known as V-Ki-ras2 Kirsten rat sarcoma viral oncogene homolog and KRAS, performs an essential function in normal tissue signaling, and the mutation of a KRAS gene is an essential step in the development of many cancers. Like other members of the ras subfamily, the KRAS protein is a GTPase and is an early player in many signal transduction pathways. KRAS is usually tethered to cell membranes because of the presence of an isoprene group on its C-terminus. The amino acid sequence of KRas is shown in the following:

[SEQ ID NO: 30]
MTEYKLVVVGAGGVGKSALTIQLIQNHFVDEYDPT
IEDSYRKQWIDGETCLLDILDTAGQEEYSAMRDQY
MRTGEGFLCVFAINNTKSFEDIHHYREQIKRVKDS
EDVPMVLVGNKCDLPSRTVDTKQAQDLARSYGIPF
IETSAKTRQRVEDAFYTLVREIRQYRLKKISKEEK
TPGCVKIKKCIIM

Accordingly, a preferred complex of the first component (K) (for use) according to the present invention comprises an amino acid sequence according to SEQ ID NO: 30 or a fragment or a variant thereof as described herein.

Several epitopes of Kirsten Ras are known to the skilled person. A preferred Kirsten Ras epitope, which is preferably comprised by the complex of the first component (K) (for use) according to the present invention, includes the following epitope (the epitope sequence shown in the following is a fragment of the above Kirsten Ras sequence and is, thus, shown in the above Kirsten Ras sequence underlined; the following epitope sequence may refer to one epitope or more than one (overlapping) epitopes):

[SEQ ID NO: 31]
VVVGAGGVG

Accordingly, a preferred complex of the first component (K) (for use) according to the present invention comprises an amino acid sequence according to SEQ ID NO: 31.

### O-Linked N-Acetylqlucosamine (GIcNAc) Transferase (OGT)

OGT (O-Linked N-Acetylglucosamine (GIcNAc) Transferase, O-GIcNAc transferase, OGTase, O-linked N-acetylglucosaminyltransferase, uridine diphospho-N-acetylglucosamine:polypeptide beta-N-acetylglucosaminyltransferase, protein O-linked beta-N-acetylglucosamine transferase, UniProtKB 015294) is an enzyme with system name UDP-N-acetyl-D-glucosamine:protein-O-beta-N-acetyl-D-glucosaminyl transferase) is an enzyme with system name “UDP-N-acetyl-D-glucosamine:protein-O-beta-N-acetyl-D-glucosaminyl transferase”. OGT catalyzes the addition of a single N-acetylglucosamine in O-glycosidic linkage to serine or threonine residues of intracellular proteins. OGT is a part of a host of biological functions within the human body. OGT is involved in the resistance of insulin in muscle cells and adipocytes by inhibiting the Threonine 308 phosphorylation of AKT1, increasing the rate of IRS1 phosphorylation (at Serine 307 and Serine 632/635), reducing insulin signaling, and glycosylating components of insulin signals. Additionally, OGT catalyzes intracellular glycosylation of serine and threonine residues with the addition of N-acetylglucosamine. Studies show that OGT alleles are vital for embryogenesis, and that OGT is necessary for intracellular glycosylation and embryonic stem cell vitality. OGT also catalyzes the posttranslational modification that modifies transcription factors and RNA polymerase II, however the specific function of this modification is mostly unknown. The sequence of OGT is shown below:

[SEQ ID NO: 32]
MASSVGNVADSTEPTKRMLSFQGLAELAHREYQAG
DFEAAERHCMQLWRQEPDNTGVLLLLSSIHFQCRR
LDRSAHFSTLAIKQNPLLAEAYSNLGNVYKERGQL
QEAIEHYRHALRLKPDFIDGYINLAAALVAAGDME
GAVQAYVSALQYNPDLYCVRSDLGNLLKALGRLEE
AKACYLKAIETQPNFAVAWSNLGCVFNAQGEIWLA
IHHFEKAVTLDPNFLDAYINLGNVLKEARIFDRAV
AAYLRALSLSPNHAVVHGNLACVYYEQGLIDLAID
TYRRAIELQPHFPDAYCNLANALKEKGSVAEAEDC
YNTALRLCPTHADSLNNLANIKREQGNIEEAVRLY
RKALEVFPEFAAAHSNLASVLQQQGKLQEALMHYK
EAIRISPTFADAYSNMGNTLKEMQDVQGALQCYTR
AIQINPAFADAHSNLASIHKDSGNIPEAIASYRTA
LKLKPDFPDAYCNLAHCLQIVCDWTDYDERMKKLV
SIVADQLEKNRLPSVHPHHSMLYPLSHGFRKAIAE
RHGNLCLDKINVLHKPPYEHPKDLKLSDGRLRVGY
VSSDFGNHPTSHLMQSIPGMHNPDKFEVFCYALSP
DDGTNFRVKVMAEANHFIDLSQIPCNGKAADRIHQ
DGIHILVNMNGYTKGARNELFALRPAPIQAMWLGY
PGTSGALFMDYIITDQETSPAEVAEQYSEKLAYMP
HTFFIGDHANMFPHLKKKAVIDFKSNGHIYDNRIV
LNGIDLKAFLDSLPDVKIVKMKCPDGGDNADSSNT
ALNMPVIPMNTIAEAVIEMINRGQIQITINGFSIS
NGLATTQINNKAATGEEVPRTIIVTTRSQYGLPED
AIVYCNFNQLYKIDPSTLQMWANILKRVPNSVLWL
LRFPAVGEPNIQQYAQNMGLPQNRIIFSPVAPKEE
HVRRGQLADVCLDTPLCNGHTTGMDVLWAGTPMVT
MPGETLASRVAASQLTCLGCLELIAKNRQEYEDIA
VKLGTDLEYLKKVRGKVWKQRISSPLFNTKQYTME
LERLYLQMWEHYAAGNKPDHMIKPVEVTESA

Accordingly, a preferred complex of the first component (K) of the vaccine for use according to the present invention comprises an amino acid sequence according to SEQ ID NO: 32 or a fragment or a variant thereof as described herein, comprising at least one antigen for use in the antigenic domain of the invention.

### Caspase 5 (CASP5)

Caspase 5 (UniProtKB P51878) is an enzyme that proteolytically cleaves other proteins at an aspartic acid residue, and belongs to a family of cysteine proteases called caspases. It is an inflammatory caspase, along with caspase 1, caspase 4 and the murine caspase 4 homolog caspase 11, and has a role in the immune system. The amino acid sequence of CASP5 is shown below:

[SEQ ID NO: 33]
MAEDSGKKKRRKNFEAMFKGILQSGLDNFVINHMLK
NNVAGQTSIQTLVPNTDQKSTSVKKDNHKKKTVKM
LEYLGKDVLHGVFNYLAKHDVLTLKEEEKKKYYDT
KIEDKALILVDSLRKNRVAHQMFTQTLLNMDQKIT
SVKPLLQIEAGPPESAESTNILKLCPREEFLRLCK
KNHDEIYPIKKREDRRRLALIICNTKFDHLPARNG
AHYDIVGMKRLLQGLGYTVVDEKNLTARDMESVLR
AFAARPEHKSSDSTFLVLMSHGILEGICGTAHKKK
KPDVLLYDTIFQIFNNRNCLSLKDKPKVIIVQACR
GEKHGELWVRDSPASLALISSQSSENLEADSVCKI
HEEKDFIAFCSSTPHNVSWRDRTRGSIFITELITC
FQKYSCCCHLMEIFRKVQKSFEVPQAKAQMPTIER
ATLTRDFYLFPGN

Accordingly, a preferred complex of the first component (K) of the vaccine for use according to the present invention comprises an amino acid sequence according to SEQ ID NO: 33 or a fragment or a variant thereof as described herein, comprising at least one antigen for use in the antigenic domain of the invention.

### Colorectal Tumor-Associated Antigen-1 (COA-1)

COA-1 was identified in 2003 by Maccalli et al. (Maccalli, C., et al., Identification of a colorectal tumor-associated antigen (COA-1) recognized by CD4(+) T lymphocytes. Cancer Res, 2003. 63(20): p. 6735-43) as strongly expressed by colorectal and melanoma cells (no data available). Its mutation may interfere with the differential recognition of tumor and normal cells. The amino acid sequence of COA-1 (UniProtKB Q5T124) is shown below:

[SEQ ID NO: 34]
MSSPLASLSKTRKVPLPSEPMNPGRRGIRIYGDEDE
VDMLSDGCGSEEKISVPSCYGGIGAPVSRQVPASH
DSELMAFMTRKLWDLEQQVKAQTDEILSKDQKIAA
LEDLVQTLRPHPAEATLQRQEELETMCVQLQRQVR
EMERFLSDYGLQWVGEPMDQEDSESKTVSEHGERD
WMTAKKFWKPGDSLAPPEVDFDRLLASLQDLSELV
VEGDTQVTPVPGGARLRTLEPIPLKLYRNGIMMFD
GPFQPFYDPSTQRCLRDILDGFFPSELQRLYPNGV
PFKVSDLRNQVYLEDGLDPFPGEGRVVGRQLMHKA
LDRVEEHPGSRMTAEKFLNRLPKFVIRQGEVIDIR
GPIRDTLQNCCPLPARIQEIVVETPTLAAERERSQ
ESPNTPAPPLSMLRIKSENGEQAFLLMMQPDNTIG
DVRALLAQARVMDASAFEIFSTFPPTLYQDDTLTL
QAAGLVPKAALLLRARRAPKSSLKFSPGPCPGPGP
GPSPGPGPGPSPGPGPGPSPCPGPSPSPQ

Accordingly, a preferred complex of the first component (K) of the vaccine for use according to the present invention comprises an amino acid sequence according to SEQ ID NO: 34 or a fragment or a variant thereof as described herein, comprising at least one antigen for use in the antigenic domain of the invention.

### Squamous Cell Carcinoma Antigen Recognized by T-Cells (SART)

Within the SART family, SART-3/SART1-3 is most preferred. Thus, the complex of the first component (K) (for use) according to the present invention preferably comprises an antigen of the SART-family (a “SART” antigen) or an epitope thereof; the complex for use according to the present invention more preferably comprises SART-3 or an epitope thereof. Squamous cell carcinoma antigen recognized by T-cells 3 possesses tumor epitopes capable of inducing HLA-A24-restricted and tumor-specific cytotoxic T lymphocytes in cancer patients. SART-3 is thought to be involved in the regulation of mRNA splicing.

### IL13Ralpha2

IL13Ralpha2 binds interleukin 13 (IL-13) with very high affinity (and can therefore sequester it) but does not allow IL-4 binding. It acts as a negative regulator of both IL-13 and IL-4, however the mechanism of this is still undetermined. The amino acid sequence of IL13Ralpha2 is shown in the following:

[SEQ ID NO: 35]
MAFVCLAIGCLYTFLISTTFGCTSSSDTEIKVNPP
QDFEIVDPGYLGYLYLQWQPPLSLDHFKECTVEYE
LKYRNIGSETWKTIITKNLHYKDGFDLNKGIEAKI
HTLLPWQCTNGSEVQSSWAETTYWISPQGIPETKV
QDMDCVYYNWQYLLCSWKPGIGVLLDTNYNLFYWY
EGLDHALQCVDYIKADGQNIGCRFPYLEASDYKDF
YICVNGSSENKPIRSSYFTFQLQNIVKPLPPVYLT
FTRESSCEIKLKWSIPLGPIPARCFDYEIEIREDD
TTLVTATVENETYTLKTTNETRQLCFVVRSKVNIY
CSDDGIWSEWSDKQCWEGEDLSKKTLLRFWLPFGF
ILILVIFVTGLLLRKPNTYPKMIPEFFCDT

Accordingly, a preferred complex of the first component (K) of the vaccine for use according to the present invention comprises an amino acid sequence according to SEQ ID NO: 35 or a fragment or a variant thereof as described herein, comprising at least one antigen for use in the antigenic domain of the invention.

Several epitopes of IL13Ralpha2 are known to the skilled person. A preferred IL13Ralpha2 epitope, which is preferably comprised by the complex of the first component (K) (for use) according to the present invention, includes the following epitope (the epitope sequence shown in the following is a fragment of the above IL13Ralpha2 sequence and is, thus, shown in the above IL13Ralpha2 sequence underlined; the following epitope sequence may refer to one epitope or more than one (overlapping) epitopes):

[SEQ ID NO: 36]
LPFGFIL

Accordingly, a preferred complex of the first component (K) (for use) according to the present invention comprises an amino acid sequence according to SEQ ID NO: 36.

### KOC1

KOC1 (UniProtKB 000425), also known as insulin-like growth factor 2 mRNA-binding protein 3 (IGF2BP3), IMP3, KOC1, VICKZ3 is an mRNA binding protein. No expression data are however available, the sequence of which is as depicted below:

[SEQ ID NO: 37]
MNKLYIGNLSENAAPSDLESIFKDAKIPVSGPFLV
KTGYAFVDCPDESWALKAIEALSGKIELHGKPIEV
EHSVPKRQRIRKLQIRNIPPHLQWEVLDSLLVQYG
VVESCEQVNTDSETAVVNVTYSSKDQARQALDKLN
GFQLENFTLKVAYIPDEMAAQQNPLQQPRGRRGLG
QRGSSRQGSPGSVSKQKPCDLPLRLLVPTQFVGAI
IGKEGATIRNITKQTQSKIDVHRKENAGAAEKSIT
ILSTPEGTSAACKSILEIMHKEAQDIKFTEEIPLK
ILAHNNFVGRLIGKEGRNLKKIEQDTDTKITISPL
QELTLYNPERTITVKGNVETCAKAEEEIMKKIRES
YENDIASMNLQAHLIPGLNLNALGLFPPTSGMPPP
TSGPPSAMTPPYPQFEQSETETVHLFIPALSVGAI
IGKQGQHIKQLSRFAGASIKIAPAEAPDAKVRMVI
ITGPPEAQFKAQGRIYGKIKEENFVSPKEEVKLEA
HIRVPSFAAGRVIGKGGKTVNELQNLSSAEVVVPR
DQTPDENDQVVVKITGHFYACQVAQRKIQEILTQV
KQHQQQKALQSGPPQSRRK

Accordingly, a preferred complex of the first component (K) of the vaccine for use according to the present invention comprises an amino acid sequence according to SEQ ID NO: 37 or a fragment or a variant thereof as described herein, comprising at least one antigen for use in the antigenic domain of the invention.

### TOMM34

TOMM34 (UniProtKB Q15785) is involved in the import of precursor proteins into mitochondria. Many epitopes thereof are known to the skilled person, which can selected from the amino acid sequence shown below:

[SEQ ID NO: 38]
MAPKFPDSVEELRAAGNESFRNGQYAEASALYGRA
LRVLQAQGSSDPEEESVLYSNRAACHLKDGNCRDC
IKDCTSALALVPFSIKPLLRRASAYEALEKYPMAY
VDYKTVLQIDDNVTSAVEGINRMTRALMDSLGPEW
RLKLPSIPLVPVSAQKRWNSLPSENHKEMAKSKSK
ETTATKNRVPSAGDVEKARVLKEEGNELVKKGNHK
KAIEKYSESLLCSNLESATYSNRALCYLVLKQYTE
AVKDCTEALKLDGKNVKAFYRRAQAHKALKDYKSS
FADISNLLQIEPRNGPAQKLRQEVKQNLH

Accordingly, a preferred complex of the first component (K) of the vaccine for use according to the present invention comprises an amino acid sequence according to SEQ ID NO: 38 or a fragment or a variant thereof as described herein, comprising at least one antigen for use in the antigenic domain of the invention.

### RNF 43

RNF43 (UniProtKB Q68DV7) is a RING-type E3 ubiquitin ligase and is predicted to contain a transmembrane domain, a protease-associated domain, an ectodomain, and a cytoplasmic RING domain. RNF43 is thought to negatively regulate Wnt signaling, and expression of RNF43 results in an increase in ubiquitination of frizzled receptors, an alteration in their subcellular distribution, resulting in reduced surface levels of these receptors. Many epitopes thereof are known to the skilled person, with the amino acid sequence of RNF43 shown below:

[SEQ ID NO: 39]
MSGGHQLQLAALWPWLLMATLQAGFGRTGLVLAAA
VESERSAEQKAIIRVIPLKMDPTGKLNLTLEGVFA
GVAEITPAEGKLMQSHPLYLCNASDDDNLEPGFIS
IVKLESPRRAPRPCLSLASKARMAGERGASAVLFD
ITEDRAAAEQLQQPLGLTWPVVLIWGNDAEKLMEF
VYKNQKAHVRIELKEPPAWPDYDVWILMTVVGTIF
VIILASVLRIRCRPRHSRPDPLQQRTAWAISQLAT
RRYQASCRQARGEWPDSGSSCSSAPVCAICLEEFS
EGQELRVISCLHEFHRNCVDPWLHQHRTCPLCMFN
ITEGDSFSQSLGPSRSYQEPGRRLHLIRQHPGHAH
YHLPAAYLLGPSRSAVARPPRPGPFLPSQEPGMGP
RHHRFPRAAHPRAPGEQQRLAGAQHPYAQGWGLSH
LQSTSQHPAACPVPLRRARPPDSSGSGESYCTERS
GYLADGPASDSSSGPCHGSSSDSVVNCTDISLQGV
HGSSSTFCSSLSSDFDPLVYCSPKGDPQRVDMQPS
VTSRPRSLDSWPTGETQVSSHVHYHRHRHHHYKKR
FQWHGRKPGPETGVPQSRPPIPRTQPQPEPPSPDQ
QVTRSNSAAPSGRLSNPQCPRALPEPAPGPVDASS
ICPSTSSLFNLQKSSLSARHPQRKRRGGPSEPTPG
SRPQDATVHPACQIFPHYTPSVAYPWSPEAHPLIC
GPPGLDKRLLPETPGPCYSNSQPVWLCLTPRQPLE
PHPPGEGPSEWSSDTAEGRPCPYPHCQVLSAQPGS
EEELEELCEQAV

Accordingly, a preferred complex of the first component (K) of the vaccine for use according to the present invention comprises an amino acid sequence according to SEQ ID NO: 39 or a fragment or a variant thereof as described herein, comprising at least one antigen for use in the antigenic domain of the invention.

### Vascular Endothelial Growth Factor (VEGF)/Vascular Endothelial Growth Factor Receptor (VEGFR)

Vascular endothelial growth factor (VEGF, UniProtKB P15692), originally known as vascular permeability factor (VPF), is a signal protein produced by cells that stimulates vasculogenesis and angiogenesis. It is part of the system that restores the oxygen supply to tissues when blood circulation is inadequate. VEGF's normal function is to create new blood vessels during embryonic development, new blood vessels after injury, muscle following exercise, and new vessels (collateral circulation) to bypass blocked vessels. There are three main subtypes of the receptors for VEGF (VEGFR), namely VEGFR1 (UniProtKB P17948), VEGFR2 (UniProtKB P35968) and VEGFR3 (UniProtKB P35916). The sequences of VEGF, VEGFR1, VEGFR2 and VEGFR3 are hereby incorporated by reference.

Accordingly, a preferred complex of the first component (K) of the vaccine for use according to the present invention comprises an amino acid sequence according to UniProtKB P17948, UniProtKB P35968) and VEGFR3 (UniProtKB P35916) or a fragment of these sequences or a sequence variant thereof, comprising at least one antigen for use in the antigenic domain of the invention.

Beta Subunit of Human Chorionic Gonadotropin (phCG)

Human chorionic gonadotropin (hCG) is a hormone produced by the embryo following implantation. Some cancerous tumors produce this hormone; therefore, elevated levels measured when the patient is not pregnant can lead to a cancer diagnosis. hCG is heterodimeric with an a (alpha) subunit identical to that of luteinizing hormone (LH), follicle-stimulating hormone (FSH), thyroid-stimulating hormone (TSH), and β (beta) subunit that is unique to hCG. The β-subunit of hCG gonadotropin (beta-hCG) contains 145 amino acids and is encoded by six highly homologous genes.

### EpCAM

EpCAM (UniProtKB P16422) is a glycoprotein mediating cellular adhesion. The amino acid sequence of EpCAM is shown in the following:

[SEQ ID NO: 40]
MAPPQVLAFGLLLAAATATFAAAQEECVCENYKLA
VNCFVNNNRQCQCTSVGAQNTVICSKLAAKCLVMK
AEMNGSKLGRRAKPEGALQNNDGLYDPDCDESGLF
KAKQCNGTSMCWCVNTAGVRRTDKDTEITCSERVR
TYWIIIELKHKAREKPYDSKSLRTALQKEITTRYQ
LDPKFITSILYENNVITIDLVQNSSQKTQNDVDIA
DVAYYFEKDVKGESLFHSKKMDLTVNGEQLDLDPG
QTLIYYVDEKAPEFSMQGLKAGVIAVIVVVVIAWA
GIVVLVISRKKRMAKYEKAEIKEMGEMHRELNA

Accordingly, a preferred complex of the first component (K) (for use) according to the present invention comprises an amino acid sequence according to SEQ ID NO: 40 or a fragment or a variant thereof as described herein.

Several epitopes of EpCAM are known to the skilled person. A preferred EpCAM epitope, which is preferably comprised by the complex of the first component (K) (for use) according to the present invention, includes the following epitope (the epitope sequence shown in the following is a fragment of the above EpCAM sequence and is, thus, shown in the above EpCAM sequence underlined; the following epitope sequence may refer to one epitope or more than one (overlapping) epitopes):

[SEQ ID NO: 41]
GLKAGVIAV

Accordingly, a preferred complex of the first component (K) (for use) according to the present invention comprises an amino acid sequence according to SEQ ID NO: 41 or a fragment or a variant thereof as described herein.

### HER-2/neu

Her-2 belongs to the EGFR (epidermal growth factor receptor) family. Many HLA-A epitopes are known to the skilled person. The amino acid sequence of HER2 is shown in the following:

[SEQ ID NO: 42]
MELAALCRWGLLLALLPPGAASTQVCTGTDMKLRL
PASPETHLDMLRHLYQGCQVVQGNLELTYLPTNAS
LSFLQDIQEVQGYVLIAHNQVRQVPLQRLRIVRGT
QLFEDNYALAVLDNGDPLNNTTPVTGASPGGLREL
QLRSLTEILKGGVLIQRNPQLCYQDTILWKDIFHK
NNQLALTLIDTNRSRACHPCSPMCKGSRCWGESSE
DCQSLTRTVCAGGCARCKGPLPTDCCHEQCAAGCT
GPKHSDCLACLHFNHSGICELHCPALVTYNTDTFE
SMPNPEGRYTFGASCVTACPYNYLSTDVGSCTLVC
PLHNQEVTAEDGTQRCEKCSKPCARVCYGLGMEHL
REVRAVTSANIQEFAGCKKIFGSLAFLPESFDGDP
ASNTAPLQPEQLQVFETLEEITGYLYISAWPDSLP
DLSVFQNLQVIRGRILHNGAYSLTLQGLGISWLGL
RSLRELGSGLALIHHNTHLCFVHTVPWDQLFRNPH
QALLHTANRPEDECVGEGLACHQLCARGHCWGPGP
TQCVNCSQFLRGQECVEECRVLQGLPREYVNARHC
LPCHPECQPQNGSVTCFGPEADQCVACAHYKDPPF
CVARCPSGVKPDLSYMPIWKFPDEEGACQPCPINC
THSCVDLDDKGCPAEQRASPLTSIISAVVGILLVV
VLGVVFGILIKRRQQKIRKYTMRRLLQETELVEPL
TPSGAMPNQAQMRILKETELRKVKVLGSGAFGTVY
KGIWIPDGENVKIPVAIKVLRENTSPKANKEILDE
AYVMAGVGSPYVSRLLGICLTSTVQLVTQLMPYGC
LLDHVRENRGRLGSQDLLNWCMQIAKGMSYLEDVR
LVHRDLAARNVLVKSPNHVKITDFGLARLLDIDET
EYHADGGKVPIKWMALESILRRRFTHQSDVWSYGV
TVWELMTFGAKPYDGIPAREIPDLLEKGERLPQPP
ICTIDVYMIMVKCWMIDSECRPRFRELVSEFSRMA
RDPQRFVVIQNEDLGPASPLDSTFYRSLLEDDDMG
DLVDAEEYLVPQQGFFCPDPAPGAGGMVHHRHRSS
STRSGGGDLTLGLEPSEEEAPRSPLAPSEGAGSDV
FDGDLGMGAAKGLQSLPTHDPSPLQRYSEDPTVPL
PSETDGYVAPLTCSPQPEYVNQPDVRPQPPSPREG
PLPAARPAGATLERPKTLSPGKNGVVKDVFAFGGA
VENPEYLTPQGGAAPQPHPPPAFSPAFDNLYYWDQ
DPPERGAPPSTFKGTPTAENPEYLGLDVPV

Accordingly, a preferred complex of the first component (K) (for use) according to the present invention comprises an amino acid sequence according to SEQ ID NO: 42 or a fragment or a variant thereof as described herein. As described above, suitable cancer/tumor epitopes of Her-2 are known from the literature or can be identified by using cancer/tumor epitope databases, e.g. from van der Bruggen P, Stroobant V, Vigneron N, Van den Eynde B. Peptide database: T cell-defined tumor antigens. Cancer Immun 2013; URL: www.cancerimmunity.org/peptide/, wherein human tumor antigens recognized by CD4⁺ or CD8⁺ T cells are classified into four major groups on the basis of their expression pattern, or from the database “Tantigen” (TANTIGEN version 1.0, Dec. 1, 2009; developed by Bioinformatics Core at Cancer Vaccine Center, Dana-Farber Cancer Institute; URL: cvc.dfci.harvard.edu/tadb/).

### WT1

WT1 (Wilms tumor protein, UniProtKB P19544) Transcription factor that plays an important role in cellular development and cell survival. The gene encoding WT1 is characterized by an complex structure, is located on chromosome 11. It is involved in cell growth and differentiation, and has a strong impact on consecutive stages of the functioning of the body. The WT1 gene may e,g. undergo many different mutations, as well as may be overexpressed without a mutation. The molecular basis of diseases such as Wilms tumor are congenital WT1 mutations, while somatic mutations of this gene occur in acute and chronic myeloid leukemia, myelodysplastic syndrome and also in some other blood neoplasms, as acute lymphoblood leukemia. Increased expression of this gene without its mutation is observed in leukemias and solid tumors. The amino acid sequence of VT 1 is shown below:

[SEQ ID NO: 43]
MGSDVRDLNALLPAVPSLGGGGGCALPVSGAAQWA
PVLDFAPPGASAYGSLGGPAPPPAPPPPPPPPPHS
FIKQEPSWGGAEPHEEQCLSAFTVHFSGQFTGTAG
ACRYGPFGPPPPSQASSGQARMFPNAPYLPSCLES
QPAIRNQGYSTVTFDGTPSYGHTPSHHAAQFPNHS
FKHEDPMGQQGSLGEQQYSVPPPVYGCHTPTDSCT
GSQALLLRTPYSSDNLYQMTSQLECMTWNQMNLGA
TLKGVAAGSSSSVKWTEGQSNHSTGYESDNHTTPI
LCGAQYRIHTHGVFRGIQDVRRVPGVAPTLVRSAS
ETSEKRPFMCAYPGCNKRYFKLSHLQMHSRKHTGE
KPYQCDFKDCERRFSRSDQLKRHQRRHTGVKPFQC
KTCQRKFSRSDHLKTHTRTHTGKTSEKPFSCRWPS
CQKKFARSDELVRHHNMHQRNMTKLQLAL

Accordingly, a preferred complex of the first component (K) of the vaccine for use according to the present invention comprises an amino acid sequence according to SEQ ID NO: 43 or a fragment or a variant thereof as described herein, comprising at least one antigen for use in the antigenic domain of the invention.

Preferably, the complex of the first component (K) according to the present invention comprises at least one tumor epitope, which is an epitope of an antigen selected from the group consisting of EpCAM, HER-2, MUC-1, TOMM34, RNF 43, KOC1, VEGFR, βhCG, survivin, CEA, TGFβR2, p53, KRas, OGT, CASP5, COA-1, MAGE, SART and IL13Ralpha2. More preferably, the complex for use according to the present invention comprises at least one tumor epitope, which is an epitope of an antigen selected from the group consisting of ASCL2, EpCAM, HER-2, MUC-1, TOMM34, RNF 43, KOC1, VEGFR, βhCG, survivin, CEA, TGFβR2, p53, KRas, OGT, CASP5, COA-1, MAGE, SART and IL13Ralpha2. Those antigens are particularly useful in the context of colorectal cancer. It is also preferred that the complex for use according to the present invention comprises at least one tumor antigen selected from the group consisting of EpCAM, HER-2, MUC-1, TOMM34, RNF 43, KOC1, VEGFR, βhCG, survivin, CEA, TGFβR2, p53, KRas, OGT, CASP5, COA-1, MAGE, SART and IL13Ralpha2, or a fragment thereof, or a sequence variant of a tumor antigen or a sequence variant of a fragment thereof. It is also preferred that the complex for use according to the present invention comprises at least one tumor antigen selected from the group consisting of ASCL2, EpCAM, HER-2, MUC-1, TOMM34, RNF 43, KOC1, VEGFR, βhCG, survivin, CEA, TGFβR2, p53, KRas, OGT, CASP5, COA-1, MAGE, SART and IL13Ralpha2, or a fragment thereof, or a sequence variant of a tumor antigen or a sequence variant of a fragment thereof.

Preferably, the complex of the first component (K) (for use) according to the present invention comprises at least one tumor epitope, which is an epitope of an antigen selected from the group consisting of EpCAM, MUC-1, survivin, CEA, KRas, MAGE-A3 and IL13Ralpha2, such as an epitope according to any of SEQ ID NOs 48, 50, 51, 22, 26, 27, 31, 44 and 36; preferably, the complex for use according to the present invention comprises at least one tumor epitope, which is an epitope of an antigen selected from the group consisting of EpCAM, MUC-1, survivin, CEA, KRas, MAGE-A3, IL13Ralpha2, and ASCL2, such as an epitope according to any of SEQ ID NOs 41, 20, 21, 22, 26, 27, 31, 44, 36, 16 and 17; more preferably the at least one tumor epitope is an epitope of an antigen selected from the group consisting of EpCAM, MUC-1, survivin, CEA, KRas and MAGE-A3, such as an epitope according to any of SEQ ID NOs 48, 20, 21, 22, 26, 27, 31 and 44; more preferably the at least one tumor epitope is an epitope of an antigen selected from the group consisting of EpCAM, MUC-1, survivin, CEA, KRas, MAGE-A3, and ASCL2, such as an epitope according to any of SEQ ID NOs 41, 20, 21, 22, 26, 27, 31, 44, 16 and 17; even more preferably the at least one tumor epitope is an epitope of an antigen selected from the group consisting of EpCAM, MUC-1, survivin and CEA, such as an epitope according to any of SEQ ID NOs 41, 20, 21, 23, 26 and 27; even preferably the at least one tumor epitope is an epitope of an antigen selected from the group consisting of EpCAM, MUC-1, survivin, CEA, and ASCL2 such as an epitope according to any of SEQ ID NOs 41, 20, 21, 22, 26, 27, 16 and 17; and most preferably the at least one tumor epitope is an epitope of an antigen selected from the group consisting of EpCAM, survivin, CEA, and ASCL2 such as an epitope according to any of SEQ ID NOs 41, 22, 26, 27, 16 and 16.

In a preferred embodiment, the at least one tumor epitope of the antigenic domain of the complex of the first component (K) of the vaccine according to the invention as disclosed above is an epitope of an antigen selected from the group consisting of MAGE-A3, MUC-1, PRAME, ASCL2, and NY-ESO-1, preferably the at least one tumor epitope of the antigenic domain of the vaccine according to the invention as disclosed above is an epitope of an antigen selected from the group consisting of MAGE-A3, MUC-1, PRAME, ASCL2, preferably the at least one tumor epitope of the antigenic domain of the vaccine according to the invention as disclosed above is an epitope of an antigen selected from the group consisting of MAGE-A3, MUC-1, PRAME, preferably the at least one tumor epitope of the antigenic domain of the vaccine according to the invention as disclosed above is an epitope of an antigen selected from the group consisting of MAGE-A3, MUC-1, ASCL2, preferably the at least one tumor epitope of the antigenic domain of the vaccine according to the invention as disclosed above is an epitope of an antigen selected from the group consisting of MAGE-A3, ASCL2, PRAME, preferably the at least one tumor epitope of the antigenic domain of the vaccine according to the invention as disclosed above is an epitope of an antigen selected from the group consisting of MAGE-A3, MUC-1, NY-ESO-1, preferably the at least one tumor epitope of the antigenic domain of the vaccine according to the invention as disclosed above is an epitope of an antigen selected from the group consisting of MAGE-A3, ASCL2, NY-ESO-1. In one embodiment, it is more preferred the antigenic domain of the vaccine according to the invention comprises at least one epitope of the antigen MAGE-A3, or ASCL2, or MUC1, or PRAME, or NY-ESO-1.

Preferably, the antigenic domain of the vaccine according to the invention preferably comprises
a) one or more epitopes of EpCAM or functional sequence variants thereof;
b) one or more epitopes of MUC-1 or functional sequence variants thereof;
c) one or more epitopes of survivin or functional sequence variants thereof;
d) one or more epitopes of CEA or functional sequence variants thereof;
e) one or more epitopes of KRas or functional sequence variants thereof; and/or
f) one or more epitopes of MAGE-A3 or functional sequence variants thereof.
g) one or more epitopes of ASCL2 or functional sequence variants thereof.

It is also preferred that the complex of the first component (K) (for use) according to the present invention comprises
i) one or more epitopes of EpCAM (such as the epitope according to SEQ ID NO: 41) or functional sequence variants thereof;
ii) one or more epitopes of MUC-1 (such as the epitope according to SEQ ID NO: 20 and/or the epitope according to SEQ ID NO: 21) or functional sequence variants thereof;
iii) one or more epitopes of survivin (such as the epitope according to SEQ ID NO: 22) or functional sequence variants thereof;
iv) one or more epitopes of CEA (such as the epitope according to SEQ ID NO: 26 and/or the epitope according to SEQ ID NO: 27) or functional sequence variants thereof;
v) one or more epitopes of KRas (such as the epitope according to SEQ ID NO: 31) or functional sequence variants thereof; and/or
vi) one or more epitopes of MAGE-A3 (such as the epitope according to SEQ ID NO: 44) or functional sequence variants thereof.

It is also preferred that the complex of the first component (K) (for use) according to the present invention comprises
i) one or more epitopes of EpCAM (such as the epitope according to SEQ ID NO: 41) or functional sequence variants thereof;
ii) one or more epitopes of MUC-1 (such as the epitope according to SEQ ID NO: 20 and/or the epitope according to SEQ ID NO: 21) or functional sequence variants thereof;
iii) one or more epitopes of survivin (such as the epitope according to SEQ ID NO: 22) or functional sequence variants thereof;
iv) one or more epitopes of CEA (such as the epitope according to SEQ ID NO: 26 and/or the epitope according to SEQ ID NO: 27) or functional sequence variants thereof;
v) one or more epitopes of KRas (such as the epitope according to SEQ ID NO: 31) or functional sequence variants thereof;
vi) one or more epitopes of MAGE-A3 (such as the epitope according to SEQ ID NO: 44) or functional sequence variants thereof; and/or
vii) one or more epitopes of ASCL2 (such as the epitope according to SEQ ID NO: 16 and/or the epitope according to SEQ ID NO: 17) or functional sequence variants thereof.

It is also preferred that the complex of the first component (K) (for use) according to the present invention comprises
a fragment of EpCAM comprising one or more epitopes or a functional sequence variant thereof;
a fragment of MUC-1 comprising one or more epitopes or a functional sequence variant thereof;
a fragment of survivin comprising one or more epitopes or a functional sequence variant thereof;
a fragment of CEA comprising one or more epitopes or a functional sequence variant thereof;
a fragment of KRas comprising one or more epitopes or a functional sequence variant thereof; and/or
a fragment of MAGE-A3 comprising one or more epitopes or a functional sequence variant thereof.

It is also preferred that the complex of the first component (K) (for use) according to the present invention comprises
a fragment of EpCAM comprising one or more epitopes or a functional sequence variant thereof;
a fragment of MUC-1 comprising one or more epitopes or a functional sequence variant thereof;
a fragment of survivin comprising one or more epitopes or a functional sequence variant thereof;
a fragment of CEA comprising one or more epitopes or a functional sequence variant thereof;
a fragment of KRas comprising one or more epitopes or a functional sequence variant thereof;
a fragment of MAGE-A3 comprising one or more epitopes or a functional sequence variant thereof; and/or
a fragment of ASCL2 comprising one or more epitopes or a functional sequence variant thereof.

As used herein, a “fragment” of an antigen comprises at least 10 consecutive amino acids of the antigen, preferably at least 15 consecutive amino acids of the antigen, more preferably at least 20 consecutive amino acids of the antigen, even more preferably at least 25 consecutive amino acids of the antigen and most preferably at least 30 consecutive amino acids of the antigen. Accordingly, a fragment of EpCAM comprises at least 10 consecutive amino acids of EpCAM (SEQ ID NO: 40), preferably at least 15 consecutive amino acids of EpCAM (SEQ ID NO: 40), more preferably at least 20 consecutive amino acids of EpCAM (SEQ ID NO: 40), even more preferably at least 25 consecutive amino acids of EpCAM (SEQ ID NO: 40) and most preferably at least 30 consecutive amino acids of EpCAM (SEQ ID NO: 40); a fragment of MUC-1 comprises at least 10 consecutive amino acids of MUC-1 (SEQ ID NO: 19), preferably at least 15 consecutive amino acids of MUC-1 (SEQ ID NO: 19), more preferably at least 20 consecutive amino acids of MUC-1 (SEQ ID NO: 19), even more preferably at least 25 consecutive amino acids of MUC-1 (SEQ ID NO: 19) and most preferably at least 30 consecutive amino acids of MUC-1 (SEQ ID NO: 19); a fragment of survivin comprises at least 10 consecutive amino acids of survivin (SEQ ID NO: 12), preferably at least 15 consecutive amino acids of survivin (SEQ ID NO: 12), more preferably at least 20 consecutive amino acids of survivin (SEQ ID NO: 12), even more preferably at least 25 consecutive amino acids of survivin (SEQ ID NO: 12) and most preferably at least 30 consecutive amino acids of survivin (SEQ ID NO: 12); a fragment of CEA comprises at least 10 consecutive amino acids of CEA (SEQ ID NO: 24), preferably at least 15 consecutive amino acids of CEA (SEQ ID NO: 24), more preferably at least 20 consecutive amino acids of CEA (SEQ ID NO: 24), even more preferably at least 25 consecutive amino acids of CEA (SEQ ID NO: 24) and most preferably at least 30 consecutive amino acids of CEA (SEQ ID NO: 24); a fragment of KRas comprises at least 10 consecutive amino acids of KRas (SEQ ID NO: 30), preferably at least 15 consecutive amino acids of KRas (SEQ ID NO: 30), more preferably at least 20 consecutive amino acids of KRas (SEQ ID NO: 30), even more preferably at least 25 consecutive amino acids of KRas (SEQ ID NO: 57) and most preferably at least 30 consecutive amino acids of KRas (SEQ ID NO: 30); and a fragment of MAGE-A3 comprises at least 10 consecutive amino acids of MAGE-A3 (SEQ ID NO: 10), preferably at least 15 consecutive amino acids of MAGE-A3 (SEQ ID NO: 10), more preferably at least 20 consecutive amino acids of MAGE-A3 (SEQ ID NO: 10), even more preferably at least 25 consecutive amino acids of MAGE-A3 (SEQ ID NO: 10) and most preferably at least 30 consecutive amino acids of MAGE-A3 (SEQ ID NO: 10). Moreover, a fragment of ASCL2 comprises at least 10 consecutive amino acids of ASCL2 (SEQ ID NO: 15), preferably at least 15 consecutive amino acids of ASCL2 (SEQ ID NO: 15), more preferably at least 20 consecutive amino acids of ASCL2 (SEQ ID NO: 15), even more preferably at least 25 consecutive amino acids of ASCL2 (SEQ ID NO: 15) and most preferably at least 30 consecutive amino acids of ASCL2 (SEQ ID NO: 15).

A functional sequence variant of such a fragment has an (amino acid) sequence, which is at least 70%, at least 75%, preferably at least 80%, more preferably at least 85%, even more preferably at least 90%, particularly preferably at least 95%, most preferably at least 99% identical to the reference sequence, and the epitope function of at least one, preferably all, epitope(s) comprised by the fragment is maintained.

Such a complex does preferably not comprise any epitope of HER-2, MUC-1, TOMM34, RNF 43, KOC1, VEGFR, βhCG, survivin, TGFβR2, p53, KRas, OGT, CASP5, COA-1, SART or IL13Ralpha2. More preferably, such a complex does not comprise any epitope of ASCL2, HER-2, MUC-1, TOMM34, RNF 43, KOC1, VEGFR, βhCG, survivin, TGFβR2, p53, KRas, OGT, CASP5, COA-1, SART or IL13Ralpha2.

It is also preferred that such a complex of the first component (K) of the invention comprises
viii) one or more epitopes of EpCAM (such as the epitope according to SEQ ID NO: 41) or functional sequence variants thereof;
ix) one or more epitopes of MUC-1 (such as the epitope according to SEQ ID NO: 20 and/or the epitope according to SEQ ID NO: 21) or functional sequence variants thereof;
x) one or more epitopes of CEA (such as the epitope according to SEQ ID NO: 26 and/or the epitope according to SEQ ID NO: 27) or functional sequence variants thereof; and
xi) one or more epitopes of MAGE-A3 (such as the epitope according to SEQ ID NO: 44) or functional sequence variants thereof.

In other words, the antigenic domain of said first component (K) preferably comprises
a) one or more epitopes of EpCAM or functional sequence variants thereof;
b) one or more epitopes of MUC-1 or functional sequence variants thereof;
d) one or more epitopes of CEA or functional sequence variants thereof; and
f) one or more epitopes of MAGE-A3 or functional sequence variants thereof.

Such a complex does preferably not comprise any epitope of HER-2, TOMM34, RNF 43, KOC1, VEGFR, βhCG, survivin, TGFβR2, p53, KRas, OGT, CASP5, COA-1, SART or IL13Ralpha2. More preferably, such a complex does not comprise any epitope of ASCL2, HER-2, TOMM34, RNF 43, KOC1, VEGFR, βhCG, survivin, TGFβR2, p53, KRas, OGT, CASP5, COA-1, SART or IL13Ralpha2.

It is also preferred that such a complex of the first component (K) of the invention comprises
xii) one or more epitopes of EpCAM (such as the epitope according to SEQ ID NO: 41) or functional sequence variants thereof;
xiii) one or more epitopes of MUC-1 (such as the epitope according to SEQ ID NO: 20 and/or the epitope according to SEQ ID NO: 21) or functional sequence variants thereof;
xiv) one or more epitopes of CEA (such as the epitope according to SEQ ID NO: 26 and/or the epitope according to SEQ ID NO: 27) or functional sequence variants thereof; and
xv) one or more epitopes of KRas (such as the epitope according to SEQ ID NO: 31) or functional sequence variants thereof.

In other words, the antigenic domain of said first component (K) preferably comprises
a) one or more epitopes of EpCAM or functional sequence variants thereof;
b) one or more epitopes of MUC-1 or functional sequence variants thereof;
d) one or more epitopes of CEA or functional sequence variants thereof; and
e) one or more epitopes of KRas or functional sequence variants thereof.

Such a complex does preferably not comprise any epitope of HER-2, TOMM34, RNF 43, KOC1, VEGFR, βhCG, survivin, TGFβR2, p53, OGT, CASP5, COA-1, MAGE, SART or IL13Ralpha2. More preferably, such a complex does not comprise any epitope of ASCL2, HER-2, TOMM34, RNF 43, KOC1, VEGFR, βhCG, survivin, TGFβR2, p53, OGT, CASP5, COA-1, MAGE, SART or IL13Ralpha2.

It is also preferred that such a complex of the first component (K) of the invention comprises
xvi) one or more epitopes of EpCAM (such as the epitope according to SEQ ID NO: 41) or functional sequence variants thereof;
xvii) one or more epitopes of survivin (such as the epitope according to SEQ ID NO: 22) or functional sequence variants thereof;
xviii) one or more epitopes of CEA (such as the epitope according to SEQ ID NO: 26 and/or the epitope according to SEQ ID NO: 27) or functional sequence variants thereof; and
xix) one or more epitopes of MAGE-A3 (such as the epitope according to SEQ ID NO: 44) or functional sequence variants thereof.

In other words, the antigenic domain of said first component (K) preferably comprises
a) one or more epitopes of EpCAM or functional sequence variants thereof;
c) one or more epitopes of survivin or functional sequence variants thereof;
d) one or more epitopes of CEA or functional sequence variants thereof; and
f) one or more epitopes of MAGE-A3 or functional sequence variants thereof.

Such a complex does preferably not comprise any epitope of HER-2, MUC-1, TOMM34, RNF 43, KOC1, VEGFR, βhCG, TGFβR2, p53, KRas, OGT, CASP5, COA-1, SART or IL13Ralpha2. More preferably, such a complex does not comprise any epitope of ASCL2, HER-2, MUC-1, TOMM34, RNF 43, KOC1, VEGFR, βhCG, TGFβR2, p53, KRas, OGT, CASP5, COA-1, SART or IL13Ralpha2.

In some embodiments, the antigenic domain of said first component (K) preferably comprises
a) one or more epitopes of EpCAM or functional sequence variants thereof;
d) one or more epitopes of CEA or functional sequence variants thereof; and
f) one or more epitopes of MAGE-A3 or functional sequence variants thereof.

It is also preferred that such a complex of the first component (K) of the invention comprises
xx) one or more epitopes of MUC-1 (such as the epitope according to SEQ ID NO: 20 and/or the epitope according to SEQ ID NO: 21) or functional sequence variants thereof;
xxi) one or more epitopes of survivin (such as the epitope according to SEQ ID NO: 22) or functional sequence variants thereof; and
xxii) one or more epitopes of MAGE-A3 (such as the epitope according to SEQ ID NO: 44) or functional sequence variants thereof.

In other words, the antigenic domain of said first component (K) preferably comprises
b) one or more epitopes of MUC-1 or functional sequence variants thereof;
c) one or more epitopes of survivin or functional sequence variants thereof; and/or
f) one or more epitopes of MAGE-A3 or functional sequence variants thereof.

Such a complex does preferably not comprise any epitope of EpCAM, HER-2, TOMM34, RNF 43, KOC1, VEGFR, βhCG, CEA, TGFβR2, p53, KRas, OGT, CASP5, COA-1, SART or IL13Ralpha2. More preferably, such a complex does not comprise any epitope of ASCL2, EpCAM, HER-2, TOMM34, RNF 43, KOC1, VEGFR, βhCG, CEA, TGFβR2, p53, KRas, OGT, CASP5, COA-1, SART or IL13Ralpha2.

More preferably, such a complex of the first component (K) of the invention comprises
xxiii) one or more epitopes of EpCAM (such as the epitope according to SEQ ID NO: 41) or functional sequence variants thereof;
xxiv) one or more epitopes of MUC-1 (such as the epitope according to SEQ ID NO: 20 and/or the epitope according to SEQ ID NO: 21) or functional sequence variants thereof;
xxv) one or more epitopes of survivin (such as the epitope according to SEQ ID NO: 22) or functional sequence variants thereof; and/or
xxvi) one or more epitopes of CEA (such as the epitope according to SEQ ID NO: 26 and/or the epitope according to SEQ ID NO: 27) or functional sequence variants thereof.

In other words, the antigenic domain of said first component (K) preferably comprises
a) one or more epitopes of EpCAM or functional sequence variants thereof;
b) one or more epitopes of MUC-1 or functional sequence variants thereof;
c) one or more epitopes of survivin or functional sequence variants thereof; and/or
d) one or more epitopes of CEA or functional sequence variants thereof.

Such a complex does preferably not comprise any epitope of HER-2, TOMM34, RNF 43, KOC1, VEGFR, βhCG, TGFβR2, p53, KRas, OGT, CASP5, COA-1, MAGE, SART or IL13Ralpha2. More preferably, such a complex does not comprise any epitope of ASCL2, HER-2, TOMM34, RNF 43, KOC1, VEGFR, βhCG, TGFβR2, p53, KRas, OGT, CASP5, COA-1, MAGE, SART or IL13Ralpha2.

More preferably, such a complex of the first component (K) of the invention comprises
xxvii) one or more epitopes of EpCAM (such as the epitope according to SEQ ID NO: 41) or functional sequence variants thereof;
xxviii) one or more epitopes of MUC-1 (such as the epitope according to SEQ ID NO: 20 and/or the epitope according to SEQ ID NO: 21) or functional sequence variants thereof;
xxix) one or more epitopes of survivin (such as the epitope according to SEQ ID NO: 22) or functional sequence variants thereof;
xxx) one or more epitopes of ASCL2 (such as the epitope according to SEQ ID NO: 16 and/or the epitope according to SEQ ID NO: 17) or functional sequence variants thereof; and/or
xxxi) one or more epitopes of CEA (such as the epitope according to SEQ ID NO: 26 and/or the epitope according to SEQ ID NO: 27) or functional sequence variants thereof.

Such a complex does preferably not comprise any epitope of HER-2, TOMM34, RNF 43, KOC1, VEGFR, βhCG, TGFβR2, p53, KRas, OGT, CASP5, COA-1, MAGE, SART or IL13Ralpha2.

More preferably, such a complex of the first component (K) of the invention comprises
xxxii) one or more epitopes of EpCAM (such as the epitope according to SEQ ID NO: 41) or functional sequence variants thereof;
xxxiii) one or more epitopes of survivin (such as the epitope according to SEQ ID NO: 22) or functional sequence variants thereof;
xxxiv) one or more epitopes of ASCL2 (such as the epitope according to SEQ ID NO: 16 and/or the epitope according to SEQ ID NO: 17) or functional sequence variants thereof; and/or
xxxv) one or more epitopes of CEA (such as the epitope according to SEQ ID NO: 26 and/or the epitope according to SEQ ID NO: 27) or functional sequence variants thereof.

In other words, the antigenic domain of said first component (K) preferably comprises
a) one or more epitopes of EpCAM or functional sequence variants thereof;
c) one or more epitopes of survivin or functional sequence variants thereof; d) one or more epitopes of CEA or functional sequence variants thereof; and/or g) one or more epitopes of ASCL2 or functional sequence variants thereof.

Such a complex does preferably not comprise any epitope of HER-2, MUC-1, TOMM34, RNF 43, KOC1, VEGFR, βhCG, TGFβR2, p53, KRas, OGT, CASP5, COA-1, MAGE, SART or IL13Ralpha2.

Particularly preferably, such a complex of the first component (K) of the invention comprises
xxxvi) one or more epitopes of EpCAM (such as the epitope according to SEQ ID NO: 41) or functional sequence variants thereof;
xxxvii) one or more epitopes of MUC-1 (such as the epitope according to SEQ ID NO: 20 and/or the epitope according to SEQ ID NO: 21) or functional sequence variants thereof;
xxxviii) one or more epitopes of survivin (such as the epitope according to SEQ ID NO: 22) or functional sequence variants thereof; and
xxxix) one or more epitopes of CEA (such as the epitope according to SEQ ID NO: 26 and/or the epitope according to SEQ ID NO: 27) or functional sequence variants thereof.

Such a complex does preferably not comprise any epitope of HER-2, TOMM34, RNF 43, KOC1, VEGFR, βhCG, TGFβR2, p53, KRas, OGT, CASP5, COA-1, MAGE, SART or IL13Ralpha2. More preferably, such a complex does not comprise any epitope of ASCL2, HER-2, TOMM34, RNF 43, KOC1, VEGFR, βhCG, TGFβR2, p53, KRas, OGT, CASP5, COA-1, MAGE, SART or IL13Ralpha2.

It is also particularly preferred that such a complex of the first component (K) of the invention comprises
x) one or more epitopes of CEA (such as the epitope according to SEQ ID NO: 26 and/or the epitope according to SEQ ID NO: 27) or functional sequence variants thereof;
xli) one or more epitopes of survivin (such as the epitope according to SEQ ID NO: 22) or functional sequence variants thereof;
xlii) one or more epitopes of EpCAM (such as the epitope according to SEQ ID NO: 41) or functional sequence variants thereof; and
xliii) one or more epitopes of ASCL2 (such as the epitope according to SEQ ID NO: 16 and/or the epitope according to SEQ ID NO: 17) or functional sequence variants thereof.

Such a complex does preferably not comprise any epitope of HER-2, MUC-1, TOMM34, RNF 43, KOC1, VEGFR, βhCG, TGFβR2, p53, KRas, OGT, CASP5, COA-1, MAGE, SART or IL13Ralpha2.

It is also particularly preferred that such a complex of the first component (K) of the invention comprises
xliv) one or more epitopes of EpCAM (such as the epitope according to SEQ ID NO: 41) or functional sequence variants thereof;
xlv) one or more epitopes of MUC-1 (such as the epitope according to SEQ ID NO: 20 and/or the epitope according to SEQ ID NO: 21) or functional sequence variants thereof; and
xlvi) one or more epitopes of CEA (such as the epitope according to SEQ ID NO: 26 and/or the epitope according to SEQ ID NO: 27) or functional sequence variants thereof.

In other words, the antigenic domain of said first component (K) preferably comprises
a) one or more epitopes of EpCAM or functional sequence variants thereof;
b) one or more epitopes of MUC-1 or functional sequence variants thereof;
and/or
d) one or more epitopes of CEA or functional sequence variants thereof.

Such a complex does preferably not comprise any epitope of HER-2, TOMM34, RNF 43, KOC1, VEGFR, βhCG, survivin, TGFβR2, p53, KRas, OGT, CASP5, COA-1, MAGE, SART or IL13Ralpha2. More preferably, such a complex does not comprise any epitope of ASCL2, HER-2, TOMM34, RNF 43, KOC1, VEGFR, βhCG, survivin, TGFβR2, p53, KRas, OGT, CASP5, COA-1, MAGE, SART or IL13Ralpha2.

More preferably, the antigenic domain of said first component (K) preferably comprises
a) one or more epitopes of EpCAM or functional sequence variants thereof;
and/or
d) one or more epitopes of CEA or functional sequence variants thereof.

It is also particularly preferred that such a complex of the first component (K) of the invention comprises
xlvii) one or more epitopes of CEA (such as the epitope according to SEQ ID NO: 26 and/or the epitope according to SEQ ID NO: 27) or functional sequence variants thereof;
xlviii) one or more epitopes of survivin (such as the epitope according to SEQ ID NO: 22) or functional sequence variants thereof; and
xlix) one or more epitopes of ASCL2 (such as the epitope according to SEQ ID NO: 16 and/or the epitope according to SEQ ID NO: 17) or functional sequence variants thereof.

Such a complex does preferably not comprise any epitope of HER-2, EpCAM, MUC-1, TOMM34, RNF 43, KOC1, VEGFR, βhCG, TGFβR2, p53, KRas, OGT, CASP5, COA-1, MAGE, SART or IL13Ralpha2.

Even more preferably, the complex of the first component (K) of the invention comprises in N- to C-terminal direction:
one or more epitopes of CEA (such as the epitope according to SEQ ID NO: 26 and/or the epitope according to SEQ ID NO: 27) or functional sequence variants thereof;
one or more epitopes of survivin (such as the epitope according to SEQ ID NO: 22) or functional sequence variants thereof; and
one or more epitopes of ASCL2 (such as the epitope according to SEQ ID NO: 16 and/or the epitope according to SEQ ID NO: 17) or functional sequence variants thereof.

Such a complex does preferably not comprise any epitope of HER-2, EpCAM, MUC-1, TOMM34, RNF 43, KOC1, VEGFR, βhCG, TGFβR2, p53, KRas, OGT, CASP5, COA-1, MAGE, SART or IL13Ralpha2

Even more preferably, the complex of the first component (K) of the invention comprises in N- to C-terminal direction:
i) a peptide having an amino acid sequence according to SEQ ID NO: 24, or a fragment thereof having a length of at least 10 amino acids (preferably at least 15 amino acids, more preferably at least 20 amino acids, even more preferably at least 25 amino acids and most preferably at least 30 amino acids), or a functional sequence variant thereof having at least 70% sequence identity (preferably at least 75%, more preferably at least 80%, even preferably at least 85%, still more preferably at least 90%, particularly preferably at least 95%, most preferably at least 99% sequence identity);
ii) a peptide having an amino acid sequence according to SEQ ID NO: 12, or a fragment thereof having a length of at least 10 amino acids (preferably at least 15 amino acids, more preferably at least 20 amino acids, even more preferably at least 25 amino acids and most preferably at least 30 amino acids), or a functional sequence variant thereof having at least 70% sequence identity (preferably at least 75%, more preferably at least 80%, even preferably at least 85%, still more preferably at least 90%, particularly preferably at least 95%, most preferably at least 99% sequence identity); and
iii) a peptide having an amino acid sequence according to SEQ ID NO: 15, or a fragment thereof having a length of at least 10 amino acids (preferably at least 15 amino acids, more preferably at least 20 amino acids, even more preferably at least 25 amino acids and most preferably at least 30 amino acids), or a functional sequence variant thereof having at least 70% sequence identity (preferably at least 75%, more preferably at least 80%, even preferably at least 85%, still more preferably at least 90%, particularly preferably at least 95%, most preferably at least 99% sequence identity).

Such a complex does preferably not comprise any further antigen or further epitopes of antigens other than CEA, survivin and ASCL2, more preferably such a complex does not comprise any other (tumor) epitope.

Preferably, in such a complex of the first component (K) of the invention, the C-terminus of (i) the peptide having an amino acid sequence according to SEQ ID NO: 24 or the fragment or variant thereof is directly linked to the N-terminus of (ii) the peptide having an amino acid sequence according to SEQ ID NO: 12 or the fragment or variant thereof; and the C-terminus of (ii) the peptide having an amino acid sequence according to SEQ ID NO: 12 or the fragment or variant thereof is directly linked to the N-terminus of (iii) the peptide having an amino acid sequence according to SEQ ID NO: 15 or the fragment or variant thereof.

Still more preferably, the complex of the first component (K) of the invention comprises in N- to C-terminal direction:
i) a peptide having an amino acid sequence according to SEQ ID NO: 25, or a functional sequence variant thereof having at least 70% sequence identity (preferably at least 75%, more preferably at least 80%, even preferably at least 85%, still more preferably at least 90%, particularly preferably at least 95%, most preferably at least 99% sequence identity);
ii) a peptide having an amino acid sequence according to SEQ ID NO: 23, or a functional sequence variant thereof having at least 70% sequence identity (preferably at least 75%, more preferably at least 80%, even preferably at least 85%, still more preferably at least 90%, particularly preferably at least 95%, most preferably at least 99% sequence identity); and
iii) a peptide having an amino acid sequence according to SEQ ID NO: 18, or a functional sequence variant thereof having at least 70% sequence identity (preferably at least 75%, more preferably at least 80%, even preferably at least 85%, still more preferably at least 90%, particularly preferably at least 95%, most preferably at least 99% sequence identity).

Such a complex does preferably not comprise any further antigen or further epitopes of antigens other than CEA, survivin and ASCL2, more preferably such a complex does not comprise any other (tumor) epitope.

Preferably, in such a complex of the first component (K) of the invention, the C-terminus of (i) the peptide having an amino acid sequence according to SEQ ID NO: 25 or the variant thereof is directly linked to the N-terminus of (ii) the peptide having an amino acid sequence according to SEQ ID NO: 23 or the variant thereof; and the C-terminus of (ii) the peptide having an amino acid sequence according to SEQ ID NO: 23 or the variant thereof is directly linked to the N-terminus of (iii) the peptide having an amino acid sequence according to SEQ ID NO: 18 or the variant thereof.

Most preferably, the complex of the first component (K) of the vaccine of the invention comprises in its antigenic domain a peptide having an amino acid sequence according to SEQ ID NO: 45 or a functional sequence variant thereof having at least 70% sequence identity (preferably at least 75%, more preferably at least 80%, even preferably at least 85%, still more preferably at least 90%, particularly preferably at least 95%, most preferably at least 99% sequence identity). Such a complex does preferably not comprise any further antigen or further epitopes of antigens other than CEA, survivin and ASCL2, more preferably such a complex does not comprise any other (tumor) epitope.

It is also particularly preferred that the complex of the first component (K) of the vaccine of the invention as disclosed herein, may consist of a polypeptide which e.g. comprises the amino acid sequences according to SEQ ID NO: 3, SEQ ID NO: 45, SEQ ID NO:6, or SEQ ID NO: 4, SEQ ID NO: 45, SEQ ID NO: 7, or SEQ ID NO: 5, SEQ ID NO: 45, SEQ ID NO: 7, or e.g. SEQ ID NO: 3, SEQ ID NO: 45, SEQ ID NO:7, or SEQ ID NO: 4, SEQ ID NO: 45, SEQ ID NO: 7, or SEQ ID NO: 5, SEQ ID NO: 45, SEQ ID NO: 7, or e.g., SEQ ID NO: 3, SEQ ID NO: 45, SEQ ID NO:8, or SEQ ID NO: 4, SEQ ID NO: 45, SEQ ID NO: 8, or SEQ ID NO: 5, SEQ ID NO: 45, SEQ ID NO: 8. Alternatively, the first component (K) of the vaccine of the invention as disclosed above may e.g. comprise combinations of the TLR agonist ANAXA or a sequence variant thereof (e.g. SEQ ID NO: 6, SEQ ID NO: 7) with the TLR agonist Δ30-HMGB1 (SEQ ID NO: 9), for example, the complex of the first component may comprise SEQ ID NO: 2, SEQ ID NO: 45, SEQ ID NO: 6, SEQ ID NO: 9, or SEQ ID NO: 3, SEQ ID NO: 45, SEQ ID NO: 6, SEQ ID NO: 9, or SEQ ID NO: 4, SEQ ID NO: 45, SEQ ID NO: 6, SEQ ID NO: 9, or SEQ ID NO: 5, SEQ ID NO: 45, SEQ ID NO: 6, SEQ ID NO: 9, or SEQ ID NO: 2, SEQ ID NO: 45, SEQ ID NO: 7, SEQ ID NO: 9, or SEQ ID NO: 3, SEQ ID NO: 45, SEQ ID NO: 7, SEQ ID NO: 9, or SEQ ID NO: 4, SEQ ID NO: 45, SEQ ID NO: 7, SEQ ID NO: 9, or SEQ ID NO: 5, SEQ ID NO: 45, SEQ ID NO: 7, SEQ ID NO: 9, or a functional sequence variants of any of the above sequences having at least 70% sequence identity (preferably at least 75%, more preferably at least 80%, even preferably at least 85%, still more preferably at least 90%, particularly preferably at least 95%, most preferably at least 99% sequence identity). It is e.g. preferred that the complex of the first component (K) of the invention comprises the above amino acid sequences in N- to C-terminal direction linked via peptide bonds. The sequences as disclosed above may, e.g. also comprise linker or spacer sequences between the individual amino acid sequences.

Specifically preferably, the complex of the first component (K) of the vaccine of the invention consists of an amino acid sequence according to SEQ ID NO: 60 or a functional sequence variant thereof having at least 70% sequence identity, preferably at least 75%, more preferably at least 80%, even preferably at least 85%, still more preferably at least 90%, particularly preferably at least 95%, most preferably at least 99% sequence identity. Such a complex does preferably not comprise any further antigen or further epitopes of antigens other than CEA, survivin and ASCL2, more preferably such a complex does not comprise any other (tumor) epitope.

It is also particularly preferred that such a complex of the first component (K) according to the invention comprises
one or more epitopes of EpCAM (such as the epitope according to SEQ ID NO: 41) or functional sequence variants thereof; and
one or more epitopes of CEA (such as the epitope according to SEQ ID NO: 26 and/or the epitope according to SEQ ID NO: 27) or functional sequence variants thereof.

Such a complex does preferably not comprise any epitope of HER-2, MUC-1, TOMM34, RNF 43, KOC1, VEGFR, βhCG, survivin, TGFβR2, p53, KRas, OGT, CASP5, COA-1, MAGE, SART or IL13Ralpha2. More preferably, such a complex does not comprise any epitope of ASCL2, HER-2, MUC-1, TOMM34, RNF 43, KOC1, VEGFR, βhCG, survivin, TGFβR2, p53, KRas, OGT, CASP5, COA-1, MAGE, SART or IL13Ralpha2.

It is also particularly preferred that such a complex of the first component (K) according to the invention comprises
one or more epitopes of EpCAM (such as the epitope according to SEQ ID NO: 41) or functional sequence variants thereof; and
one or more epitopes of ASCL2 (such as the epitope according to SEQ ID NO: 16 and/or the epitope according to SEQ ID NO: 17) or functional sequence variants thereof.

Such a complex does preferably not comprise any epitope of HER-2, MUC-1, CEA, TOMM34, RNF 43, KOC1, VEGFR, βhCG, survivin, TGFβR2, p53, KRas, OGT, CASP5, COA-1, MAGE, SART or IL13Ralpha2.

It is also particularly preferred that such a complex of the first component (K) according to the invention comprises
one or more epitopes of ASCL2 (such as the epitope according to SEQ ID NO: 16 and/or the epitope according to SEQ ID NO: 17) or functional sequence variants thereof; and
one or more epitopes of CEA (such as the epitope according to SEQ ID NO: 26 and/or the epitope according to SEQ ID NO: 27) or functional sequence variants thereof.

In other words, the antigenic domain of the first component (K) preferably comprises
one or more epitopes of CEA or functional sequence variants thereof; and
one or more epitopes of ASCL2 or functional sequence variants thereof.

Such a complex does preferably not comprise any epitope of HER-2, EpCAM, MUC-1, TOMM34, RNF 43, KOC1, VEGFR, βhCG, survivin, TGFβR2, p53, KRas, OGT, CASP5, COA-1, MAGE, SART or IL13Ralpha2.

It is also particularly preferred that such a complex of the first component (K) according to the invention comprises
one or more epitopes of survivin (such as the epitope according to SEQ ID NO: 22) or functional sequence variants thereof; and
one or more epitopes of ASCL2 (such as the epitope according to SEQ ID NO: 16 and/or the epitope according to SEQ ID NO: 17) or functional sequence variants thereof.

In other words, the antigenic domain of the first component (K) preferably comprises
one or more epitopes of survivin or functional sequence variants thereof; and
one or more epitopes of ASCL2 or functional sequence variants thereof.

Such a complex does preferably not comprise any epitope of HER-2, MUC-1, EpCAM, CEA, TOMM34, RNF 43, KOC1, VEGFR, βhCG, TGFβR2, p53, KRas, OGT, CASP5, COA-1, MAGE, SART or IL13Ralpha2.

It is also particularly preferred that such a complex of the first component (K) according to the invention comprises
one or more epitopes of survivin (such as the epitope according to SEQ ID NO: 22) or functional sequence variants thereof; and
one or more epitopes of CEA (such as the epitope according to SEQ ID NO: 26 and/or the epitope according to SEQ ID NO: 27) or functional sequence variants thereof.

In other words, the antigenic domain of the first component (K) preferably comprises
one or more epitopes of survivin or functional sequence variants thereof; and
one or more epitopes of CEA or functional sequence variants thereof.

Such a complex does preferably not comprise any epitope of ASCL2, HER-2, EpCAM, MUC-1, TOMM34, RNF 43, KOC1, VEGFR, βhCG, TGFβR2, p53, KRas, OGT, CASP5, COA-1, MAGE, SART or IL13Ralpha2.

Particularly preferably, the antigenic domain of the first component (K) preferably comprises
one or more epitopes of survivin or functional sequence variants thereof;
one or more epitopes of CEA or functional sequence variants thereof; and
one or more epitopes of ASCL2 or functional sequence variants thereof.

It is also particularly preferred that such a complex of the first component (K) according to the invention comprises
one or more epitopes of EpCAM (such as the epitope according to SEQ ID NO: 41) or functional sequence variants thereof.

Such a complex does preferably not comprise any epitope of HER-2, MUC-1, TOMM34, RNF 43, KOC1, VEGFR, βhCG, survivin, CEA, TGFβR2, p53, KRas, OGT, CASP5, COA-1, MAGE, SART or IL13Ralpha2. More preferably, such a complex does not comprise any epitope of ASCL2, HER-2, MUC-1, TOMM34, RNF 43, KOC1, VEGFR, βhCG, survivin, CEA, TGFβR2, p53, KRas, OGT, CASP5, COA-1, MAGE, SART or IL13Ralpha2.

It is also particularly preferred that such a complex of the first component (K) according to the invention comprises
one or more epitopes of CEA (such as the epitope according to SEQ ID NO: 26 and/or the epitope according to SEQ ID NO: 27) or functional sequence variants thereof.

Such a complex does preferably not comprise any epitope of EpCAM, HER-2, MUC-1, TOMM34, RNF 43, KOC1, VEGFR, βhCG, survivin, TGFβR2, p53, KRas, OGT, CASP5, COA-1, MAGE, SART or IL13Ralpha2. More preferably, such a complex does not comprise any epitope of ASCL2, EpCAM, HER-2, MUC-1, TOMM34, RNF 43, KOC1, VEGFR, βhCG, survivin, TGFβR2, p53, KRas, OGT, CASP5, COA-1, MAGE, SART or IL13Ralpha2.

It is also particularly preferred that such a complex of the first component (K) according to the invention comprises
one or more epitopes of ASCL2 (such as the epitope according to SEQ ID NO: 16 and/or the epitope according to SEQ ID NO: 17) or functional sequence variants thereof.

Such a complex does preferably not comprise any epitope of EpCAM, HER-2, MUC-1, CEA, TOMM34, RNF 43, KOC1, VEGFR, βhCG, survivin, TGFβR2, p53, KRas, OGT, CASP5, COA-1, MAGE, SART or IL13Ralpha2.

### Rhabdoviruses

The family of rhabdoviruses includes 18 genera and 134 species with negative-sense, single-stranded RNA genomes of approximately 10-16 kb (Walke et al., ICTV Virus Taxonomy Profile: Rhabdoviridae, Journal of General Virology, 99:447-448 (2018)).

Characterizing features of members of the family of rhabdoviruses include one or more of the following: A bullet-shaped or bacilliform particle 100-430 nm in length and 45-100 nm in diameter comprised of a helical nucleocapsid surrounded by a matrix layer and a lipid envelope, a negative-sense, single-stranded RNA of 10.8-16.1 kb, which is unsegmented; a genome encoding for at least 5 genes encoding the structural proteins nucleoprotein (N), large protein (L), phosphoprotein (P), matrix protein (M), and glycoprotein (G).

According to one embodiment, the second component (V) of the vaccine of the invention as disclosed above is a recombinant rhabdovirus. In other words, the rhabdovirus, preferably the oncolytic rhabdovirus, of the second component (V) is preferably a recombinant rhabdovirus. The term “recombinant” as used herein refers to the fact that the rhabdovirus does not occur naturally.

The rhabdovirus according to the invention can belong to the genus of: almendravirus, curiovirus, cytorhabdovirus, dichorhavirus, ephemerovirus, Hapavirus, ledantevirus, lyssavirus, novirhabdovirus, nucleorhabdovirus, perhabdovirus, sigmavirus, sprivivirus, sripuvirus, tibrovirus, tupavirus, varicosavirus or vesiculovirus. Preferably, the rhabdovirus according to the invention belongs to the genus of vesiculovirus, e.g. the rhabdovirus for use according to the invention may be one of Alagoas vesiculovirus, American bat vesiculovirus, Carajas vesiculovirus, Chandipura vesiculovirus, Cocal vesiculovirus, Indiana vesiculovirus, Isfahan vesiculovirus, Jurona vesiculovirus, Malpais Spring vesiculovirus, Maraba vesiculovirus, Morreton vesiculovirus, New Jersey vesiculovirus, Perinet vesiculovirus, Piry vesiculovirus, Radi vesiculovirus, Yug Bogdanovac vesiculovirus, or Moussa virus.

Preferably, the recombinant rhabdovirus of the invention is an oncolytic rhabdovirus. In this respect, oncolytic has its regular meaning known in the art and refers to the ability of a rhabdovirus to infect and lyse (break down) cancer cells, while non-cancerous cells are not lysed to any significant extend.

Preferably, the rhabdovirus, preferably the oncolytic rhabdovirus, of the invention is replication competent and capable of replicating within cancer cells. In particular, the recombinant vesicular stomatitis virus, preferably the oncolytic recombinant vesicular stomatitis virus, may be replication-competent. Oncolytic activity of the recombinant rhabdovirus of the invention may be tested in different assay systems known to the skilled artisan (an exemplary in vitro assay is described by Muik et al., Cancer Res., 74(13), 3567-78, 2014). It is to be understood that an oncolytic rhabdovirus may infect and lyse only specific types of cancer cells. Also, the oncolytic effect may vary depending on the type of cancer cells.

In a preferred embodiment, the recombinant rhabdovirus, preferably the oncolytic recombinant rhabdovirus, of the invention belongs to the genus of vesiculovirus. Vesiculovirus species have been defined primarily by serological means coupled with phylogenetic analysis of the genomes. Biological characteristics such as host range and mechanisms of transmission are also used to distinguish viral species within the genus. As such, the genus of vesiculovirus form a distinct monophyletic group well-supported by Maximum Likelihood trees inferred from complete L sequences.

Viruses assigned to different species within the genus vesiculovirus may have one or more of the following characteristics: A) a minimum amino acid sequence divergence of 20% in L; B) a minimum amino acid sequence divergence of 10% in N; C) a minimum amino acid sequence divergence of 15% in G; D) can be distinguished in serological tests; and E) occupy different ecological niches as evidenced by differences in hosts and or arthropod vectors. Preferred is the vesicular stomatitis virus (VSV) and in particular the VSV-GP (recombinant VSV with GP of LCMV as disclosed in WO2010/040526). Advantageous properties of the VSV-GP include one or more of the following: very potent and fast killer (<8 h); oncolytic virus; systemic application possible; significantly reduced neurotropism with abolished neurotoxicity; it reproduces lytically; strong activation of innate immunity; about 3 kb space for immunomodulatory cargos and antigens; recombinant with an arenavirus glycoprotein from the lymphocytic-choriomeningitis-virus (LCMV); favorable safety features in terms of reduced neurotoxicity and less sensitive to neutralizing antibody responses and complement destruction as compared to the wild type VSV (VSV-G); specifically replicates in tumor cells, which have lost the ability to mount and respond to anti-viral innate immune responses (e.g. type-I IFN signaling); abortive replication in “healthy cells” so is rapidly excluded from normal tissues; viral replication in tumor cells leads to the cell death, and assumed to result in the release of tumor associated antigens, local inflammation and the induction of anti-tumor immunity.

It is preferred that the recombinant vesiculovirus, preferably the oncolytic recombinant vesiculovirus, of the invention is selected from the group comprising: Vesicular stomatitis alagoas virus (VSAV), Carajes virus (CJSV), Chandipura virus (CHPV), Cocal virus (COCV), Vesicular stomatitis Indiana virus (VSIV), Isfahan virus (ISFV), Maraba virus (MARAV), Vesicular stomatitis New Jersey virus (VSNJV), or Piry virus (PIRYV), more preferably, the recombinant vesiculovirus of the invention is selected from one of Vesicular stomatitis Indiana virus (VSIV) or Vesicular stomatitis New Jersey virus (VSNJV).

In one preferred embodiment, the recombinant Vesicular stomatitis Indiana virus (VSIV) or Vesicular stomatitis New Jersey virus (VSNJV), preferably the oncolytic recombinant Vesicular stomatitis Indiana virus (VSIV) or Vesicular stomatitis New Jersey virus (VSNJV), of the invention are replication competent. The terms “replication competent” or “replication competent virus” as used herein refer to a virus which contains all the information within its genome to allow it to replicate within a cell. For example, replication competence of the recombinant vesicular stomatitis virus of the invention may be assessed according to the methods disclosed in Tani et al. JOURNAL OF VIROLOGY, August 2007, p. 8601-8612; or Garbutt et al. JOURNAL OF VIROLOGY, May 2004, p. 5458-5465.

In a preferred embodiment of the invention, the RNA genome of the vesicular stomatitis virus comprises or consists of an RNA sequence identical to SEQ ID NO: 80. In another preferred embodiment of the invention, the RNA genome of the vesicular stomatitis virus may also consist of or comprise those sequences, wherein nucleic acids of the RNA genome are exchanged according to the degeneration of the genetic code, without leading to an alteration of respective amino acid sequence. In a further preferred embodiment of the invention, the RNA genome of the vesicular stomatitis virus comprises or consists of an RNA sequence identical or at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 80.

In a further embodiment, the invention provides for a vesicular stomatitis virus, wherein the RNA genome of the vesicular stomatitis virus comprises or consists of an RNA sequence identical or at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 80, wherein the vesicular stomatitis virus encodes in its genome a phosphoprotein (P) comprising the amino acid consisting of SEQ ID NO: 50, a nucleoprotein (N) comprising the amino acid sequence consisting of SEQ ID NO: 49, a matrix protein (M) comprising the amino acid sequence consisting of SEQ ID NO: 52, a large protein (L) comprising the amino acid sequence consisting of SEQ ID NO: 51, a glycoprotein (GP) comprising the amino acid sequence consisting of SEQ ID NO: 53, and an antigenic domain which comprises the amino acid sequence consisting of SEQ ID NO: 45 or SEQ ID NO: 59.

It is known that certain wildtype rhabdovirus strains such as wildtype VSV strains are considered to be neurotoxic. It is also reported that infected individuals are able to rapidly mount a strong humoral response with high antibody titers directed mainly against the glycoprotein. Neutralizing antibodies targeting the glycoprotein G of rhabdoviruses in general and VSV specifically are able to limit virus spread and thereby mediate protection of individuals from virus re-infection. Virus neutralization, however, would limit repeated application of the recombinant rhabdovirus, preferably the oncolytic recombinant rhabdovirus, of the invention as comprised in the vaccine as disclosed herein.

To eliminate these drawbacks the rhabdovirus wild type glycoprotein G may e.g. be replaced with the glycoprotein from another virus. In this respect replacing the glycoprotein refers to (i) replacement of the gene coding for the wild type glycoprotein G with the gene coding for the glycoprotein GP of another virus, and/or (ii) replacement of the wild type glycoprotein G with the glycoprotein GP of another virus.

For example, the glycoprotein G of the recombinant VSV, preferably the oncolytic recombinant VSV, as disclosed herein may be replaced by the glycoprotein GP of Ebola virus (EBOV) or of its reported strains (e.g. Sudan, Reston, Zaire or Tai Forest) which is a member of the family Filoviridae that are enveloped, single-stranded RNA viruses.

In one embodiment, the gene coding for the glycoprotein GP of the EBOV encodes for an amino acid sequence of one of the EBOV strains Sudan, Reston, Zaire or Tai Forrest or a sequence having at least 80, 85, 90 or 95% sequence identity to any of said amino acid sequences while the functional properties of the recombinant VSV, preferably the oncolytic recombinant VSV, comprising a glycoprotein GP as disclosed above are maintained. Corresponding methods to assess the functional properties of the glycoproteins and respective variants as disclosed herein may be done as disclosed in e.g. J Virol. 2010 January; 84(2): 983-992, or Cancer Res. 2014 Jul. 1; 74(13):3567-78.

In one embodiment, the wild type glycoprotein G of VSV as disclosed herein may e.g. be replaced by a glycoprotein (GP) of an arenavirus. The Arenaviridae family consists of a unique Arenavirus genus that currently contains 22 recognized virus species. Arenaviruses are enveloped single-stranded RNA viruses, with a genome consisting of two RNA segments, designated large (L) and small (S). The L genomic segment (˜7.2 kb) encodes the viral RNA-dependent RNA polymerase and a zinc-binding protein. The S genomic segment (˜3.5 kb) encodes the nucleocapsid protein and envelope glycoproteins in nonoverlapping open reading frames of opposite polarities. The genes on both S and L segments are separated by an intergenic noncoding region with the potential of forming one or more hairpin configurations. The 5′ and 3′ untranslated terminal sequences of each RNA segment possess a relatively conserved reverse complementary sequence spanning 19 nucleotides at each extremity. Nucleocapsid antigens are shared by most arenaviruses, and quantitative relationships show the basic split between viruses of Africa and viruses of the Western Hemisphere. Individual viruses are immunologically distinct by neutralization assays, which depend on the specificity of epitopes contained in the envelope glycoproteins. Thus, the wild type glycoprotein of VSV may be e.g. replaced by a glycoprotein of the family members of the arenaviridae, e.g. Allpahuayo (ALLV), Amapari (AMAV), Bear canyon (BCNV), Cupixi (CPXV), Flexal (FLEV), Guanarito (GTOV), Ippy (IPPYV), Junin (JUNV), Lassa (LASV), Latino (LATV), Lymphocytic choriomeningitis (LCMV), Machupo (MACV), Mobala (MOBV), Mopeia (MOPV), Oliveros (OLVV), Parana (PARV), Pichinde (PICV), Pirital (PIRV), Sabia (SABV), Tacaribe (TCRV), Tamiami (TAMV), or Whitewater Arroyo (WWAV).

In one embodiment, the glycoprotein G of the recombinant VSV, preferably the oncolytic recombinant VSV, as disclosed herein may be replaced by the (mature) glycoprotein GP of Lassa virus comprising the amino acid sequence according to SEQ ID NO: 72, or a sequence having at least 80, 85, 90 or 95% sequence identity to the amino acid sequence of SEQ ID NO: 72 while the functional properties of the recombinant VSV, preferably the oncolytic recombinant VSV, comprising a glycoprotein GP encoding an amino acid sequence as shown in SEQ ID NO:72 are maintained. Lassa virus (LASV) is a member of the family Arenaviridae, of which Lymphocytic choriomeningitis virus (LCMV) is the prototype.

In a preferred embodiment the recombinant rhabdovirus glycoprotein G is replaced with the glycoprotein GP of the Dandenong virus (DANDV) or Mopeia (MOPV) virus. In a more preferred embodiment, the recombinant rhabdovirus is a vesicular stomatitis virus wherein the glycoprotein G is replaced with the glycoprotein GP of the Dandenong virus (DANDV) or Mopeia (MOPV) virus.

Advantages offered by the replacement of the wild type glycoprotein of VSV with any of the glycoproteins disclosed above are (i) the loss of VSV-G mediated neurotoxicity and (ii) a lack of vector neutralization by antibodies (as shown in mice).

The Dandenong virus (DANDV) is an old world arenavirus. To date, there is only a single strain known to the person skilled in the art, which comprise a glycoprotein GP and which may be employed within the present invention as donor of the glycoprotein GP comprised in the recombinant rhabdovirus of the invention. The DANDV glycoprotein GP comprised in the recombinant rhabdovirus of the invention has more than 6 glycosylation sites, in particular 7 glycosylation sites. An exemplary preferred glycoprotein GP is that as comprised in DANDV as accessible under Genbank number EU136038. In one embodiment, the gene coding for the glycoprotein GP of the DNADV encodes for an amino acid sequence as shown in SEQ ID NO: 47 or a sequence having at least 80, 85, 90 or 95% sequence identity to the amino acid sequence of SEQ ID NO:47 while the functional properties of the recombinant rhabdovirus comprising a glycoprotein GP encoding an amino acid sequence as shown in SEQ ID NO:47 are maintained.

The Mopeia virus (MOPV) is an old world arenavirus. There are several strains known to the person skilled in the art, which comprise a glycoprotein GP and which may be employed within the present invention as donor of the glycoprotein GP comprised in the recombinant rhabdovirus of the invention. The MOPV glycoprotein GP comprised in the recombinant rhabdovirus of the invention has more than 6 glycosylation sites, in particular seven glycosylation sites. An exemplary preferred glycoprotein GP is that as comprised in Mopeia virus as accessible under Genbank number AY772170. In one embodiment, the gene coding for glycoprotein GP of the MOPV encodes for an amino acid sequence according to SEQ ID NO:48 or a sequence having at least 60, 65, 70, 75, 80, 85, 90 or 95% sequence identity to the amino acid sequence of SEQ ID NO:48 while the functional properties of the recombinant rhabdovirus comprising a glycoprotein GP encoding an amino acid sequence as shown in SEQ ID NO:48 are maintained. Functional properties of the glycoprotein and variants thereof as disclosed herein may e.g. be assessed according to methods known in the art such as those disclosed in J Virol. 2014 May; 88(9):4897-907.

In a particularly preferred embodiment the rhabdovirus glycoprotein G is replaced with the glycoprotein GP of the lymphocytic choriomeningitis virus (LCMV), preferably with the strain WE-HPI. In an even more preferred embodiment, the rhabdovirus is a vesicular stomatitis virus with the glycoprotein GP of the lymphocytic choriomeningitis virus (LCMV), preferably with the strain WE-HPI. Such VSV is for example described in WO2010/040526 or WO2006/008074 and referred to as “VSV-GP”. Advantages offered are (i) the loss of VSV-G mediated neurotoxicity and (ii) a lack of vector neutralization by antibodies (as shown in mice). The envelope glycoproteins of LCMV (LCMV GP) are initially expressed as a precursor polypeptide, GP-C, which is posttranslationally processed by a cellular protease into GP-1 and GP-2. GP-1 interacts with the cellular receptor for LCMV, which has been identified as alpha-dystroglycan. GP-2 contains the fusion peptide and the transmembrane domain.

The glycoprotein GP of the lymphocytic choriomeningitis virus (LCMV) may be GP1 or GP2. The invention includes glycoproteins from different LCMV strains. In particular, LCMV-GP can be derived from LCMV wild-type or LCMV strains LCMV-WE, LCMV-WE-HPI, LCMV-WE-HPlopt. In a preferred embodiment, the gene coding for the glycoprotein GP of the LCMV encodes for a protein with an amino acid sequence as shown in SEQ ID NO: 46 or an amino acid sequence having at least 80, 85, 90, 95%, 98%, 99% sequence identity to the amino acid sequence of SEQ ID NO: 46 while the functional properties of the recombinant rhabdovirus, preferably the oncolytic recombinant rhabdovirus, more specifically of the recombinant vesicular stomatitis virus, preferably the oncolytic recombinant vesicular stomatitis virus, comprising a glycoprotein GP encoding an amino acid sequence as shown in SEQ ID NO: 11 are maintained.

In a preferred embodiment the recombinant vesicular stomatitis virus, preferably the oncolytic recombinant vesicular stomatitis virus, of the invention encodes in its genome at least for a vesicular stomatitis virus nucleoprotein (N) comprising an amino acid sequence as set forth in SEQ ID NO:49 or a functional variant at least 80%, 85%, 90%, 92%, 94%, 96%, 98% identical to SEQ ID NO:49, a phosphoprotein (P) comprising an amino acid sequence as set forth in SEQ ID NO: 50 or a functional variant at least 80%, 85%, 90%, 92%, 94%, 96%, 98% identical to SEQ ID NO:50, a large protein (L) comprising an amino acid sequence as set forth in SEQ ID NO:51 or a functional variant at least 80%, 85%, 90%, 92%, 94%, 96%, 98% identical to SEQ ID NO: 51, and a matrix protein (M) comprising an amino acid sequence as set forth in SEQ ID NO: 52 or a functional variant at least 80%, 85%, 90%, 92%, 94%, 96%, 98% identical to SEQ ID NO: 52.

More preferably, the recombinant vesicular stomatitis virus, preferably the oncolytic recombinant vesicular stomatitis virus, of the second component (V) encodes in its genome a vesicular stomatitis virus nucleoprotein (N), large protein (L), phosphoprotein (P), matrix protein (M), glycoprotein (G) and at least one antigen or antigenic epitope according to any one of claims 22 to 54, wherein the gene coding for the glycoprotein G of the vesicular stomatitis virus is replaced by the gene coding for the glycoprotein GP of lymphocyte choriomeningitis virus (LCMV), and/or the glycoprotein G is replaced by the glycoprotein GP of LCMV, and
the nucleoprotein (N) comprises an amino acid as set forth in SEQ ID NO:49 or a functional variant at least 80%, 85%, 90%, 92%, 94%, 96%, 98% identical to SEQ ID NO: 49;
wherein the phosphoprotein (P) comprises an amino acid as set forth in SEQ ID NO:50 or a functional variant at least 80%, 85%, 90%, 92%, 94%, 96%, 98% identical to SEQ ID NO: 50;
wherein the large protein (L) comprises an amino acid as set forth in SEQ ID NO:51 or a functional variant at least 80%, 85%, 90%, 92%, 94%, 96%, 98% identical to SEQ ID NO: 51; and
the matrix protein (M) comprises an amino acid as set forth in SEQ ID NO:52 or a functional variant at least 80%, 85%, 90%, 92%, 94%, 96%, 98% identical to SEQ ID NO: 52.

For example, the above functional variants constitute modifications to the vesicular stomatitis virus nucleoprotein (N), large protein (L), phosphoprotein (P), matrix protein (M), or glycoprotein (G) sequence without losing the basic functions of those proteins. Such functional variants as used herein retain all or part of their basic function or activity. The protein L for example is the polymerase and has an essential function during transcription and replication of the virus. A functional variant thereof must retain at least part of this ability. A good indication for retention of basic functionality or activity is the successful production of viruses, including these functional variants, that are still capable to replicate and infect tumor cells. Production of viruses and testing for infection and replication in tumor cells may be tested in different assay systems known to the skilled artisan (an exemplary in vitro assay is described by Muik et al., Cancer Res., 74(13), 3567-78, 2014). Accordingly, the vaccine of the invention may comprise a recombinant vesicular stomatitis virus (V) encoding in its genome the virus nucleoprotein (N), large protein (L), phosphoprotein (P), matrix protein (M), or glycoprotein (G) sequence as disclosed above.

It is preferred that the recombinant vesicular stomatitis virus, preferably the oncolytic recombinant vesicular stomatitis virus, of the invention as disclosed above encodes in its genome a second antigenic domain which comprises the amino acid sequence of the antigenic domain of the complex of first component (K) of the invention as disclosed above. In particular, the antigenic domain encoded in the genome of the recombinant vesicular stomatitis virus, preferably the oncolytic recombinant vesicular stomatitis virus, of the invention as disclosed above, comprising the identical amino acid sequence to that of the antigenic domain of the complex of the first component (K) as disclosed above. For example, antigenic domains and their respective amino acid sequence as disclosed in the context of the recombinant vesicular stomatitis virus, preferably the oncolytic recombinant vesicular stomatitis virus, herein condition that the antigenic domain of said first component (K) of the invention comprises a antigenic domain of identical amino acid sequence, alternatively, antigenic domains as disclosed in the context of the first component (K) of the invention condition that the antigenic domain of the recombinant vesicular stomatitis virus, preferably the oncolytic recombinant vesicular stomatitis virus, of the invention comprises a antigenic domain of identical amino acid sequence as that of the first component (K) of the invention as disclosed above.

It is understood that a number of different antigens or antigenic epitopes relating to the cancer types as disclosed above, e.g. colorectal cancer, breast cancer, or pancreatic cancer may e.g. be distributed to subsets of different antigens or antigenic epitopes, in particular subsets complementing each other in the context of colorectal cancer, breast cancer, or pancreatic cancer which can be comprised by different antigenic domains such as the antigenic domain of the first component (K) as disclosed above and to the antigenic domain of the recombinant vesicular stomatitis virus, preferably the oncolytic recombinant vesicular stomatitis virus, of the invention.

Accordingly, the antigenic domain of the complex of the first component (K) as disclosed herein and the antigenic domain encoded by the recombinant rhabdovirus, preferably the oncolytic recombinant rhabdovirus, or recombinant vesiculovirus, preferably oncolytic recombinant vesiculovirus, as disclosed herein both comprise at least one identical antigen or antigenic epitope whereby the antigenic domain of the complex of the first component of the invention and/or the antigenic domain of the recombinant rhabdovirus, preferably the oncolytic recombinant rhabdovirus, or recombinant vesiculovirus, preferably oncolytic recombinant vesiculovirus, of the invention as disclosed herein comprise one, two, three, four, five, six, seven, eight, nine, or ten additional antigens or antigenic epitopes that are non-identical in sequence. The term “non-identical” as used herein refers to sequences that differ in more than 10%, 15%, 20%, 30%, 35%, 40%, 45%, 50%, 60%, or 70% of the amino acids of the respective antigen or antigenic epitope. The relative sequence differences between two antigens or antigenic epitopes such as e.g. those disclosed herein can be determined using the “BLAST” algorithm as disclosed herein.

Thus, in some embodiments the antigenic domain of the complex of the first component (K) as disclosed herein and the antigenic domain encoded in the genome of the recombinant vesiculovirus, preferably oncolytic recombinant vesiculovirus, both comprise at least one antigen or antigenic epitope that is identical in sequence, whereby the complex of the first component (K) and/or the antigenic domain of the recombinant vesiculovirus, preferably oncolytic recombinant vesiculovirus, preferably of the Vesicular stomatitis Indiana virus (VSIV) or Vesicular stomatitis New Jersey virus (VSNJV), additionally comprise one, two, three, four, five, six or more antigens or antigenic epitopes as disclosed herein which are non-identical.

In some embodiments, the antigenic domain of the complex of the first component (K) of the invention and the antigenic domain encoded by the genome of the recombinant Vesicular stomatitis Indiana virus, preferably the oncolytic recombinant Vesicular stomatitis Indiana virus, as disclosed herein comprise one, two, three, four or more antigens or antigenic epitopes which are non-identical, provided that at least one antigen or antigenic epitope comprised in the antigenic domains of the complex of the first component and as encoded in the genome of the recombinant VSV, preferably the oncolytic recombinant VSV, are identical in sequence.

For example, the antigenic domain of the complex of the first component (K) and the antigenic domain of the recombinant Vesicular stomatitis Indiana virus, preferably the oncolytic recombinant Vesicular stomatitis Indiana virus, of the invention comprise one antigen or antigenic epitope the sequence of which comprises 1, 2, 3, 4, 5, 6, or more amino acid substitutions, or e.g. which is about 80% identical to the respective sequence in the corresponding antigenic domain, preferably about 85% identical, more preferably about 90%, more preferably 95%, or 98% identical.

Particularly preferably, the recombinant vesicular stomatitis virus, preferably the oncolytic recombinant vesicular stomatitis virus, of the invention as disclosed above encodes in its genome at least one antigen or antigenic epitope comprising the amino acid sequence consisting of SEQ ID NO: 45, or SEQ ID NO: 59.

The vaccine or the kit of the invention may be for use in medicine. According to one embodiment the vaccine/kit of the invention as disclosed above is for use in modulating a cellular cytotoxic immune response in a mammal, preferably in a patient in need thereof afflicted with a tumor or neoplastic disease. The term “cellular cytotoxic immune response” as used herein refers to at least one or more cytotoxic T cells also known as TC, cytotoxic T lymphocyte, CTL, T-killer cell, cytolytic T cell, CD8⁺ T-cell or killer T cell, that kills cells, e.g. cells that are infected (particularly with viruses), or cells that are damaged in other ways such as e.g. cancerous cells, or tumor cells (see e.g. Halle et al., Trends Immunol. 2017 June; 38(6):432-443.). More specifically preferred, the vaccine/kit according to the invention as disclosed above is for use in modulating a cellular cytotoxic immune response against a tumor in a mammal, e.g. a patient in need thereof afflicted with a tumor or neoplastic disease.

Preferably, the vaccine/kit of the invention is for use to modulate a cellular cytotoxic immune response against breast cancer, including triple-negative breast cancer, biliary tract cancer; bladder cancer; brain cancer including glioblastomas and medulloblastomas; cervical cancer; choriocarcinoma; colon cancer; endometrial cancer; esophageal cancer; gastric cancer; gastrointestinal stromal tumor (GIST), appendix cancer, cholangiocarcinoma, carcinoid tumor, gastrointestinal colon cancer, extrahepatic bile duct cancer, gallbladder cancer, gastric (stomach) cancer, gastrointestinal carcinoid tumor, colorectal cancer, or metastatic colorectal cancer, hematological neoplasms including acute lymphocytic and myelogenous leukemia; T-cell acute lymphoblastic leukemia/lymphoma; hairy cell leukemia; chronic myelogenous leukemia, multiple myeloma; AIDS-associated leukemias and adult T-cell leukemia lymphoma; intraepithelial neoplasms including Bowen's disease and Paget's disease; liver cancer; lung cancer, including non-small cell lung cancer, lymphomas including Hodgkin's disease and lymphocytic lymphomas; neuroblastomas; glioblastoma, oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, and osteosarcoma; skin cancer including melanoma, Merkel cell carcinoma, Kaposi's sarcoma, basal cell carcinoma, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; and renal cancer including adenocarcinoma and Wilms tumor, more preferably against colorectal cancer, or metastatic colorectal cancer, pancreatic cancer, including pancreatic adenocarcinoma, and breast cancer, including triple-negative breast cancer, even more preferred against colorectal cancer, or metastatic colorectal cancer, whereby colorectal cancer or metastatic colorectal cancer includes all the cell types and stages according to the TMN system as disclosed above.

Accordingly, the present invention also provides a first component (K) comprising a complex,
wherein the complex comprises:
   (i) a cell penetrating peptide;
   (ii) an antigenic domain, comprising at least one antigen or antigenic epitope; and
   (iii) at least one TLR peptide agonist,
wherein the components i)-iii) are covalently linked,
for use in medicine,
wherein the first component (K) is administered in combination with a second component (V), which comprises a rhabdovirus, preferably an oncolytic rhabdovirus.

In other words, the present invention also provides a second component (V) comprising a rhabdovirus, preferably an oncolytic rhabdovirus for use in medicine,
wherein the second component (V) is administered in combination with a first component (K) comprising a complex, wherein the complex comprises:
   (i) a cell penetrating peptide;
   (ii) an antigenic domain, comprising at least one antigen or antigenic epitope; and
   (iii) at least one TLR peptide agonist,
wherein the components i)-iii) are covalently linked.

The detailed description provided above for the first component (K) and the second component (V) of the vaccine applies accordingly. In particular, the rhabdovirus, preferably the oncolytic rhabdovirus, of the second component (V) may encode the antigenic domain, or at least one antigen (fragment) or an antigenic epitope thereof, of the complex of the first component (K). In other words, at least one corresponding antigen (fragment) or epitope may be (1) comprised in the complex of the first component (K) and (2) encoded by (e.g., in the genome of) the rhabdovirus, preferably the oncolytic rhabdovirus, of the second component (V). Further details regarding the first component (K) and the second component (V) of the vaccine apply accordingly. Moreover, further details regarding the medical use and administration of the first component (K) and the second component (V) of the vaccine apply accordingly.

In particular, a “combination” of the first component (K) and the second component (V) as described herein usually means that the treatment with the first component (K) as described herein is combined with the treatment with second component (V) as described herein. In other words, even if one component (the first or the second) is not administered, e.g., at the same day as the other component, their treatment schedules are intertwined. In particular, one, e.g. the first, component may be administered first (as “prime”), while the other, e.g. the second, component may be administered later (as “boost”); e.g. 1, 2, 3, 4, 5, 6, 7, 8, 9, 10, 11, 12, 13, 14, 15, 16, 17, 18, 19, 20 or more days or weeks after the “prime”. Thereby, the interval between prime and boost is usually selected such, that a strong immune response can be found. In some embodiments, the first component (K) and/or the second component (V) may be administered repeatedly. In this context, the “prime” component (e.g., the first component (K)) may be administered again after administration of the “boost” component (e.g., the second component (V)). Thus, the administration of the first component (K) and/or the second component (V) may be repeated at least two times.

In one embodiment, the first component (K) and the second component (V), respectively, e.g., of the vaccine (for use) according to the invention, are each administered at least once to a mammal, preferably a human in need thereof afflicted with a tumor or neoplastic disease. Accordingly, the first component (K) of the vaccine of the invention is at least administered once to a human or a patient in need thereof afflicted with a tumor, or cancer or neoplastic disease as disclosed above, followed by the administration of the second component (V) of the vaccine. For example, the first component (K) and second component (V) of the vaccine for use according to the invention may be administered at least once, twice, three times, or four times or more.

The first component (K) and the second component (V) of the vaccine of the invention as disclosed herein may be administered in the order K-V or V-K, whereby the “K-V” refers to the administration of the first component (K) followed by the administration of the second component “V” of the vaccine as disclosed herein. It was found, however, that a prime vaccination using the first component (K) followed by a boost using the second component (V) of the inventive vaccine results in a stronger immune response, for example as assessed by e.g. multi-epitopic CD8 CTLs and CD4 Tₕ cells.

It is particularly preferred that the first component (K) and second component (V) of the vaccine for use according to the invention are administered in the order of first component (K) followed by the administration of the second component (V).

It was found that increasing the number of administrations of the first component (K) and/or second component (V) of the vaccine according to the invention results in an enhanced cytotoxic T-cell response. In particular, when the first component (K) of the vaccine of the invention is administered repeatedly, preferably in the order of K-V-K. The first component (K) and second component (V) of the vaccine for use according to the invention may be administered according to different administration schedules such as K-V-V-K, K-K-V, V-K-K, however, administration schemes using the first component (K) of the vaccine as a prime followed by a boost using the second component (V) of the invention are preferred as they result in an advantageous increase of the CD8 T cell response against the at least one tumor or cancer epitope comprised in the antigenic domain of the first component (K) and second component (V) of the vaccine as disclosed herein.

It is to be understood that the above administration scheme does not preclude further and/or repeated administrations of the first component K of the inventive vaccine. Thus, according to one embodiment, the first component (K) and second component (V) of the vaccine for use according to the invention as disclosed herein may be administered according to one of the following administration schemes: K-V-K, K-V-K-K, K-V-V-K, preferably K-V-K, K-V-K-K.

According to one embodiment, the first component (K) and second component (V) of the vaccine may be administered sequentially, e.g. the first component (K) and second component (V) of the vaccine for use according to the invention are administered between 2, 3, 4, 5, 6, 7, 8, 9, 10, 11, 12, 13, 14, 15, 16, 18, 19, 20, 21 days apart, preferably between about 5, 6, 7, 8, 9, 10 days to about 11, 12, 13, 14, 15, 16, 17, 18, 19, 20 days apart from each other, preferably between about 11, 12, 13, 14 days to about 15, 16, 17, 18, 19, 20, 21 days. For example, The first component K for use according to the invention as disclosed above may be administered on day 0 followed by the administration of the second component (V) of the invention 2 days, 3 days, 4 days, 5 days, 6 days, 7 days, 8 days, 9 days, 10 days, 11 days, 12 days, 13 days, 14 days, 15 days, 16 days, 18 days, 19 days, 20 days, 21 days later. It is preferred that the second component (V), i.e. the recombinant vesicular stomatitis virus as disclosed herein, is administered at least 7 days, 8 days, 9, days, 10 days, 11 days, 12 days, 13 days, 14 days, 18 days, 21 days following the administration of the first component (K) of the invention.

According to one embodiment, the first component (K) for use according to the invention is administered at least once about 10, 11, 12, 13, 14 days to about 20, 21, 22, 24, 26, 28, 30, 35, 42, 49, or 56 days following the last administration of the first component (K) according to the invention. It is preferred that the time intervals between the sequential administration of the first component (K) and second component (V) according to the invention is at least 5 days, 6 days, 7 days, 8 days, 9, days, 10 days, 11 days, 12 days, 13 days, 14 days, or e.g. at least 7 days, 14 days, or 21 days, or 28 days. For example, the first component (K) according to the invention may be administered on day 0 followed by an administration of the second component (V) of the invention on day 7, day 14, day 21, or day 28, followed by a second administration of the first component (K) of the vaccine of the invention on day 14, 21, day 28, or day 35. The first component (K) for use according to the invention may e.g. additionally be administered as a boost e.g. 14 days, 21 days, 28 days, 35 days, 42 days, 49 day or 56 days or later following the last administration of the first component (K) of the invention as disclosed herein.

For example, the vaccine for use according to invention may be administered according to the following administration schemes, whereby “K” denotes the first component (K) and “V” the second component (V) of the vaccine of the invention as disclosed herein:
(1) Day 0: K, Day 7: V, Day 14: K, Day 21: K
(2) Day 0: K, Day 14 V, Day 21 K, Day 28: K
(3) Day 0: K, Day 14 V, Day 28 K, Day 35: K
(4) Day 0: K, Day 14 V, Day 28 K, Day 42: K
(5) Day 0: K Day 21: V, Day 28: K, Day 35 K
(6) Day 0: K Day 21: V, Day 35: K, Day 42 K
(7) Day 0: K, Day 28 V, Day 35: K, Day 49: K
(8) Day 0: K, Day 28 V, Day 35: K, Day 56: K
(9) Day 0: K, Day 21 V, Day 28: K, Day 35: V, Day 42: K

In some embodiments, the first component (K) of the vaccine for use according to the invention may be administered as a a maintenance therapy to the patient in need thereof following the initial vaccination of a patient with the vaccine for use according to the invention according to the administration schedule K-V-K, e.g. the first component (K) for use according to the invention may be administered according to the following administration schedule: K-V-K-Kₙ, wherein n is an integer between 1 and 20, e.g. 2, 3, 4, 5, 6, 7, 8, 9, 10, 11, 12, 13, 14, 15, 16, 18, 19 indicating the number of administrations of the first component K for use according to the invention as disclosed herein, whereby the time interval between the administration of Kₙ and Kₙ₊₁ are from about 7 days, 14 days, 21 days, 28 days to about 35 days, 42, days, 60 days, 70 days, 80 days, 90 days, 120 days, 180 days, or from about 35 days, 42, days, 60 days, 70 days, 80 days, 90 days, 120 days to about 200 days, 365 days and wherein the administration according to the administration scheme K-V-K is as disclosed above.

According to one embodiment the present invention provides a first pharmaceutical composition comprising the complex of the first component (K) or the first component (K) of the invention. As used herein, the first component (K) of the invention as disclosed herein may also refer to a pharmaceutical composition comprising the inventive complex as disclosed herein comprising a CPP, an antigenic domain and a TLR peptide agonist formulated into a pharmaceutical composition suited for administration to humans or animals, preferably humans. Typical formulations can e.g. be prepared by mixing e.g. the complex of the invention as disclosed herein with physiologically acceptable carriers, excipients or stabilizers, in the form of aqueous solutions or aqueous or non-aqueous suspensions. Carriers, excipients, modifiers or stabilizers are nontoxic at the dosages and concentrations employed. They can include buffer systems such as phosphate, citrate, acetate and other inorganic or organic acids and their salts; antioxidants including ascorbic acid and methionine; preservatives such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol; proteins, such as serum albumin, gelatin, or immunoglobulins; hydrophilic polymers such as polyvinylpyrrolidone or polyethylene glycol (PEG); amino acids such as glycine, glutamine, asparagine, histidine, arginine, or lysine; monosaccharides, disaccharides, oligosaccharides or polysaccharides and other carbohydrates including glucose, mannose, sucrose, trehalose, dextrins or dextrans; chelating agents such as EDTA; sugar alcohols such as, mannitol or sorbitol; salt-forming counter-ions such as sodium; metal complexes (e.g., Zn-protein complexes); and/or ionic or non-ionic surfactants such as TWEEN™ (polysorbates), PLURONICS™ or fatty acid esters, fatty acid ethers or sugar esters. The excipients may also have a release-modifying or absorption-modifying function.

For example, pharmaceutical compositions of the invention comprising the first component (K) as disclosed herein may comprise from about 0.001 mg/ml, 0.01 mg/ml, 0.5 mg/ml, 0.75 mg/ml, 1 mg/ml 1.5 mg/ml 2 mg/ml to about 2.5 mg/ml, 5 mg/ml, 7.5 mg/ml, 10 mg/ml, 15 mg/ml, 20 mg/ml, 25 mg/ml, 50 mg/ml of the first component K of the invention. The first pharmaceutical compositions of the invention comprising the first component (K) of the invention may e.g. comprise from about 1 nmol, 1.5 nmol, 2 nmol, 3 nmol, 4 nmol, 5 nmol to about 6 nmol, 7.5 nmol, 10 nmol, 12.5 nmol, 15 nmol, 20 nmol, 50 nmol, 100 nmol, 150 nmol, 200 nmol, of the first component (K) of the invention in a volume from about 10 μl, 25 μl, 50 μl, 75 μl to about 100 μl, 150 μl, 200 μl, 250 μl, 500 μl, 750 μl, 1 ml, 1.5 ml, 2 ml, 2.5 ml, 3 ml, 3.5 ml, 4 ml, 4.5 ml, 5 ml, 7.5 ml, or 10 ml.

In one embodiment, the first pharmaceutical composition of the invention as disclosed above is e.g. a pH-buffered solution at a pH from about 4-8, e.g., pH 4.0, pH 4.5, pH 5.0, pH 5.5, pH 6.0, pH 6.5, pH 7.0, pH 7.5, and pH 8.0. Exemplary buffers in this regard include histidine, phosphate, Tris, citrate, acetate, sodium acetate, phosphate, succinate and other organic acids. The buffer concentration can be from about 1 mM to about 30 mM, or from about 3 mM to about 20 mM, depending, for example, on the buffer and the desired isotonicity of the formulation (e.g., of the reconstituted formulation). In some embodiments, a suitable buffering agent is present at a concentration of approximately 1 mM, 5 mM, 10 mM, 15 mM, 20 mM, 25 mM, 30 mM, or 50 mM.

According to one embodiment the present invention provides a second pharmaceutical composition comprising the recombinant vesicular stomatitis virus, preferably the oncolytic recombinant vesicular stomatitis virus, of the second component (V) of the vaccine of the invention. Accordingly, the recombinant rhabdovirus, preferably the oncolytic recombinant rhabdovirus, of the invention is formulated into pharmaceutical compositions for its use according to the invention to facilitate administration to animals or humans. Typical formulations can e.g. be prepared by mixing the recombinant virus with physiologically acceptable carriers, excipients or stabilizers, in the form of aqueous solutions or aqueous or non-aqueous suspensions. Carriers, excipients, modifiers or stabilizers are nontoxic at the dosages and concentrations employed. They include buffer systems such as phosphate, citrate, acetate and other inorganic or organic acids and their salts; antioxidants including ascorbic acid and methionine; preservatives such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol; proteins, such as serum albumin, gelatin, or immunoglobulins; hydrophilic polymers such as polyvinylpyrrolidone or polyethylene glycol (PEG); amino acids such as glycine, glutamine, asparagine, histidine, arginine, or lysine; monosaccharides, disaccharides, oligosaccharides or polysaccharides and other carbohydrates including glucose, mannose, sucrose, trehalose, dextrins or dextrans; chelating agents such as EDTA; sugar alcohols such as, mannitol or sorbitol; salt-forming counter-ions such as sodium; metal complexes (e.g., Zn-protein complexes); and/or ionic or non-ionic surfactants such as TWEEN™ (polysorbates), PLURONICS™ or fatty acid esters, fatty acid ethers or sugar esters. The excipients may also have a release-modifying or absorption-modifying function.

In one embodiment the recombinant rhabdovirus, in particular the oncolytic recombinant rhabdovirus, preferably the recombinant VSV, in particular the oncolytic recombinant VSV, of the invention as disclosed above is formulated into a pharmaceutical composition comprising Tris, arginine and optionally citrate. Tris is preferably used in a concentration of about 1 mM to about 100 mM. Arginine is preferably used in a concentration of about 1 mM to about 100 mM. Citrate may be present in a concentration up to 100 mM. A preferred formulation comprises about 50 mM Tris and 50 mM arginine. The pharmaceutical composition may be provided as a liquid, a frozen liquid or in a lyophilized form. The frozen liquid may be stored at temperatures between about 0° C. and about −85° C. including temperatures between −70° C. and −85° C. and of about −15° C., −16° C., −17° C., −18° C., −19° C., −20° C., −21° C., −22° C., −23° C., −24° C. or about −25° C.

Depending on the intended use of the second pharmaceutical composition of the invention comprising the recombinant vesiculovirus of the invention as disclosed herein about 10⁸ to 10¹³ infectious particles measured by TCID₅₀ of the recombinant rhabdovirus can be an initial candidate dosage for administration to the human or patient in need thereof which may e.g. be administered by one more separate administrations, preferably by one administration.

For example, the recombinant vesiculovirus of the invention may be administered in an effective concentration of from about 10⁸, 10⁹, 10¹⁰, 10¹¹ to about 10¹², 10¹³ infectious particles measured by TCID₅₀, or from about 10⁷, 10⁸ to about 10⁹, 10¹⁰, 10¹¹, 10¹², 10¹³ infectious particles measured by TCID₅₀. For example, an initial higher loading dose, followed by one or more lower doses according to the administration schedule of the invention as disclosed above or vice versa may be useful depending on the cancer type to be treated. The progress of this therapy is easily monitored by conventional techniques and assays. The effective amount, or effective target concentration of a recombinant rhabdovirus or recombinant vesiculovirus of the invention may be expressed with the TCID₅₀. The TCID₅₀ can be determined for example by using the method of Spearman-Karber (see e.g. World J Virol 2016 May 12; 5(2): 85-86). Desirably ranges include an effective target concentration between 1×10⁸/ml and 1×10¹⁴/ml TCID₅₀. Preferably, the effective target concentration is from about 1×10⁹ to about 1×10¹²/ml, and more preferably from about 1×10⁹ to about 1×10¹¹/ml. In one embodiment, the effective target concentration is about 1×10¹⁰/ml. In a preferred embodiment the target concentration is 5×10¹⁰/ml. In another embodiment, the effective target concentration is about 1.5×10¹¹/ml. In one embodiment, the effective target concentration is about 1×10¹²/ml. In another embodiment, the effective target concentration is about 1.5×10¹³/ml. Alternatively, an effective concentration of a recombinant rhabdovirus desirably ranges between about 10⁸ and 10¹⁴ vector genomes per milliliter (vg/mL), e.g. between about 10⁹ vg/ml, 10¹⁰ vg/ml, 10¹¹ vg/ml to about 10¹² vg/ml, 10¹³ vg/ml The infectious units may be measured as described in McLaughlin et al., J Virol.; 62(6):1963-73 (1988).

According to one embodiment, the first component (K) and second component (V) of the vaccine for use according to the invention as disclosed herein, or the first and second pharmaceutical compositions of the invention may each be administered independently intratumorally (“it.”), intravenously (“i.v.”), subcutaneously (“s.c.”), intramuscularly (“i.m.”), or intraperitoneally (“i.p.”), at an effective dose. It is preferred, however, that the first component (K) of the vaccine for use according to the invention is administered intratumorally (“it.”), subcutaneously (“s.c.”), intramuscular (“i.m.”), or intraperitoneally (“i.p.”), more preferably subcutaneously (“s.c.”), or intramuscular (“i.m.”). It is preferred that the second component (V) of the vaccine for use according to the invention as disclosed herein, or the second pharmaceutical composition of the invention is administered intravenously (i.v.).

In another related embodiment, the recombinant rhabdovirus, preferably the oncolytic recombinant rhabdovirus, the recombinant vesicular stomatitis virus, preferably the oncolytic recombinant vesicular stomatitis virus, or the second pharmaceutical composition of the invention as disclosed herein are administered at least once intratumorally and subsequently intravenously. In a further related embodiment, the subsequent intravenous administration of the recombinant rhabdovirus, in particular the oncolytic recombinant rhabdovirus, preferably the recombinant vesicular stomatitis virus, in particular the oncolytic recombinant vesicular stomatitis virus, or of the second pharmaceutical composition of the invention may e.g. be according to the administration schedule disclosed herein.

An effective dose of the recombinant rhabdovirus, preferably the oncolytic recombinant rhabdovirus, as disclosed herein, or of the recombinant VSV, preferably the oncolytic recombinant VSV, as disclosed herein, or of the second component (V) of the invention may e.g.be delivered in a volume of from about 50 μl, 100 μl, 150 μl, 200 μl, 250 μl, 350 μl, 500 μl, 1 ml to about 2 ml, 2.5 ml, 3 ml, 4 ml, 5 ml, 7.5 ml, 10 ml including all numbers within the range, depending on the size of the area to be treated, the viral titer used, the route of administration, and the desired effect of the method. For intratumoral delivery of second component (V) of the invention the delivery or administration of smaller volumes may be desirable and/or advantageous as the volume that can be delivered intratumorally is limited. In instances in which only a small volume of the second component (V) of the invention can be injected into the tumor it may e.g. be advantageous to target the tumor with several injections to deliver an effective amount of the recombinant rhabdovirus, in particular the oncolytic recombinant rhabdovirus, or of the recombinant VSV, in particular the oncolytic recombinant VSV, or of the second component (V) of the invention. The amount of a given pharmaceutical composition that can be injected into the tumor can be limited such that insufficient amounts of e.g. the second pharmaceutical composition can only be administered which do not achieve the desired therapeutical effect. In those instances it may be advantageous to include recombinant hyaluronidases in the second pharmaceutical composition e.g. such as those disclosed in WO 2013/102144 to increase the injectable volume of the second pharmaceutical composition of the invention.

For systemic administration e.g. by means of infusion of the second component (V) as disclosed herein, or of the second pharmaceutical composition of the invention the volumes of administration may be naturally larger. For example, for intravenous administration the volume is preferably between 1 ml and 100 ml including volumes of about 2 ml, 3 ml, 4 ml, 5 ml, 6 ml, 7 ml, 8 ml, 9 ml, 10 ml, 11 ml, 12 ml, 13 ml, 14 ml, 15 ml, 16 ml, 17 ml, 18 ml, 19 ml, 20 ml, 25 ml, 30 ml, 35 ml, 40 ml, 45 ml, 50 ml, 55 ml, 60 ml, 70 ml, 75 ml, 80 ml, 85 ml, 90 ml, 95 ml, or about 100 ml. In a preferred embodiment the volume is between about 5 ml and 15 ml, more preferably the volume is about 6 ml, 7 ml, 8 ml, 9 ml, 10 ml, 11 ml, 12 ml, 13 ml, or about 14 ml. The term “effective dose” as used herein is an amount, or concentration of the first component (K) and/or second component (V) of the inventive vaccine that produces the desired therapeutic effect.

Preferably the same formulation, or e.g. pharmaceutical composition is used for intratumoral administration and intravenous administration of the first component (K) and the second component (V) of the vaccine of the invention, or of the first and second pharmaceutical composition of the invention. The doses and/or volume ratio between intratumoral and intravenous administration may be about 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19 or about 1:20. For example, a doses and/or volume ratio of 1:1 means that the same doses and/or volume is administered intratumorally as well as intravenously, whereas e.g. a doses and/or volume ratio of about 1:20 means an intravenous administration dose and/or volume that is twenty times higher than the intratumoral administration dose and/or volume. Preferably, the doses and/or volume ratio between intratumoral and intravenous administration is about 1:9.

The dosage of the vaccine of the invention, e.g. the dosage of the first component (K) and the second component (V) of the vaccine, that will be administered, as single or multiple doses, to an individual will vary depending upon a variety of factors, including pharmacokinetic properties, subject conditions and characteristics (sex, age, body weight, health, size), extent of symptoms, concurrent treatments, frequency of treatment and the effect desired.

In one embodiment the present invention provides for an recombinant vesicular stomatitis virus (“rVSV”), preferably an oncolytic recombinant vesicular stomatitis virus, as disclosed herein. In particular, the rVSV of the invention is selected from the group comprising: Vesicular stomatitis alagoas virus (VSAV), Carajes virus (CJSV), Chandipura virus (CHPV), Cocal virus (COCV), Vesicular stomatitis Indiana virus (VSIV), Isfahan virus (ISFV), Maraba virus (MARAV), Vesicular stomatitis New Jersey virus (VSNJV), or Piry virus (PIRYV), more preferably, the recombinant vesiculovirus of the invention is selected from one of Vesicular stomatitis Indiana virus (VSIV) or Vesicular stomatitis New Jersey virus (VSNJV), particularly preferably, the rVSV according to the invention is a recombinant Vesicular stomatitis Indiana virus (VSIV) or a Vesicular stomatitis New Jersey virus (VSNJV).

In one embodiment, the rVSV of the invention as disclosed above encodes in its genome at least for a vesicular stomatitis virus nucleoprotein (N), phosphoprotein (P), large protein (L) and a matrix protein (M), including the respective functional variants which are at least 80%, 85%, 90%, 92%, 94%, 96%, 98% identical to the respective sequences of SEQ ID NO: 49, 50, 51, 52.

In one embodiment, the wild type glycoprotein G of the rVSV as disclosed herein may e.g. be replaced by a glycoprotein (GP) of an arenavirus as disclosed above, preferably by the glycoprotein of Lassa Virus (LASV), Dandenong virus (DANDV), Mopeia (MOPV) virus, or lymphocytic choriomeningitis virus (LCMV) as disclosed above, particularly preferred by the glycoprotein of lymphocytic choriomeningitis virus (LCMV) as disclosed above.

It is preferred that the VSV glycoprotein G of the rVSV of the invention is replaced with the glycoprotein GP of the lymphocytic choriomeningitis virus (LCMV), preferably with the GP of the strain WE-HPI as disclosed in WO 2010/04052 or WO2006/008074.

The glycoprotein GP of the lymphocytic choriomeningitis virus (LCMV) as encoded by the rVSV of the invention as disclosed above may be GP1 or GP2. The glycoprotein GP of the inventive rVSV may e.g. also include glycoproteins from different LCMV strains which can be derived from LCMV wild-type or LCMV strains LCMV-WE, LCMV-WE-HPI, LCMV-WE-HPlopt as disclosed above. In a preferred embodiment, the gene coding for the glycoprotein GP of the LCMV encodes for a protein with an amino acid sequence as shown in SEQ ID NO:53 or a functional variant comprising an amino acid sequence having at least 80, 85, 90, 95%, 98%, 99% sequence identity to the amino acid sequence of SEQ ID NO: 53 having the functional properties of the glycoprotein GP encoding an amino acid sequence as shown in SEQ ID NO: 53. The glycoprotein GP for use in the rVSV according to the invention may e.g. also be derived from Lassa virus (LASV), or Mopeia virus (MOPV) as disclosed above.

According to one embodiment the rVSV according to the invention encodes in its genome a antigenic domain as defined and disclosed above which comprises at least one antigen or antigenic epitope as disclosed above of an antigen selected from the group consisting of ASCL2, EpCAM, HER-2, MUC-1, TOMM34, RNF 43, KOC1, VEGFR, βhCG, survivin, CEA, TGFβR2, p53, KRas, OGT, CASP5, COA-1, MAGE A3, SART, mesothelin, NY-ESO-1, PRAME, WT-1 or a fragment thereof, or a sequence variant of a tumor antigen or a sequence variant of a fragment thereof, preferably at least one epitope as disclosed above of an antigen selected from the group consisting of ASCL2, EpCAM, MUC-1, survivin, CEA, KRas, MAGE-A3 and IL13Ralpha2, preferably the at least one tumor epitope is an epitope of an antigen selected from the group consisting of ASCL2, EpCAM, MUC-1, survivin, CEA, KRas and MAGE-A3, more preferably the at least one tumor epitope is an epitope of an antigen selected from the group consisting of ASCL2, EpCAM, MUC-1, survivin and CEA and even more preferably the at least one tumor epitope is an epitope of an antigen selected from the group consisting of ASCL2, EpCAM, survivin and CEA, more preferably the at least one epitope as disclosed above of an antigen selected from the group consisting of ASCL2, survivin and CEA.

In a preferred embodiment, the rVSV according the invention as defined above encodes in its genome an antigenic domain which comprises an epitope of survivin, which preferably comprises a peptide having an amino acid sequence according to SEQ ID NO: 12, or a fragment thereof having a length of at least 10 amino acids, or a functional sequence variant thereof having at least 70%, 75%, 80%, 85%, 90% or 95% sequence identity, more preferably an amino acid sequence according to SEQ ID NO: 22, more preferably an amino acid sequence according to SEQ ID NO: 23 or a functional sequence variant thereof having at least 70%, 75%, 80%, 85%, 90% or 95% sequence identity.

In a preferred embodiment, the rVSV according the invention as disclosed above encodes in its genome an antigenic domain which comprises an epitope of CEA, which preferably comprises a peptide having an amino acid sequence according to SEQ ID NO: 24, or a fragment thereof having a length of at least 10 amino acids, or a functional sequence variant thereof having at least 70%, 75%, 80%, 85%, 90% or 95% sequence identity, more preferably an amino acid sequence according to SEQ ID NO: 26 and or SEQ ID NO: 27, more preferably an amino acid sequence according to SEQ ID NO: 25 or a functional sequence variant thereof having at least 70%, 75%, 80%, 85%, 90% or 95% sequence identity.

In a preferred embodiment, the rVSV according the invention as disclosed above encodes in its genome an antigenic domain which comprises an epitope of ASCL2, which preferably comprises a peptide having an amino acid sequence according to SEQ ID NO: 15, or a fragment thereof having a length of at least 10 amino acids, or a functional sequence variant thereof having at least 70%, 75%, 80%, 85%, 90% or 95% sequence identity, more preferably an amino acid sequence according to SEQ ID NO: 16 and or SEQ ID NO: 17, more preferably an amino acid sequence according to SEQ ID NO: 18 or a functional sequence variant thereof having at least 70%, 75%, 80%, 85%, 90% or 95% sequence identity.

In a most preferred embodiment of the invention, the vesicular stomatitis virus comprises or consists of an RNA sequence identical or at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 80.

In a further most preferred embodiment of the invention, the vesicular stomatitis virus comprises or consists of an RNA sequence identical or at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 80, wherein the vesicular stomatitis virus encodes in its genome a phosphoprotein (P) comprising the amino acid consisting of SEQ ID NO: 50, a nucleoprotein (N) comprising the amino acid sequence consisting of SEQ ID NO: 49, a matrix protein (M) comprising the amino acid sequence consisting of SEQ ID NO: 52, a large protein (L) comprising the amino acid sequence consisting of SEQ ID NO: 51, a glycoprotein (GP) comprising the amino acid sequence consisting of SEQ ID NO: 53, and an antigenic domain which comprises the amino acid sequence consisting of SEQ ID NO: 45 or SEQ ID NO: 59.

According to a preferred embodiment, the rVSV of the invention as disclosed above encodes in its genome an antigenic domain which comprises in N- to C-terminal direction one or more epitopes of CEA or functional sequence variants thereof; one or more epitopes of survivin or functional sequence variants thereof; and one or more epitopes of ASCL2 or functional sequence variants thereof.

Preferably, the second antigenic domain of the recombinant vesicular stomatitis virus, preferably the oncolytic recombinant vesicular stomatitis virus, of the second component (V) comprises, preferably in N- to C-terminal direction:
a peptide having an amino acid sequence according to SEQ ID NO: 24, or a fragment thereof having a length of at least 10 amino acids, or a functional sequence variant thereof having at least 70% sequence identity;
a peptide having an amino acid sequence according to SEQ ID NO: 12, or a fragment thereof having a length of at least 10 amino acids, or a functional sequence variant thereof having at least 70% sequence identity; and
a peptide having an amino acid sequence according to SEQ ID NO: 15, or a fragment thereof having a length of at least 10 amino acids, or a functional sequence variant thereof having at least 70% sequence identity.

In other words, it is preferred that the recombinant vesicular stomatitis virus, preferably the oncolytic recombinant vesicular stomatitis virus, encodes in its genome a (second) antigenic domain comprising, preferably in N- to C-terminal direction:
a peptide having an amino acid sequence according to SEQ ID NO: 24, or a fragment thereof having a length of at least 10 amino acids, or a functional sequence variant thereof having at least 70%, 75%, 80%, 85%, 90% or 95% sequence identity;
a peptide having an amino acid sequence according to SEQ ID NO: 12, or a fragment thereof having a length of at least 10 amino acids, or a functional sequence variant thereof having at least 70%, 75%, 80%, 85%, 90% or 95% sequence identity; and
a peptide having an amino acid sequence according to SEQ ID NO: 15, or a fragment thereof having a length of at least 10 amino acids, or a functional sequence variant thereof having at least 70%, 75%, 80%, 85%, 90% or 95% sequence identity.

More specifically, the rVSV according the invention as disclosed above encodes in its genome (a second) antigenic domain which comprises a peptide consisting of an amino acid sequence according to SEQ ID NO: 24 or the fragment or variant thereof wherein the its C-terminus is directly linked to the N-terminus of the peptide consisting of an amino acid sequence according to SEQ ID NO: 12 or the fragment or variant thereof; and the C-terminus of the peptide consisting of an amino acid sequence according to SEQ ID NO: 12 or the fragment or variant thereof is directly linked to the N-terminus of the peptide consisting of an amino acid sequence according to SEQ ID NO: 15 or the fragment or variant thereof.

More specifically, the rVSV according the invention as disclosed above encodes in its genome (a second) antigenic domain which comprises a peptide consisting of an amino acid sequence according to SEQ ID NO: 25 or a functional sequence variant thereof having at least 70%, 75%, 80%, 85%, 90% or 95% sequence identity; a peptide consisting of an amino acid sequence according to SEQ ID NO: 23 or a functional sequence variant thereof having at least 70%, 75%, 80%, 85%, 90% or 95% sequence identity; and a peptide consisting of an amino acid sequence according to SEQ ID NO: 18 or a functional sequence variant thereof having at least 70%, 75%, 80%, 85%, 90% or 95% sequence identity.

Particularly preferably, the rVSV according to the invention as disclosed above encodes in its genome (a second) antigenic domain of the rVSV of the invention comprises a peptide consisting of an amino acid sequence according to SEQ ID NO: 45 or a functional sequence variant thereof having at least 70%, 75%, 80%, 85%, 90% or 95% sequence identity.

According to some embodiments the rVSV according to the invention as disclosed above may e.g. encode in its genome a polypeptide comprising the antigenic domain as disclosed above and a TLR peptide agonist covalently linked to its N-terminus, or C-terminus, preferably to its C-terminus. For example, the polypeptide encoded in the genome of the rVSV as disclosed above comprises the antigenic domain comprising the amino acid sequence consisting of SEQ ID NO: 45 and a TLR peptide agonist as disclosed above, more specifically the polypeptide encoded in the genome of the rVSV comprises the antigenic domain comprising the amino acid sequence consisting of SEQ ID NO: 45 and the TLR peptide agonist anaxa consisting of the amino acid sequence according to SEQ ID NO: 7. More preferably, the rVSV according to the invention as disclosed above may e.g. encode a polypeptide comprising the amino acid sequence according to SEQ ID NO: 71.

Particularly preferably, the rVSV according to the invention as disclosed above encodes in its genome a phosphoprotein (P, VSV-P) comprising the amino acid consisting of

[SEQ ID NO: 54]
MDNLTKVREYLKSYSRLDQAVGEIDEIEAQRAEKS
NYELFQEDGVEEHTKPSYFQAADDSDTESEPEIED
NQGLYAPDPEAEQVEGFIQGPLDDYADEEVDVVFT
SDWKQPELESDEHGKTLRLTSPEGLSGEQKSQWLS
TIKAVVQSAKYWNLAECTFEASGEGVIMKERQITP
DVYKVTPVMNTHPSQSEAVSDVWSLSKTSMTFQPK
KASLQPLTISLDELFSSRGEFISVGGDGRMSHKEA
ILLGLRYKKLYNQARVKYSL,

It is further preferred that rVSV according to the invention as disclosed above encodes in its genome a nucleoprotein (N, VSV-N) comprising the amino acid sequence consisting of

[SEQ ID NO: 55]
MSVTVKRIIDNTVVVPKLPANEDPVEYPADYFRKS
KEIPLYINTTKSLSDLRGYVYQGLKSGNVSIIHVN
SYLYGALKDIRGKLDKDWSSFGINIGKAGDTIGIF
DLVSLKALDGVLPDGVSDASRTSADDKWLPLYLLG
LYRVGRTQMPEYRKKLMDGLTNQCKMINEQFEPLV
PEGRDIFDVWGNDSNYTKIVAAVDMFFHMFKKHEC
ASFRYGTIVSRFKDCAALATFGHLCKITGMSTEDV
TTWILNREVADEMVQMMLPGQEIDKADSYMPYLID
FGLSSKSPYSSVKNPAFHFWGQLTALLLRSTRARN
ARQPDDIEYTSLTTAGLLYAYAVGSSADLAQQFCV
GDNKYTPDDSTGGLTTNAPPQGRDVVEWLGWFEDQ
NRKPTPDMMQYAKRAVMSLQGLREKTIGKYAKSEF
DK

It is further preferred that rVSV according to the invention as disclosed above encodes in its genome a matrix protein (M) comprising the amino acid sequence consisting of

[SEQ ID NO: 56]
MSSLKKILGLKGKGKKSKKLGIAPPPYEEDTSMEY
APSAPIDKSYFGVDEMDTYDPNQLRYEKFFFTVKM
TVRSNRPFRTYSDVAAAVSHWDHMYIGMAGKRPFY
KILAFLGSSNLKATPAVLADQGQPEYHAHCEGRAY
LPHRMGKTPPMLNVPEHFRRPFNIGLYKGTIELTM
TIYDDESLEAAPMIWDHFNSSKFSDFREKALMFGL
IVEKKASGAVVVLDSIGHFK*

It is further preferred that rVSV according to the invention as disclosed above encodes in its genome a large protein (L) comprising the amino acid sequence consisting of

[SEQ ID NO: 57]
MEVHDFETDEFNDFNEDDYATREFLNPDERMTYLN
HADYNLNSPLISDDIDNLIRKFNSLPIPSMWDSKN
WDGVLEMLTSCQANPIPTSQMHKWMGSWLMSDNHD
ASQGYSFLHEVDKEAEITFDVVETFIRGWGNKPIE
YIKKERWTDSFKILAYLCQKFLDLHKLTLILNAVS
EVELLNLARTFKGKVRRSSHGTNICRIRVPSLGPT
FISEGWAYFKKLDILMDRNFLLMVKDVIIGRMQTV
LSMVCRIDNLFSEQDIFSLLNIYRIGDKIVERQGN
FSYDLIKMVEPICNLKLMKLARESRPLVPQFPHFE
NHIKTSVDEGAKIDRGIRFLHDQIMSVKTVDLTLV
IYGSFRHWGHPFIDYYTGLEKLHSQVTMKKDIDVS
YAKALASDLARIVLFQQFNDHKKWFVNGDLLPHDH
PFKSHVKENTWPTAAQVQDFGDKWHELPLIKCFEI
PDLLDPSIIYSDKSHSMNRSEVLKHVRMNPNTPIP
SKKVLQTMLDTKATNWKEFLKEIDEKGLDDDDLII
GLKGKERELKLAGRFFSLMSWKLREYFVITEYLIK
THFVPMFKGLTMADDLTAVIKKMLDSSSGQGLKSY
EAICIANHIDYEKWNNHQRKLSNGPVFRVMGQFLG
YPSLIERTHEFFEKSLIYYNGRPDLMRVHNNTLIN
STSQRVCWQGQEGGLEGLRQKGWSILNLLVIQREA
KIRNTAVKVLAQGDNQVICTQYKTKKSRNWELQGA
LNQMVSNNEKIMTAIKIGTGKLGLLINDDETMQSA
DYLNYGKIPIFRGVIRGLETKRWSRVTCVTNDQIP
TCANIMSSVSTNALTVAHFAENPINAMIQYNYFGT
FARLLLMMHDPALRQSLYEVQDKIPGLHSSTFKYA
MLYLDPSIGGVSGMSLSRFLIRAFPDPVTESLSFW
RFIHVHARSEHLKEMSAVFGNPEIAKFRITHIDKL
VEDPTSLNIAMGMSPANLLKTEVKKCLIESRQTIR
NQVIKDATIYLYHEEDRLRSFLWSINPLFPRFLSE
FKSGTFLGVADGLISLFQNSRTIRNSFKKKYHREL
DDLIVRSEVSSLTHLGKLHLRRGSCKMWTCSATHA
DTLRYKSWGRTVIGTTVPHPLEMLGPQHRKETPCA
PCNTSGFNYVSVHCPDGIHDVFSSRGPLPAYLGSK
TSESTSILQPWERESKVPLIKRATRLRDAISWFVE
PDSKLAMTILSNIHSLTGEEWTKRQHGFKRTGSAL
HRFSTSRMSHGGFASQSTAALTRLMATTDTMRDLG
DQNFDFLFQATLLYAQITTTVARDGWITSCTDHYH
IACKSCLRPIEEITLDSSMDYTPPDVSHVLKTWRN
GEGSWGQEIKQIYPLEGNWKNLAPAEQSYQVGRCI
GFLYGDLAYRKSTHAEDSSLFPLSIQGRIRGRGFL
KGLLDGLMRASCCQVIHRRSLAHLKRPANAVYGGL
IYLIDKLSVSPPFLSLTRSGPIRDELETIPHKIPT
SYPTSNRDMGVIVRNYFKYQCRLIEKGKYRSHYSQ
LWLFSDVLSIDFIGPFSISTTLLQILYKPFLSGKD
KNELRELANLSSLLRSGEGWEDIHVKFFTKDILLC
PEEIRHACKFGIAKDNNKDMSYPPWGRESRGTITT
IPVYYTTTPYPKMLEMPPRIQNPLLSGIRLGQLPT
GAHYKIRSILHGMGIHYRDFLSCGDGSGGMTAALL
RENVHSRGIFNSLLELSGSVMRGASPEPPSALETL
GGDKSRCVNGETCWEYPSDLCDPRTWDYFLRLKAG
LGLQIDLIVMDMEVRDSSTSLKIETNVRNYVHRIL
DEQGVLIYKTYGTYICESEKNAVTILGPMFKTVDL
VQTEFSSSQTSEVYMVCKGLKKLIDEPNPDWSSIN
ESWKNLYAFQSSEQEFARAKKVSTYFTLTGIPSQF
IPDPFVNIETMLQIFGVPTGVSHAAALKSSDRPAD
LLTISLFYMAIISYYNINHIRVGPIPPNPPSDGIA
QNVGIAITGISFWLSLMEKDIPLYQQCLAVIQQSF
PIRWEAVSVKGGYKQKWSTRGDGLPKDTRISDSLA
PIGNWIRSLELVRNQVRLNPFNEILFNQLCRTVDN
HLKWSNLRRNTGMIEWINRRISKEDRSILMLKSDL
HEENSWRD

It is further preferred that rVSV according to the invention as disclosed above encodes in its genome a glycoprotein (GP) comprising the amino acid sequence consisting of

[SEQ ID NO: 58]
MGQIVTMFEALPHIIDEVINIVIIVLIIITSIKAV
YNFATCGILALVSFLFLAGRSCGMYGLNGPDIYKG
VYQFKSVEFDMSHLNLTMPNACSANNSHHYISMGS
SGLELTFTNDSILNHNFCNLTSAFNKKTFDHTLMS
IVSSLHLSIRGNSNHKAVSCDFNNGITIQYNLSFS
DPQSAISQCRTFRGRVLDMFRTAFGGKYMRSGWGW
AGSDGKTTWCSQTSYQYLIIQNRTWENHCRYAGPF
GMSRILFAQEKTKFLTRRLAGTFTWTLSDSSGVEN
PGGYCLTKWMILAAELKCFGNTAVAKCNVNHDEEF
CDMLRLIDYNKAALSKFKQDVESALHVFKTTVNSL
ISDQLLMRNHLRDLMGVPYCNYSKFWYLEHAKTGE
TSVPKCWLVTNGSYLNETHFSDQIEQEADNMITEM
LRKDYIKRQGSTPLALMDLLMFSTSAYLISIFLHL
VKIPTHRHIKGGSCPKPHRLTNKGICSCGAFKVPG
VKTIWKRR*

It is further preferred that rVSV according to the invention as disclosed above encodes in its genome an antigenic domain which comprises the amino acid sequence consisting of

[SEQ ID NO: 59]
MANRTLTLFNVTRNDARAYVSGIQNSVSANRSDPV
TLDVLPDSSYLSGANLNLSCHSASPQYSWRINGIP
QQHTQVLFIAKITPNNNGTYACFVSNLATGRNNSI
VKSITVSASGTSPGLSAAPTLPPAWQPFLKDHRIS
TFKNWPFLEGSAVKKQFEELTLGEFLKLDRERAAV
ARRNERERNRVKLVNLGFQALRQHVPHGGASKKLS
KVETLRSAVEYIRALQRLLAEHDAVRNALAGGLRP
QAVRPSAPRGPSEGALSPAERELLDFSSWLGGY,
pVSV-GP128_-_MAD_domain.

The rVSV according to the invention may e.g. comprise functional sequence variants of each of the sequences SEQ ID NO: 54, SEQ ID NO: 55, SEQ ID NO: 56, SEQ ID NO: 57, SEQ ID NO: 58, SEQ ID NO: 59, as disclosed above, which are at least 90%, 92.5%, 95%, 97%, 98%, 99% identical to the respective sequence above. It is understood that the rVSV of the invention may encode in its genome one, two, three, four or all sequence variants of SEQ ID NO: 54, SEQ ID NO: 55, SEQ ID NO: 56, SEQ ID NO: 57, SEQ ID NO: 58, SEQ ID NO: 59, as disclosed above.

In some embodiments, the rVSV of the invention as disclosed above encodes in its genome an antigenic domain which comprises at least one antigen or antigenic domain which is identical in its amino acid sequence to at least one antigen or antigenic domain of the first component (K) of the vaccine according to the invention. The rVSV of the invention as disclosed above may e.g. additionally encode in its genome one, two, three, four, five, six, seven, eight, nine, or ten additional antigens or antigenic epitopes that are non-identical in sequence to the corresponding antigens or antigenic epitopes comprised in the antigenic domain of the complex of the first component (K) of the invention as disclosed herein. The term “non-identical” as used herein refers to sequences that differ in more than 10%, 15%, 20%, 30%, 35%, 40%, 45%, 50%, 60%, or 70% of the amino acids of the respective antigen or antigenic epitope. The relative sequence differences between two antigens or antigenic epitopes such as e.g. those disclosed herein can be determined using the “BLAST” algorithm as disclosed herein.

In some embodiments, the recombinant rVSV of the invention as disclosed above, or the complex of the first component (K) as disclosed herein comprises in addition to the at least one antigen or antigenic sequence which is identical in sequences one, two, three, four, five, six, seven, eight, nine, or ten antigens or antigenic epitopes the sequences of which are not comprised, or which are not identical in sequence to the respective antigenic domain of the either the complex of the first component (K) of the invention or the antigenic domain of the rVSV as disclosed herein. Preferably, the rVSV of the invention encodes e.g. in its genome an antigenic domain which comprises at least one antigen or antigenic epitope of CEA as disclosed above which is identical in sequence to the antigen or antigenic epitope of CEA as comprised in the antigenic domain of the complex of the first component (K) according to the invention, or the rVSV of the invention encodes in its genome an antigenic domain which comprises at least one antigen or antigenic epitope of survivin as disclosed above which is identical in sequence to the antigen or antigenic epitope of survivin as comprised in the antigenic domain of the complex of the first component (K) according to the invention, or the rVSV of the invention encodes in its genome an antigenic domain which comprises at least one antigen or antigenic epitope of ASCL2 as disclosed above which is identical in sequence to the antigen or antigenic epitope of ASCL2 as comprised in the antigenic domain of the complex of the first component (K) according to the invention, more preferably the rVSV of the invention encodes in its genome an antigenic domain which comprises at least two antigens or antigenic epitopes of CEA and survivin as disclosed above which are identical in sequence to the antigen or antigenic epitope of CEA and survivin as comprised in the antigenic domain of the complex of the first component (K) according to the invention, or the rVSV of the invention encodes in its genome an antigenic domain which comprises at least two antigens or antigenic epitopes of CEA and ASCL2 as disclosed above which are identical in sequence to the antigen or antigenic epitope of CEA and ASCL2 as comprised in the antigenic domain of the complex of the first component (K) according to the invention, or the rVSV of the invention encodes in its genome an antigenic domain which comprises at least two antigens or antigenic epitopes of survivin and ASCL2 as disclosed above which are identical in sequence to the antigen or antigenic epitope of survivin and ASCL2 as comprised in the antigenic domain of the complex of the first component (K) according to the invention, particular preferably, the rVSV of the invention encodes in its genome an antigenic domain which comprises at least three antigens or antigenic epitopes of CEA, survivin and ASCL2 as disclosed above which are identical in sequence to the antigens or antigenic epitopes of CEA, survivin and ASCL2 as comprised in the antigenic domain of the complex of the first component (K) according to the invention. It is to be understood, that the rVSV according to the invention as disclosed above, or the complex of the first component (K) according to the invention as disclosed above can comprise additional antigens or antigenic epitopes which are not comprised, or which are non-identical in sequence in the complex of the first component (K) or the rVSV of the invention.

The present invention also provides a recombinant vesicular stomatitis virus, preferably an oncolytic recombinant vesicular stomatitis virus, as described above, in particular, for use in the vaccine of the present invention. It is understood that embodiments and preferred or detailed aspects of the virus described above in the context of the vaccine, apply accordingly to the recombinant vesicular stomatitis virus, preferably the oncolytic recombinant vesicular stomatitis virus, of the present invention. In particular, the present invention also provides a recombinant vesicular stomatitis virus, preferably an oncolytic recombinant vesicular stomatitis virus, encoding in its genome at least one antigen or antigenic epitope as described above for the complex/first component (K), wherein the gene coding for the glycoprotein G of the vesicular stomatitis virus is replaced by the gene coding for the glycoprotein GP of lymphocyte choriomeningitis virus (LCMV), and/or the glycoprotein G is replaced by the glycoprotein GP of LCMV. Said recombinant vesicular stomatitis virus, preferably the oncolytic recombinant vesicular stomatitis virus, may encode in its genome a vesicular stomatitis virus nucleoprotein (N), large protein (L), phosphoprotein (P), matrix protein (M), glycoprotein (G) and at least one antigen or antigenic epitope as described above for the complex/first component (K), wherein the gene coding for the glycoprotein G of the vesicular stomatitis virus is replaced by the gene coding for the glycoprotein GP of lymphocyte choriomeningitis virus (LCMV), and/or the glycoprotein G is replaced by the glycoprotein GP of LCMV.

In some embodiments, the rVSV of the invention as disclosed herein is for use in the vaccine as disclosed above. Accordingly, the recombinant vesicular stomatitis virus for use according to the invention is preferably comprised in/constitutes the second component (V) of the vaccine of the invention. For example, the recombinant vesicular stomatitis virus according the invention as comprised in the vaccine of the invention may e.g. be comprised in a pharmaceutical composition as disclosed above.

In some embodiments, the vesicular stomatitis virus, preferably the oncolytic vesicular stomatitis virus, according to the invention as described herein may be for use in combination with the complex of the first component (K) as described herein, optionally in combination with a chemotherapeutic agent, immunotherapeutic agent, such as an immune checkpoint inhibitor, or targeted drug. Accordingly, the complex of the first component (K) according to the invention as described herein may be for use in combination with the recombinant vesicular stomatitis virus, preferably the oncolytic recombinant vesicular stomatitis virus, of the vaccine as described herein, optionally in combination with a chemotherapeutic agent, immunotherapeutic agent, such as an immune checkpoint inhibitor, or targeted drug as described herein.

In some embodiments of the vaccine of the invention the complex of the first component (K) consists of the amino acid sequence according to SEQ ID NO: 60 and the recombinant vesicular stomatitis virus, preferably the oncolytic recombinant vesicular stomatitis virus, of the second component (V) encodes in its genome
a phosphoprotein (P) comprising the amino acid consisting of SEQ ID NO: 54,
a nucleoprotein (N) comprising the amino acid sequence consisting of SEQ ID NO: 55,
a matrix protein (M) comprising the amino acid sequence consisting of SEQ ID NO: 56,
a large protein (L) comprising the amino acid sequence consisting of SEQ ID NO: 57
a glycoprotein (GP) comprising the amino acid sequence consisting of SEQ ID NO: 58, and
an antigenic domain which comprises the amino acid sequence consisting of SEQ ID NO: 59.

### Kit of Parts

In a further aspect, the present invention also provides a kit of parts comprising:
(1) a first component (K) comprising a complex, said complex comprising or consisting of:
   (i) a cell penetrating peptide;
   (ii) an antigenic domain, comprising at least one antigen or antigenic epitope; and
   (iii) at least one TLR peptide agonist,
      wherein the components i)-iii) are covalently linked, and
(2) a second component (V) comprising a rhabdovirus, preferably an oncolytic rhabdovirus.

In general, the detailed description and embodiments described herein for the first component (K) of the vaccine apply accordingly to the first component (K) of the kit of parts. Likewise, the detailed description and embodiments described herein for the second component (V) of the vaccine apply accordingly to the second component (V) of the kit of parts. In particular, the rhabdovirus, preferably the oncolytic rhabdovirus, of the second component (V) may encode the antigenic domain, or at least one antigen (fragment) or an antigenic epitope thereof, of the complex of the first component (K), as described herein for the vaccine. In other words, at least one corresponding antigen (fragment) or epitope may be (i) comprised in the complex of the first component (K) and (ii) encoded by (e.g., in the genome of) the rhabdovirus, preferably the oncolytic rhabdovirus, of the second component (V) (i.e., the virus may be capable of expressing said antigen (fragment) or epitope). Further details regarding the first and second component, including the corresponding antigens, as well as regarding its medical use and administration, as described herein for the vaccine, also apply to the kit.

In some embodiments, the kit comprises distinct containers with a first container comprising the first component (K) (but not the second component (V)) and a second container comprising the second component (V) (but not the first component (K)).

For example, the kit of parts may comprise:
(1) the complex of the first component (K) comprising or consisting of the amino acid sequence according to SEQ ID NO: 60; and
(2) the recombinant vesicular stomatitis virus, preferably the oncolytic recombinant vesicular stomatitis virus, of the second component (V) encoding in its genome
   a phosphoprotein (P) comprising the amino acid consisting of SEQ ID NO: 54,
   a nucleoprotein (N) comprising the amino acid sequence consisting of SEQ ID NO: 55,
   a matrix protein (M) comprising the amino acid sequence consisting of SEQ ID NO: 56,
   a large protein (L) comprising the amino acid sequence consisting of SEQ ID NO: 57
   a glycoprotein (GP) comprising the amino acid sequence consisting of SEQ ID NO: 58, and
   an antigenic domain which comprises the amino acid sequence consisting of SEQ ID NO: 59.

In some embodiments, the kit of parts may comprise:
(1) the complex of the first component (K) comprising or consisting of the amino acid sequence according to SEQ ID NO: 60; and
(2) the rhabdovirus, preferably the oncolytic rhabdovirus, of the second component (V) (being a vesicular stomatitis virus), wherein the RNA genome of the vesicular stomatitis virus comprises or consists of an RNA sequence identical or at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 80, preferably wherein the vesicular stomatitis virus encodes in its genome
   a phosphoprotein (P) comprising the amino acid consisting of SEQ ID NO: 50,
   a nucleoprotein (N) comprising the amino acid sequence consisting of SEQ ID NO: 49,
   a matrix protein (M) comprising the amino acid sequence consisting of SEQ ID NO: 52,
   a large protein (L) comprising the amino acid sequence consisting of SEQ ID NO: 51,
   a glycoprotein (GP) comprising the amino acid sequence consisting of SEQ ID NO: 53, and
   an antigenic domain which comprises the amino acid sequence consisting of SEQ ID NO: 45 or SEQ ID NO: 59.

In a further aspect, the invention provides for a kit of parts comprising a polypeptide and a vesicular stomatitis virus, wherein the polypeptide comprises or consists of an amino acid sequence of SEQ ID NO: 60, and wherein the RNA genome of the vesicular stomatitis virus comprises or consists of an RNA sequence identical or at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 80.

In a preferred embodiment, the present invention provides for a kit of parts comprising a polypeptide and a vesicular stomatitis virus, wherein the polypeptide comprises or consists of an amino acid sequence of SEQ ID NO: 60, and wherein the RNA genome of the vesicular stomatitis virus comprises or consists of an RNA sequence identical or at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 80, wherein the vesicular stomatitis virus encodes in its genome a phosphoprotein (P) comprising the amino acid consisting of SEQ ID NO: 50, a nucleoprotein (N) comprising the amino acid sequence consisting of SEQ ID NO: 49, a matrix protein (M) comprising the amino acid sequence consisting of SEQ ID NO: 52, a large protein (L) comprising the amino acid sequence consisting of SEQ ID NO: 51, a glycoprotein (GP) comprising the amino acid sequence consisting of SEQ ID NO: 53, and an antigenic domain which comprises the amino acid sequence consisting of SEQ ID NO: 45 or SEQ ID NO: 59.

In addition, the kit of parts may comprise a further component, e.g. as described with regard to the combinations below. In particular, the kit of parts may comprise a chemotherapeutic agent, immunotherapeutic agent (such as an immune checkpoint inhibitor), or targeted drug as described herein (e.g., for combination). Preferably, the kit of parts further comprises an immune checkpoint inhibitor of the PD-1/PD-L1 pathway, which may be selected from the group consisting of pembrolizumab; nivolumab; pidilizumab; cemiplimab; PDR-001; atezolizumab; avelumab; durvalumab, an antibody comprising a heavy chain comprising the amino acid sequence of SEQ ID NO:61 and a light chain comprising the amino acid sequence of SEQ ID NO:62; an antibody comprising a heavy chain comprising the amino acid sequence of SEQ ID NO:63 and a light chain comprising the amino acid sequence of SEQ ID NO:64; an antibody comprising a heavy chain comprising the amino acid sequence of SEQ ID NO:65 and a light chain comprising the amino acid sequence of SEQ ID NO:66; an antibody comprising a heavy chain comprising the amino acid sequence of SEQ ID NO:67 and a light chain comprising the amino acid sequence of SEQ ID NO:68; and an antibody comprising a heavy chain comprising the amino acid sequence of SEQ ID NO:69 and a light chain comprising the amino acid sequence of SEQ ID NO:70.

In some embodiments, the kit of parts comprises an antibody comprising a heavy chain comprising the amino acid sequence of SEQ ID NO:61 and a light chain comprising the amino acid sequence of SEQ ID NO:62. In some embodiments, the kit of parts comprises an antibody comprising a heavy chain comprising the amino acid sequence of SEQ ID NO:63 and a light chain comprising the amino acid sequence of SEQ ID NO:64. In some embodiments, the kit of parts comprises an antibody comprising a heavy chain comprising the amino acid sequence of SEQ ID NO:65 and a light chain comprising the amino acid sequence of SEQ ID NO:66. In some embodiments, the kit of parts comprises an antibody comprising a heavy chain comprising the amino acid sequence of SEQ ID NO:67 and a light chain comprising the amino acid sequence of SEQ ID NO:68. In some embodiments, the kit of parts comprises an antibody comprising a heavy chain comprising the amino acid sequence of SEQ ID NO:69 and a light chain comprising the amino acid sequence of SEQ ID NO:70.

In some embodiments, the kit further comprises at least one of a chemotherapeutic agent, an immune checkpoint inhibitor, targeted drug, or immunotherapeutic agent as described herein, e.g. for use in combination with the vaccine.

### Medical Uses

The vaccine of the invention efficiently induces tumor cell lysis and is characterized by a high immunogenicity, i.e. a long lasting CD8 T cell immune responses against the at least one antigen or antigenic epitope comprised in the antigenic domain of the vaccine according to the invention. Accordingly, the vaccine of the invention is useful in the treatment and/or prevention of tumors as disclosed herein.

Thus, in one aspect the present invention pertains to a method of treating a patient in need thereof which is afflicted with a tumor or cancer wherein the method comprises administering to said patient the vaccine of the invention as disclosed herein. In other words, the present invention provides a method of treating (a patient in need thereof afflicted with) a tumor or cancer, wherein the method comprises administering to said patient, (an effective amount of) the vaccine.

The present invention also provides
the vaccine as described herein,
the (oncolytic recombinant vesicular stomatitis) virus as described herein,
the kit as described herein

for use in medicine. In particular, the vaccine, the (oncolytic recombinant vesicular stomatitis) virus or the kit as described herein may be for use in the treatment and/or prevention (or prophylaxis) of a tumor or cancer, in particular in a patient in need thereof.

For the uses and methods described herein, the tumor may be selected from endocrine tumors, gastrointestinal tumors, genitourinary and gynecologic tumors, breast cancer, head and neck tumors, hematopoietic tumors, skin tumors, thoracic and respiratory tumors. In some embodiments, the tumor or cancer is selected from the group of gastrointestinal tumors comprising anal cancer, appendix cancer, cholangiocarcinoma, carcinoid tumor, gastrointestinal colon cancer, extrahepatic bile duct cancer, gallbladder cancer, gastric (stomach) cancer, gastrointestinal carcinoid tumor, gastrointestinal stromal tumor (GIST), hepatocellular cancer, pancreatic cancer, rectal cancer, colorectal cancer, or metastatic colorectal cancer. Preferably, the tumor is one of colorectal cancer, or metastatic colorectal cancer.

In some embodiments, the first component (K) and the second component (V) of the vaccine are each administered at least once. In some embodiments, the first component (K) is administered prior to the administration of the second component (V). In particular, the first component (K) may be administered at least twice, preferably prior to and subsequent to the administration of the second component (V). Preferably, the first component (K) and second component (V) are administered in the order K-V-K, K-V-K-K, K-V-V-K, more preferably in the order K-V-K, or K-V-K-K. In some instances, the first component (K) and the second component (V) of the vaccine may be administered in the order first component (K), followed by the second component (V), preferably in the order K-V-K.

In some embodiments, the first component (K) and the second component (V) of the vaccine are administered sequentially, for example the first component (K) and second component (V) are administered between 2, 3, 4, 5, 6, 7, 8, 9, 10, 11, 12, 13, 14, 15, 16, 18, 19, 20, 21 days apart, preferably between about 5, 6, 7, 8, 9, 10 days to about 11, 12, 13, 14, 15, 16, 17, 18, 19, 20 days apart from each other, preferably between about 11, 12, 13, 14 days to about 15, 16, 17, 18, 19, 20, 21 days. In some embodiments, the first component (K) and the second component (V) are administered from about 7 days to about 30 days apart from each other. Preferably, the first component (K) is administered at least once about 10, 11, 12, 13, 14 days to about 20, 22, 24, 26, 28, 30 days following the administration of the second component (V). In some embodiments, the first component (K) may be administered at least once from about 21 days to about 180 days following the last administration of the first component (K).

In some embodiments, the vaccine is co-administered with one or more of an immunotherapeutic agent, such as an immune checkpoint inhibitor, a chemotherapeutic agent, or a targeted drug as described herein. The checkpoint modulator may be administered concomitantly, sequentially, alternately or following the administration of the vaccine. In some embodiments, the checkpoint modulator is administered from about 1 to about 14 days prior to the administration of the vaccine.

Preferably, the first component (K) and second component (V) of the vaccine are administered intravenously, subcutaneously, or intramuscularly. The first component (K) and the second component (V) of the vaccine are preferably administered by different routes of administration. In some embodiments, the first component (K) of the vaccine is administered intramuscular and the second component (V) of the vaccine is administered intravenously, or intratumorally, preferably, intravenously.

In some embodiments, the dose of the complex in the first component (K) may be from about 0.5 nmol to about 10 nmol. In other words, about 0.5 nmol to about 10 nmol of the complex of the first component (K) of the vaccine are preferably administered.

In some embodiments, the dose of the rVSV of the second component (V) of the vaccine may be from about 10⁶ TCID₅₀ to about 10¹¹ TCID₅₀ In other words, the recombinant VSV of the second component (V) of the vaccine is dosed from about 10⁶ TCID₅₀ to about 10¹¹ TCID₅₀.

In particular (in the context of the vaccine for use according to the invention as described herein; the virus for use according to the invention as described herein; the complex of the first component (K) for use according to the invention as described herein; the method for use according to the invention as described herein; the kit for use according to the invention as described herein; or the polypeptide for use according to the invention as described herein), the first component (K) and the second component (V) are administered as heterologous prime-boost vaccine.

In one aspect of the invention, the patient to be treated with the vaccine as disclosed herein is afflicted with breast cancer, including triple-negative breast cancer, biliary tract cancer; bladder cancer; brain cancer including glioblastomas and medulloblastomas; cervical cancer; choriocarcinoma; colon cancer; endometrial cancer; esophageal cancer; gastric cancer; gastrointestinal stromal tumor (GIST), appendix cancer, cholangiocarcinoma, carcinoid tumor, gastrointestinal colon cancer, extrahepatic bile duct cancer, gallbladder cancer, gastric (stomach) cancer, gastrointestinal carcinoid tumor, colorectal cancer, or metastatic colorectal cancer, hematological neoplasms including acute lymphocytic and myelogenous leukemia; T-cell acute lymphoblastic leukemia/lymphoma; hairy cell leukemia; chronic myelogenous leukemia, multiple myeloma; AIDS-associated leukemias and adult T-cell leukemia lymphoma; intraepithelial neoplasms including Bowen's disease and Paget's disease; liver cancer; lung cancer, including non-small cell lung cancer, lymphomas including Hodgkin's disease and lymphocytic lymphomas; neuroblastomas; glioblastoma, oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, and osteosarcoma; skin cancer including melanoma, Merkel cell carcinoma, Kaposi's sarcoma, basal cell carcinoma, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; and renal cancer including adenocarcinoma and Wilms tumor.

In one aspect, the patient to be treated with the vaccine of the invention is afflicted with late stage colorectal cancer (CRC), or late stage metastatic colorectal cancer (mCRC), whereby the term “late stage” CRC, mCRC includes Stage IllC: T4a, N2a, M0 or T3-T4a, N2b, M0 or T4b, N1-N2, M0; Stage IVA: any T, any N, M1a and Stage IVB: any T, any N, M1b (according to TNM staging), whereby the CRC or mCRC tumor may e.g. be “Microsatellite Stable” (MSS), or microsatellite instable” (MSI), preferably the CRC or mCRC tumor is MSS: Accordingly, the vaccine of the invention as disclosed above may e.g. be administered to a patient afflicted with a tumor/cancer according to the administration scheme as disclosed herein.

In one aspect, the first component (K) and second component (V) of the vaccine of the invention may each be administered independently to the patient in need thereof, e.g. intratumorally (“it.”), intravenously (“i.v.”), subcutaneously (“s.c.”), intramuscularly (“i.m.”), or intraperitoneally (“i.p.”), at an effective dose. It is preferred, however, that the first component (K) of the vaccine for use according to the invention is administered intratumorally (“it.”), subcutaneously (“s.c.”), intramuscular (“i.m.”), or intraperitoneally (“i.p.”), more preferably subcutaneously (“s.c.”), intramuscular (“i.m.”) as disclosed above and in accordance with the administration schedule as disclosed herein. The patient to be treated may e.g. be administered the first component (K) and second component (V) of the vaccine as disclosed above.

### Combinations

The present invention also provides combination treatments of the vaccine as disclosed herein and methods providing certain advantages compared to treatments or methods currently used and/or known in the prior art. These advantages may e.g. include in vivo efficacy (e.g. improved clinical response, extend of the response, increase of the rate of response, duration of response, disease stabilization rate, duration of stabilization, time to disease progression, progression free survival (PFS) and/or overall survival (OS), later occurrence of resistance and the like), safe and well tolerated administration and reduced frequency and severity of adverse events.

The vaccine for use according to the invention may e.g. be used in combination with other pharmacologically active agents, such as state-of-the-art or standard-of-care compounds. Such compounds include e.g. cytostatic or cytotoxic substances, cell proliferation inhibitors, anti-angiogenic substances, steroids, immune modulators/checkpoint modulators all as disclosed above.

It is to be understood that the above pharmacologically active agents may be administered concomitantly, sequentially, alternately with the vaccine for use according to the invention as disclosed herein. The pharmacologically active agents may e.g. also be administered as standard-of-care treatment prior to or subsequent to the treatment with the vaccine of the invention.

In the context of the present invention it is preferred that the vaccine for use according to the invention is combined with one or more chemotherapeutic agents such as e.g. those as disclosed above.

According to one embodiment, it is preferred that the vaccine for use according to the invention, or its first (K) or second (V) component, or the rVSV of the invention as disclosed herein, is combined with (i.e., for use in combination with) a chemotherapeutic agent, immunotherapeutic agent, such as an immune checkpoint inhibitor, or targeted drug as disclosed herein.

The term “immunotherapeutic agent” as used in the context of the present invention refers to any substance that induces, enhances, restores or suppresses the host's immune system, or to an agent that utilizes or is derived from a component of the immune system. Immunotherapeutic agents for use (in medicine) in combination with the vaccine of the invention may be selected from known immunotherapeutic agents. Preferably, the immunotherapeutic agent is selected from the group comprising interferon, interleukin, or tumor necrosis factor, chimeric antigen receptors (CARs), or checkpoint modulators.

As used herein, the term “checkpoint modulator” (also referred to as “immune checkpoint modulator”) refers to a molecule or to a compound that modulates (e.g., totally or partially reduces, inhibits, interferes with, activates, stimulates, increases, reinforces or supports) the function of one or more checkpoint molecules. Thus, an immune checkpoint modulator may be an “immune checkpoint inhibitor” (also referred to as “checkpoint inhibitor” or “inhibitor”) or an “immune checkpoint activator” (also referred to as “checkpoint activator” or “activator”). An “immune checkpoint inhibitor” (also referred to as “checkpoint inhibitor” or “inhibitor”) totally or partially reduces, inhibits, interferes with, or negatively modulates the function of one or more checkpoint molecules. An “immune checkpoint activator” (also referred to as “checkpoint activator” or “activator”) totally or partially activates, stimulates, increases, reinforces, supports or positively modulates the function of one or more checkpoint molecules. Immune checkpoint modulators are typically able to modulate (i) self-tolerance and/or (ii) the amplitude and/or the duration of the immune response. Preferably, the immune checkpoint modulator used according to the present invention modulates the function of one or more human checkpoint molecules and is, thus, a “human checkpoint inhibitor”.

Checkpoint molecules are molecules, such as proteins, are typically involved in immune pathways and, for example, regulate T-cell activation, T-cell proliferation and/or T-cell function. Accordingly, the function of checkpoint molecules, which is modulated (e.g., totally or partially reduced, inhibited, interfered with, activated, stimulated, increased, reinforced or supported) by checkpoint modulators, is typically the (regulation of) T-cell activation, T-cell proliferation and/or T cell function. Immune checkpoint molecules thus regulate and maintain self-tolerance and the duration and amplitude of physiological immune responses. Many of the immune checkpoint molecules belong to the B7:CD28 family or to the tumor necrosis factor receptor (TNFR) super family and, by the binding of specific ligands, activate signaling molecules that are recruited to the cytoplasmic domain.

Artificial T cell receptors (also known as chimeric T cell receptors, chimeric immunoreceptors, chimeric antigen receptors (CARs)) are engineered receptors, which graft an arbitrary specificity onto an immune effector cell. Artificial T cell receptors (CARs) are preferred in the context of adoptive cell transfer. To this end, T cells are removed from a patient and modified so that they express receptors specific to colorectal cancer. The T cells, which can then recognize and kill the cancer cells, are reintroduced into the patient.

Preferably, the immune checkpoint modulator (for use in combination with the vaccine of the invention, the first component (K) according to the invention, the second component (V) according to the invention, or the rVSV of the invention as disclosed herein for use in medicine, in particular in the treatment and/or prevention of a tumor or cancer as disclosed herein) is an activator or an inhibitor of one or more immune checkpoint point molecule(s) selected from CD27, CD28, CD40, CD122, CD137, OX40, GITR, ICOS, A2AR, B7-H3, B7-H4, BTLA, CD40, CTLA-4, IDO, KIR, LAG3, PD-1, TIM-3, VISTA, CEACAM1, GARP, PS, CSF1R, CD94/NKG2A, TDO, GITR, TNFR and/or FasR/DcR3; or an activator or an inhibitor of one or more ligands thereof.

More preferably, the immune checkpoint modulator is an activator of a (co-)stimulatory checkpoint molecule or an inhibitor of an inhibitory checkpoint molecule or a combination thereof. Accordingly, the immune checkpoint modulator is more preferably (i) an activator of CD27, CD28, CD40, CD122, CD137, OX40, GITR and/or ICOS or (ii) an inhibitor of A2AR, B7-H3, B7-H4, BTLA, CD40, CTLA-4, IDO, KIR, LAG3, PD-1, PDL-1, PD-L2, TIM-3, VISTA, CEACAM1, GARP, PS, CSF1R, CD94/NKG2A, TDO, TNFR and/or FasR/DcR3.

Even more preferably, the immune checkpoint modulator is an inhibitor of an inhibitory checkpoint molecule (but preferably no inhibitor of a stimulatory checkpoint molecule). Accordingly, the immune checkpoint modulator is even more preferably an inhibitor of A2AR, B7-H3, B7-H4, BTLA, CTLA-4, IDO, KIR, LAG3, PD-1, PDL-1, PD-L2, TIM-3, VISTA, CEACAM1, GARP, PS, CSF1R, CD94/NKG2A, TDO, TNFR and/or DcR3 or of a ligand thereof.

It is also preferred that the immune checkpoint modulator is an activator of a stimulatory or costimulatory checkpoint molecule (but preferably no activator of an inhibitory checkpoint molecule). Accordingly, the immune checkpoint modulator is more preferably an activator of CD27, CD28, CD40, CD122, CD137, OX40, GITR and/or ICOS or of a ligand thereof.

It is even more preferred that the immune checkpoint modulator is a modulator of the CD40 pathway, of the IDO pathway, of the CTLA-4 pathway and/or of the PD-1 pathway. In particular, the immune checkpoint modulator is preferably a modulator of CD40, CTLA-4, PD-L1, PD-L2, PD-1 and/or IDO, more preferably the immune checkpoint modulator is an inhibitor of CTLA-4, PD-L1, PD-L2, PD-1 and/or IDO or an activator of CD40, even more preferably the immune checkpoint modulator is an inhibitor of CTLA-4, PD-L1, PD-1 and/or IDO and most preferably the immune checkpoint modulator is an inhibitor of CTLA-4 and/or PD-1.

It is even more preferred that the immune checkpoint modulator is a modulator of the CD40 pathway, of the IDO pathway, of the LAG3 pathway, of the CTLA-4 pathway and/or of the PD-1 pathway. In particular, the immune checkpoint modulator is preferably a modulator of CD40, LAG3, CTLA-4, PD-L1, PD-L2, PD-1 and/or IDO, more preferably the immune checkpoint modulator is an inhibitor of CTLA-4, PD-L1, PD-L2, PD-1, LAG3, and/or IDO or an activator of CD40, even more preferably the immune checkpoint modulator is an inhibitor of CTLA-4, PD-L1, PD-1, LAG3 and/or IDO, even more preferably the immune checkpoint modulator is an inhibitor of LAG3, CTLA-4 and/or PD-1, and most preferably the immune checkpoint modulator is an inhibitor of CTLA-4 and/or PD-1.

Accordingly, the checkpoint modulator (for use in combination with the vaccine of the invention, the first component (K) according to the invention, the second component (V) according to the invention, or the rVSV of the invention as disclosed herein for use in medicine, in particular in the treatment and/or prevention of a tumor or cancer as disclosed herein) may be selected from known modulators of the CD40 pathway, the CTLA-4 pathway or the PD-1 pathway. Preferably, the checkpoint modulator for combination with the complex as defined herein for the treatment of a tumor or cancer may be selected from known modulators of the CD40 pathway, the LAG3 pathway, the CTLA-4 pathway or the PD-1 pathway. Particularly preferably, the immune checkpoint modulator is a PD-1 inhibitor. Preferred inhibitors of the CTLA-4 pathway and of the PD-1 pathway include the monoclonal antibodies Yervoy® (Ipilimumab; Bristol Myers Squibb) and Tremelimumab (Pfizer/MedImmune) as well as Opdivo® (Nivolumab; Bristol Myers Squibb), Keytruda® (Pembrolizumab; MSD), Durvalumab (MedImmune/AstraZeneca), MED14736 (AstraZeneca; cf. WO 2011/066389 A1), MPDL3280A (Roche/Genentech; cf. U.S. Pat. No. 8,217,149 B2), Pidilizumab (CT-011; CureTech), MED10680 (AMP-514; AstraZeneca), avelumab (Merck KGaaA/Pfizer), MIH1 (Affymetrix) and Lambrolizumab (e.g. disclosed as hPD109A and its humanized derivatives h409AII h409A16 and h409A17 in WO2008/156712; Hamid et al., 2013; N. Engl. J. Med. 369: 134-144). More preferred checkpoint inhibitors include the CTLA-4 inhibitors Yervoy® (Ipilimumab; Bristol Myers Squibb) and Tremelimumab (Pfizer/MedImmune) as well as the PD-1 inhibitors Opdivo® (Nivolumab; Bristol Myers Squibb), Keytruda® (Pembrolizumab; MSD), Pidilizumab (CT-011; CureTech), MEDI0680 (AMP-514; AstraZeneca), AMP-224 and Lambrolizumab (e.g. disclosed as hPD109A and its humanized derivatives h409AII, h409A16 and h409A17 in WO2008/156712; Hamid O. et al., 2013; N. Engl. J. Med. 369: 134-144). As described above, a preferred example of a LAG3 inhibitor is the anti-LAG3 monoclonal antibody BMS-986016 (Bristol-Myers Squibb). Other preferred examples of a LAG3 inhibitor include LAG525 (Novartis), IMP321 (Immutep) and LAG3-Ig as disclosed in WO 2009/044273 A2 and in Brignon et al., 2009, Clin. Cancer Res. 15: 6225-6231 as well as mouse or humanized antibodies blocking human LAG3 (e.g., IMP701 as described in WO 2008/132601 A1), or fully human antibodies blocking human LAG3 (such as disclosed in EP 2320940 A2).

Particularly preferably are checkpoint inhibitors of the PD-1/PD-L1 pathway (for use, e.g. in medicine, in combination with the vaccine of the invention, the first component (K) according to the invention, the second component (V) according to the invention, the kit according to the invention, or the rVSV of the invention as disclosed herein). Preferred examples of checkpoint inhibitors of the PD-1/PD-L1 pathway (for use in combination according to the invention) include e.g pembrolizumab (anti-PD-1 antibody); nivolumab (anti-PD-1 antibody); pidilizumab (anti-PD-1 antibody); cemiplimab (anti-PD-1 antibody), PDR-001 (anti-PD-1 antibody); PD1-1, PD1-2, PD1-3, PD1-4, and PD1-5 as disclosed herein below (anti-PD-1 antibodies), atezolizumab (anti-PD-L1 antibody); avelumab (anti-PD-L1 antibody); durvalumab (anti-PD-L1 antibody). In other words, the immune checkpoint inhibitor may be selected from the group consisting of pembrolizumab; nivolumab; pidilizumab; cemiplimab; PDR-001; atezolizumab; avelumab; durvalumab, an antibody comprising a heavy chain comprising the amino acid sequence of SEQ ID NO:61 and a light chain comprising the amino acid sequence of SEQ ID NO:62; an antibody comprising a heavy chain comprising the amino acid sequence of SEQ ID NO:63 and a light chain comprising the amino acid sequence of SEQ ID NO:64; an antibody comprising a heavy chain comprising the amino acid sequence of SEQ ID NO:65 and a light chain comprising the amino acid sequence of SEQ ID NO:66; an antibody comprising a heavy chain comprising the amino acid sequence of SEQ ID NO:67 and a light chain comprising the amino acid sequence of SEQ ID NO:68; and an antibody comprising a heavy chain comprising the amino acid sequence of SEQ ID NO:69 and a light chain comprising the amino acid sequence of SEQ ID NO:70.

Pembrolizumab (formerly also known as lambrolizumab; trade name Keytruda; also known as MK-3475) disclosed e.g. in Hamid, O. et al. (2013) New England Journal of Medicine 369(2):134-44, is a humanized IgG4 monoclonal antibody that binds to PD-1; it contains a mutation at C228P designed to prevent Fc-mediated cytotoxicity. Pembrolizumab is e.g. disclosed in U.S. Pat. No. 8,354,509 and WO2009/114335. It is approved by the FDA for the treatment of patients suffering from unresectable or metastatic melanoma and patients with metastatic NSCLC.

Nivolumab (CAS Registry Number: 946414-94-4; BMS-936558 or MDX1106b) is a fully human IgG4 monoclonal antibody which specifically blocks PD-1, lacking detectable antibody-dependent cellular toxicity (ADCC). Nivolumab is e.g. disclosed in U.S. Pat. No. 8,008,449 and WO2006/121168. It has been approved by the FDA for the treatment of patients suffering from unresectable or metastatic melanoma, metastatic NSCLC and advanced renal cell carcinoma.

Pidilizumab (CT-011; Cure Tech) is a humanized IgG1k monoclonal antibody that binds to PD-1. Pidilizumab is e.g. disclosed in WO2009/101611.

PDR-001 or PDR001 is a high-affinity, ligand-blocking, humanized anti-PD-1 IgG4 antibody that blocks the binding of PD-L1 and PD-L2 to PD-1. PDR-001 is disclosed in WO2015/112900 and WO2017/019896.

Antibodies PD1-1 to PD1-5 are antibody molecules defined by the sequences as shown in Table 2, wherein HC denotes the (full length) heavy chain and LC denotes the (full length) light chain:

Cemiplimab (also known as “REGN-2810”) is a fully human monoclonal antibody to PD-1 and is e.g. disclosed in WO2015/112800.

Avelumab (MSB0010718C) is a fully human anti-PD immunoglobulin G1 (IgG1) lambda monoclonal antibody and is disclosed e.g. in WO2013/079174

Atezolizumab (also known as MPDL3280A) is a phage-derived human IgG1k monoclonal antibody targeting PD-L1 and is described e.g. in Deng et al. mAbs 2016; 8:593-603. It has been approved by the FDA for the treatment of patients suffering from urothelial carcinoma.

Durvalumab (MED14736) is a human IgG1k monoclonal antibody with high specificity to PD-L1 and described in e.g. Stewart et al. Cancer Immunol. Res. 2015; 3:1052-1062 or in Ibrahim et al. Semin. Oncol. 2015; 42:474-483.

Further PD-1 antagonists disclosed by Li et al. (supra), or known to be in clinical trials, such as AMP-224, MED10680 (AMP-514), BMS-936559, JS001-PD-1, SHR-1210, BMS-936559, TSR-042, JNJ-63723283, MED14736, MPDL3280A, may be used as alternative or in addition to the above mentioned antagonists.

The INNs as used herein are meant to also encompass all biosimilar antibodies having the same, or substantially the same, amino acid sequences as the originator antibody, including but not limited to those biosimilar antibodies authorized under 42 USC § 262 subsection (k) in the US and equivalent regulations in other jurisdictions.

The PD-1 antagonists listed above (for use, e.g. in medicine, in combination with the vaccine according to the invention) are known in the art with their respective manufacture, therapeutic use and properties.

TABLE 2

| SEQ | | |
|---|---|---|
| ID | Sequence | |
| NO: | name | Amino acid sequence |
| 61 | HC of | EVMLVESGGGLVQPGGSLRL |
| | PD1-1 | SCTASGFTFSASAMSWVRQA |
| | | PGKGLEWVAYISGGGGDTYY |
| | | SSSVKGRFTISRDNAKNSLY |
| | | LQMNSLRAEDTAVYYCARHS |
| | | NVNYYAMDYWGQGTLVTVSS |
| | | ASTKGPSVFPLAPCSRSTSE |
| | | STAALGCLVKDYFPEPVTVS |
| | | WNSGALTSGVHTFPAVLQSS |
| | | GLYSLSSVVTVPSSSLGTKT |
| | | YTCNVDHKPSNTKVDKRVES |
| | | KYGPPCPPCPAPEFLGGPSV |
| | | FLFPPKPKDTLMISRTPEVT |
| | | CVVVDVSQEDPEVQFNWYVD |
| | | GVEVHNAKTKPREEQFNSTY |
| | | RVVSVLTVLHQDWLNGKEYK |
| | | CKVSNKGLPSSIEKTISKAK |
| | | GQPREPQVYTLPPSQEEMTK |
| | | NQVSLTCLVKGFYPSDIAVE |
| | | WESNGQPENNYKTTPPVLDS |
| | | DGSFFLYSRLTVDKSRWQEG |
| | | NVFSCSVMHEALHNHYTQKS |
| | | LSLSLG |
| 62 | LC of | EIVLTQSPATLSLSPGERAT |
| | PD1-1 | MSCRASENIDTSGISFMNWY |
| | | QQKPGQAPKLLIYVASNQGS |
| | | GIPARFSGSGSGTDFTLTIS |
| | | RLEPEDFAVYYCQQSKEVPW |
| | | TFGQGTKLEIKRTVAAPSVF |
| | | IFPPSDEQLKSGTASVVCLL |
| | | NNFYPREAKVQWKVDNALQS |
| | | GNSQESVTEQDSKDSTYSLS |
| | | STLTLSKADYEKHKVYACEV |
| | | THQGLSSPVTKSFNRGEC |
| 63 | HC of | EVMLVESGGGLVQPGGSLRL |
| | PD1-2 | SCTASGFTFSASAMSWVRQA |
| | | PGKGLEWVAYISGGGGDTYY |
| | | SSSVKGRFTISRDNAKNSLY |
| | | LQMNSLRAEDTAVYYCARHS |
| | | NPNYYAMDYWGQGTLVTVSS |
| | | ASTKGPSVFPLAPCSRSTSE |
| | | STAALGCLVKDYFPEPVTVS |
| | | WNSGALTSGVHTFPAVLQSS |
| | | GLYSLSSVVTVPSSSLGTKT |
| | | YTCNVDHKPSNTKVDKRVES |
| | | KYGPPCPPCPAPEFLGGPSV |
| | | FLFPPKPKDTLMISRTPEVT |
| | | CVVVDVSQEDPEVQFNWYVD |
| | | GVEVHNAKTKPREEQFNSTY |
| | | RVVSVLTVLHQDWLNGKEYK |
| | | CKVSNKGLPSSIEKTISKAK |
| | | GQPREPQVYTLPPSQEEMTK |
| | | NQVSLTCLVKGFYPSDIAVE |
| | | WESNGQPENNYKTTPPVLDS |
| | | DGSFFLYSRLTVDKSRWQEG |
| | | NVFSCSVMHEALHNHYTQKS |
| | | LSLSLG |
| 64 | LC of | EIVLTQSPATLSLSPGERAT |
| | PD1-2 | MSCRASENIDTSGISFMNWY |
| | | QQKPGQAPKLLIYVASNQGS |
| | | GIPARFSGSGSGTDFTLTIS |
| | | RLEPEDFAVYYCQQSKEVPW |
| | | TFGQGTKLEIKRTVAAPSVF |
| | | IFPPSDEQLKSGTASVVCLL |
| | | NNFYPREAKVQWKVDNALQS |
| | | GNSQESVTEQDSKDSTYSLS |
| | | STLTLSKADYEKHKVYACEV |
| | | THQGLSSPVTKSFNRGEC |
| 65 | HC of | EVMLVESGGGLVQPGGSLRL |
| | PD1-3 | SCTASGFTFSKSAMSWVRQA |
| | | PGKGLEWVAYISGGGGDTYY |
| | | SSSVKGRFTISRDNAKNSLY |
| | | LQMNSLRAEDTAVYYCARHS |
| | | NVNYYAMDYWGQGTLVTVSS |
| | | ASTKGPSVFPLAPCSRSTSE |
| | | STAALGCLVKDYFPEPVTVS |
| | | WNSGALTSGVHTFPAVLQSS |
| | | GLYSLSSVVTVPSSSLGTKT |
| | | YTCNVDHKPSNTKVDKRVES |
| | | KYGPPCPPCPAPEFLGGPSV |
| | | FLFPPKPKDTLMISRTPE |
| | | VTCVVVDVSQEDPEV |
| | | QFNWYVDGVEVHNAKTKPRE |
| | | EQFNSTYRVVSVLTVLHQDW |
| | | LNGKEYKCKVSNKGLPSSIE |
| | | KTISKAKGQPREPQVYTLPP |
| | | SQEEMTKNQVSLTCLVKGFY |
| | | PSDIAVEWESNGQPENNYKT |
| | | TPPVLDSDGSFFLYSRLTVD |
| | | KSRWQEGNVFSCSVMHEALH |
| | | NHYTQKSLSLSLG |
| 66 | LC of | EIVLTQSPATLSLSPGERAT |
| | PD1-3 | MSCRASENIDVSGISFMNWY |
| | | QQKPGQAPKLLIYVASNQGS |
| | | GIPARFSGSGSGTDFTLTIS |
| | | RLEPEDFAVYYCQQSKEVPW |
| | | TFGQGTKLEIKRTVAAPSVF |
| | | IFPPSDEQLKSGTASVVCLL |
| | | NNFYPREAKVQWKVDNALQS |
| | | GNSQESVTEQDSKDSTYSLS |
| | | STLTLSKADYEKHKVYACEV |
| | | THQGLSSPVTKSFNRGEC |
| 67 | HC of | EVMLVESGGGLVQPGGSLRL |
| | PD1-4 | SCTASGFTFSKSAMSWVRQA |
| | | PGKGLEWVAYISGGGGDTYY |
| | | SSSVKGRFTISRDNAKNSLY |
| | | LQMNSLRAEDTAVYYCARHS |
| | | NVNYYAMDYWGQGTLVTVSS |
| | | ASTKGPSVFPLAPCSRSTSE |
| | | STAALGCLVKDYFPEPVTVS |
| | | WNSGALTSGVHTFPAVLQSS |
| | | GLYSLSSVVTVPSSSLGTKT |
| | | YTCNVDHKPSNTKVDKRVES |
| | | KYGPPCPPCPAPEFLGGPSV |
| | | FLFPPKPKDTLMISRTPEVT |
| | | CVVVDVSQEDPEVQFNWYVD |
| | | GVEVHNAKTKPREEQFNSTY |
| | | RVVSVLTVLHQDWLNGKEYK |
| | | CKVSNKGLPSSIEKTISKAK |
| | | GQPREPQVYTLPPSQEEMTK |
| | | NQVSLTCLVKGFYPSDIAVE |
| | | WESNGQPENNYKTTPPVLDS |
| | | DGSFFLYSRLTVDKSRWQEG |
| | | NVFSCSVMHEALHNHYTQKS |
| | | LSLSLG |
| 68 | LC of | EIVLTQSPATLSLSPGERAT |
| | PD1-4 | MSCRASENIDVSGISFMNWY |
| | | QQKPGQAPKLLIYVASNQGS |
| | | GIPARFSGSGSGTDFTLTIS |
| | | RLEPEDFAVYYCQQSKEVPW |
| | | TFGQGTKLEIKRTVAAPSVF |
| | | IFPPSDEQLKSGTASVVCLL |
| | | NNFYPREAKVQWKVDNALQS |
| | | GNSQESVTEQDSKDSTYSLS |
| | | STLTLSKADYEKHKVYACEV |
| | | THQGLSSPVTKSFNRGEC |
| 69 | HC of | EVMLVESGGGLVQPGGSLRL |
| | PD1-5 | SCTASGFTFSKSAMSWVRQA |
| | | PGKGLEWVAYISGGGGDTYY |
| | | SSSVKGRFTISRDNAKNSLY |
| | | LQMNSLRAEDTAVYYCARHS |
| | | NVNYYAMDYWGQGTLVTVSS |
| | | ASTKGPSVFPLAPCSRSTSE |
| | | STAALGCLVKDYFPEPVTVS |
| | | WNSGALTSGVHTFPAVLQSS |
| | | GLYSLSSVVTVPSSSLGTKT |
| | | YTCNVDHKPSNTKVDKRVES |
| | | KYGPPCPPCPAPEFLGGPSV |
| | | FLFPPKPKDTLMISRTPEVT |
| | | CVVVDVSQEDPEVQFNWYVD |
| | | GVEVHNAKTKPREEQFNSTY |
| | | RVVSVLTVLHQDWLNGKEYK |
| | | CKVSNKGLPSSIEKTISKAK |
| | | GQPREPQVYTLPPSQEEMTK |
| | | NQVSLTCLVKGFYPSDIAVE |
| | | WESNGQPENNYKTTPPVLDS |
| | | DGSFFLYSRLTVDKSRWQEG |
| | | NVFSCSVMHEALHNHYTQKS |
| | | LSLSLG |
| 70 | LC of | EIVLTQSPATLSLSPGERAT |
| | PD1-5 | MSCRASENIDVSGISFMNWY |
| | | QQKPGQAPKLLIYVASNQGS |
| | | GIPARFSGSGSGTDFTLTIS |
| | | RLEPEDFAVYYCQQSKEVPW |
| | | TFGQGTKLEIKRTVAAPSVF |
| | | IFPPSDEQLKSGTASVVCLL |
| | | NNFYPREAKVQWKVDNALQS |
| | | GNSQESVTEQDSKDSTYSLS |
| | | STLTLSKADYEKHKVYACEV |
| | | THQGLSSPVTKSFNRGEC |

Specifically, the anti-PD-1 antibody molecules PD1-1.-PD1-5 as described herein above are defined by:
(PD1-1:) a heavy chain comprising the amino acid sequence of SEQ ID NO:61 and a light chain comprising the amino acid sequence of SEQ ID NO:62; or
(PD1-2:) a heavy chain comprising the amino acid sequence of SEQ ID NO:63 and a light chain comprising the amino acid sequence of SEQ ID NO:64; or
(PD1-3:) a heavy chain comprising the amino acid sequence of SEQ ID NO:65 and a light chain comprising the amino acid sequence of SEQ ID NO:66; or
(PD1-4:) a heavy chain comprising the amino acid sequence of SEQ ID NO:67 and a light chain comprising the amino acid sequence of SEQ ID NO:68; or
(PD1-5:) a heavy chain comprising the amino acid sequence of SEQ ID NO:69 and a light chain comprising the amino acid sequence of SEQ ID NO:70.

In some embodiments (in particular of the complex of the first component (K) for use as described herein, the virus for use as described herein, the vaccine for use as described herein, the polypeptide for use or the kit as described herein), the immune checkpoint inhibitor is selected from the group consisting of pembrolizumab; nivolumab; pidilizumab; cemiplimab; PDR-001; atezolizumab; avelumab; durvalumab, an antibody comprising a heavy chain comprising the amino acid sequence of SEQ ID NO:61 and a light chain comprising the amino acid sequence of SEQ ID NO:62; an antibody comprising a heavy chain comprising the amino acid sequence of SEQ ID NO:63 and a light chain comprising the amino acid sequence of SEQ ID NO:64; an antibody comprising a heavy chain comprising the amino acid sequence of SEQ ID NO:65 and a light chain comprising the amino acid sequence of SEQ ID NO:66; an antibody comprising a heavy chain comprising the amino acid sequence of SEQ ID NO:67 and a light chain comprising the amino acid sequence of SEQ ID NO:68; and an antibody comprising a heavy chain comprising the amino acid sequence of SEQ ID NO:69 and a light chain comprising the amino acid sequence of SEQ ID NO:70.

Accordingly, the vaccine of the present invention or the kit of the present invention may further comprise an immune checkpoint inhibitor of the PD-1/PD-L1 pathway, preferably selected from the group consisting of pembrolizumab; nivolumab; pidilizumab; cemiplimab; PDR-001; atezolizumab; avelumab; durvalumab, an antibody comprising a heavy chain comprising the amino acid sequence of SEQ ID NO:61 and a light chain comprising the amino acid sequence of SEQ ID NO:62; an antibody comprising a heavy chain comprising the amino acid sequence of SEQ ID NO:63 and a light chain comprising the amino acid sequence of SEQ ID NO:64; an antibody comprising a heavy chain comprising the amino acid sequence of SEQ ID NO:65 and a light chain comprising the amino acid sequence of SEQ ID NO:66; an antibody comprising a heavy chain comprising the amino acid sequence of SEQ ID NO:67 and a light chain comprising the amino acid sequence of SEQ ID NO:68; and an antibody comprising a heavy chain comprising the amino acid sequence of SEQ ID NO:69 and a light chain comprising the amino acid sequence of SEQ ID NO:70.

In some embodiments, the vaccine as described herein comprising:
(1) the complex of the first component (K) comprising or consisting of the amino acid sequence according to SEQ ID NO: 60; and
(2) the recombinant vesicular stomatitis virus, preferably the oncolytic recombinant vesicular stomatitis virus, of the second component (V) encoding in its genome
   a phosphoprotein (P) comprising the amino acid consisting of SEQ ID NO: 54,
   a nucleoprotein (N) comprising the amino acid sequence consisting of SEQ ID NO: 55,
   a matrix protein (M) comprising the amino acid sequence consisting of SEQ ID NO: 56,
   a large protein (L) comprising the amino acid sequence consisting of SEQ ID NO: 57
   a glycoprotein (GP) comprising the amino acid sequence consisting of SEQ ID NO: 58, and
   an antigenic domain which comprises the amino acid sequence consisting of SEQ ID NO: 59

may further comprise an immune checkpoint inhibitor of the PD-1/PD-L1 pathway, preferably selected from the group consisting of pembrolizumab; nivolumab; pidilizumab; cemiplimab; PDR-001; atezolizumab; avelumab; durvalumab, an antibody comprising a heavy chain comprising the amino acid sequence of SEQ ID NO:61 and a light chain comprising the amino acid sequence of SEQ ID NO:62; an antibody comprising a heavy chain comprising the amino acid sequence of SEQ ID NO:63 and a light chain comprising the amino acid sequence of SEQ ID NO:64; an antibody comprising a heavy chain comprising the amino acid sequence of SEQ ID NO:65 and a light chain comprising the amino acid sequence of SEQ ID NO:66; an antibody comprising a heavy chain comprising the amino acid sequence of SEQ ID NO:67 and a light chain comprising the amino acid sequence of SEQ ID NO:68; and an antibody comprising a heavy chain comprising the amino acid sequence of SEQ ID NO:69 and a light chain comprising the amino acid sequence of SEQ ID NO:70.

Preferably, the vaccine as described herein comprising:
(1) the complex of the first component (K) comprising or consisting of the amino acid sequence according to SEQ ID NO: 60; and
(2) the rhabdovirus, preferably the oncolytic rhabdovirus, of the second component (V) (being a vesicular stomatitis virus), wherein the RNA genome of the vesicular stomatitis virus comprises or consists of an RNA sequence identical or at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 80, preferably wherein the vesicular stomatitis virus encodes in its genome
   a phosphoprotein (P) comprising the amino acid consisting of SEQ ID NO: 50,
   a nucleoprotein (N) comprising the amino acid sequence consisting of SEQ ID NO: 49,
   a matrix protein (M) comprising the amino acid sequence consisting of SEQ ID NO: 52,
   a large protein (L) comprising the amino acid sequence consisting of SEQ ID NO: 51,
   a glycoprotein (GP) comprising the amino acid sequence consisting of SEQ ID NO: 53, and
   an antigenic domain which comprises the amino acid sequence consisting of SEQ ID NO: 45 or SEQ ID NO: 59

may further comprise an immune checkpoint inhibitor of the PD-1/PD-L1 pathway, preferably selected from the group consisting of pembrolizumab; nivolumab; pidilizumab; cemiplimab; PDR-001; atezolizumab; avelumab; durvalumab, an antibody comprising a heavy chain comprising the amino acid sequence of SEQ ID NO:61 and a light chain comprising the amino acid sequence of SEQ ID NO:62; an antibody comprising a heavy chain comprising the amino acid sequence of SEQ ID NO:63 and a light chain comprising the amino acid sequence of SEQ ID NO:64; an antibody comprising a heavy chain comprising the amino acid sequence of SEQ ID NO:65 and a light chain comprising the amino acid sequence of SEQ ID NO:66; an antibody comprising a heavy chain comprising the amino acid sequence of SEQ ID NO:67 and a light chain comprising the amino acid sequence of SEQ ID NO:68; and an antibody comprising a heavy chain comprising the amino acid sequence of SEQ ID NO:69 and a light chain comprising the amino acid sequence of SEQ ID NO:70.

In some embodiments, said vaccine comprises an antibody comprising a heavy chain comprising the amino acid sequence of SEQ ID NO:61 and a light chain comprising the amino acid sequence of SEQ ID NO:62. In some embodiments, said vaccine comprises an antibody comprising a heavy chain comprising the amino acid sequence of SEQ ID NO:63 and a light chain comprising the amino acid sequence of SEQ ID NO:64. In some embodiments, said vaccine comprises an antibody comprising a heavy chain comprising the amino acid sequence of SEQ ID NO:65 and a light chain comprising the amino acid sequence of SEQ ID NO:66. In some embodiments, said vaccine comprises an antibody comprising a heavy chain comprising the amino acid sequence of SEQ ID NO:67 and a light chain comprising the amino acid sequence of SEQ ID NO:68. In some embodiments, said vaccine comprises an antibody comprising a heavy chain comprising the amino acid sequence of SEQ ID NO:69 and a light chain comprising the amino acid sequence of SEQ ID NO:70.

In the context of the present invention more than one immune checkpoint modulator (e.g., checkpoint inhibitor) may be used in combination with the vaccine of the invention, the first component (K) and/or second component (V), as disclosed herein, or the rVSV according to the invention as disclosed herein, in particular at least 2, 3, 4, 5, 6, 7, 8, 9 or 10 distinct immune checkpoint modulators (e.g., checkpoint inhibitors) are used, preferably 2, 3, 4 or 5 distinct immune checkpoint modulators (e.g., checkpoint inhibitors) are used, more preferably 2, 3 or 4 distinct immune checkpoint modulators (e.g., checkpoint inhibitors) are used, even more preferably 2 or 3 distinct immune checkpoint modulators (e.g., checkpoint inhibitors) are used and most preferably 2 distinct immune checkpoint modulators (e.g., checkpoint inhibitors) are used. Thereby, “distinct” immune checkpoint modulators (e.g., checkpoint inhibitors) means in particular that they modulate (e.g., inhibit) different checkpoint molecule pathways.

Accordingly, preferred combinations of immune checkpoint modulators of the PD-1 pathway and of the CTLA-4 pathway are (i) Nivolumab (anti-PD1) and Ipilimumab (anti-CTLA4) or (ii) Durvalumab (MED14736; anti-PD-L1) and Tremelimumab (anti-CTLA4), PD1-1, PD1-2, PD1-3, PD1-4, PD1-5 as disclosed herein and Ipilimumab.

Other preferred combinations of at least two distinct immune checkpoint modulators in the context of the present invention may comprise a combination selected from (i) a combination of a KIR inhibitor and a CTLA-4 inhibitor, such as Lirilumab/Ipilimumab; (ii) a combination of a KIR inhibitor and an inhibitor of the PD-1 pathway, such as a PD-1 inhibitor, for example Lirilumab/Nivolumab; or PD1-1, PD1-2, PD1-3, PD1-4, PD1-5 as disclosed herein (iii) a combination of a LAG3 inhibitor and an inhibitor of the PD-1 pathway, such as a PD-1 inhibitor or a PD-L1 inhibitor, e.g. PD1-1, PD1-2, PD1-3, PD1-4, PD1-5 as disclosed herein, or e.g. as described in Woo et al., 2012, Cancer Res. 72: 917-27 or in Butler N. S. et al., 2011, Nat Immunol. 13: 188-95) and preferred examples of such a combination include Nivolumab/BMS-986016 and PDR001/LAG525; (iv) a combination of checkpoint modulators targeting ICOS and an inhibitor of the CTLA-4, for example as described in Fu et al., 2011, Cancer Res. 71: 5445-54; (v) a combination of checkpoint modulators modulating 4-1 BB and inhibitors of CTLA-4, such as described in Curran et al., 2011, PLoS One 6(4): el 9499); (vi) a combination of checkpoint modulators targeting PD1 and CD27, such as Nivolumab/Varlilumab and Atezolizumab/Varlilumab; (vii) a combination of checkpoint modulators targeting OX40 and CTLA-4, such as MED16469/Tremelimumab; (viii) a combination of checkpoint modulators targeting OX40 and PD-1, such as MED16469/MED14736, MOXR0916/MPDL3280A, MED16383/MED14736 and GSK3174998/Pembrolizumab; (ix) a combination of checkpoint modulators targeting PD-1 and 4-1BB, such as Nivolumab/Urelumab, Pembrolizumab/PF-05082566 and Avelumab/PF-05082566; PD1-1, PD1-2, PD1-3, PD1-4, PD1-5 as disclosed herein and PF-05082566 (x) a combination of checkpoint modulators targeting PD-1 and IDO, such as Ipilimumab/Indoximod, Pembrolizumab/INCB024360, MED14736/INCB024360, MPDL3280A/GDC-0919 and Atezolizumab/INCB024360; PD1-1, PD1-2, PD1-3, PD1-4, PD1-5 as disclosed herein and PF-05082566 (xi) a combination of checkpoint modulators targeting PD-1 and CSF1R, such as Pembrolizumab/PLX3397, Nivolumab/FPA008 and MPDL3280A/RO5509554; (xii) a combination of checkpoint modulators targeting PD-1 and GITR, such as Nivolumab/BMS-986156 and Pembrolizumab/MK-4166 or PD1-1, PD1-2, PD1-3, PD1-4, PD1-5 as disclosed herein and BMS-986156; (xiii) a combination of checkpoint modulators targeting PD-1 and CD40, such as MPDL3280A/RO7009789; (xiv) a combination of checkpoint modulators targeting PD-1 and B7-H3, such as Pembrolizumab/enoblituzumab or PD1-1, PD1-2, PD1-3, PD1-4, PD1-5 as disclosed herein and enoblituzumab; (xv) a combination of checkpoint modulators targeting CTLA-4 and B7-H3, such as Ipilimumab/enoblituzumab and (xvi) a combination of checkpoint modulators targeting KIR and 4-1BB, such as Lirilumab/Urelumab.

Particularly preferably, the combination of the immune checkpoint modulator and the vaccine of the invention, the first component (K) and/or second component (V), as disclosed herein, or the rVSV according to the invention as disclosed herein for use according to the present invention comprises at least (i) an inhibitor of CTLA-4 and (ii) an inhibitor of PD-1, PD-L1 or PD-L2, preferably at least (i) an inhibitor of CTLA-4 and (ii) an inhibitor of PD-1. Examples of such a preferred combination include a combination of Yervoy® (Ipilimumab; Bristol Myers Squibb) and Opdivo® (Nivolumab; Bristol Myers Squibb), a combination of Yervoy® (Ipilimumab; Bristol Myers Squibb) and PD1-1, PD1-2, PD1-3, PD1-4, PD1-5 as disclosed herein, a combination of Yervoy® (Ipilimumab; Bristol Myers Squibb) and Keytruda® (Pembrolizumab; MSD), a combination of Yervoy® (Ipilimumab; Bristol Myers Squibb) and Durvalumab (MedImmune/AstraZeneca), a combination of Yervoy® (Ipilimumab; Bristol Myers Squibb) and MED14736 (AstraZeneca; cf. WO 2011/066389 A1), a combination of Yervoy® (Ipilimumab; Bristol Myers Squibb) and MPDL3280A (Roche/Genentech; cf. U.S. Pat. No. 8,217,149 B2), a combination of Yervoy® (Ipilimumab; Bristol Myers Squibb) and Pidilizumab (CT-011; CureTech), a combination of Yervoy® (Ipilimumab; Bristol Myers Squibb) and MED10680 (AMP-514; AstraZeneca), a combination of Yervoy® (Ipilimumab; Bristol Myers Squibb) and avelumab (Merck KGaA/Pfizer), a combination of Yervoy® (Ipilimumab; Bristol Myers Squibb) and MIH1 (Affymetrix), a combination ofYervoy® (Ipilimumab; Bristol Myers Squibb) and AMP-224, a combination of Yervoy® (Ipilimumab; Bristol Myers Squibb) and Lambrolizumab, a combination of Tremelimumab (Pfizer/MedImmune) and Opdivo® (Nivolumab; Bristol Myers Squibb), a combination of Tremelimumab (Pfizer/MedImmune) and Keytruda® (Pembrolizumab; Merck), a combination of Tremelimumab (Pfizer/MedImmune) and Durvalumab (MedImmune/AstraZeneca), a combination of Tremelimumab (Pfizer/MedImmune) and MED14736 (AstraZeneca; cf. WO 2011/066389 A1), a combination of Tremelimumab (Pfizer/MedImmune) and MPDL3280A (Roche/Genentech; cf. U.S. Pat. No. 8,217,149 B2), a combination of Tremelimumab (Pfizer/MedImmune) and Pidilizumab (CT-011; CureTech), a combination of Tremelimumab (Pfizer/MedImmune) and MED10680 (AMP-514; AstraZeneca), a combination of Tremelimumab (Pfizer/MedImmune) and avelumab (Merck KGaA/Pfizer), a combination of Tremelimumab (Pfizer/MedImmune) and MIH1 (Affymetrix), a combination of Tremelimumab (Pfizer/MedImmune) and AMP-224 and a combination of Tremelimumab (Pfizer/MedImmune) and Lambrolizumab.

It is also preferred that the vaccine of the invention (i.e., the first component (K) and/or second component (V), as disclosed herein) is combined (i) with an inhibitor of the PD-1 pathway as described above, e.g. an inhibitor of PD-1, PD-L1 or PD-L2, and (ii) with an inhibitor of T-cell immunoglobulin mucin-3 (TIM-3). In some embodiments, the inhibitor of the PD-1 pathway and the inhibitor of TIM-3 may be administered at about the same time and via the same or distinct routes of administration. In other embodiments, the inhibitor of the PD-1 pathway is administered prior to the inhibitor of TIM-3. Without being bound to any theory, it is assumed that targeting of an additional checkpoint, such as TIM-3, in addition to the PD-1 pathway, may prevent relapses due to upregulation of the additional checkpoint, such as TIM-3, during treatment.

According to one embodiment, it is preferred that the vaccine for use according to the invention, or any of its components (K) or (V), or the rVSV of the invention as disclosed herein is combined with a targeted drug. Targeted drugs (for use in combination with the rVSV of the invention as disclosed herein, or the vaccine of the invention as disicosed herein, or any of its components (K) or (V) for use in medicine, in particular in the treatment of a tumor as disclosed herein) include VEGF-targeted drugs, EGFR-targeted drugs, or antibody-drug-conjugates. Preferred examples of VEGF-targeted drugs include Bevacizumab (Avastin®), ramucirumab (Cyramza®) or ziv-aflibercept (Zaltrap®). Preferred examples of EGFR-targeted drugs include Cetuximab (Erbitux®), panitumumab (Vectibix®) or Regorafenib (Stivarga®). Preferred examples of antibody-drug conjugates include Ado-Trastuzumab emtansine (Kadcyla®), SYD985, Trastuzumab vc-seco-DUBA, ABT-414, Depatuxizumab mafodotin, AMG 595, IMGN289, Laprituximab emtansine, ABBV-221, SGN-75, MDX-1203, BMS-936561, SGN-CD70A, AMG 172, Gemtuzumab Ozogamicin (GO), SAR3419, coltuximab ravtansine, BAY 94-9343, anetumab ravtansine, Pinatuzumab vedotin, labetuzumab govitecan, Sacituzumab govitecan, MLN2704, Naratuximab emtansine, brentuximab vedotin, or Rovalpituzumab tesirine.

The combined treatments as disclosed above may e.g. be administered as a non-fixed (e.g. free) combination of the substances or in the form of a fixed combination, including kit-of-parts. In this context, “combination” or “combined” within the meaning of this invention includes, without being limited, a product that results from the mixing or combining of more than one active agent and includes both fixed and non-fixed (e.g. free) combinations (including kits) and uses, such as e.g. the simultaneous, concurrent, sequential, successive, alternate or separate use of the components or agents. The term “fixed combination” means that the active agents are both administered to a patient simultaneously in the form of a single entity or dosage. The term “non-fixed combination” means that the active agents are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more active agents.

The combination treatment with immune checkpoint inhibitors and the vaccine for use according to the invention, or any of its components (K) or (V), or the rVSV of the invention as disclosed herein, may e.g. be advantageous in tumors that are characterized by an activation of immune checkpoint pathways that suppress antitumor immune responses thereby evading immunosurveillance. Typically, such tumors are characterized by an increased expression of the above immune checkpoint pathway genes, which may e.g. be detected by immunohistochemistry on tissue or tumor biopsies. For example, expression of PD-1 or PD-L1 may be done using companion diagnostic kits which utilize specific antibodies, such as e.g. 28-8, 22C3, SP142, or SP263, to assess PD-1 expression in tumor tissue in situ. Typically, the higher the number of PD-1 positive cells in a tumor the greater the likelihood that a patient afflicted with that tumor will respond to such treatment with an immune checkpoint inhibitor. For example, a patient is considered to have a low expression if in 1%-5% of the tumor cells PD-1 (or PD-L1) expression is detected, medium if in between about 5% to about 10% PD-1 (or PD-L1) expression is detected, or high (strong) if PD-1 (PD-L1) expression is detected in about 10% to about more than 50% of the tumor cells (e.g. in about 15%, 20%, 25%, 30%, 35% to about more than 50%, 60%, 75% of the cells). Corresponding kits or assays are commercially available and include e.g. the antibodies SP1452, SP263, 22C3, or 28-8 (see e.g. Expert Review of Molecular Diagnostics, 16:2, 131-133). Combinations of the vaccine of the invention and PD-1/PD-L1 immune-checkpoint inhibitors may thus be advantageous or desirable to treat tumors that are characterized by at least a low PD-1 or PD-L1 expression, preferably by a medium PD-1 or PD-L1 expression, more preferably by a high PD-1 or PD-L1 expression. For example, the vaccine for use according to the invention, or any of its components (K) or (V), or the rVSV of the invention as disclosed herein may be combined with a PD-1 or PD-L1 checkpoint inhibitor as disclosed herein for use in the treatment and/or ameliorating one or more symptoms of cancer, whereby the vaccine for use according to the invention, or any of its components (K) or (V), or the rVSV of the invention as disclosed herein and the PD-1 or PD-L1 pathway inhibitor be administered concomitantly, sequentially or alternately. The PD-1 or PD-L1 pathway inhibitor is thereby administered in a therapeutically effective dose, e.g. from about 0.05 mg/kg body weight, 0.1 mg/kg body weight, 0.5 mg/kg body weight, 1 mg/kg body weight to about 5 mg/kg body weight, 7.5 mg/kg body weight, 10 mg/kg body weight, or at about 2.5 mg/kg body weight, 5 mg/kg body weight and the vaccine for use according to the invention, or any of its components (K) or (V), or the rVSV of the invention as disclosed herein of the invention may be administered at doses as disclosed above.

In another embodiment, the PD-1 pathway inhibitor is administered intravenously and the second component (V) of the vaccine, i.e. the recombinant vesiculovirus, preferably oncolytic recombinant vesiculovirus, for use according to the invention, is administered at least once intratumorally, or at least once intravenously.

In one embodiment, the PD-1 pathway inhibitor may e.g. be administered at least once, twice or three times 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days or 31 days prior to the administration of the vaccine according to the invention, or any of its components (K) or (V), or the rVSV of the invention as disclosed herein. Alternatively, the PD-1 or PD-L1 pathway inhibitor may e.g. be administered concomitantly, or alternately with the vaccine for use according to the invention, or any of its components (K) or (V), or the rVSV of the invention as disclosed herein. The term “concomitant” administration refers to administering the first component (K) and the second component (V) of the vaccine and the PD-1 or PD-L1 pathway inhibitor within the same general time period, for example on the same day(s) but not necessarily at the same time. The term “alternate administration” generally refers to the administration of one the vaccine for use according to the invention, or any of its components (K) or (V), or the rVSV of the invention as disclosed herein or the PD-1/PD-L1 pathway inhibitor during a time period, for example over the course of a few days or a week, followed by administration of the respective other therapeutic agent during a subsequent period of time, for example over the course of a few days or a week, and then repeating the pattern for one or more cycles.

The vaccine for use according to the invention, or any of its components (K) or (V), or the rVSV of the invention as disclosed herein and the PD-1 or PD-L1 pathway inhibitor may e.g. also be administered sequentially which may e.g. also be referred to as successive administration. For example, the vaccine for use according to the invention, or any of its components (K) or (V), or the rVSV of the invention as disclosed herein or the PD-1/PD-L1 pathway inhibitor may be administered during a first time period (for example over the course of a few days or a week) using one or more doses, followed by administration of the other respective other therapeutic agent (PD-1, PD-L1 inhibitor or the vaccine for use according to the invention, or any of its components (K) or (V), or the rVSV of the invention as disclosed herein) during a second time period (for example over the course of a few days or a week) using one or more doses. An overlapping administration scheme may also be employed, which includes administration of the PD-1/PD-L1 pathway inhibitor or of the vaccine for use according to the invention, or any of its components (K) or (V), or the rVSV of the invention as disclosed herein on different days over the treatment period, not necessarily according to a regular sequence. Variations on these general guidelines may also be employed, e.g. according to the agents used and the condition of the subject.

In one embodiment, the vaccine for use according to the invention, or any of its components (K) or (V), or the rVSV of the invention as disclosed herein may e.g. be administered according to the administration scheme as disclosed herein, whereby a PD-1 or PD-L1 pathway inhibitor may be administered intermittently between the administration of the first component (K) and second component (V) of the vaccine. Accordingly, the method of treatment according to the invention comprises administering to a patient in need thereof the vaccine of the invention and a PD-1 or PD-L1 pathway inhibitor as disclosed herein.

According to one embodiment, the first component (K) for use according to the invention is used in combination with the second component (V) of the vaccine of the invention or the rVSV of the invention as disclosed herein. For example, the first component (K) for use according to the invention may be provided as a vaccine in combination with the second component (V) of the invention, or the rVSV of the invention as disclosed herein. It is to be understood that the combined use of the first component (K) of the invention as disclosed herein with the second component (V) of the invention, or the rVSV according to the invention as disclosed herein may further be combined with one or more chemotherapeutic agents as disclosed herein, one or more immunotherapeutic agents as disclosed herein, or with one or more targeted drugs as disclosed herein. According to one embodiment, the second component (V) of the invention as disclosed herein, or the rVSV according to the invention as defined herein, is for use in combination (e.g., in medicine) with the first component (K) of the invention as disclosed herein. It is to be understood that the combined use of the second component (V), or of the rVSV according to the invention as disclosed herein with the first component (K) of the invention may further be combined with one or more chemotherapeutic agents as disclosed herein, one or more immunotherapeutic agents as disclosed herein, or with one or more targeted drugs as disclosed herein. Exemplary combined used as disclosed above may include providing combinations of the first component (K) of the invention and/or the second component (V) according to the invention, or the first and second pharmaceutical compositions as disclosed above in a kit. Such combinations may be limited to the provision of the first component (K) of the invention as disclosed herein, or a first pharmaceutical composition as disclosed herein in combination with (i) one or more chemotherapeutic agents as disclosed herein, (ii) one or more immunetherapeutic agents as disclosed above, (iii) one or more targeted drugs, or to the provision of the second component (V) of the invention as disclosed herein, the rVSV of the invention as disclosed herein or a first pharmaceutical composition as disclosed herein in combination with (iv) one or more chemotherapeutic agents as disclosed herein, (v) one or more immunetherapeutic agents as disclosed above, (vi) one or more targeted drugs.

In one aspect, the present invention provides a combination comprising a first component (K) and a second component (V) according to the invention as defined herein. According to one embodiment, the combination comprising the first component (K) of the invention and the second component (V) of the invention is for use as a vaccine. The combination according to the invention is thus for use as disclosed above and may e.g. be administered according to any of the administration schemes disclosed above, or may e.g. be combined with one or more further therapeutically active agents as disclosed above (e.g. chemotherapeutic agents, targeted drugs, or immunotherapeutic agents, such as immune checkpoint inhibitors, all as disclosed above).

For example, in such combinations, kits and uses, it may be preferred that the following components are combined:
(1) the complex of the first component (K) comprising or consisting of the amino acid sequence according to SEQ ID NO: 60; and
(2) the rhabdovirus, preferably the oncolytic rhabdovirus, of the second component (V) (being a vesicular stomatitis virus), wherein the RNA genome of the vesicular stomatitis virus comprises or consists of an RNA sequence identical or at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 80, preferably wherein the vesicular stomatitis virus encodes in its genome
   a phosphoprotein (P) comprising the amino acid consisting of SEQ ID NO: 50,
   a nucleoprotein (N) comprising the amino acid sequence consisting of SEQ ID NO: 49,
   a matrix protein (M) comprising the amino acid sequence consisting of SEQ ID NO: 52,
   a large protein (L) comprising the amino acid sequence consisting of SEQ ID NO: 51,
   a glycoprotein (GP) comprising the amino acid sequence consisting of SEQ ID NO: 53, and
   an antigenic domain which comprises the amino acid sequence consisting of SEQ ID NO: 45 or SEQ ID NO: 59.

In addition, such combinations, kits and uses may further comprise an immune checkpoint inhibitor of the PD-1/PD-L1 pathway, preferably selected from the group consisting of pembrolizumab; nivolumab; pidilizumab; cemiplimab; PDR-001; atezolizumab; avelumab; durvalumab, an antibody comprising a heavy chain comprising the amino acid sequence of SEQ ID NO:61 and a light chain comprising the amino acid sequence of SEQ ID NO:62; an antibody comprising a heavy chain comprising the amino acid sequence of SEQ ID NO:63 and a light chain comprising the amino acid sequence of SEQ ID NO:64; an antibody comprising a heavy chain comprising the amino acid sequence of SEQ ID NO:65 and a light chain comprising the amino acid sequence of SEQ ID NO:66; an antibody comprising a heavy chain comprising the amino acid sequence of SEQ ID NO:67 and a light chain comprising the amino acid sequence of SEQ ID NO:68; and an antibody comprising a heavy chain comprising the amino acid sequence of SEQ ID NO:69 and a light chain comprising the amino acid sequence of SEQ ID NO:70.

In accordance therewith, the combination for use according to the invention may e.g. also be used in a method of treating a patient afflicted with cancer which comprises administering to such patient the combination according to the administration schemes and dosing regimens as disclosed herein.

Moreover, the present invention also provides a kit for use in vaccination for treating, preventing and/or stabilizing a tumor or cancer as disclosed herein, comprising the pharmaceutical composition as described herein or the vaccine as described herein and instructions for use of said pharmaceutical composition or of said vaccine for use according to the invention in the prevention and/or treatment of a tumor or cancer, such as e.g. colorectal cancer, or metastatic colorectal cancer as disclosed herein.

In one aspect, the invention provides for a method of increasing the tumor infiltration with tumor antigen-specific T-cells, whereby the method comprises the administration of the vaccine of the invention to a mammal, preferably a human, as disclosed herein. For example, the vaccine of the invention for use according to the method of the invention may e.g. be administered in combination with an immune checkpoint inhibitor as disclosed herein.

In a further aspect, the present invention provides a vaccination kit for use in treating, preventing and/or stabilizing a tumor or cancer, preferably for use in the prevention and/or treatment of cancer, the kit comprising at least one of:
(i) the vaccine of the invention as defined above,
(ii) at least one pharmacologically active agent as described above.

In particular, the kit of the invention may comprise more than one component of each of its parts (i), (ii). For example, the kit according to the present invention may comprise at least two different vaccines of the invention as part (i), and/or more than one pharmacologically active agent as disclosed above.

For example, the kit of the invention may comprise two different vaccines of the invention as part (i), e.g. it may comprise two first components of the vaccine which comprise a different antigenic domain which may comprise different antigens as disclosed herein. The kit as disclosed herein may, e.g. also comprise more than one vaccine of the invention as part (i) which comprise different TLR agonists, or which comprise a different CPP. For example, the kit as disclosed herein may also comprise more than one, e.g. two, three or four pharmaceutically active agents as disclosed herein. The combination of such pharmaceutically agents will be dependent on the cancer and/or disease state to be treated.

The various components of the kit as disclosed herein may be packaged in one or more containers. The above components may be provided in a lyophilized or dry form or dissolved in a suitable buffer. In addition, the kit according to the present invention may optionally contain instructions of use.

In a preferred embodiment the kit of the invention is for use in treatment of colorectal cancer, metastatic colorectal cancer, triple negative breast cancer, or pancreatic cancer.

Preferably, such a kit further comprises a package insert or instruction leaflet with directions to treat colorectal cancer by using the vaccine (for use) according to the present invention as described herein, and/or the pharmaceutical composition as described herein.

In one embodiment, the invention also provides a virus producing cell, characterized in that the cell produces a the recombinant rhabdovirus or recombinant vesicular stomatitis virus according to the invention.

The cell which may e.g. be used for the production of the recombinant rhabdovirus or recombinant vesicular stomatitis virus according to the invention may be of any origin and may be present as isolated cell or as a cell comprised in a cell population. It is preferred that the cell producing the recombinant rhabdovirus or recombinant vesicular stomatitis virus of the invention is a mammalian cell. In a more preferred embodiment, the virus producing cell of the invention is characterized in that the mammalian cell is a multipotent adult progenitor cell (MAPC), a neural stem cell (NSC), a mesenchymal stem cell (MSC), a HeLa cell, a HEK cell, any HEK293 cell (e.g. HEK293F or HEK293T), a Chinese hamster ovary cell (CHO), a baby hamster kidney (BHK) cell or a Vero cell or a bone marrow derived tumor infiltrating cell (BM-TIC).

Alternatively, the virus producing cell of the invention may be a human cell, monkey cell, mouse cell or hamster cell. The skilled person is aware of methods suitable for use in testing whether a given cell produces a virus and, thus, whether a particular cell falls within the scope of this invention. In this respect, the amount of virus produced by the cell of the invention is not particularly limited. Preferred viral titers are ≥1×10⁷ TCID50/ml or ≥1×10⁸ genome copies/ml in the crude supernatants of the given cell culture after infection without further downstream processing.

In a particular embodiment, the virus producing cell of the invention is characterized in that the cell comprises one or more expression cassettes for the expression of at least one of the genes selected from the group consisting of genes n, l, p and m coding for proteins N, L, P and M of the VSV and a gene gp coding for LCMV-GP, Dandenong-GP or Mopeia-GP glycoprotein.

Virus producing cells in the meaning of the invention include classical packaging cells for the production of recombinant rhabdovirus or recombinant vesicular stomatitis virus according to the invention from non-replicable vectors as well as producer cells for the production of recombinant rhabdovirus from vectors capable of reproduction. Packaging cells usually comprise one or more plasmids for the expression of essential genes which lack in the respective vector to be packaged and/or are necessary for the production of virus. Such cells are known to the skilled person who can select appropriate cell lines suitable for the desired purpose.

The recombinant rhabdovirus or vesicular stomatitis virus according to the invention may e.g. be produced according to methods known to the skilled artisan and include without limitation (1) using cDNAs transfected into a cell or (2) a combination of cDNAs transfected into a helper cell, or (3) cDNAs transfected into a cell, which is further infected with a helper/minivirus providing in trans the remaining components or activities needed to produce either an infectious or non-infectious recombinant rhabdovirus. Using any of these methods (e.g., helper/minivirus, helper cell line, or cDNA transfection only), the minimum components required are a DNA molecule containing the cis-acting signals for (1) encapsidation of the genomic (or antigenomic) RNA by the Rhabdovirus N protein, P protein and L protein and (2) replication of a genomic or antigenomic (replicative intermediate) RNA equivalent.

A replicating element or replicon is a strand of RNA minimally containing at the 5′ and 3′ ends the leader sequence and the trailer sequence of a rhabdovirus. In the genomic sense, the leader is at the 3′ end and the trailer is at the 5′ end. Any RNA-placed between these two replication signals will in turn be replicated. The leader and trailer regions further must contain the minimal cis-acting elements for purposes of encapsidation by the N protein and for polymerase binding which are necessary to initiate transcription and replication. For preparing recombinant rhabdovirus a minivirus containing the G gene would also contain a leader region, a trailer region and a G gene with the appropriate initiation and termination signals for producing a G protein mRNA. If the minivirus further comprises an M gene, the appropriate initiation and termination signals for producing the M protein mRNA must also present.

For any gene contained within the recombinant rhabdovirus genome, the gene would be flanked by the appropriate transcription initiation and termination signals which will allow expression of those genes and production of the protein products (Schnell et al., Journal of Virology, p. 2318-2323, 1996). To produce “non-infectious” recombinant rhabdovirus, the recombinant rhabdovirus must have the minimal replicon elements and the N, P, and L proteins and it must contain the M gene. This produces virus particles that are budded from the cell, but are non-infectious particles. To produce “infectious” particles, the virus particles must additionally comprise proteins that can mediate virus particle binding and fusion, such as through the use of an attachment protein or receptor ligand. The native receptor ligand of rhabdoviruses is the G protein.

Any cell that would permit assembly of the recombinant rhabdovirus can be used. One method to prepare infectious virus particles comprises an appropriate cell line infected with a plasmid encoding for a T7 RNA polymerase or other suitable bacteriophage polymerase such as the T3 or SP6 polymerases. The cells may then be transfected with individual cDNA containing the genes encoding the G, N, P, L and M rhabdovirus proteins. These cDNAs will provide the proteins for building a recombinant rhabdovirus particle. Cells can be transfected by any method known in the art.

Also transfected into the cell line is a “polycistronic cDNA” containing the rhabdovirus genomic RNA equivalent. If the infectious, recombinant rhabdovirus particle is intended to be lytic in an infected cell, then the genes encoding for the N, P, M and L proteins must be present as well as any heterologous nucleic acid segment. If the infectious, recombinant rhabdovirus particle is not intended to be lytic, then the gene encoding the M protein is not included in the polycistronic DNA. By “polycistronic cDNA” it is meant a cDNA comprising at least transcription units containing the genes which encode the N, P and L proteins. The recombinant rhabdovirus polycistronic DNA may also contain a gene encoding a protein variant or polypeptide fragment thereof, or a therapeutic nucleic acid or protein. Alternatively, any protein to be initially associated with the viral particle first produced or fragment thereof may be supplied in trans.

Also contemplated is a polycistronic cDNA encoding the antigenic domain according to the invention as disclosed above. The polycistronic cDNA contemplated may contain a gene encoding a protein variant, a gene encoding a reporter, a therapeutic nucleic acid, and/or either the N-P-L genes or the N-P-L-M genes. The first step in generating a recombinant rhabdovirus is expression of an RNA that is a genomic or antigenomic equivalent from a cDNA. Then that RNA is packaged by the N protein and then replicated by the P/L proteins.

The recombinant virus thus produced can be recovered. If the G protein is absent from the recombinant RNA genome, then it is typically supplied in trans. If both the G and the M proteins are absent, then both are supplied in trans. For preparing “non-infectious rhabdovirus” particles, the procedure may be the same as above, except that the polycistronic cDNA transfected into the cells would contain the N, P and L genes of the rhabdovirus only. The polycistronic cDNA of non-infectious rhabdovirus particles may additionally contain a gene encoding a protein.

Transfected cells are usually incubated for at least 24 hr at the desired temperature, usually about 37 degrees. For non-infectious virus particles, the supernatant is collected and the virus particles isolated. For infectious virus particles, the supernatant containing virus is harvested and transferred to fresh cells. The fresh cells are incubated for approximately 48 hours, and the supernatant is collected.

A cell line which may e.g. be used alternatively for the production of the recombinant rhabdovirus or recombinant vesicular stomatitis virus according to the invention is the human cell line 293SF-3F6 according to the method as disclosed in Journal of Biotechnology 289 (2019) 144-149.

The present invention also provides for polynucleotides encoding the complex of the first component (K) of the vaccine as disclosed herein, preferably, the polynucleotides according to the invention encode an amino acid sequence according to SEQ ID NO: 45, or SEQ ID NO: 60 or functional variants thereof having at least a sequence identity of 75%, 80%, 85%, more preferably of at least 90%, 95%, 98%. The term “polynucleotide” as used in the present invention denotes a single- or double-stranded polymer of deoxyribonucleotide or ribonucleotide bases read from the 5′ to the 3′ end. Polynucleotides include RNA and DNA, and may be isolated from natural sources, synthesized in vitro, or prepared from a combination of natural and synthetic molecules. The length of a polynucleotide molecule is given herein in terms of nucleotides (abbreviated “nt”) or base pairs (abbreviated “bp”). The term “nucleotides” is used for both single- and double-stranded molecules where the context permits. When the term is applied to double-stranded molecules it is used to denote overall length and will be understood to be equivalent to the term “base pairs”.

In one aspect the present invention also provides for a host cell comprising the polynucleotides of the invention. The term “host cell” as used herein refers to any prokaryotic (prokaryotic host cell) or eukaryotic cell (eukaryotic host cell). For example, a host cell for use according to the invention may be a yeast cell, insect cell or mammalian cell. For example, the host cell of the invention may be an insect cell selected from Sf9, Sf21, S2, Hi5, or BTI-TN-5B1-4 cells, or e.g. the host cell of the invention may be a yeast cell selected from Saccharomyces cerevisiae, Hansenula polymorpha, Schizosaccharomyces pombe, Schwanniomyces occidentafis, Kluyveromyceslactis, Yarrowia lipolytica and Pichia pastoris, or e.g. the host cell of the invention may be a mammalian cell selected from HEK293, HEK293T, HEK293E, HEK 293F, NS0, per.C6, MCF-7, HeLa, Cos-1, Cos-7, PC-12, 3T3, Vera, vero-76, PC3, U87, SAOS-2, LNCAP, DU145, A431, A549, B35, H1299, HUVEC, Jurkat, MDA-MB-231, MDA-B-468, MDA-MB-435, Caco-2, CHO, CHO-K1, CHO-B11, CHO-DG44, BHK, AGE1.HN, Namalwa, WI-38, MRC-5, HepG2, L-929, RAB-9, SIRC, RK13, 1 1 B1 1, 1 D3, 2.4G2, A-10, B-35, C-6, F4/80, IEC-18, L2, MH1 C1, NRK, NRK-49F, NRK-52E, RMC, CV-1, BT, MDBK, CPAE, MDCK.1, MDCK.2, and D-17. Prokaryotic cells for use according to the invention may e.g. include bacteria such as E. coli, including BL21, Lemo21, E. coli K12. The host cells according to the invention may e.g. be used for recombinant production of the complex of the first component (K) of the invention as disclosed herein according to standard protocols, e.g. LaVallie, Current Protocols in Protein Science (1995) 5.1.1-5.1.8; Chen et al., Current Protocols in Protein Science (1998) 5.10.1-5.10.41).

In a further aspect, the present invention provides for a polypeptide comprising or consisting of an amino acid sequence of SEQ ID NO: 60 for use in medicine, in particular for use in an immunization regimen, in combination with a vesicular stomatitis virus, wherein the RNA genome of the vesicular stomatitis virus comprises or consists of an RNA sequence identical or at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 80.

In a preferred embodiment, the present invention provides for a polypeptide comprising or consisting of an amino acid sequence of SEQ ID NO: 60 for use in medicine, in particular for use in an immunization regimen, in combination with a vesicular stomatitis virus, wherein the RNA genome of the vesicular stomatitis virus comprises or consists of an RNA sequence identical or at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 80, wherein the vesicular stomatitis virus encodes in its genome a phosphoprotein (P) comprising the amino acid consisting of SEQ ID NO: 50, a nucleoprotein (N) comprising the amino acid sequence consisting of SEQ ID NO: 49, a matrix protein (M) comprising the amino acid sequence consisting of SEQ ID NO: 52, a large protein (L) comprising the amino acid sequence consisting of SEQ ID NO: 51, a glycoprotein (GP) comprising the amino acid sequence consisting of SEQ ID NO: 53, and an antigenic domain which comprises the amino acid sequence consisting of SEQ ID NO: 45 or SEQ ID NO: 59.

In another related aspect, the present invention provides for a vesicular stomatitis virus, wherein the RNA genome of the vesicular stomatitis virus comprises or consists of an RNA sequence identical or at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 80 for use in medicine, in particular for use in an immunization regimen, in combination with a polypeptide comprising or consisting of an amino acid sequence of SEQ ID NO: 60.

In a preferred embodiment, the present invention provides for a vesicular stomatitis virus, wherein the RNA genome of the vesicular stomatitis virus comprises or consists of an RNA sequence identical or at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 80 for use in medicine, in particular for use in an immunization regimen, in combination with a polypeptide comprising or consisting of an amino acid sequence of SEQ ID NO: 60, wherein the vesicular stomatitis virus encodes in its genome a phosphoprotein (P) comprising the amino acid consisting of SEQ ID NO: 50, a nucleoprotein (N) comprising the amino acid sequence consisting of SEQ ID NO: 49, a matrix protein (M) comprising the amino acid sequence consisting of SEQ ID NO: 52, a large protein (L) comprising the amino acid sequence consisting of SEQ ID NO: 51, a glycoprotein (GP) comprising the amino acid sequence consisting of SEQ ID NO: 53, and an antigenic domain which comprises the amino acid sequence consisting of SEQ ID NO: 45 or SEQ ID NO: 59.

Preferably, in such polypeptides and viruses for use, the following components are combined:
(1) the polypeptide comprising or consisting of the amino acid sequence according to SEQ ID NO: 60; and
(2) the vesicular stomatitis virus, wherein the RNA genome of the vesicular stomatitis virus comprises or consists of an RNA sequence identical or at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 80, preferably wherein the vesicular stomatitis virus encodes in its genome
   a phosphoprotein (P) comprising the amino acid consisting of SEQ ID NO: 50,
   a nucleoprotein (N) comprising the amino acid sequence consisting of SEQ ID NO: 49,
   a matrix protein (M) comprising the amino acid sequence consisting of SEQ ID NO: 52,
   a large protein (L) comprising the amino acid sequence consisting of SEQ ID NO: 51,
   a glycoprotein (GP) comprising the amino acid sequence consisting of SEQ ID NO: 53, and
   an antigenic domain which comprises the amino acid sequence consisting of SEQ ID NO: 45 or SEQ ID NO: 59.

In addition, such polypeptides and viruses for use may further include the combination with an immune checkpoint inhibitor of the PD-1/PD-L1 pathway, preferably selected from the group consisting of pembrolizumab; nivolumab; pidilizumab; cemiplimab; PDR-001; atezolizumab; avelumab; durvalumab, an antibody comprising a heavy chain comprising the amino acid sequence of SEQ ID NO:61 and a light chain comprising the amino acid sequence of SEQ ID NO:62; an antibody comprising a heavy chain comprising the amino acid sequence of SEQ ID NO:63 and a light chain comprising the amino acid sequence of SEQ ID NO:64; an antibody comprising a heavy chain comprising the amino acid sequence of SEQ ID NO:65 and a light chain comprising the amino acid sequence of SEQ ID NO:66; an antibody comprising a heavy chain comprising the amino acid sequence of SEQ ID NO:67 and a light chain comprising the amino acid sequence of SEQ ID NO:68; and an antibody comprising a heavy chain comprising the amino acid sequence of SEQ ID NO:69 and a light chain comprising the amino acid sequence of SEQ ID NO:70.

The present invention is not to be limited in scope by the specific embodiments described herein. Indeed, various modifications of the invention in addition to those described herein will become apparent to those skilled in the art from the foregoing description and accompanying figures and detailed Examples which illustrate, by way of example, the principles of the invention.

### Materials and Methods

### Ethics Statement

All animal experiments were approved by Austrian Federal Ministry of Science, Research and Economy and by institutional and cantonal Geneva veterinary authorities in accordance with Swiss Federal law on animal protection and performed according to institutional guidelines of the Medical University of Innsbruck, Austria and of Swiss Federal law on animal protection, respectively.

### Mice

Six- to eight-week-old female C57BL/6Rj or B6(C)/Rj-Tyrc/c mice were obtained from Janvier (Le Genest St Isle, France) or Charles River (L'Arbresles, France).

### Tumor Cell Lines for Implantation

E.G7-OVA cells were purchased from ATCC and maintained in complete RPMI 1640 medium with 0.4 mg/ml geneticin (Life Technologies). B16-OVA cells were provided by Bertrand Huard (University of Grenoble-Alpes, Grenoble, France) and maintained in complete RPMI 1640 medium with 1 mg/ml geneticin. TC-1 cells were provided by T. C. Wu (Johns Hopkins University, Maryland, US) and cultured in complete RPMI 1640 with 0.4 mg/ml geneticin. MC-38 cells were a kind gift from Gottfried Baier (Medical University of Innsbruck, Innsbruck, Austria) and were maintained in complete DMEM containing 5% gentamicin. For tumor implantation, mice were injected subcutaneously with 3×10⁵ E.G7-OVA cells, 2×10⁵ B16-OVA or 2×10⁵ MC-38 cells in the right flank or with 1×10⁵ TC-1 cells in the back. For monitoring tumor growth, tumor diameter was measured 2-3 times per week using a caliper and volume was calculated using the formula: 0.4×length×width². Mice were sacrificed when tumor-size reached the size specified by the respective institutional veterinary authorities or tumors showed signs of ulcerations. Animals were euthanized by CO₂ asphyxiation and cervical dislocation.

### Generation of Vaccine Constructs

Recombinant protein vaccine constructs of the complex of the first component (K) were designed in-house and produced in E. coli by Genscript. During purification process, endotoxins were removed from vaccines through extensive washes with Triton-X114 followed by subsequent affinity chromatography. Endotoxin content was quantified in each vaccine batch using a LAL chromogenic assay. Only the batches with endotoxin level below 10 EU/mg protein (according to the guidelines) were used for further in vitro and in vivo experiments, the first component (K) OVA vaccine contains both CD8 and CD4 H-2b epitopes from Ovalbumin, whereas the first component (K) comprising Mad24 (multi-antigenic domain 24) contains the immunogenic neo-epitopes Adpgk and Reps1. With regard to HPV antigens, the first component (K) comprises Mad25 (multi-antigenic domain 25), which contains both CD8 and CD4 H-2b epitopes from E7 HPV.

Recombinant viruses VSV-GP, VSV-GP-OVA and VSV-GP-Luciferase (VSV-GP-Luc) have been described previously (Muik et al., 2014, Cancer Res. 74(13): 3567-3578; Tober et al., 2014, J. Virol. 88(9): 4897-4907; Dold et al., 2016, Mol. Ther. Oncolytics 3: 16021) whereas VSV-GP-Mad24 and VSV-GP-HPV were generated de novo. VSV-GP-Mad24 (multi-antigenic domain 24) expresses the immunogenic neo-epitopes Adpgk and Reps1 (Yadav et al., 2014, Nature 515(7528): 572-576) and VSV-GP-HPV encodes the attenuated E6/E7 fusion construct (Cassetti et al., 2004, Vaccine 22(3-4): 520-527) in addition to wild-type E2. All the recombinant virus variants were rescued and produced in house as described previously (Heilmann et al., 2019, Viruses 11(11): 989). The viruses were purified using sucrose cushion and titered on BHK-21 cells (ATCC).

### Immunization and Checkpoint Blockade

For immunization in non-tumor bearing animals, mice were randomly assigned to different treatment groups. For the E.G7-OVA model, mice received the first vaccination on day 5 post tumor implantation followed by 3 boost immunizations at 7 days interval. Mice implanted with MC-38 tumors were vaccinated on days 3, 10, 17 and 24 after tumor implantation. Mice bearing TC-1 tumors were grouped based on tumor size prior to first immunization on day 7 post tumor implantation so that the mean tumor size of each treatment group was comparable. Vaccination was repeated on days 14, 28 and 49 after tumor implantation. Mice were vaccinated either with 2 nmol of the complex of the first component (K) (targeting the relevant TAA) administered s.c. at the tail base or with 1×10⁷ TCID₅₀ of the respective virus of the second component (V) (VSV-GP-TAA or VSV-GP) injected i.v. into a lateral tail vein on days indicated above.

For checkpoint blockade, E.G7-OVA tumor-bearing mice were treated intravenously with 200 μg of αPD-1 antibody (clone RMP1-14, BioXcell) every 4 days starting on day 7 post tumor implantation. For the MC-38 tumor model, 200 μg of αPD-L1 antibody (clone 10F.9G2, BioXCell) was injected intraperitoneally on days 6, 10, 13, 17, 20, 24 and 27 post tumor implantation. Mice bearing TC-1 tumors received intravenous injections of 200 μg αPD-1 antibody 7, 15, 28 and 49 days after tumor implantation.

### Flow Cytometry

Single cell suspensions were prepared from spleen and bone marrow by mechanical dissociation using a 40 μM cell strainer. This was followed by lysis of erythrocyte using Pharm Lyse™ Lysing buffer (BD Biosciences). For whole blood, lysis was carried out after surface staining. Tumor-infiltrating leucocytes (TILs) were purified using mouse tumor dissociation kit (Miltenyi), following manufacturer instruction. Briefly, TC-1 tumor tissues were cut into 2-4 mm pieces with a scalpel, suspended in plain DMEM medium containing tumor dissociating enzymes (Miltenyi) and digested on a Gentle MACS with heating system (Miltenyi) using solid tumor program. Enzymatic digestion was stopped by cooling the cells with a cold PBS 0.5% BSA solution. After filtration through a 70 mm cell strainer, CD45⁺ cells were purified using CD45 TIL microbeads (Miltenyi) following manufacturer protocol and used for flow cytometry analysis.

For the detection of antigen specific CD8⁺ T cells, whole blood or single cell suspensions from spleen, bone marrow or tumors were labelled with one or more of the following fluorescently-labelled peptide-MHC multimers: H2-Kb-SIINFEKL (OVA), H2-Db-ASMTNMELM (Adpgk), H2-Kb-RGYVYQGL (VSV-N), all from MBL International (Woburn, MA, US) or H2-Db-RAHYNIVTF (E7) which was from Immudex (Copenhagen, Denmark). This was followed by surface staining with the following antibodies: CD8 (53-6.7), CD90.2 (30-H12), CD44 (IM7); CD62L (MEL-14), CD127 (SB/199), KLRG1 (2F1), CD4 (GK1.5), CD19 (6D5), CD14 (Sa14-2) all from BioLegend (San Diego, CA, US). Dead cells were labelled using LIVE/DEAD™ Fixable Near-IR Dead Cell Stain Kit (Thermo Fischer Scientific, Waltham, MA, US).

For phenotyping tumor infiltrating leukocyte subsets, the following monoclonal antibodies (mAbs) were used: CD45 (30F11), CD11b (M1/70), CD3 (17A2), CD4 (RMA4-4), CD8 (53-6.7), CD25 (3C7), KLRG1 (2F1), CD279 (29F.1A12), CD366 (RMT3-23), Ly6C (AL-21), Ly6G (1A8), Ly6C/G (RB6-8C5), CD335 (29A1.4), CD11c (HL3), CD103 (M290), I-A/1-E (M5/114.5.2), FoxP3 (FJK-16s), CD206 (C068C2), CD68 (FA-11), all from BD Biosciences (San Jose, CA, US) except CD279, CD366, CD68, CD206 and Ly6C/G which are from Biolegend and FoxP3 from eBioscience. Dead cells were identified with LIVE/DEAD yellow or aqua fluorescent reactive dye from Life Technologies and were excluded from analyses.

Intracellular staining was performed after stimulation with the indicated peptides and in presence of CD107a mAb (1D4B, BD Biosciences) for 6 h in the presence of Brefeldin A (GolgiPlug, BD Bioscences). Intracellular staining was done with mAbs to IFN-γ (XMG1.2, BD Biosciences), TNF-α (MP6-XT22, BD Biosciences), and corresponding isotype controls (BD Biosciences). For Granzyme B intracellular staining, cells were cultured for 4 h in the presence of Brefeldin A (GolgiPlug, BD Biosciences). Intracellular staining was done with mAbs to Granzyme B (REA226, Miltenyi). Fixation and permeabilization was carried out using the BD Bioscience kit according to manufacturer's instructions. Samples were acquired on FACS Canto II (BD Biosciences), Gallios flow cytometer (Beckman Coulter), or Attune (ThermoFisher).

Flow cytometry data was analyzed with FlowJo software version 10.5.3 (FlowJo, LLC, Oregon, US) or Kaluza (Beckman Coulter) software.

### Transcriptome Analysis

Tumor tissue was snap frozen in liquid nitrogen upon harvest and homogenates were prepared using RLT buffer (Qiagen) and SpeedMill PLUS (Analytik Jena, Jena, Germany) followed by Phenol/Chloroform extraction. The aqueous phase containing RNA was then processed and RNA was isolated using RNeasy Mini kit (Qiagen) according to manufacturer's instructions. Quality of extracted RNA was assessed using RNA ScreenTape Assay (Agilent Technologies, Waldbronn, Germany) on the Tapestation 4200 (Agilent Technologies). Extracted RNA was analysed for differential expression by means of the nCounter PanCancer Immune Profiling Panel and the nCounter FLEX Analysis System (NanoString Technologies, Seattle, WA, USA). Profiled data were pre-processed following manufacturer's recommendations (Kulkarni (2011) “Digital multiplexed gene expression analysis using the NanoString nCounter system.” Curr Protoc Mol Biol Chapter 25: Unit25B.10) and heatmaps were generated using nSolver 4.0 software. Normalized gene counts from nSolver software was used to calculate the principal component analysis (PCA) using ClustVis (Metsalu and Vilo (2015) “ClustVis: a web tool for visualizing clustering of multivariate data using Principal Component Analysis and heatmap.” Nucleic Acids Res 43(W1): W566-570). Venn diagrams were generated using the webtool (http://bioinformatics.psb.ugent.be/webtools/Venn/).

### Multiplex ELISA

Cytokines and chemokines present in plasma of immunized animals were analyzed using LEGENDplex™ Mouse Anti-Virus Response Panel (13-plex) (BioLegend), according to manufacturer's instructions. Data were analyzed using LEGENDplex™ Cloud-based Data Analysis Software (BioLegend).

### Immunohistochemistry

Tumors were fixed in 4% buffered formaldehyde solution and embedded in paraffin. 2-3 μm thick sections were stained with hematoxylin and eosin (HE). Immunohistochemistry (IHC) was used to assess T-cells using primary antibodies against CD8 (Cell Signaling, #98941, dilution 1:2000). For antigen retrieval sections were heated in citrate buffer. The following steps were performed either manually or automatically in an autostainer (Lab Vision AS 360, Thermo Scientific, Freemont, USA): Blocking of endogenous peroxidase by incubation in H₂O₂, reducing background by application of a protein blocking reagent and applying the respective primary antibody. A secondary antibody formulation conjugated to an enzyme-labelled polymer and Di-amino-benzidine as chromogen were used. Sections were counterstained with hematoxylin. An experienced pathologist blinded to treatment regimens evaluated sections with an Olympus BX-53 microscope (Olympus, Tokyo, Japan).

### Statistics

Statistical analyses were performed using Prism software (GraphPad) and considered statistically significant if P<0.05. Used statistical tests included unpaired 2-tailed t test, one-way ANOVA with Tukey's multiple comparison, 2-way ANOVA test with Sidak's multiple comparison, Kruskal-Wallis test, Mann-Whitney test, and Log-rank test as indicated in the figure legend. Benjamini-Yekutieli procedure was used to calculate the FDR from the p-values returned by the t-test.

### EXAMPLES

Except where otherwise stated, in the Examples described here below, the first component (K), also referred to as KISIMA, is a complex of (i) a cell penetrating peptide (Z13) according to SEQ ID NO: 2, (ii) a (multi)antigenic domain (Mad) which comprises at least one antigen or antigenic epitope and (iii) a TLR peptide agonist Anaxa according to either SEQ ID NO: 6 or the sequence variant SEQ ID NO: 7, in which the components (i) to (iii) are covalently linked in N-terminal to C-terminal order. In the constructs Z13Mad5Anaxa, Z13Mad10Anaxa and Z13Mad12Anaxa, the TLR peptide agonist Anaxa has a sequence according to SEQ ID NO: 6. In the constructs Z13Mad24Anaxa, Z13Mad25Anaxa and Z13Mad39Anaxa, the TLR peptide agonist Anaxa has a sequence according to SEQ ID NO: 7. KISIMA-OVA is Z13Mad5Anaxa.

### Example 1

### Heterologous Prime-Boost Vaccination Using a First Component (K) for Priming and a Second Component (V) Elicit the Highest Antigen-Specific CD8 T Cell Response.

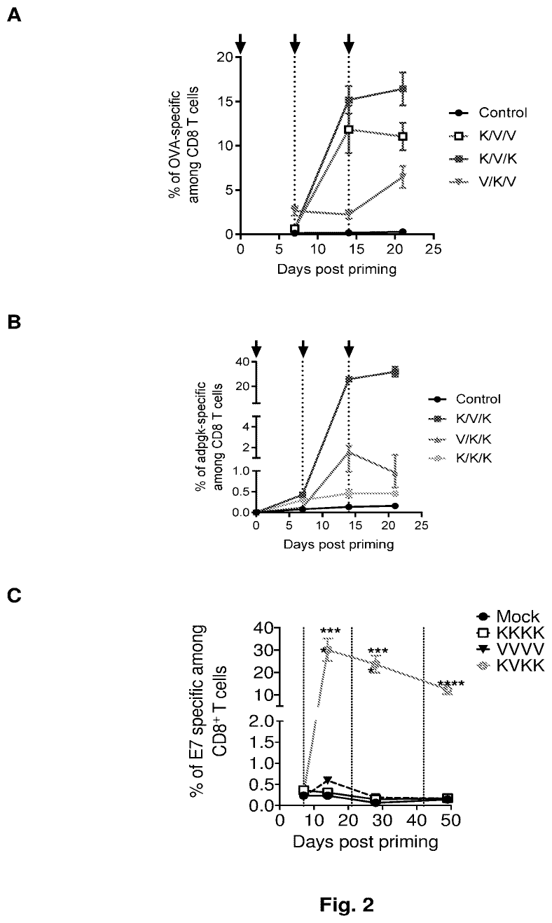

To characterize the heterologous combination of KISIMA peptide vaccine with VSV-GP-TAA oncolytic vaccine, a number of immunogenicity studies were performed with a model antigen (OVA), a neoantigen (Adpgk) and a viral antigen (HPV-E7). Non-tumor bearing C57BL/6 mice were vaccinated three times using one prime and two boosts, on days 0, 7 and 14 for OVA and Adpgk antigens (FIGS. 2A and 2B), or at days 0, 7, 21 and 42 for HPV-E7 antigen (FIG. 2C), with KISIMA-Ag (K) or VSV-Ag (V), or VSV-GP-empty (V(ø)). Mice were bled 1 week after each vaccination and multimer staining detecting Ag-specific CD8 T cells was performed and analyzed by flow cytometry (5 mice per group for OVA and Adpgk antigens, and 3 mice per group for HPV antigen). Z13Mad5Anaxa (s.c.); VSV-GP-OVA (administrated intramuscularly (i.m.)) (FIG. 2A); Z13Mad12Anaxa and VSV-GP-Mad24 (i.v.) (FIG. 2B), Z13Mad10Anaxa (s.c.) and VSV-GP-HPV-E2-E6-E7 (i.v.) (FIG. 2C).

### Example 2

### Route of Administration for VSV-GP-Antigen

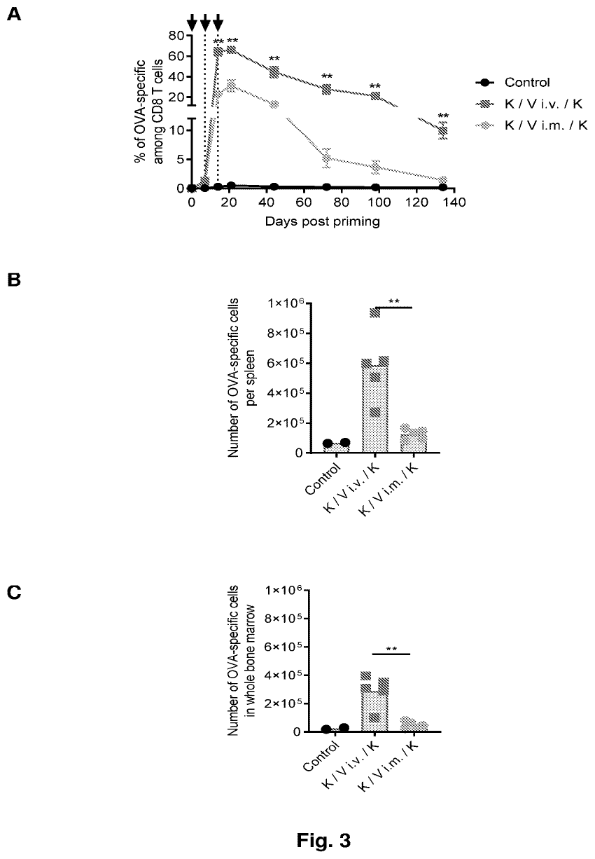

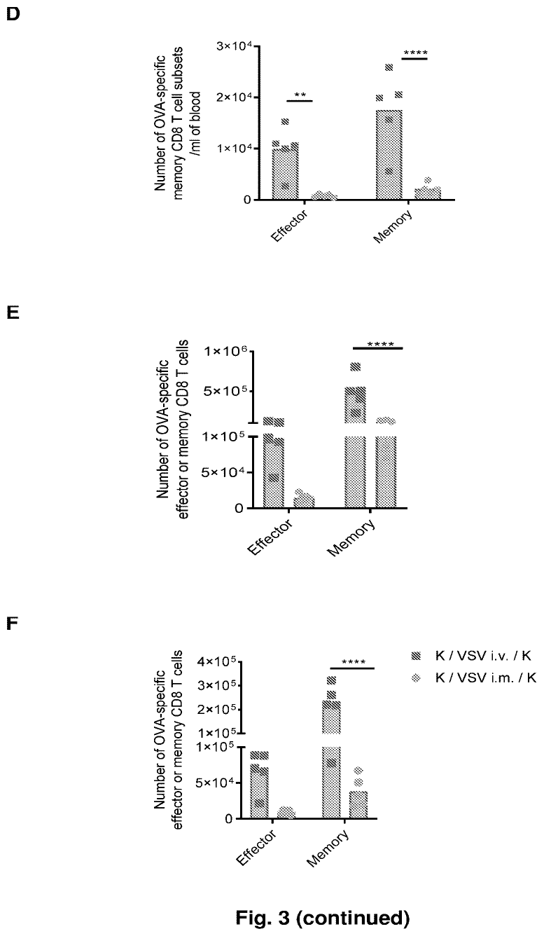

Two vaccination routes, intravenous (i.v.) administration and intramuscular (i.m.) administration, were compared for VSV-GP-OVA in combination with Z13Mad5Anaxa (s.c.) in naive mice. Boosting with VSV-GP-OVA i.v. induces the strongest circulating OVA-specific immune response, that decreases in time much less rapidly than when injected by i.m. route (see FIG. 3). At day 134, at the end of the observation period, numbers of OVA-specific CD8 T cells were also increased in the i.v. versus i.m. group in spleen and bone marrow (see FIG. 3). In addition, effector and memory OVA-specific CD8 T cells were significantly higher after i.v. versus i.m. VSV-GP-OVA injections, in the circulation as well as in lymphoid organs (spleen and bone marrow)

Antigen-specific T cell response was also assessed in mice receiving VSV-GP-OVA via the s.c., i.m., i.v. or intraperitoneal (i.p.) routes. VSV-GP-OVA administrated s.c., i.p. or i.m. led to an inferior OVA-specific CD8 T cell response compared to the i.v. route.

### Example 3

### Immunogenicity of the Vaccine According to the Invention

Naïve C57BL/6 mice (5 mice per group) were vaccinated at days 0 and 28 (weeks 0 and 4) subcutaneously with 10 nmol of the first component of the vaccine of the invention (K, SEQ ID NO: 60) at the tail base and at day 14 (week 2) intravenously with 10⁷ TCID₅₀ of one of the different VSV-GP constructs, i.e. VSV-GP-empty virus (VSV4ϕ), VSV-GP-Mad128 (SEQ ID NO: 80) which is a VSV-GP encoding an antigenic domain according to SEQ ID NO: 45, VSV-GP-Mad128Anaxa which is a VSV-GP encoding an amino acid sequence comprising SEQ ID NO: 71 which comprises an antigenic domain comprising SEQ ID NO: 45 and an immunomodulatory fragment of annexin II (SEQ ID NO: 7) or VSV-GP-ATP128 which is a VSV-GP encoding in its genome the complex comprising the amino acid sequence according to SEQ ID NO: 60.

Multimer staining (A) was performed on blood cells at day 35 (week 5) to quantify CEA-specific CD8 T cells. Expression of PD-1 and KLRG1 (B) was also assessed by flow cytometry.

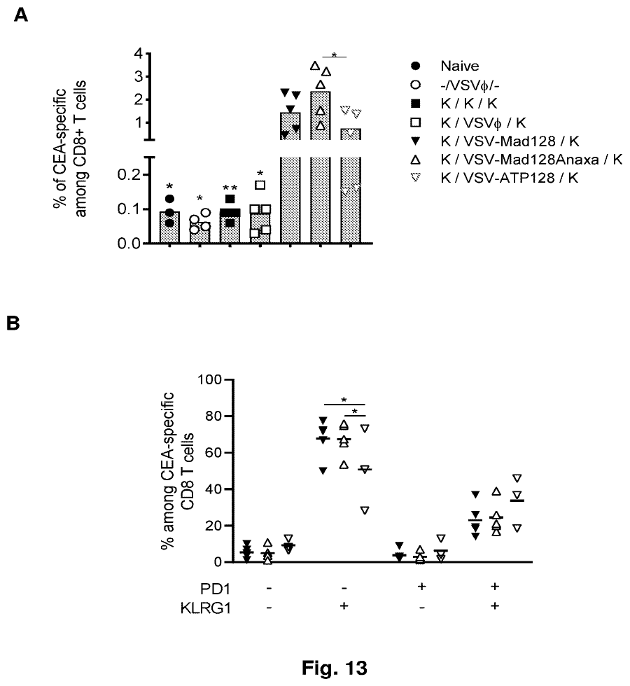

FIG. 13 A shows the percentage of multimer-positive cells (in % of CD8 T cells) for the different experimental groups and FIG. 13 B shows the percentage of PD-1⁻KLRG1⁻, PD-1⁻KLRG1⁺, PD-1⁺KLRG1⁻, PD-1⁺KLRG1⁺ among multimer-positive cells.

### Example 4

### CEA-Specific Immune Response in the Periphery

Naïve C57BL/6 mice (5 mice per group) were vaccinated at days 0 and 28 (weeks 0 and 4) subcutaneously with 10 nmol of the first component of the vaccine of the invention (K, SEQ ID NO: 60) at the tail base and at day 14 (week 2) intravenously with 10⁷ TCID50 of one of the different VSV-GP constructs (cf. Example 3).

ELISpot assay (A) was performed on spleen cells at day 35 (week 5) to quantify CEA-specific IFN-γ-producing T cells. Briefly, spleen cells were incubated for 24 h with CEA peptide pools. Intracellular staining (B) was performed on spleen cells at day 35 (week 5) to quantify CEA-specific cytokine-producing CD8 T cells. Briefly, spleen cells were incubated for 6 h with CEA peptide pools, including 5 h with a protein transport inhibitor before staining and analysis by flow cytometry.

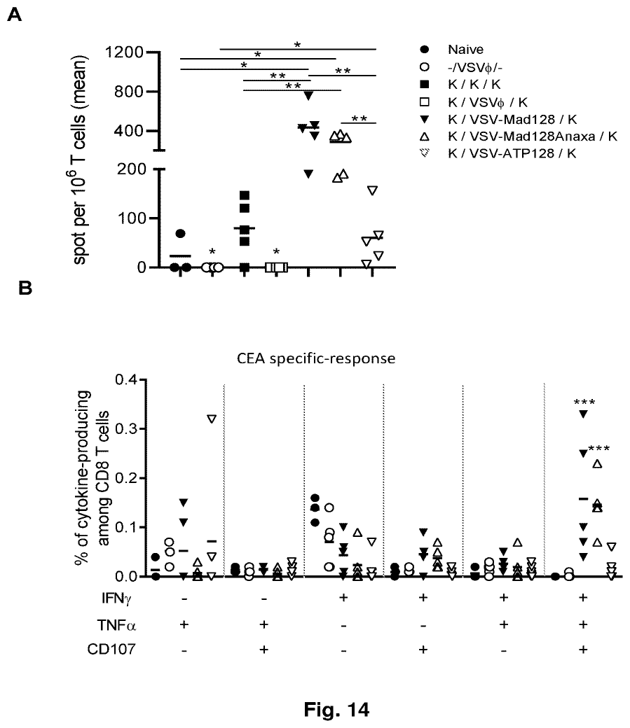

FIG. 14A shows the number of CEA-specific IFN-γ-producing cells (per million of T cells) for the different experimental groups and FIG. 14B shows the percentage of cytokine-producing cells among CD8 T cells.

“ATP128” refers to the complex of the first component (K) of the vaccine of the invention which consists of a polypeptide consisting of the amino acid sequence according to SEQ ID NO: 60, “Mad128” refers to the antigenic domain of the invention comprising the amino acid sequence according to SEQ ID NO: 45, “VSVϕ” refers to VSV-GP that expresses only the GP of LCMV, but does not encode a antigenic domain in its genome.

### Example 5

### Frequencies of Granzyme B Positive Circulating CEA-Specific CD8 T Cells

Naïve C57BL/6 mice (5 mice per group) were vaccinated at days 0 and 28 (weeks 0 and 4) subcutaneously with 10 nmol of the first component K of the vaccine of the invention (K, SEQ ID NO: 60, (“ATP128”)) at the tail base and at day 14 (week 2) intravenously with 10⁷ TCID₅₀ of one of the different VSV-GP constructs.

Intracellular staining was performed on blood cells at day 35 (week 5) to quantify CEA-specific Granzyme B-producing CD8 T cells. Briefly, blood cells were incubated for 4 h with a protein transport inhibitor before multimer and intracellular staining and analysis by flow cytometry.

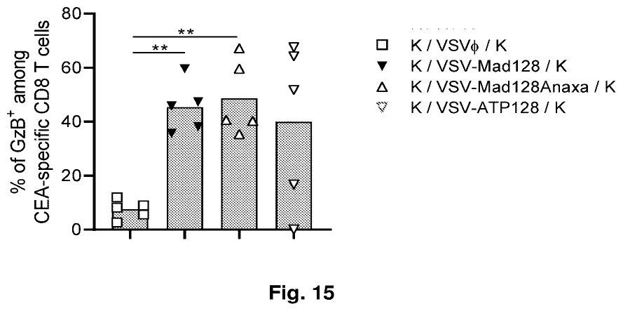

FIG. 15 shows the percentage of Granzyme B-producing cells among CEA-specific CD8 T cells) for the different experimental groups.

As can be seen in FIG. 15 recombinant VSVs according to the invention comprising an antigenic domain without CPP functionality display superior multifunctional CEA-specific CD8 T cell responses in the periphery.

### Example 6

### Heterologous Vaccination Reverses Immunosuppression in Tumor Microenvironment (TME)

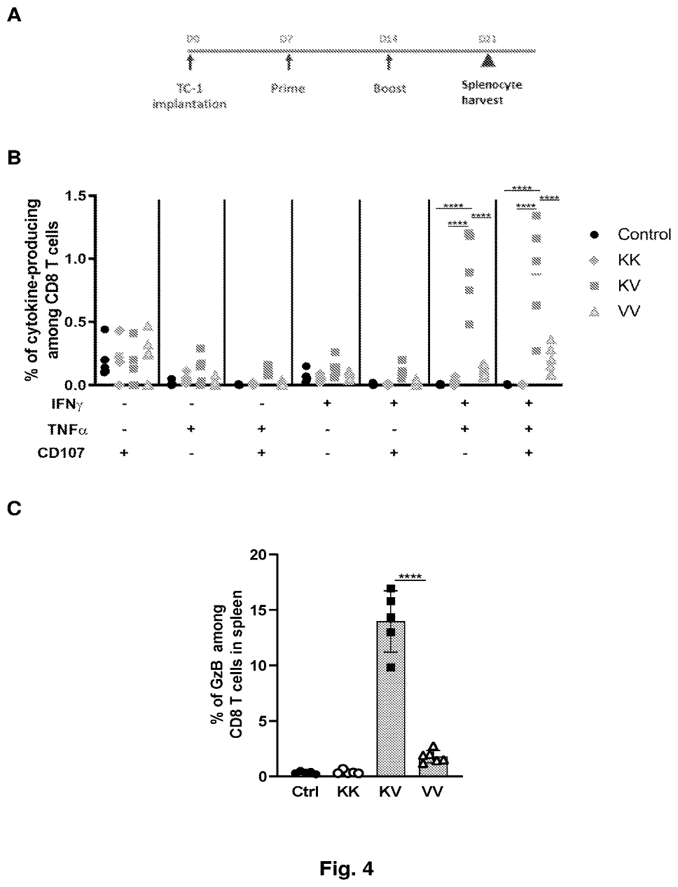

Mice were vaccinated as shown in FIG. 4A and described in Example 7. Analysis of TME and tumor-infiltrating leukocytes (TILs) after heterologous prime boost using a vaccine comprising an antigenic domain (Mad25, SEQ ID NO: 75) which comprises HPV E7 antigenic epitopes and VSV-GP-E7 encoding in its genome the same E7 antigenic epitopes in the TC-1 tumor model which was assessed at day 27, one week after administration of the second component (V) (“VSV-GP-HPV boost”), by NanoString® based transcriptome analysis, by flow cytometry, and by immunohistochemistry.

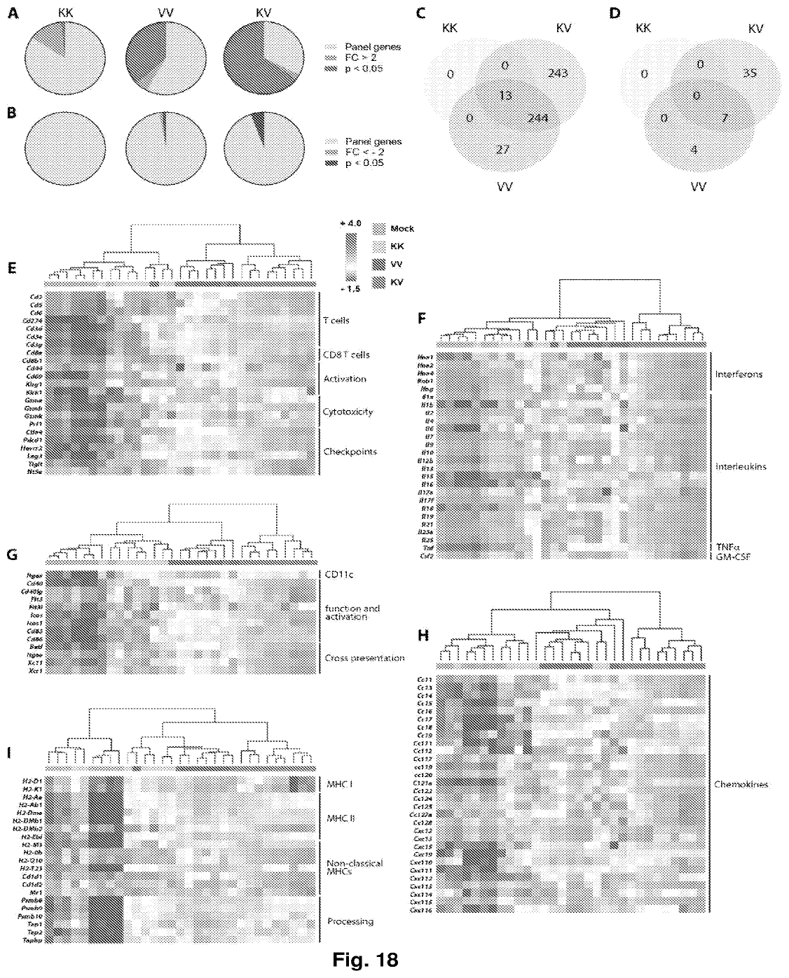

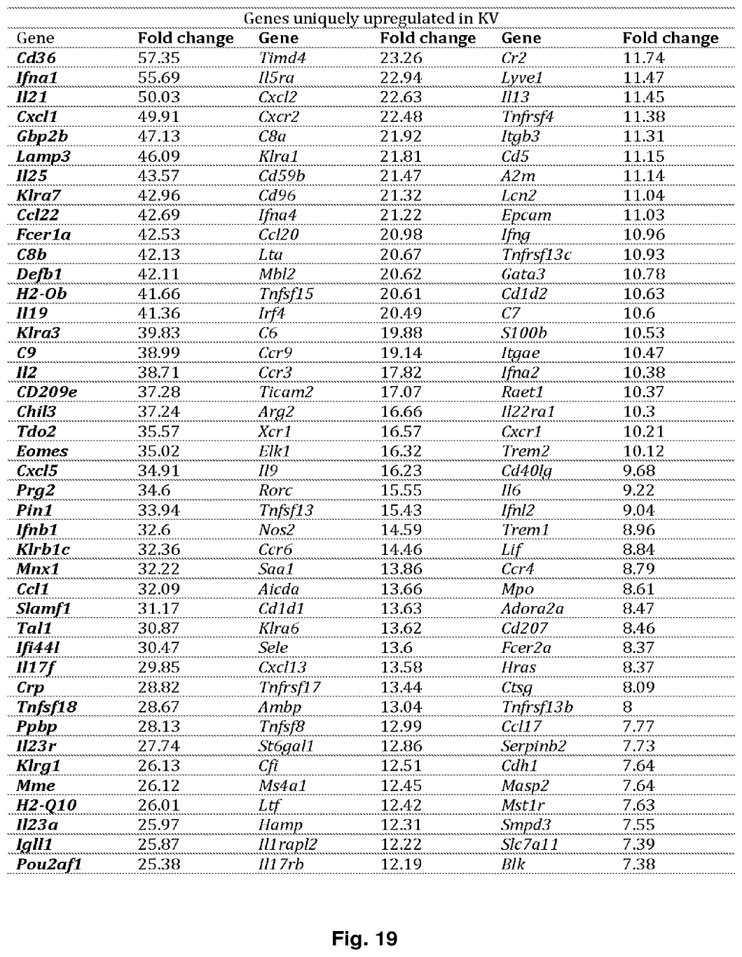

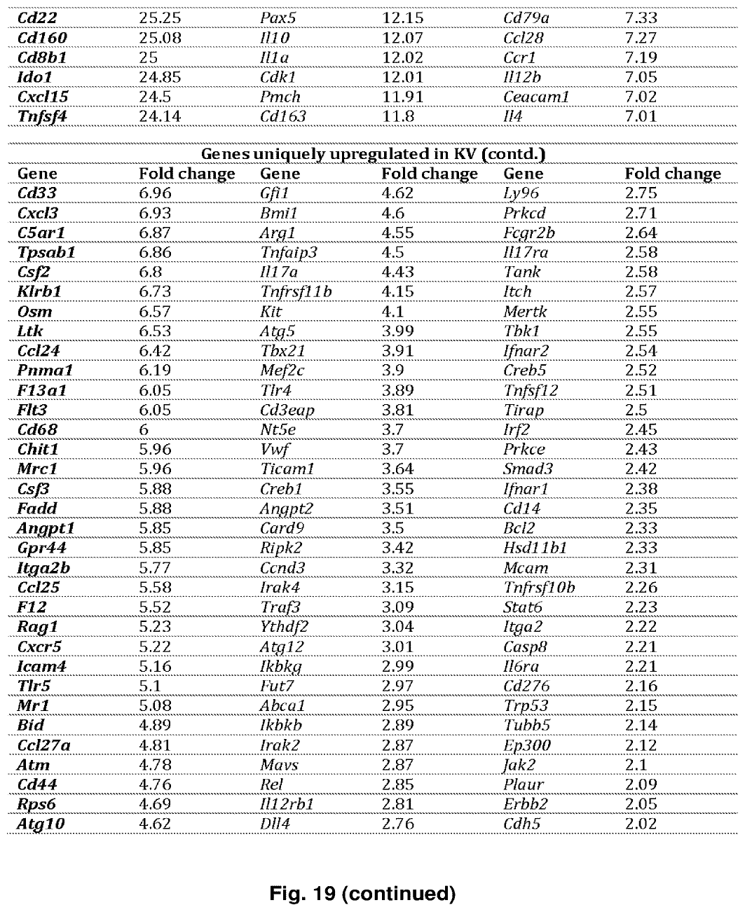

After heterologous KV vaccination, dramatic changes in the TC-1 TME were observed upon NanoString® based transcriptome analysis as highlighted by the differential expression of several genes (FIGS. 18, A and B). 64.9% of all panel genes were upregulated in KV treated tumors, compared to 36.8% after homologous VV vaccination; indicating stronger activation of multiple immune pathways (FIG. 18A). While 244 of these genes could be attributed to the immune activating effects of VSV-GP-HPV, a set of 243 genes was upregulated only in the heterologous vaccination group (FIG. 18C). The genes uniquely upregulated in KV treatment are involved in both innate and adaptive immune responses (FIG. 19). Interestingly, heterologous vaccination also negatively regulated the expression of 35 genes (FIGS. 18, B and D) including Cdkn1a and MsIn which are involved in cancer progression. In addition, heterologous KV vaccination activated multiple immune genes associated with cytotoxic T cells (FIG. 18E), dendritic cells (DCs) (FIG. 18G), cytokines (FIG. 18F), chemokines (FIG. 18H) and antigen processing and presentation (FIG. 18I). Hierarchical clustering revealed that tumors from mice receiving a specific vaccine combination had a similar transcriptome and thus were more likely to cluster together. Biologically, increased CTL infiltration along with elevated levels of cytotoxic genes such as granzymes (Grzma, Grzmb and Grzmk) and perforin (Prf1) (FIG. 18E) and antigen presentation (FIG. 18I) suggested enhanced tumor cell killing as a result of heterologous vaccination. Besides, more genes indicative of DC function and maturation including cross-presentation were upregulated in heterologous treated tumors, which supports anti-tumor immunity (FIG. 18G). All vaccine combinations upregulated components of the antigen processing machinery but homologous VV vaccination had a stronger impact on genes encoding MHC I and MHC II molecules; whereas KV vaccination positively regulated non-classical MHCs (FIG. 18I).

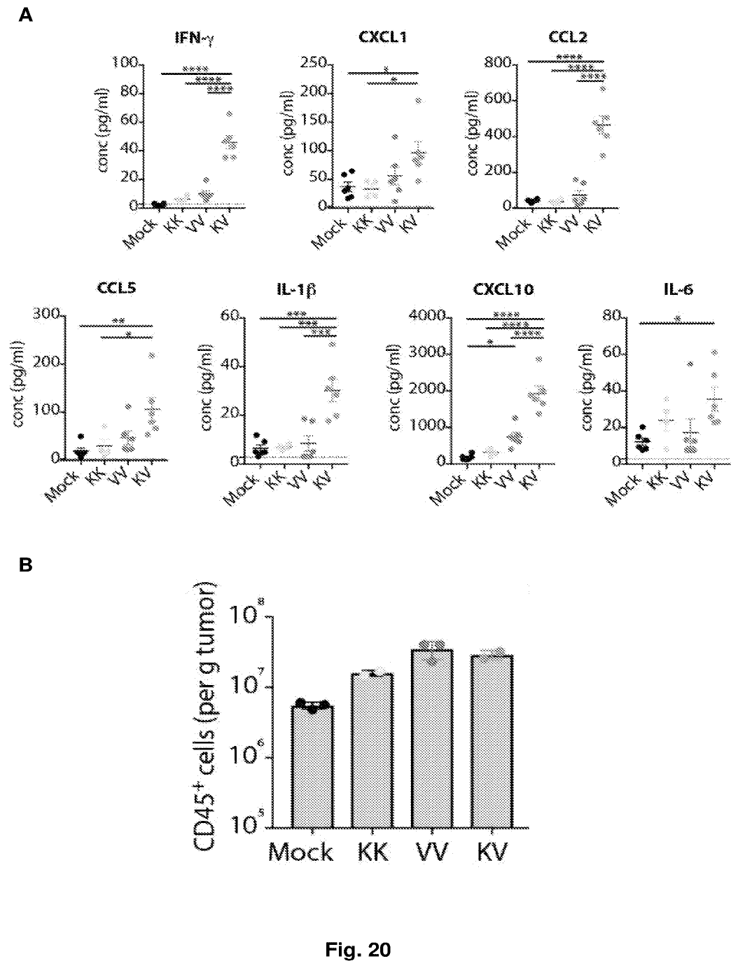

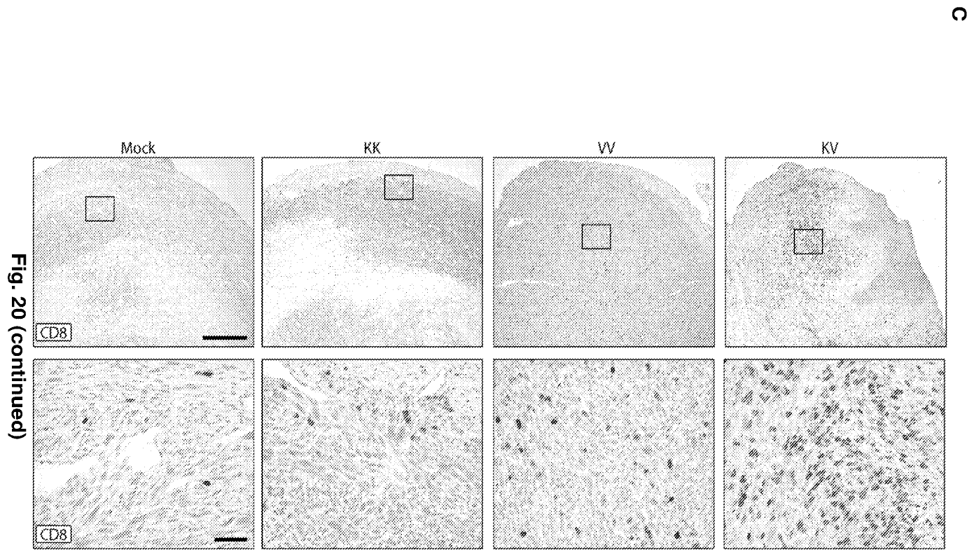

Both pro-inflammatory and anti-inflammatory cytokines were upregulated as a result of the immune activating effect of KV vaccination. Of note, elevated levels of Type I and Type II interferons (FIG. 18F) would explain upregulation of genes involved in antigen presentation pathway. Also, cytokines such as Ifng and Tnf important for T-cell effector functions were elevated in TC-1 tumors after heterologous vaccination (FIG. 18F). Finally, both VV and KV vaccination induced the expression of several chemokines in treated TC-1 tumors. Interestingly, some of the cytokines and chemokines upregulated in the tumor were also elevated in the plasma of mice receiving heterologous KV vaccine, including increased levels of IFN-γ, CCL5, CXCL10, CCL2, IL-6, CXCL1 and IL-1β one day after VSV-GP-HPV boost (FIG. 20A).

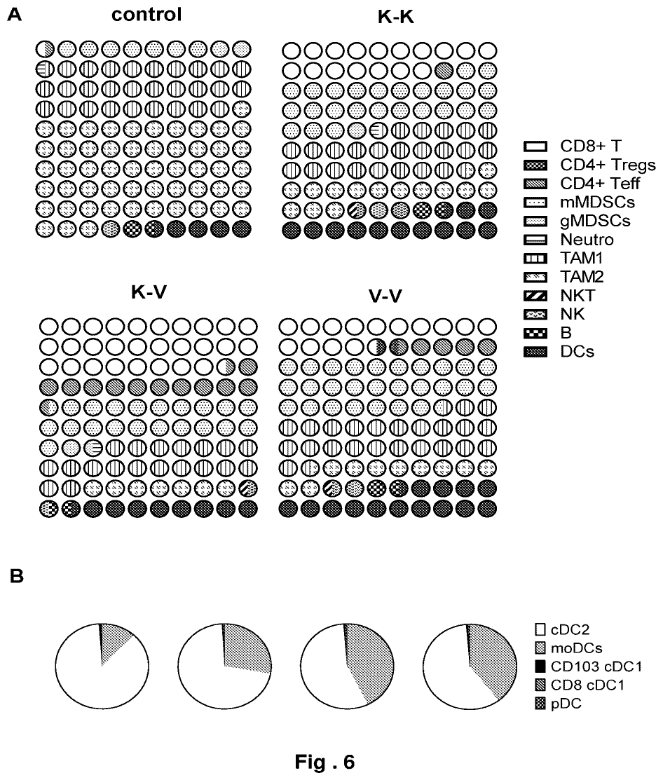

Observations from transcriptome analysis were further supported by the analysis of the number (FIG. 20B) and types (FIG. 6) of tumor-infiltrating leukocytes (TILs). Major leucocyte populations were quantified by several marker combinations and repartition in the tumors represented in FIG. 6. Immune suppressive cells, such as Myeloid-derived suppressor cells (MDSCs), a heterogeneous population of immature myeloid cells, regulatory T cells (Tregs) and Tumor-Associated Macrophages-2 (TAM2, unlike the proinflammatory, TAM1), are a major obstacle for immunotherapy. TILs from untreated TC-1 tumors are predominantly composed of immunosuppressive cells such as M2-like tumor-associated macrophages (TAM-2) and myeloid-derived suppressor cells (MDSCs), which together represent over 80% of tumor-infiltrating immune cells, while T cells constitute only 1% of the infiltrate (FIG. 6A). Therapeutic vaccination induced deep changes in the TILs, with a striking influx of both CD8+ and CD4+ T cells populations and a drastic decrease of TAM-2, resulting in an enhanced TAM-1:TAM-2 ratio suggesting repolarization. Furthermore, heterologous KV vaccination promoted the strongest influx of CD8+ T cells, which represented over 25% of tumor infiltrating immune cells (FIG. 6A). Thus, while both vaccination regimens promoted trafficking of immune cells into the tumor, KV vaccination attracted the highest proportion of CTLs, CD4+ T helper cells and increased TAM-1:TAM-2 ratio thereby remodeling the TME and creating a favorable environment for the clearance of tumor cells. The use of a vaccine as disclosed above increases the proportion of CD8 and effector CD4 T cells and displays a favourable TME characterized by major improvement of TAM1/TAM2 ratio.

Next, immunohistochemistry was performed to confirm the location of immune infiltrates. CD8 staining of tumors harvested 9 days post boost confirmed the general immune-excluded phenotype of untreated TC-1 tumors with few CD8+ T cells confined to the tumor margin (FIG. 20C). While CD8+ T cell infiltration increased with homologous KK and VV vaccine regimen, the heterologous combination KV displayed a massive cytotoxic T cell presence in the deepest parts of the tumor.

### Example 7

Priming with a First Component (K) of the Vaccine Improves the Functionality of Peripheral Antigen-Specific CTLs

Mice with palpable TC-1 tumors were vaccinated either with 2 nmol of the first component (K) in which the antigenic domain comprises Mad25, administered s.c. on day 7 and 1×10⁷ TCID₅₀ of the second component (V) (VSV-GP-HPV-E2-E6-E7 administered i.v.) on day 14, or twice with 2 nmol of the first component (K) on days 7 and 14, or twice with the second component (V) on days 7 and 14 (FIG. 4). Spleens were harvested on day 21 post implantation of the TC-1 tumors for analysis of CD8 lymphocytes. The used VSV-GP-HPV-E2-E6-E7 contains the full-length genes encoding the three different antigens E2, E6* and E7* (containing the same antigenic domain of Mad25) derived from HPV16. The original E6 and E7 sequences were mutated to carry point mutations that abrogate their oncogenicity. Cytokine production by ex vivo restimulated HPV-specific CD8 T cells was measured by intracellular flow cytometry staining (FIG. 4B) which shows significant increases in IFNγ+-CD107+ cells, IFNγ+-TNFα+ cells and IFNγ+-TNFα+-CD107+ cells compared to control conditions, and demonstrates that heterologous vaccination (KV) leads to stronger responses than homologous vaccination (VV). (C) Granzyme B expression by splenic CD8 T cells was measured by flow cytometry, which demonstrates that heterologous vaccination (KV) leads to stronger responses than homologous vaccination (either VV or KK).

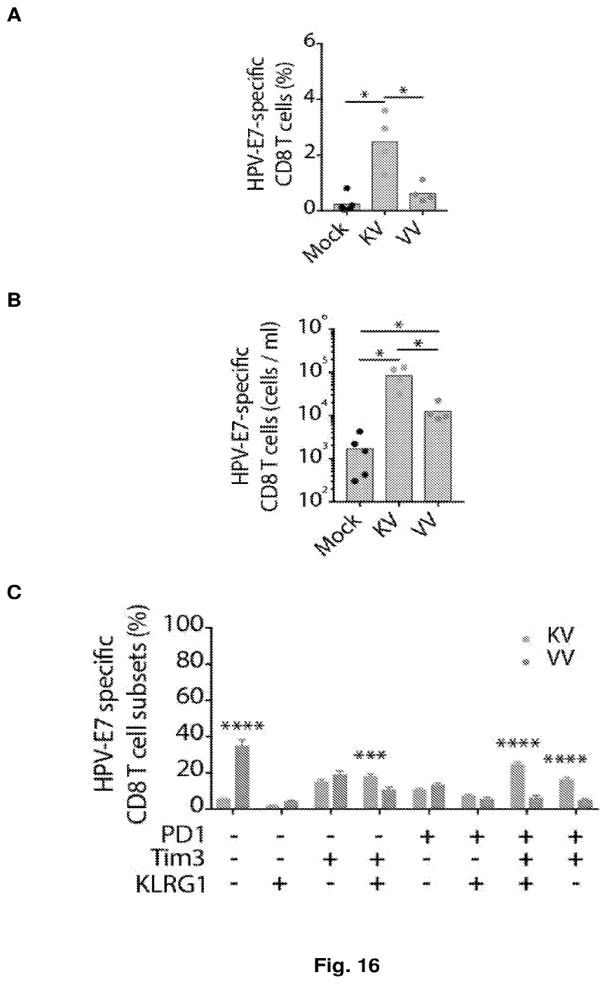

Consistent with the results in non-tumor bearing animals, the first component (K)-HPV prime followed by VSV-GP-HPV boost resulted in significantly higher frequency (FIG. 16A) and absolute numbers (FIG. 16B) of HPV-E7 specific CD8+ T cells in the periphery, compared to homologous VSV-GP-HPV treatment. As the immunosuppressive tumor microenvironment is well known to induce a rapid exhaustion of T cells, the phenotype of circulating antigen specific CD8+ T cells was assessed. As shown in FIG. 16C, only a small portion of HPV-E7 specific CD8⁺ T cells displayed an exhausted phenotype in the periphery, characterized by the expression of PD-1 and Tim-3.

### Example 8

Priming with a First Component (K) of the Vaccine Improves the Functionality of Intratumoral Antigen-Specific CTLs

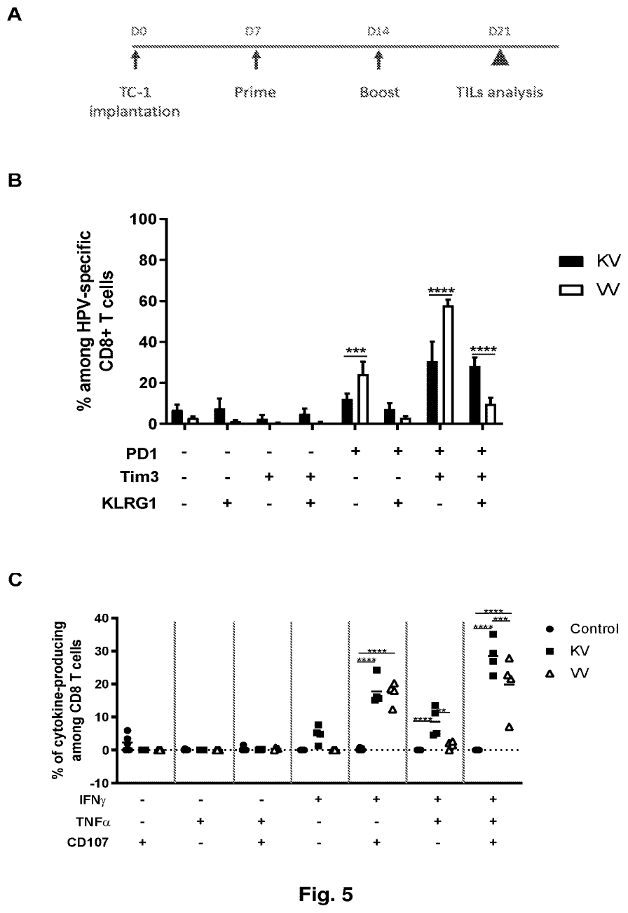

Mice with palpable TC-1 tumors were vaccinated with either 2 nmol of the first component (K) of the vaccine with the antigenic domain Mad25 (administered s.c.) on day 7 and 1×10 TCID₅₀ of the second component (V) (VSV-GP-HPV-E2-E6-E7) comprising the same antigenic domain Mad25 (administered i.v.) on day 14 after TC-1 tumor implantation, or twice with the second component (V) (VSV-GP-HPV-E2-E6-E7, administered i.v.) on days 7 and 14 after TC-1 tumor implantation (FIG. 5). Tumors were harvested on day 21 post implantation for analysis of tumor-infiltrating lymphocytes (TILs). The frequency of activation and exhaustion marker expression (PD1, Tim3, KLRG1) by HPV-specific CD8 T cells was measured by flow cytometry (FIG. 5B). However, while only a small portion of HPV-E7 specific displayed an exhausted phenotype in the periphery (FIG. 16C, see Example 7 above), the majority of tumor infiltrating CD8+ T cells expressed both markers—suggesting their exhaustion (FIG. 5B). Interestingly, a higher proportion of intratumoral PD-1+ Tim-3+CD8+ T cells in KV vaccinated mice still expressed the early activation marker KLRG-1, suggesting a less advanced exhaustion status compared to the homologous VV treated mice. Since T cell exhaustion is a progressive process, which initiates with the expression of markers and continues with loss of function and eventually cell death, CD8+ T cells functionality was assessed by measuring cytokine secretion after ex vivo restimulation. Cytokine production by ex vivo restimulated HPV-specific CD8 T cells was measured by intracellular flow cytometry staining (FIG. 5C). While in the periphery, a significantly higher proportion of splenic HPV-E7 specific CD8+ T cells in KV vaccinated mice expressed IFN-γ, TNF-α and the degranulation factor CD107a compared to VV treated mice (FIG. 4B, Example 7) and a higher frequency of Granzyme B producing CTLs were detected in KV vaccinated mice compared to VV vaccinated mice (FIG. 4C, Example 7), a much higher proportion of CD8+ T cells was found to be activated within the tumor, where most of the HPV-E7 specific CD8+ T cells were multifunctional, producing IFN-γ, TNF-α and/or CD107a (Fig. FIG. 5C). In accordance to the results from the spleen, KV vaccination induced a significantly higher proportion of multifunctional CD8+ T cells compared to VV treatment, in particular IFN-γ+TNF− α+CD107a+ triple-positive cells, confirming the phenotyping data and highlighting a highly cytotoxic, poorly exhausted phenotype of KV elicited antigen-specific CD8+ T cells.

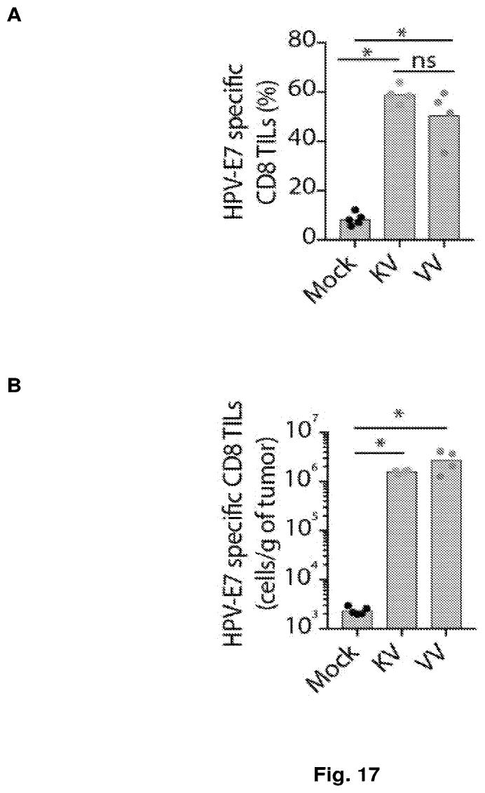

Both vaccination regimens (KV and VV) were able to induce high infiltration of CD8+ T cells within the tumor, about 60% of which were found to be HPV-E7 specific by multimer staining (FIGS. 17A and B). In contrast to the periphery, there were no differences within the tumor between the two vaccine regimens in HPV-E7 specific CD8+ T cells frequency (FIG. 17A) and numbers (FIG. 17B).

Overall, priming with the first component (K) and boosting with the second component (V) not only supports induction of higher magnitude of tumor-specific CD8+ T cells, but also promotes their recruitment into the tumor and enhances their functionality compared to homologous viral vaccination.

### Example 9

### Therapeutic Effect of a Heterologous KVKK Vaccine in Syngeneic Tumor Models Expressing Ovalbumin

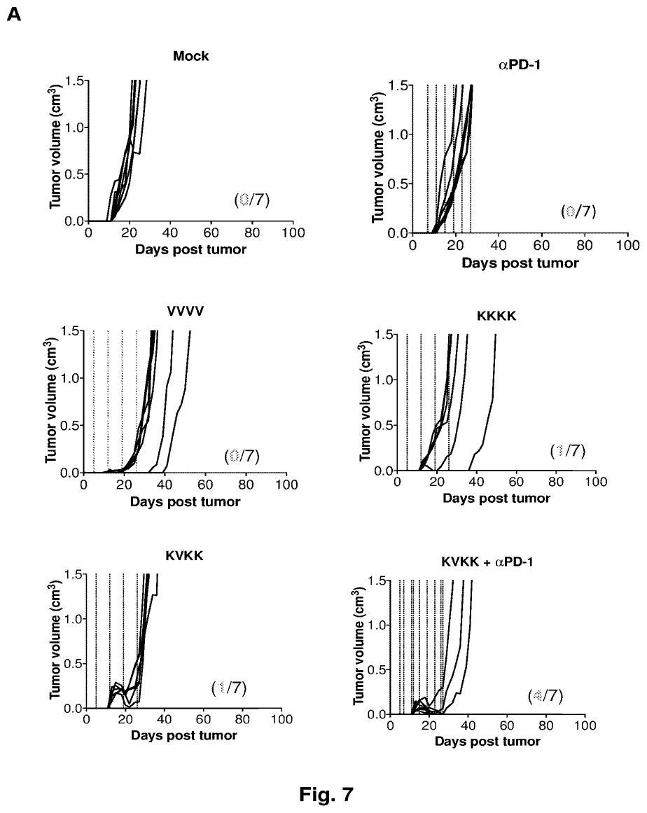

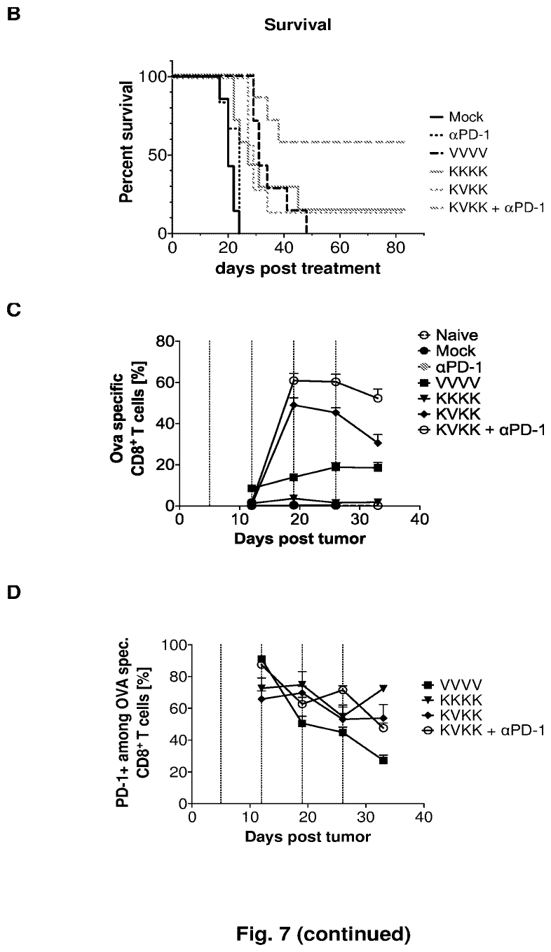

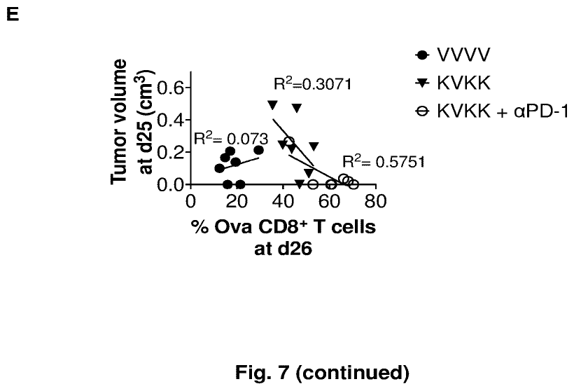

C57BL/6 mice were injected with 3×10⁵ EG.7 cells. Mice were treated with 2 nmol of a first component (K) comprising the antigenic domain Mad39 (amino acid sequence according to SEQ ID NO: 77) given s.c. (dotted lines), 1×10⁷ TCID₅₀ VSV-GP-OVA comprising the full-length gene encoding Ovalbumin (containing the antigenic domain Mad39) in its genome given i.v. (dotted lines) or 200 μg αPD-1 antibody given i.v. Blood was drawn 7 days after vaccination for tetramer analysis. Administration of either the first component (K) or the second component (V) (VSV-GP-OVA) was performed on days 5, 12, 19 and 26 post tumor implantation. Administration of the αPD-1 antibody was performed on days 7, 11, 15, 19, 23 and 27 post tumor implantation. Controls were performed with mock treatment and αPD-1 antibody only. Four different treatment regimens were tested: VVVV, KKKK, KVKK, and KVKK+αPD-1. Tumor growth (FIG. 7A) and survival (FIG. 7B) after treatment was assessed. The number of complete responders (grey), i.e. tumor-free mice, among total mice (black) is indicated in parentheses beside the tumor growth curves for every treatment group. The frequency of Ova-specific CTLs (FIG. 7C) and the proportion of PD-1 positive among Ova tetramer positive cells in peripheral blood (FIG. 7D) was analysed. The correlation between the magnitude of Ova response (at day 26) and tumor sizes (at day 25) was analysed for three different treatment groups (FIG. 7E).

### Example 10

### Efficacy of Therapeutic Cancer Vaccination Using a First Component (K) and a Second Component (V) of the Vaccine (VSV-GP-TAA) in Syngeneic Tumor Models Targeting Neoepitopes

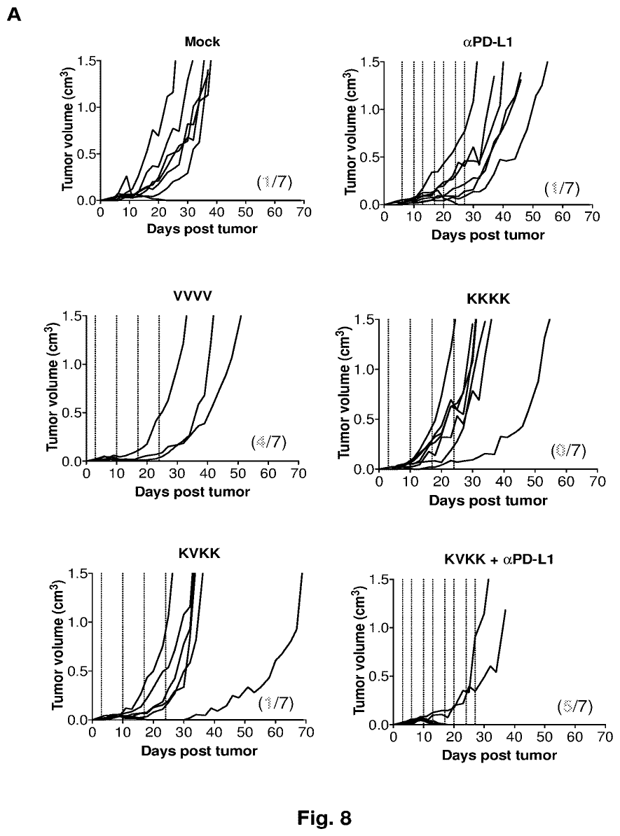

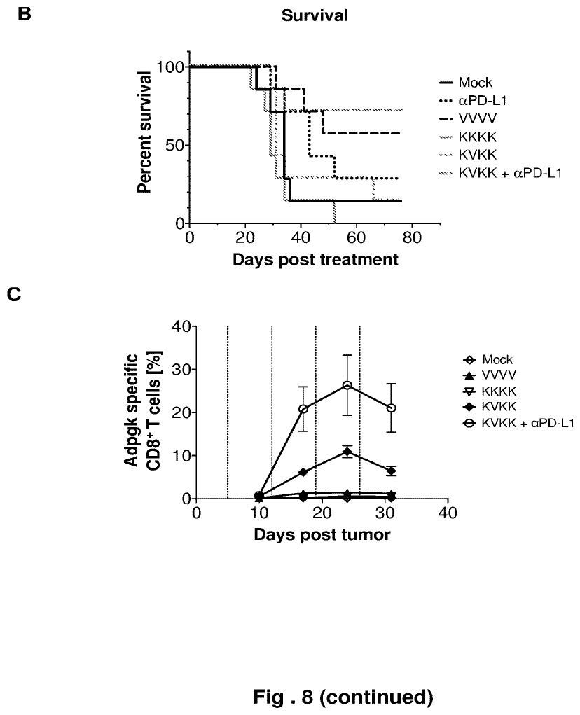

C57BL/6 mice were injected with 2×10⁵ MC-38 cells subcutaneously in the right flank. Mice were vaccinated against Adpgk and Reps1 (MC-38 neo-epitopes, antigenic domain Mad24, SEQ ID NO: 76) using 2 nmol of the first component (K) comprising Mad24 administered s.c. or 1×10⁷ TCID₅₀ of the second component (V) (VSV-GP-TAA) comprising Mad24 administered i.v. on the days indicated (dotted lines). Additionally, mice received 200 μg of αPD-L1 antibody i.p. on the days indicated (dotted lines). Administration of either the first component (K) or the second component (V) (VSV-GP-TAA) was performed on days 3, 10, 17 and 24 post MC-38 cells injection. Administration of the αPD-1 antibody was performed on days 6, 10, 13, 17, 20, 24 and 27 post MC-38 cells injection. Controls were performed with mock treatment and αPD-1 antibody only. Four different treatment regimens were tested: VVVV, KKKK, KVKK, and KVKK+αPD-1. Tumor growth (FIG. 8A) and survival (FIG. 8B) of the animals was monitored. The number of complete responders (grey), i.e. tumor-free mice, among total mice (black) is indicated in parentheses beside the tumor growth curves for every treatment group. The frequency of circulating Adpgk-specific CD8 T cells was assessed by flow cytometry 7 days after each vaccination (FIG. 8C).

### Example 11

### Efficacy of Therapeutic Cancer Vaccination Using a First Component (K) and a Second Component (V) VSV-GP-TAA in Syngeneic Tumor Models Targeting an Oncoviral Antigen

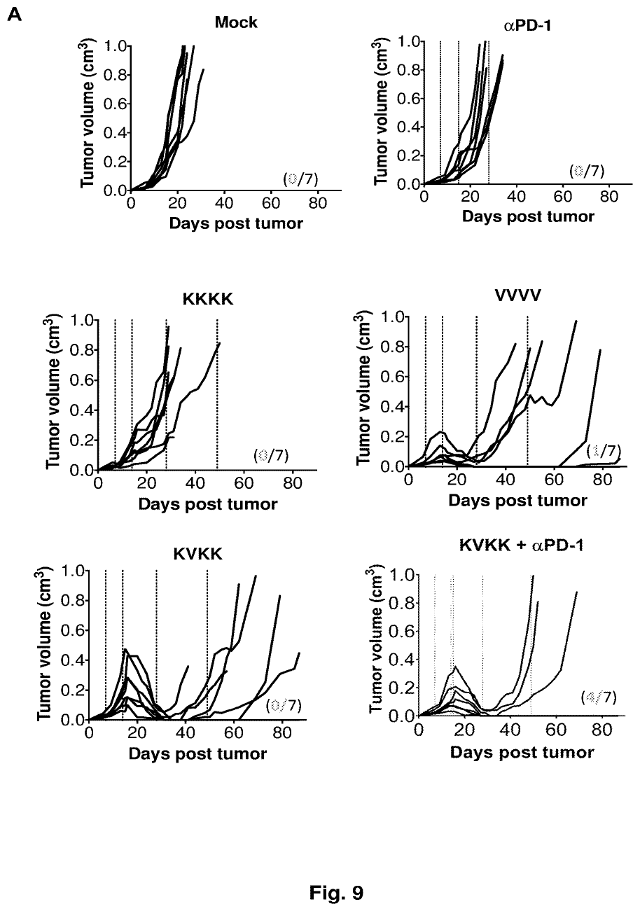

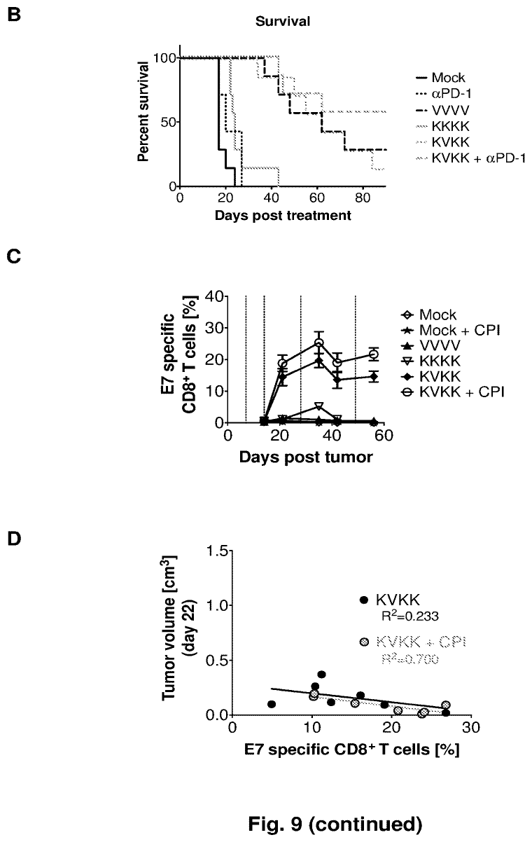

C57BL/6 mice were injected with 1.5×10⁵ TC-1 cells subcutaneously in the right flank. Mice were vaccinated against E7 (an HPV-derived oncoprotein expressed in TC-1 cells) using 2 nmol of a first component (K) comprising the antigenic domain Mad25 (SEQ ID NO: 75) administered s.c. and 1×10⁷ TCID₅₀ VSV-GP-TAA administered i.v. on the days indicated (dotted lines). Additionally, mice received 200 μg of αPD-1 antibody administered i.v. on the days indicated (dotted lines). Administration of either the first component (K) or the second component (V) (VSV-GP-TAA) was performed on days 7, 14, 28 and 49 post TC-1 cells injection. Administration of the αPD-1 antibody was performed on days 7, 14 and 28 post Tc-1 cells injection. Controls were performed with mock treatment and αPD-1 antibody only. Four different treatment regimens were tested: VVVV, KKKK, KVKK, and KVKK+αPD-1. Tumor growth curves (FIG. 9A) and survival (FIG. 9B) of the animals was monitored. The frequency of circulating HPV-E7-specific CD8 T cells was assessed by flow cytometry 7 days after each vaccination (FIG. 9C). The correlation between the proportion of antigen specific CTLs and the tumor size is shown (FIG. 9D). The number of complete responders (grey) among total mice (black) is indicated in parentheses beside the tumor growth curves for every treatment group.

### Example 12

### Heterologous Prime-Boost Vaccination Generates a Long-Lasting Immune Memory in Vaccinated Mice

To assess the immune memory in vaccinated mice the presence of circulating tumor-specific CTLs against vaccinated antigens was assessed in long-term surviving mice which had rejected the subcutaneous tumors after therapeutic vaccination. The frequency of Ova specific (FIG. 10A), Adpgk specific (FIG. 10B) and E7 specific (FIG. 10C) CD8+ T cells in the peripheral blood of mice which rejected the respective EG.7 (FIG. 10A), MC38 (FIG. 10B) and TC-1 tumors (FIG. 10C) is shown.

### Example 13

### Tumor Rechallenge Protection in Vaccinated Mice

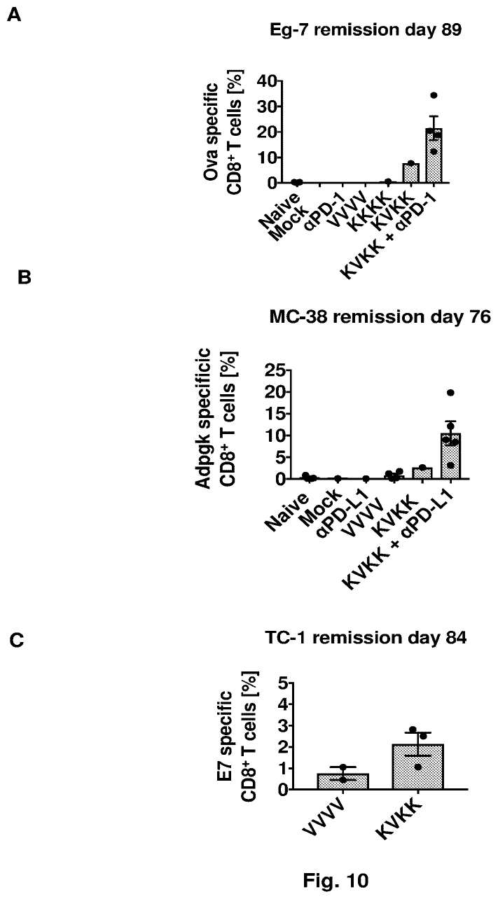

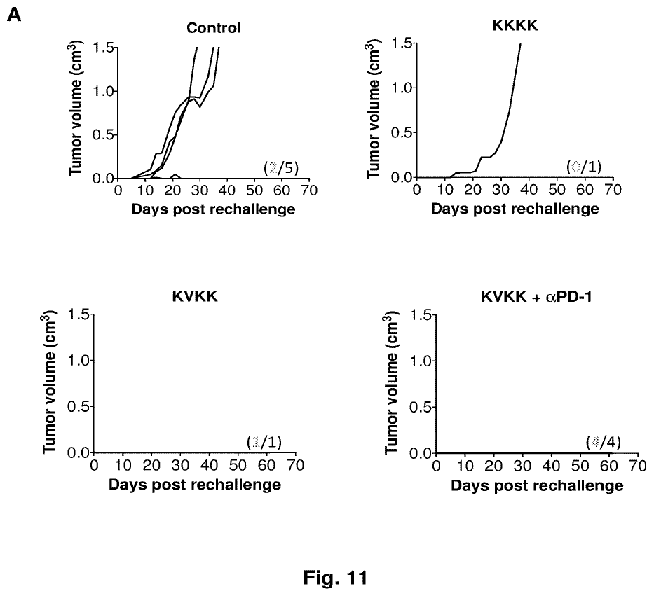

Surviving mice from different treatment groups (Example 9, Example 10 and Example 11) were rechallenged with EG.7, MC38 or TC-1 cells, respectively, on the contralateral flank and subsequent tumor growth was monitored. FIG. 11A shows the results for the EG.7-OVA group, while FIG. 11B shows data combined from three independent TC-1 experiments. Age-matched littermates (control) were included. The number of tumor-free mice (grey) among total mice (black) is indicated in parentheses beside the tumor growth curves for every tumor growth curve. Results for all three tumor models (EG.7, MC38 and TC-1) are summarized in Table 3 below:

TABLE 3
Tumor rechallenge protection of mice with long-term
remission of treated E.G7, MC-38 and TC-1 tumors.
a) Rechallenge protection in EG.7 tumor model

| | | Protected against | Ova specific CD8+ |
|---|---|---|---|
| Treatment | Long-term survivors | rechallenge (%) | T cells (%) |
| KKKK | 1 | 0 | 0.58 |
| KVKK | 1 | 100 | 7.71 |
| KVKK + αPD-1 | 4 | 100 | 21.45 |
| KV_{ϕ}KK | 1 | 100 | 0.46 |

b) Rechallenge protection in MC38 tumor model

| | | Protected against | Protected against | |
|---|---|---|---|---|
| | | rechallenge with | rechallenge with | |
| | Long-term | neoantigen | neoantigen | Adpgk specific |
| Treatment | survivors | expressing cells (%) | lacking cells (%) | CD8+ T cells (%) |
| αPD-L1 | 1 | 100 | 100 | 0.07 |
| VVVV | 4 | 75 | 50 | 0.83 |
| KVKK | 1 | 100 | 0 | 2.63 |
| KVKK + αPD-L1 | 5 | 100 | 100 | 10.94 |
| KV_{ϕ}KK | 1 | 100 | 0 | 0.31 |
| V_{ϕ}V_{ϕ}V_{ϕ}V_{ϕ} | 2 | 100 | 50 | 0.084 |

c) Rechallenge protection in Tc1 tumor model

| | | Protected against |
|---|---|---|
| Treatment | Long-term survivors | rechallenge (%) |
| VVVV | 5 | 60 |
| KVKK | 6 | 100 |
| KVKK + αPD-1 | 4 | 75 |

In summary, KVK heterologous prime-boost (both alone and in combination with checkpoint blocking antibodies) developed an effective memory response, as almost all re-challenged mice rapidly rejected the newly implanted tumor (Table 3, A-C). Interestingly, in TC-1 bearing mice, only 60% of homologous VSV-GP-HPV treated long-term survivors were protected against re-challenge, possibly reflecting the reduced formation of memory precursor cells compared to the heterologous vaccination. Similarly, only 75% of long-term survivors which had successfully rejected MC-38 tumors upon homologous VSV-GP-Mad24 vaccination remained tumor-free after rechallenge.

### Example 14

### Component (K) Promotes Formation of Memory T Cells in Vaccinated Mice

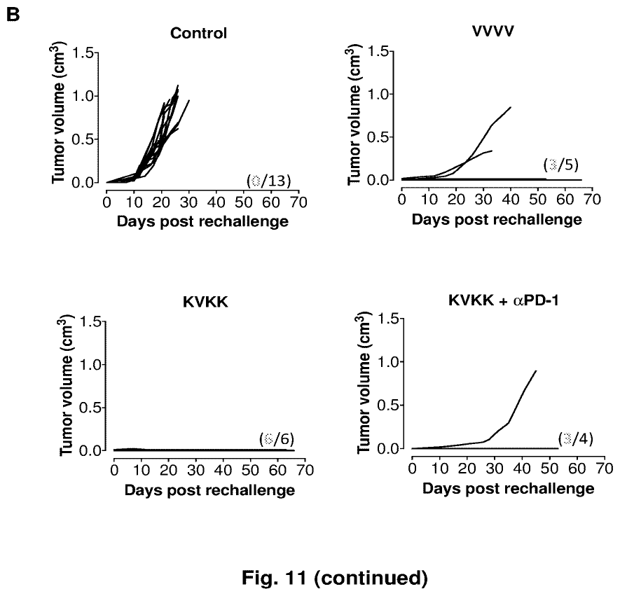

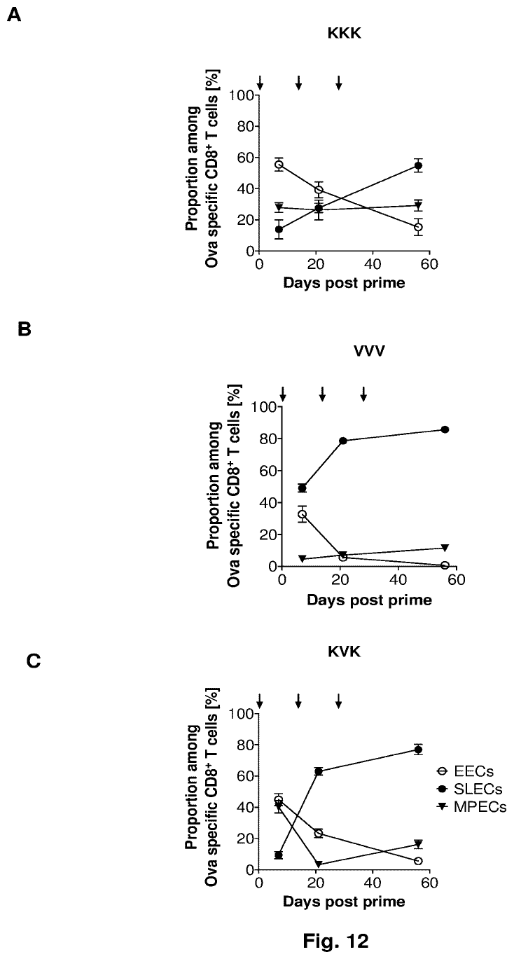

Non-tumor bearing mice were immunized with 2 nmol of a first component (K) comprising the antigenic domain Mad5 (SEQ ID NO: 74) administered s.c. or 1×10⁷ TCID₅₀ VSV-GP-Ova comprising the full-length gene encoding Ovalbumin (containing the antigenic domain Mad5) administered i.m. on days 0, 14 and 28. The proportion of CD127⁻KLRG-1⁻ early effector cells (EECs), KLRG-1⁺ short-lived effector cells (SLECs) and CD127⁺ memory precursor effector cells (MPECs) among Ova-specific CD8⁺ T cells was measured in the peripheral blood 7 days after the 2 first immunizations and 28 days after the 3^{rd} immunization for the homologous vaccination (KKK) (FIG. 12A), for the homologous VSV-GP-Ova vaccination (VVV) (FIG. 12B) and for the heterologous prime-boost vaccination (KVK) (FIG. 12C).

### Example 15

### The First Component (K) Prime is Crucial for the Therapeutic Effect of Heterologous Vaccination in TC-1 Tumor Model

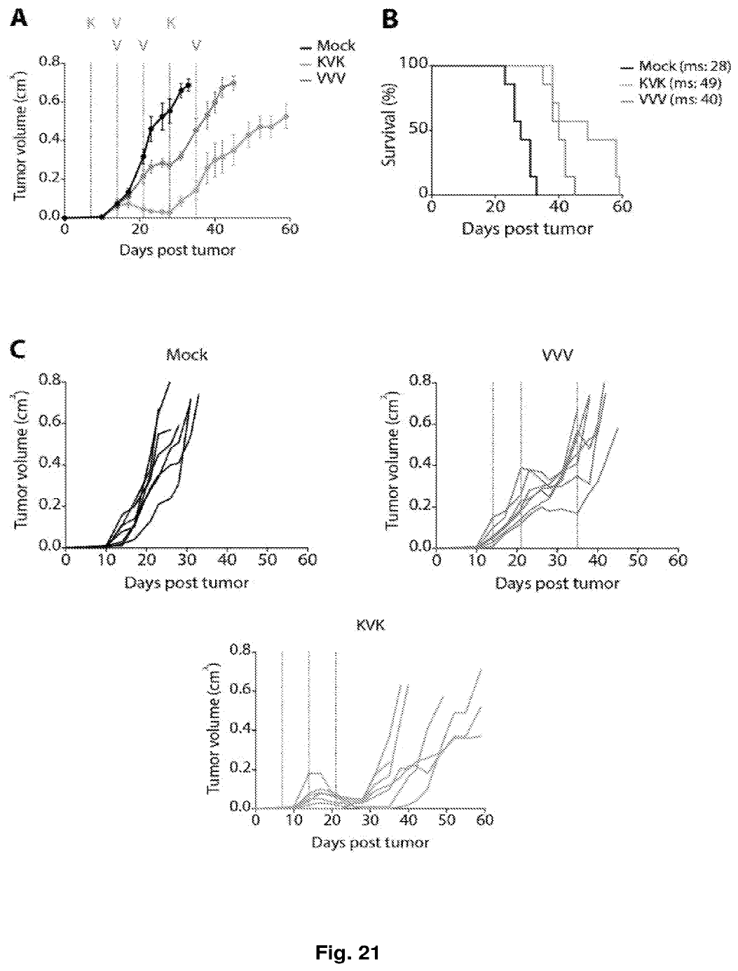

In order to address the role of the first component (K) prime, second component (VSV-GP-HPV) treatment at 14 days post tumor (time of boost) was assessed with or without first component (K) prime (FIG. 21A). Briefly, mice were injected with 1×10⁵ TC-1 cells s.c. and were immunized with 2 nmole first component (K)-(antigenic domain comprising Mad25) prime s.c. 7 days later or with 1×10⁷ TCID₅₀ VSV-GP-HPV i.v. 14 days post tumor implantation, essentially as described above for the TC-1 model. Additional doses of K and V were administered as indicated by dotted lines in FIG. 21A.

While virus treatment alone led to the slowing of tumor growth, no remission was observed (FIGS. 21, A and C). In contrast, virus treatment following the first component (K) prime led to a complete remission in all tumors; even in large tumors (FIGS. 21, B and C). This strongly indicates that priming with the first component (K) is essential to induce tumor regression of sizeable tumors treated with virus two weeks after grafting. These data suggest that the first component (K) prime lays the immunological foundation for the strong tumor remission that follows the second component (V) boost in the TC-1 tumor model.

Taken together, the data shown in the Examples strongly support the heterologous prime boost vaccine with the first component (K) and the second component (V) as described herein. This approach leads not only to significantly enhanced peripheral and intratumoral T cell levels, but also to a profound reshaping of the TME towards a more immune-supportive composition.

TABLE OF SEQUENCES AND SEQ ID NUMBERS (SEQUENCE LISTING):

| SEQ ID NO | Sequence | Remarks |
|---|---|---|
| SEQ ID NO: 1 | MMDPNSTSEDVKFTPDPYQVPFVQAFDQAT | ZEBRA is disclosed |
| | RVYQDLGGPSQAPLPCVLWPVLPEPLPQGQ | under NCBI |
| | LTAYHVSTAPTGSWFSAPQPAPENAYQAYA | accession number |
| | APQLFPVSDITQNQQTNQAGGEAPQPGDNS | YP_401673 |
| | TVQAAAVVFACPGANQGQQLADIGVPQPA | |
| | PVAAPARRTRKPQQPESLEECDSELEIKRYK | |
| | NRVASRKCRAKFKQLLQHYREVAAAKSSEN | |
| | DRLRLLLKQMCPSLDVDSIIPRTPDVLHEDLL | |
| | NF | |
| SEQ ID NO: 2 | KRYKNRVASRKSRAKFKQLLQHYREVAAAKS | CPP3 (Z13) |
| | SENDRLRLLLK | |
| SEQ ID NO: 3 | KRYKNRVASRKSRAKFKQLLQHYREVAAAK | CPP4 (Z14) |
| SEQ ID NO: 4 | KRYKNRVASRKSRAKFK | CPP5 (Z15) |
| SEQ ID NO: 5 | REVAAAKSSENDRLRLLLK | CPP8 (Z18) |
| SEQ ID NO: 6 | STVHEILCKLSLEGDHSTPPSAYGSVKPYTNF | TLR2 peptide |
| | DAE | agonist Anaxa |
| SEQ ID NO: 7 | STVHEILSKLSLEGDHSTPPSAYGSVKPYTNF | TLR peptide agonist |
| | DAE | “Anaxa” sequence |
| | | variant |
| SEQ ID NO: 8 | NIDRPKGLAFTDVDVDSIKIAWESPQGQVSRY | EDA |
| | RVTYSSPEDGIRELFPAPDGEDDTAELQGLR | |
| | PGSEYTVSVVALHDDMESQPLIGIQST | |
| SEQ ID NO: 9 | MGKGDPKKPRGKMSSYAFFVQTCREEHKKK | TLR2 agonist □30- |
| | HPDASVNFSEFSKKCSERWKTMSAKEKGKF | HMGB1 |
| | EDMAKADKARYEREMKTYIPPKGETKKKFKD | |
| | PNAPKRPPSAFFLFCSEYRPKIKGEHPGLSIG | |
| | DVAKKLGEMWNNTAADDKQPYEKKAAKLKE | |
| | KYEKDIAAYRAKGKPDAAKKGVVKAEKSKKK | |
| | K | |
| SEQ ID NO: 10 | MPLEQRSQHCKPEEGLEARGEALGLVGAQA | MAGE-A3 |
| | PATEEQEAASSSSTLVEVTLGEVPAAESPDP | (UniProtKB P43357) |
| | PQSPQGASSLPTTMNYPLWSQSYEDSSNQE | |
| | EEGPSTFPDLESEFQAALSRKVAELVHFLLLK | |
| | YRAREPVTKAEMLGSVVGNWQYFFPVIFSKA | |
| | FSSLQLVFGIELMEVDPIGHLYIFATCLGLSYD | |
| | GLLGDNQIMPKAGLLIIVLAIIAREGDCAPEEKI | |
| | WEELSVLEVFEGREDSILGDPKKLLTQHFVQ | |
| | ENYLEYRQVPGSDPACYEFLWGPRALVETSY | |
| | VKVLHHMVKISGGPHIS YPPLHEWVLREGEE | |
| SEQ ID NO: 11 | MALPTARPLLGSCGTPALGSLLFLLFSLGWV | Mesothelin |
| | QPSRTLAGETGQEAAPLDGVLANPPNISSLS | (UniProtKBQ13421) |
| | PRQLLGFPCAEVSGLSTERVRELAVALAQKN | |
| | VKLSTEQLRCLAHRLSEPPEDLDALPLDLLLF | |
| | LNPDAFSGPQACTRFFSRITKANVDLLPRGAP | |
| | ERQRLLPAALACWGVRGSLLSEADVRALGGL | |
| | ACDLPGRFVAESAEVLLPRLVSCPGPLDQDQ | |
| | QEAARAALQGGGPPYGPPSTWSVSTMDALR | |
| | GLLPVLGQPIIRSIPQGIVAAWRQRSSRDPSW | |
| | RQPERTILRPRFRREVEKTACPSGKKAREIDE | |
| | SLIFYKKWELEACVDAALLATQMDRVNAIPFT | |
| | YEQLDVLKHKLDELYPQGYPESVIQHLGYLFL | |
| | KMSPEDIRKWNVTSLETLKALLEVNKGHEMS | |
| | PQAPRRPLPQVATLIDRFVKGRGQLDKDTLD | |
| | TLTAFYPGYLCSLSPEELSSVPPSSIWAVRPQ | |
| | DLDTCDPRQLDVLYPKARLAFQNMNGSEYFV | |
| | KIQSFLGGAPTEDLKALSQQNVSMDLATFMK | |
| | LRTDAVLPLTVAEVQKLLGPHVEGLKAEERH | |
| | RPVRDWILRQRQDDLDTLGLGLQGGIPNGYL | |
| | VLDLSMQEALSGTPCLLGPGPVLTVLALLLAS | |
| | TLA | |
| SEQ ID NO: 12 | MGAPTLPPAWQPFLKDHRISTFKNWPFLEGC | survivin |
| | ACTPERMAEAGFIHCPTENEPDLAQCFFCFK | |
| | ELEGWEPDDDPIEEHKKHSSGCAFLSVKKQF | |
| | EELTLGEFLKLDRERAKNKIAKETNNKKKEFE | |
| | ETAKKVRRAIEQLAAMD | |
| SEQ ID NO: 13 | MQAEGRGTGGSTGDADGPGGPGIPDGPGG | NY-ESO-1 |
| | NAGGPGEAGATGGRGPRGAGAARASGPGG | (UniProtKB: P78358) |
| | GAPRGPHGGAASGLNGCCRCGARGPESRLL | |
| | EFYLAMPFATPMEAELARRSLAQDAPPLPVP | |
| | GVLLKEFTVSGNILTIRLTAADHRQLQLSISSC | |
| | LQQLSLLMWITQCFLPVFLAQPPSGQRR | |
| SEQ ID NO: 14 | MERRRLWGSIQSRYISMSVWTSPRRLVELAG | PRAME |
| | QSLLKDEALAIAALELLPRELFPPLFMAAFDG | |
| | RHSQTLKAMVQAWPFTCLPLGVLMKGQHLH | |
| | LETFKAVLDGLDVLLAQEVRPRRWKLQVLDL | |
| | RKNSHQDFWTVWSGNRASLYSFPEPEAAQP | |
| | MTKKRKVDGLSTEAEQPFIPVEVLVDLFLKEG | |
| | ACDELFSYLIEKVKRKKNVLRLCCKKLKIFAMP | |
| | MQDIKMILKMVQLDSIEDLEVTCTWKLPTLAK | |
| | FSPYLGQMINLRRLLLSHIHASSYISPEKEEQY | |
| | IAQFTSQFLSLQCLQALYVDSLFFLRGRLDQL | |
| | LRHVMNPLETLSITNCRLSEGDVMHLSQSPS | |
| | VSQLSVLSLSGVMLTDVSPEPLQALLERASAT | |
| | LQDLVFDECGITDDQLLALLPSLSHCSQLTTL | |
| | SFYGNSISISALQSLLQHLIGLSNLTHVLYPVP | |
| | LESYEDIHGTLHLERLAYLHARLRELLCELGR | |
| | PSMVWLSANPCPHCGDRTFYDPEPILCPCFM | |
| | PN | |
| SEQ ID NO: 15 | MDGGTLPRSAPPAPPVPVGCAARRRPASPE | ASCL2 |
| | LLRCSRRRRPATAETGGGAAAVARRNERER | |
| | NRVKLVNLGFQALRQHVPHGGASKKLSKVET | |
| | LRSAVEYIRALQRLLAEHDAVRNALAGGLRP | |
| | QAVRPSAPRGPPGTTPVAASPSRASSSPGR | |
| | GGSSEPGSPRSAYSSDDSGCEGALSPAERE | |
| | LLDFSSWLGGY | |
| SEQ ID NO: 16 | SAVEYIRALQ | ASCL2 epitope |
| SEQ ID NO: 17 | ERELLDFSSW | ASCL2 epitope |
| SEQ ID NO: 18 | AAVARRNERERNRVKLVNLGFQALRQHVPH | ASCL2 fragment |
| | GGASKKLSKVETLRSAVEYIRALQRLLAEHDA | |
| | VRNALAGGLRPQAVRPSAPRGPSEGALSPA | |
| | ERELLDFSSWLGGY | |
| SEQ ID NO: 19 | MTPGTQSPFFLLLLLTVLTVVTGSGHASSTPG | MUC-1 |
| | GEKETSATQRSSVPSSTEKNAVSMTSSVLSS | |
| | HSPGSGSSTTQGQDVTLAPATEPASGSAAT | |
| | WGQDVTSVPVTRPALGSTTPPAHDVTSAPD | |
| | NKPAPGSTAPPAHGVTSAPDTRPAPGSTAPP | |
| | AHGVTSAPDTRPAPGSTAPPAHGVTSAPDTR | |
| | PAPGSTAPPAHGVTSAPDTRPAPGSTAPPAH | |
| | GVTSAPDTRPAPGSTAPPAHGVTSAPDTRPA | |
| | PGSTAPPAHGVTSAPDTRPAPGSTAPPAHGV | |
| | TSAPDTRPAPGSTAPPAHGVTSAPDTRPAPG | |
| | STAPPAHGVTSAPDTRPAPGSTAPPAHGVTS | |
| | APDTRPAPGSTAPPAHGVTSAPDTRPAPGST | |
| | APPAHGVTSAPDTRPAPGSTAPPAHGVTSAP | |
| | DTRPAPGSTAPPAHGVTSAPDTRPAPGSTAP | |
| | PAHGVTSAPDTRPAPGSTAPPAHGVTSAPDT | |
| | RPAPGSTAPPAHGVTSAPDTRPAPGSTAPPA | |
| | HGVTSAPDTRPAPGSTAPPAHGVTSAPDTRP | |
| | APGSTAPPAHGVTSAPDTRPAPGSTAPPAHG | |
| | VTSAPDTRPAPGSTAPPAHGVTSAPDTRPAP | |
| | GSTAPPAHGVTSAPDTRPAPGSTAPPAHGVT | |
| | SAPDTRPAPGSTAPPAHGVTSAPDTRPAPGS | |
| | TAPPAHGVTSAPDTRPAPGSTAPPAHGVTSA | |
| | PDTRPAPGSTAPPAHGVTSAPDTRPAPGSTA | |
| | PPAHGVTSAPDTRPAPGSTAPPAHGVTSAPD | |
| | TRPAPGSTAPPAHGVTSAPDTRPAPGSTAPP | |
| | AHGVTSAPDTRPAPGSTAPPAHGVTSAPDTR | |
| | PAPGSTAPPAHGVTSAPDTRPAPGSTAPPAH | |
| | GVTSAPDTRPAPGSTAPPAHGVTSAPDTRPA | |
| | PGSTAPPAHGVTSAPDTRPAPGSTAPPAHGV | |
| | TSAPDTRPAPGSTAPPAHGVTSAPDTRPAPG | |
| | STAPPAHGVTSAPDNRPALGSTAPPVHNVTS | |
| | ASGSASGSASTLVHNGTSARATTTPASKSTP | |
| | FSIPSHHSDTPTTLASHSTKTDASSTHHSSVP | |
| | PLTSSNHSTSPQLSTGVSFFFLSFHISNLQFN | |
| | SSLEDPSTDYYQELQRDISEMFLQIYKQGGFL | |
| | GLSNIKFRPGSVVVQLTLAFREGTINVHDVET | |
| | QFNQYKTEAASRYNLTISDVSVSDVPFPFSA | |
| | QSGAGVPGWGIALLVLVCVLVALAIVYLIALAV | |
| | CQCRRKNYGQLDIFPARDTYHPMSEYPTYHT | |
| | HGRYVPPSSTDRSPYEKVSAGNGGSSLSYT | |
| | NPAVAATSANL | |
| SEQ ID NO: 20 | GSTAPPVHN | MUC-1 epitope |
| SEQ ID NO: 21 | TAPPAHGVTS | MUC-1 epitope |
| SEQ ID NO: 22 | RISTFKNWPF | survivin epitope |
| SEQ ID NO: 23 | APTLPPAWQPFLKDHRISTFKNWPFLEGSAV | survivin fragment |
| | KKQFEELTLGEFLKLDRER | |
| SEQ ID NO: 24 | MESPSAPPHRWCIPWQRLLLTASLLTFWNPP | CEA |
| | TTAKLTIESTPFNVAEGKEVLLLVHNLPQHLF | |
| | GYSWYKGERVDGNRQIIGYVIGTQQATPGPA | |
| | YSGREIIYPNASLLIQNIIQNDTGFYTLHVIKSD | |
| | LVNEEATGQFRVYPELPKPSISSNNSKPVEDK | |
| | DAVAFTCEPETQDATYLWWVNNQSLPVSPR | |
| | LQLSNGNRTLTLFNVTRNDTASYKCETQNPV | |
| | SARRSDSVILNVLYGPDAPTISPLNTSYRSGE | |
| | NLNLSCHAASNPPAQYSWFVNGTFQQSTQE | |
| | LFIPNITVNNSGSYTCQAHNSDTGLNRTTVTTI | |
| | TVYAEPPKPFITSNNSNPVEDEDAVALTCEPE | |
| | IQNTTYLWWVNNQSLPVSPRLQLSNDNRTLT | |
| | LLSVTRNDVGPYECGIQNKLSVDHSDPVILNV | |
| | LYGPDDPTISPSYTYYRPGVNLSLSCHAASNP | |
| | PAQYSWLIDGNIQQHTQELFISNITEKNSGLYT | |
| | CQANNSASGHSRTTVKTITVSAELPKPSISSN | |
| | NSKPVEDKDAVAFTCEPEAQNTTYLWWVNG | |
| | QSLPVSPRLQLSNGNRTLTLFNVTRNDARAY | |
| | VCGIQNSVSANRSDPVTLDVLYGPDTPIISPP | |
| | DSSYLSGANLNLSCHSASNPSPQYSWRINGI | |
| | PQQHTQVLFIAKITPNNNGTYACFVSNLATGR | |
| | NNSIVKSITVSASGTSPGLSAGATVGIMIGVLV | |
| | GVAL | |
| SEQ ID NO: 25 | NRTLTLFNVTRNDARAYVSGIQNSVSANRSD | CEA fragment |
| | PVTLDVLPDSSYLSGANLNLSCHSASPQYSW | |
| | RINGIPQQHTQVLFIAKITPNNNGTYACFVSNL | |
| | ATGRNNSIVKSITVSASGTSPGLSA | |
| SEQ ID NO: 26 | YLSGANLNLS | CEA epitope |
| SEQ ID NO: 27 | SWRINGIPQQ | CEA epitope |
| SEQ ID NO: 28 | MGRGLLRGLWPLHIVLWTRIASTIPPHVQKSV | TGFbR2 (UniProtKB |
| | NNDMIVTDNNGAVKFPQLCKFCDVRFSTCDN | P37137) |
| | QKSCMSNCSITSICEKPQEVCVAVWRKNDEN | |
| | ITLETVCHDPKLPYHDFILEDAASPKCIMKEKK | |
| | KPGETFFMCSCSSDECNDNIIFSEEYNTSNPD | |
| | LLLVIFQVTGISLLPPLGVAISVIIIFYCYRVNRQ | |
| | QKLSSTWETGKTRKLMEFSEHCAIILEDDRSD | |
| | ISSTCANNINHNTELLPIELDTLVGKGRFAEVY | |
| | KAKLKQNTSEQFETVAVKIFPYEEYASWKTEK | |
| | DIFSDINLKHENILQFLTAEERKTELGKQYWLI | |
| | TAFHAKGNLQEYLTRHVISWEDLRKLGSSLA | |
| | RGIAHLHSDHTPCGRPKMPIVHRDLKSSNILV | |
| | KNDLTCCLCDFGLSLRLDPTLSVDDLANSGQ | |
| | VGTARYMAPEVLESRMNLENVESFKQTDVYS | |
| | MALVLWEMTSRCNAVGEVKDYEPPFGSKVR | |
| | EHPCVESMKDNVLRDRGRPEIPSFWLNHQGI | |
| | QMVCETLTECWDHDPEARLTAQCVAERFSE | |
| | LEHLDRLSGRSCSEEKIPEDGSLNTTK | |
| SEQ ID NO: 29 | MEEPQSDPSVEPPLSQETFSDLWKLLPENNV | P53 |
| | LSPLPSQAMDDLMLSPDDIEQWFTEDPGPDE | |
| | APRMPEAAPPVAPAPAAPTPAAPAPAPSWPL | |
| | SSSVPSQKTYQGSYGFRLGFLHSGTAKSVTC | |
| | TYSPALNKMFCQLAKTCPVQLWVDSTPPPGT | |
| | RVRAMAIYKQSQHMTEVVRRCPHHERCSDS | |
| | DGLAPPQHLIRVEGNLRVEYLDDRNTFRHSV | |
| | VVPYEPPEVGSDCTTIHYNYMCNSSCMGGM | |
| | NRRPILTIITLEDSSGNLLGRNSFEVRVCACPG | |
| | RDRRTEEENLRKKGEPHHELPPGSTKRALPN | |
| | NTSSSPQPKKKPLDGEYFTLQIRGRERFEMF | |
| | RELNEALELKDAQAGKEPGGSRAHSSHLKSK | |
| | KGQSTSRHKKLMFKTEGPDSD | |
| SEQ ID NO: 30 | MTEYKLVVVGAGGVGKSALTIQLIQNHFVDEY | Kirsten Ras |
| | DPTIEDSYRKQVVIDGETCLLDILDTAGQEEY | |
| | SAMRDQYMRTGEGFLCVFAINNTKSFEDIHH | |
| | YREQIKRVKDSEDVPMVLVGNKCDLPSRTVD | |
| | TKQAQDLARSYGIPFIETSAKTRQRVEDAFYT | |
| | LVREIRQYRLKKISKEEKTPGCVKIKKCIIM | |
| SEQ ID NO: 31 | VVVGAGGVG | Kirsten Ras epitope |
| SEQ ID NO: 32 | MASSVGNVADSTEPTKRMLSFQGLAELAHRE | OGT |
| | YQAGDFEAAERHCMQLWRQEPDNTGVLLLL | |
| | SSIHFQCRRLDRSAHFSTLAIKQNPLLAEAYS | |
| | NLGNVYKERGQLQEAIEHYRHALRLKPDFIDG | |
| | YINLAAALVAAGDMEGAVQAYVSALQYNPDL | |
| | YCVRSDLGNLLKALGRLEEAKACYLKAIETQP | |
| | NFAVAWSNLGCVFNAQGEIWLAIHHFEKAVT | |
| | LDPNFLDAYINLGNVLKEARIFDRAVAAYLRAL | |
| | SLSPNHAVVHGNLACVYYEQGLIDLAIDTYRR | |
| | AIELQPHFPDAYCNLANALKEKGSVAEAEDC | |
| | YNTALRLCPTHADSLNNLANIKREQGNIEEAV | |
| | RLYRKALEVFPEFAAAHSNLASVLQQQGKLQ | |
| | EALMHYKEAIRISPTFADAYSNMGNTLKEMQ | |
| | DVQGALQCYTRAIQINPAFADAHSNLASIHKD | |
| | SGNIPEAIASYRTALKLKPDFPDAYCNLAHCL | |
| | QIVCDWTDYDERMKKLVSIVADQLEKNRLPS | |
| | VHPHHSMLYPLSHGFRKAIAERHGNLCLDKIN | |
| | VLHKPPYEHPKDLKLSDGRLRVGYVSSDFGN | |
| | HPTSHLMQSIPGMHNPDKFEVFCYALSPDDG | |
| | TNFRVKVMAEANHFIDLSQIPCNGKAADRIHQ | |
| | DCIHILVNMNGYTKGARNELFALRPAPIQAM | |
| | WLGYPGTSGALFMDYIITDQETSPAEVAEQY | |
| | SEKLAYMPHTFFIGDHANMFPHLKKKAVIDFK | |
| | SNGHIYDNRIVLNGIDLKAFLDSLPDVKIVKMK | |
| | CPDGGDNADSSNTALNMPVIPMNTIAEAVIEM | |
| | INRGQIQITINGFSISNGLATTQINNKAATGEEV | |
| | PRTIIVTTRSQYGLPEDAIVYCNFNQLYKIDPS | |
| | TLQMWANILKRVPNSVLWLLRFPAVGEPNIQ | |
| | QYAQNMGLPQNRIIFSPVAPKEEHVRRGQLA | |
| | DVCLDTPLCNGHTTGMDVLWAGTPMVTMPG | |
| | ETLASRVAASQLTCLGCLELIAKNRQEYEDIA | |
| | VKLGTDLEYLKKVRGKVWKQRISSPLFNTKQ | |
| | YTMELERLYLQMWEHYAAGNKPDHMIKPVE | |
| | VTESA | |
| SEQ ID NO: 33 | MAEDSGKKKRRKNFEAMFKGILQSGLDNFVI | CASP5 |
| | NHMLKNNVAGQTSIQTLVPNTDQKSTSVKKD | |
| | NHKKKTVKMLEYLGKDVLHGVFNYLAKHDVL | |
| | TLKEEEKKKYYDTKIEDKALILVDSLRKNRVAH | |
| | QMFTQTLLNMDQKITSVKPLLQIEAGPPESAE | |
| | STNILKLCPREEFLRLCKKNHDEIYPIKKREDR | |
| | RRLALIICNTKFDHLPARNGAHYDIVGMKRLL | |
| | QGLGYTVVDEKNLTARDMESVLRAFAARPEH | |
| | KSSDSTFLVLMSHGILEGICGTAHKKKKPDVL | |
| | LYDTIFQIFNNRNCLSLKDKPKVIIVQACRGEK | |
| | HGELWVRDSPASLALISSQSSENLEADSVCKI | |
| | HEEKDFIAFCSSTPHNVSWRDRTRGSIFITELI | |
| | TCFQKYSCCCHLMEIFRKVQKSFEVPQAKAQ | |
| | MPTIERATLTRDFYLFPGN | |
| SEQ ID NO: 34 | MSSPLASLSKTRKVPLPSEPMNPGRRGIRIYG | COA-1 |
| | DEDEVDMLSDGCGSEEKISVPSCYGGIGAPV | |
| | SRQVPASHDSELMAFMTRKLWDLEQQVKAQ | |
| | TDEILSKDQKIAALEDLVQTLRPHPAEATLQR | |
| | QEELETMCVQLQRQVREMERFLSDYGLQWV | |
| | GEPMDQEDSESKTVSEHGERDWMTAKKFW | |
| | KPGDSLAPPEVDFDRLLASLQDLSELVVEGD | |
| | TQVTPVPGGARLRTLEPIPLKLYRNGIMMFDG | |
| | PFQPFYDPSTQRCLRDILDGFFPSELQRLYPN | |
| | GVPFKVSDLRNQVYLEDGLDPFPGEGRVVG | |
| | RQLMHKALDRVEEHPGSRMTAEKFLNRZPKF | |
| | VIRQGEVIDIRGPIRDTLQNCCPLPARIQEIVVE | |
| | TPTLAAERERSQESPNTPAPPLSMLRIKSENG | |
| | EQAFLLMMQPDNTIGDVRALLAQARVMDASA | |
| | FEIFSTFPPTLYQDDTLTLQAAGLVPKAALLLR | |
| | ARRAPKSSLKFSPGPCPGPGPGPSPGPGPG | |
| | PSPGPGPGPSPCPGPSPSPQ | |
| SEQ ID NO: 35 | MAFVCLAIGCLYTFLISTTFGCTSSSDTEIKVN | IL13Ralpha2 |
| | PPQDFEIVDPGYLGYLYLQWQPPLSLDHFKE | |
| | CTVEYELKYRNIGSETWKTIITKNLHYKDGFDL | |
| | NKGIEAKIHTLLPWQCTNGSEVQSSWAETTY | |
| | WISPQGIPETKVQDMDCVYYNWQYLLCSWK | |
| | PGIGVLLDTNYNLFYWYEGLDHALQCVDYIKA | |
| | DGQNIGCRFPYLEASDYKDFYICVNGSSENK | |
| | PIRSSYFTFQLQNIVKPLPPVYLTFTRESSCEI | |
| | KLKWSIPLGPIPARCFDYEIEIREDDTTLVTAT | |
| | VENETYTLKTTNETRQLCFVVRSKVNIYCSDD | |
| | GIWSEWSDKQCWEGEDLSKKTLLRFWLPFG | |
| | FILILVIFVTGLLLRKPNTYPKMIPEFFCDT | |
| SEQ ID NO: 36 | LPFGFIL | IL13Ralpha2 epitope |
| SEQ ID NO: 37 | MNKLYIGNLSENAAPSDLESIFKDAKIPVSGPF | KOC-1 |
| | LVKTGYAFVDCPDESWALKAIEALSGKIELHG | |
| | KPIEVEHSVPKRQRIRKLQIRNIPPHLQWEVL | |
| | DSLLVQYGVVESCEQVNTDSETAVVNVTYSS | |
| | KDQARQALDKLNGFQLENFTLKVAYIPDEMA | |
| | AQQNPLQQPRGRRGLGQRGSSRQGSPGSV | |
| | SKQKPCDLPLRLLVPTQFVGAIIGKEGATIRNI | |
| | TKQTQSKIDVHRKENAGAAEKSITILSTPEGTS | |
| | AACKSILEIMHKEAQDIKFTEEIPLKILAHNNFV | |
| | GRLIGKEGRNLKKIEQDTDTKITISPLQELTLY | |
| | NPERTITVKGNVETCAKAEEEIMKKIRESYEN | |
| | DIASMNLQAHLIPGLNLNALGLFPPTSGMPPP | |
| | TSGPPSAMTPPYPQFEQSETETVHLFIPALSV | |
| | GAIIGKQGQHIKQLSRFAGASIKIAPAEAPDAK | |
| | VRMVIITGPPEAQFKAQGRIYGKIKEENFVSP | |
| | KEEVKLEAHIRVPSFAAGRVIGKGGKTVNELQ | |
| | NLSSAEVVVPRDQTPDENDQVVVKITGHFYA | |
| | CQVAQRKIQEILTQVKQHQQQKALQSGPPQS | |
| | RRK | |
| SEQ ID NO: 38 | MAPKFPDSVEELRAAGNESFRNGQYAEASAL | TOMM34 |
| | YGRALRVLQAQGSSDPEEESVLYSNRAACHL | |
| | KDGNCRDCIKDCTSALALVPFSIKPLLRRASA | |
| | YEALEKYPMAYVDYKTVLQIDDNVTSAVEGIN | |
| | RMTRALMDSLGPEWRLKLPSIPLVPVSAQKR | |
| | WNSLPSENHKEMAKSKSKETTATKNRVPSA | |
| | GDVEKARVLKEEGNELVKKGNHKKAIEKYSE | |
| | SLLCSNLESATYSNRALCYLVLKQYTEAVKDC | |
| | TEALKLDGKNVKAFYRRAQAHKALKDYKSSF | |
| | ADISNLLQIEPRNGPAQKLRQEVKQNLH | |
| SEQ ID NO: 39 | MSGGHQLQLAALWPWLLMATLQAGFGRTGL | RNF43 |
| | VLAAAVESERSAEQKAIIRVIPLKMDPTGKLNL | |
| | TLEGVFAGVAEITPAEGKLMQSHPLYLCNAS | |
| | DDDNLEPGFISIVKLESPRRAPRPCLSLASKA | |
| | RMAGERGASAVLFDITEDRAAAEQLQQPLGL | |
| | TWPVVLIWGNDAEKLMEFVYKNQKAHVRIEL | |
| | KEPPAWPDYDVWILMTVVGTIFVIILASVLRIR | |
| | CRPRHSRPDPLQQRTAWAISQLATRRYQAS | |
| | CRQARGEWPDSGSSCSSAPVCAICLEEFSE | |
| | GQELRVISCLHEFHRNCVDPWLHQHRTCPLC | |
| | MFNITEGDSFSQSLGPSRSYQEPGRRLHLIR | |
| | QHPGHAHYHLPAAYLLGPSRSAVARPPRPG | |
| | PFLPSQEPGMGPRHHRFPRAAHPRAPGEQQ | |
| | RLAGAQHPYAQGWGLSHLQSTSQHPAACPV | |
| | PLRRARPPDSSGSGESYCTERSGYLADGPA | |
| | SDSSSGPCHGSSSDSVVNCTDISLQGVHGSS | |
| | STFCSSLSSDFDPLVYCSPKGDPQRVDMQP | |
| | SVTSRPRSLDSVVPTGETQVSSHVHYHRHR | |
| | HHHYKKRFQWHGRKPGPETGVPQSRPPIPR | |
| | TQPQPEPPSPDQQVTRSNSAAPSGRLSNPQ | |
| | CPRALPEPAPGPVDASSICPSTSSLFNLQKSS | |
| | LSARHPQRKRRGGPSEPTPGSRPQDATVHP | |
| | ACQIFPHYTPSVAYPWSPEAHPLICGPPGLDK | |
| | RLLPETPGPCYSNSQPVWLCLTPRQPLEPHP | |
| | PGEGPSEWSSDTAEGRPCPYPHCQVLSAQP | |
| | GSEEELEELCEQAV | |
| SEQ ID NO: 40 | MAPPQVLAFGLLLAAATATFAAAQEECVCEN | EpCAM |
| | YKLAVNCFVNNNRQCQCTSVGAQNTVICSKL | |
| | AAKCLVMKAEMNGSKLGRRAKPEGALQNND | |
| | GLYDPDCDESGLFKAKQCNGTSMCWCVNTA | |
| | GVRRTDKDTEITCSERVRTYWIIIELKHKAREK | |
| | PYDSKSLRTALQKEITTRYQLDPKFITSILYEN | |
| | NVITIDLVQNSSQKTQNDVDIADVAYYFEKDV | |
| | KGESLFHSKKMDLTVNGEQLDLDPGQTLIYY | |
| | VDEKAPEFSMQGLKAGVIAVIVVVVIAVVAGIV | |
| | VLVISRKKRMAKYEKAEIKEMGEMHRELNA | |
| SEQ ID NO: 41 | GLKAGVIAV | EpCAM epitope |
| SEQ ID NO: 42 | MELAALCRWGLLLALLPPGAASTQVCTGTDM | HER-2/neu |
| | KLRLPASPETHLDMLRHLYQGCQVVQGNLEL | |
| | TYLPTNASLSFLQDIQEVQGYVLIAHNQVRQV | |
| | PLQRLRIVRGTQLFEDNYALAVLDNGDPLNNT | |
| | TPVTGASPGGLRELQLRSLTEILKGGVLIQRN | |
| | PQLCYQDTILWKDIFHKNNQLALTLIDTNRSR | |
| | ACHPCSPMCKGSRCWGESSEDCQSLTRTVC | |
| | AGGCARCKGPLPTDCCHEQCAAGCTGPKHS | |
| | DCLACLHFNHSGICELHCPALVTYNTDTFESM | |
| | PNPEGRYTFGASCVTACPYNYLSTDVGSCTL | |
| | VCPLHNQEVTAEDGTQRCEKCSKPCARVCY | |
| | GLGMEHLREVRAVTSANIQEFAGCKKIFGSLA | |
| | FLPESFDGDPASNTAPLQPEQLQVFETLEEIT | |
| | GYLYISAWPDSLPDLSVFQNLQVIRGRILHNG | |
| | AYSLTLQGLGISWLGLRSLRELGSGLALIHHN | |
| | THLCFVHTVPWDQLFRNPHQALLHTANRPED | |
| | ECVGEGLACHQLCARGHCWGPGPTQCVNC | |
| | SQFLRGQECVEECRVLQGLPREYVNARHCL | |
| | PCHPECQPQNGSVTCFGPEADQCVACAHYK | |
| | DPPFCVARCPSGVKPDLSYMPIWKFPDEEGA | |
| | CQPCPINCTHSCVDLDDKGCPAEQRASPLTS | |
| | IISAVVGILLVVVLGVVFGILIKRRQQKIRKYTM | |
| | RRLLQETELVEPLTPSGAMPNQAQMRILKET | |
| | ELRKVKVLGSGAFGTVYKGIWIPDGENVKIPV | |
| | AIKVLRENTSPKANKEILDEAYVMAGVGSPYV | |
| | SRLLGICLTSTVQLVTQLMPYGCLLDHVRENR | |
| | GRLGSQDLLNWCMQIAKGMSYLEDVRLVHR | |
| | DLAARNVLVKSPNHVKITDFGLARLLDIDETEY | |
| | HADGGKVPIKWMALESILRRRFTHQSDVWSY | |
| | GVTVWELMTFGAKPYDGIPAREIPDLLEKGE | |
| | RLPQPPICTIDVYMIMVKCWMIDSECRPRFRE | |
| | LVSEFSRMARDPQRFVVIQNEDLGPASPLDS | |
| | TFYRSLLEDDDMGDLVDAEEYLVPQQGFFCP | |
| | DPAPGAGGMVHHRHRSSSTRSGGGDLTLGL | |
| | EPSEEEAPRSPLAPSEGAGSDVFDGDLGMG | |
| | AAKGLQSLPTHDPSPLQRYSEDPTVPLPSET | |
| | DGYVAPLTCSPQPEYVNQPDVRPQPPSPRE | |
| | GPLPAARPAGATLERPKTLSPGKNGVVKDVF | |
| | AFGGAVENPEYLTPQGGAAPQPHPPPAFSP | |
| | AFDNLYYWDQDPPERGAPPSTFKGTPTAEN | |
| | PEYLGLDVPV | |
| SEQ ID NO: 43 | MGSDVRDLNALLPAVPSLGGGGGCALPVSG | WT1 |
| | AAQWAPVLDFAPPGASAYGSLGGPAPPPAP | |
| | PPPPPPPPHSFIKQEPSWGGAEPHEEQCLSA | |
| | FTVHFSGQFTGTAGACRYGPFGPPPPSQAS | |
| | SGQARMFPNAPYLPSCLESQPAIRNQGYSTV | |
| | TFDGTPSYGHTPSHHAAQFPNHSFKHEDPM | |
| | GQQGSLGEQQYSVPPPVYGCHTPTDSCTGS | |
| | QALLLRTPYSSDNLYQMTSQLECMTWNQMN | |
| | LGATLKGVAAGSSSSVKWTEGQSNHSTGYE | |
| | SDNHTTPILCGAQYRIHTHGVFRGIQDVRRVP | |
| | GVAPTLVRSASETSEKRPFMCAYPGCNKRYF | |
| | KLSHLQMHSRKHTGEKPYQCDFKDCERRFS | |
| | RSDQLKRHQRRHTGVKPFQCKTCQRKFSRS | |
| | DHLKTHTRTHTGKTSEKPFSCRWPSCQKKFA | |
| | RSDELVRHHNMHQRNMTKLQLAL | |
| SEQ ID NO: 44 | KVAELVHFL | MAGE A3 epitope |
| SEQ ID NO: 45 | NRTLTLFNVTRNDARAYVSGIQNSVSANRSD | antigenic cargo of |
| | PVTLDVLPDSSYLSGANLNLSCHSASPQYSW | ATP128 |
| | RINGIPQQHTQVLFIAKITPNNNGTYACFVSNL | |
| | ATGRNNSIVKSITVSASGTSPGLSAAPTLPPA | |
| | WQPFLKDHRISTFKNWPFLEGSAVKKQFEEL | |
| | TLGEFLKLDRERAAVARRNERERNRVKLVNL | |
| | GFQALRQHVPHGGASKKLSKVETLRSAVEYI | |
| | RALQRLLAEHDAVRNALAGGLRPQAVRPSAP | |
| | RGPSEGALSPAERELLDFSSWLGGY | |
| SEQ ID NO: 46 | MGQIVTMFEALPHIIDEVINIVIIVLIIITSIKAVYN | GP of LCMV |
| | FATCGILALVSFLFLAGRSCGMYGLNGPDIYK | |
| | GVYQFKSVEFDMSHLNLTMPNACSANNSHH | |
| | YISMGSSGLELTFTNDSILNHNFCNLTSAFNK | |
| | KTFDHTLMSIVSSLHLSIRGNSNHKAVSCDFN | |
| | NGITIQYNLSFSDPQSAISQCRTFRGRVLDMF | |
| | RTAFGGKYMRSGWGWAGSDGKTTWCSQTS | |
| | YQYLIIQNRTWENHCRYAGPFGMSRILFAQE | |
| | KTKFLTRRLAGTFTWTLSDSSGVENPGGYCL | |
| | TKWMILAAELKCFGNTAVAKCNVNHDEEFCD | |
| | MLRLIDYNKAALSKFKQDVESALHVFKTTVNS | |
| | LISDQLLMRNHLRDLMGVPYCNYSKFWYLEH | |
| | AKTGETSVPKCWLVTNGSYLNETHFSDQIEQ | |
| | EADNMITEMLRKDYIKRQGSTPLALMDLLMFS | |
| | TSAYLISIFLHLVKIPTHRHIKGGSCPKPHRLTN | |
| | KGICSCGAFKVPGVKTIWKRR | |
| SEQ ID NO: 47 | MGQLITMFEALPHIIDEVINIVIIVLVIITSIKAVYN | glycoprotein GP of |
| | FATCGIIALISFCLLAGRSCGLYGVTGPDIYKG | the DNADV |
| | LYQFKSVEFNMSQLNLTMPNACSANNSHHYI | |
| | SMGKSGLELTFTNDSIISHNFCNLTDGFKKKT | |
| | FDHTLMSIVASLHLSIRGNTNYKAVSCDFNNG | |
| | ITIQYNLSFSDAQSAINQCRTFRGRVLDMFRT | |
| | AFGGKYMRSGYGWKGSDGKTTWCSQTSYQ | |
| | YLIIQNRTWENHCEYAGPFGLSRVLFAQEKTK | |
| | FLTRRLAGTFTWTLSDSSGTENPGGYCLTKW | |
| | MLIAAELKCFGNTAVAKCNINHDEEFCDMLRL | |
| | IDYNKAALKKFKEDVESALHLFKTTVNSLISDQ | |
| | LLMRNHLRDLMGVPYCNYSKFWYLEHVKTG | |
| | DTSVPKCWLVSNGSYLNETHFSDQIEQEADN | |
| | MITEMLRKDYIKRQGSTPLALMDLLMFSTSAY | |
| | LISVFLHLMKIPTHRHIKGGTCPKPHRLTSKGI | |
| | CSCGAFKVPGVKTVWKRR | |
| SEQ ID NO: 48 | MGQIVTFFQEVPHILEEVMNIVLMTLSILAILKG | Glycoprotein GP of |
| | IYNVMTCGIIGLITFLFLCGRSCSSIYKDNYEFF | MOPV |
| | SLDLDMSSLNATMPLSCSKNNSHHYIQVGNE | |
| | TGLELTLTNTSIIDHKFCNLSDAHRRNLYDKAL | |
| | MSILTTFHLSIPDFNQYEAMSCDFNGGKISIQY | |
| | NLSHSNYVDAGNHCGTIANGIMDVFRRMYW | |
| | STSLSVASDISGTQCIQTDYKYLIIQNTSWEDH | |
| | CMFSRPSPMGFLSLLSQRTRNFYISRRLLGLF | |
| | TWTLSDSEGNDMPGGYCLTRSMLIGLDLKCF | |
| | GNTAIAKCNQAHDEEFCDMLRLFDFNKQAIS | |
| | KLRSEVQQSINLINKAVNALINDQLVMRNHLR | |
| | DLMGIPYCNYSKFWYLNDTRTGRTSLPKCWL | |
| | VTNGSYLNETQFSTEIEQEANNMFTDMLRKE | |
| | YEKRQSTTPLGLVDLFVFSTSFYLISVFLHLIKI | |
| | PTHRHIKGKPCPKPHRLNHMAICSCGFYKQP | |
| | GLPTQWKR | |
| SEQ ID NO: 49 | MSVTVKRIIDNTVVVPKLPANEDPVEYPADYF | vesicular stomatitis |
| | RKSKEIPLYINTTKSLSDLRGYVYQGLKSGNV | virus nucleoprotein |
| | SIIHVNSYLYGALKDIRGKLDKDWSSFGINIGK | (N) |
| | AGDTIGIFDLVSLKALDGVLPDGVSDASRTSA | |
| | DDKWLPLYLLGLYRVGRTQMPEYRKKLMDG | |
| | LTNQCKMINEQFEPLVPEGRDIFDVWGNDSN | |
| | YTKIVAAVDMFFHMFKKHECASFRYGTIVSRF | |
| | KDCAALATFGHLCKITGMSTEDVTTWILNREV | |
| | ADEMVQMMLPGQEIDKADSYMPYLIDFGLSS | |
| | KSPYSSVKNPAFHFWGQLTALLLRSTRARNA | |
| | RQPDDIEYTSLTTAGLLYAYAVGSSADLAQQF | |
| | CVGDNKYTPDDSTGGLTTNAPPQGRDVVEW | |
| | LGWFEDQNRKPTPDMMQYAKRAVMSLQGL | |
| | REKTIGKYAKSEFDK | |
| SEQ ID NO: 50 | MDNLTKVREYLKSYSRLDQAVGEIDEIEAQRA | phosphoprotein (P) |
| | EKSNYELFQEDGVEEHTKPSYFQAADDSDTE | of VSV |
| | SEPEIEDNQGLYAPDPEAEQVEGFIQGPLDD | |
| | YADEEVDVVFTSDWKQPELESDEHGKTLRLT | |
| | SPEGLSGEQKSQWLSTIKAVVQSAKYWNLAE | |
| | CTFEASGEGVIMKERQITPDVYKV | |
| | TPVMNTHPSQSEAVSDVWSLSKTSMTFQPK | |
| | KASLQPLTISLDELFSSRGEFISVGGDGRM | |
| | SHKEAILLGLRYKKLYNQARVKYSL | |
| SEQ ID NO: 51 | MEVHDFETDEFNDFNEDDYATREFLNPDER | Large protein (L) of |
| | MTYLNHADYNLNSPLISDDIDNLIRKFNSLPIP | VSV |
| | SMWDSKNWDGVLEMLTSCQANPIPTSQMHK | |
| | WMGSWLMSDNHDASQGYSFLHEVDKEAEIT | |
| | FDVVETFIRGWGNKPIEYIKKERWTDSFKILAY | |
| | LCQKFLDLHKLTLILNAVSEVELLNLARTFKGK | |
| | VRRSSHGTNICRIRVPSLGPTFISEGWAYFKK | |
| | LDILMDRNFLLMVKDVIIGRMQTVLSMVCRID | |
| | NLFSEQDIFSLLNIYRIGDKIVERQGNFSYDLIK | |
| | MVEPICNLKLMKLARESRPLVPQFPHFENHIK | |
| | TSVDEGAKIDRGIRFLHDQIMSVKTVDLTLVIY | |
| | GSFRHWGHPFIDYYTGLEKLHSQVTMKKDID | |
| | VSYAKALASDLARIVLFQQFNDHKKWFVNGD | |
| | LLPHDHPFKSHVKENTWPTAAQVQDFGDKW | |
| | HELPLIKCFEIPDLLDPSIIYSDKSHSMNRSEVL | |
| | KHVRMNPNTPIPSKKVLQTMLDTKATNWKEF | |
| | LKEIDEKGLDDDDLIIGLKGKERELKLAGRFFS | |
| | LMSWKLREYFVITEYLIKTHFVPMFKGLTMAD | |
| | DLTAVIKKMLDSSSGQGLKSYEAICIANHIDYE | |
| | KWNNHQRKLSNGPVFRVMGQFLGYPSLIER | |
| | THEFFEKSLIYYNGRPDLMRVHNNTLINSTSQ | |
| | RVCWQGQEGGLEGLRQKGWSILNLLVIQRE | |
| | AKIRNTAVKVLAQGDNQVICTQYKTKKSRNVV | |
| | ELQGALNQMVSNNEKIMTAIKIGTGKLGLLIND | |
| | DETMQSADYLNYGKIPIFRGVIRGLETKRWSR | |
| | VTCVTNDQIPTCANIMSSVSTNALTVAHFAEN | |
| | PINAMIQYNYFGTFARLLLMMHDPALRQSLYE | |
| | VQDKIPGLHSSTFKYAMLYLDPSIGGVSGMSL | |
| | SRFLIRAFPDPVTESLSFWRFIHVHARSEHLK | |
| | EMSAVFGNPEIAKFRITHIDKLVEDPTSLNIAM | |
| | GMSPANLLKTEVKKCLIESRQTIRNQVIKDATI | |
| | YLYHEEDRLRSFLWSINPLFPRFLSEFKSGTF | |
| | LGVADGLISLFQNSRTIRNSFKKKYHRELDDLI | |
| | VRSEVSSLTHLGKLHLRRGSCKMWTCSATH | |
| | ADTLRYKSWGRTVIGTTVPHPLEMLGPQHRK | |
| | ETPCAPCNTSGFNYVSVHCPDGIHDVFSSRG | |
| | PLPAYLGSKTSESTSILQPWERESKVPLIKRA | |
| | TRLRDAISWFVEPDSKLAMTILSNIHSLTGEE | |
| | WTKRQHGFKRTGSALHRFSTSRMSHGGFAS | |
| | QSTAALTRLMATTDTMRDLGDQNFDFLFQAT | |
| | LLYAQITTTVARDGWITSCTDHYHIACKSCLR | |
| | PIEEITLDSSMDYTPPDVSHVLKTWRNGEGS | |
| | WGQEIKQIYPLEGNWKNLAPAEQSYQVGRCI | |
| | GFLYGDLAYRKSTHAEDSSLFPLSIQGRIRGR | |
| | GFLKGLLDGLMRASCCQVIHRRSLAHLKRPA | |
| | NAVYGGLIYLIDKLSVSPPFLSLTRSGPIRDEL | |
| | ETIPHKIPTSYPTSNRDMGVIVRNYFKYQCRLI | |
| | EKGKYRSHYSQLWLFSDVLSIDFIGPFSISTTL | |
| | LQILYKPFLSGKDKNELRELANLSSLLRSGEG | |
| | WEDIHVKFFTKDILLCPEEIRHACKFGIAKDNN | |
| | KDMSYPPWGRESRGTITTIPVYYTTTPYPKML | |
| | EMPPRIQNPLLSGIRLGQLPTGAHYKIRSILHG | |
| | MGIHYRDFLSCGDGSGGMTAALLRENVHSR | |
| | GIFNSLLELSGSVMRGASPEPPSALETLGGD | |
| | KSRCVNGETCWEYPSDLCDPRTWDYFLRLK | |
| | AGLGLQIDLIVMDMEVRDSSTSLKIETNVRNY | |
| | VHRILDEQGVLIYKTYGTYICESEKNAVTILGP | |
| | MFKTVDLVQTEFSSSQTSEVYMVCKGLKKLI | |
| | DEPNPDWSSINESWKNLYAFQSSEQEFARA | |
| | KKVSTYFTLTGIPSQFIPDPFVNIETMLQIFGV | |
| | PTGVSHAAALKSSDRPADLLTISLFYMAIISYY | |
| | NINHIRVGPIPPNPPSDGIAQNVGIAITGISFWL | |
| | SLMEKDIPLYQQCLAVIQQSFPIRWEAVSVKG | |
| | GYKQKWSTRGDGLPKDTRISDSLAPIGNWIR | |
| | SLELVRNQVRLNPFNEILFNQLCRTVDNHLK | |
| | WSNLRRNTGMIEWINRRISKEDRSILMLKSDL | |
| | HEENSWRD | |
| SEQ ID NO: 52 | MSSLKKILGLKGKGKKSKKLGIAPPPYEEDTS | Matrix protein (M) of |
| | MEYAPSAPIDKSYFGVDEMDTYDPNQLRYEK | VSV |
| | FFFTVKMTVRSNRPFRTYSDVAAAVSHWDH | |
| | MYIGMAGKRPFYKILAFLGSSNLKATPAVLAD | |
| | QGQPEYHAHCEGRAYLPHRMGKTPPMLNVP | |
| | EHFRRPFNIGLYKGTIELTMTIYDDESLEAAPM | |
| | IWDHFNSSKFSDFREKALMFGLIVEKKASGA | |
| | VVVLDSIGHFK | |
| SEQ ID NO: 53 | MGQIVTMFEALPHIIDEVINIVIIVLIIITSIKAVYN | glycoprotein GP of |
| | FATCGILALVSFLFLAGRSCGMYGLNGPDIYK | the LCMV |
| | GVYQFKSVEFDMSHLNLTMPNACSANNSHH | |
| | YISMGSSGLELTFTNDSILNHNFCNLTSAFNK | |
| | KTFDHTLMSIVSSLHLSIRGNSNHKAVSCDFN | |
| | NGITIQYNLSFSDPQSAISQCRTFRGRVLDMF | |
| | RTAFGGKYMRSGWGWAGSDGKTTWCSQTS | |
| | YQYLIIQNRTWENHCRYAGPFGMSRILFAQE | |
| | KTKFLTRRLAGTFTWTLSDSSGVENPGGYCL | |
| | TKWMILAAELKCFGNTAVAKCNVNHDEEFCD | |
| | MLRLIDYNKAALSKFKQDVESALHVFKTTVNS | |
| | LISDQLLMRNHLRDLMGVPYCNYSKFWYLEH | |
| | AKTGETSVPKCWLVTNGSYLNETHFSDQIEQ | |
| | EADNMITEMLRKDYIKRQGSTPLALMDLLMFS | |
| | TSAYLISIFLHLVKIPTHRHIKGGSCPKPHRLTN | |
| | KGICSCGAFKVPGVKTIWKRR | |
| SEQ ID NO: 54 | MDNLTKVREYLKSYSRLDQAVGEIDEIEAQRA | phosphoprotein (P) |
| | EKSNYELFQEDGVEEHTKPSYFQAADDSDTE | of rVSV |
| | SEPEIEDNQGLYAPDPEAEQVEGFIQGPLDD | |
| | YADEEVDWFTSDWKQPELESDEHGKTLRLT | |
| | SPEGLSGEQKSQWLSTIKAVVQSAKYWNLAE | |
| | CTFEASGEGVIMKERQITPDVYKVTPVMNTH | |
| | PSQSEAVSDVWSLSKTSMTFQPKKASLQPLT | |
| | ISLDELFSSRGEFISVGGDGRMSHKEAILLGL | |
| | RYKKLYNQARVKYSL | |
| SEQ ID NO: 55 | MSVTVKRIIDNTVVVPKLPANEDPVEYPADYF | nucleoprotein (N) of |
| | RKSKEIPLYINTTKSLSDLRGYVYQGLKSGNV | rVSV |
| | SIIHVNSYLYGALKDIRGKLDKDWSSFGINIGK | |
| | AGDTIGIFDLVSLKALDGVLPDGVSDASRTSA | |
| | DDKWLPLYLLGLYRVGRTQMPEYRKKLMDG | |
| | LTNQCKMINEQFEPLVPEGRDIFDVWGNDSN | |
| | YTKIVAAVDMFFHMFKKHECASFRYGTIVSRF | |
| | KDCAALATFGHLCKITGMSTEDVTTWILNREV | |
| | ADEMVQMMLPGQEIDKADSYMPYLIDFGLSS | |
| | KSPYSSVKNPAFHFWGQLTALLLRSTRARNA | |
| | RQPDDIEYTSLTTAGLLYAYAVGSSADLAQQF | |
| | CVGDNKYTPDDSTGGLTTNAPPQGRDVVEW | |
| | LGWFEDQNRKPTPDMMQYAKRAVMSLQGL | |
| | REKTIGKYAKSEFDK | |
| SEQ ID NO: 56 | MSSLKKILGLKGKGKKSKKLGIAPPPYEEDTS | matrix protein (M) of |
| | MEYAPSAPIDKSYFGVDEMDTYDPNQLRYEK | rVSV |
| | FFFTVKMTVRSNRPFRTYSDVAAAVSHWDH | |
| | MYIGMAGKRPFYKILAFLGSSNLKATPAVLAD | |
| | QGQPEYHAHCEGRAYLPHRMGKTPPMLNVP | |
| | EHFRRPFNIGLYKGTIELTMTIYDDESLEAAPM | |
| | IWDHFNSSKFSDFREKALMFGLIVEKKASGA | |
| | WVLDSIGHFK | |
| SEQ ID NO: 57 | MEVHDFETDEFNDFNEDDYATREFLNPDER | large protein (L) of |
| | MTYLNHADYNLNSPLISDDIDNLIRKFNSLPIP | rVSV |
| | SMWDSKNWDGVLEMLTSCQANPIPTSQMHK | |
| | WMGSWLMSDNHDASQGYSFLHEVDKEAEIT | |
| | FDVVETFIRGWGNKPIEYIKKERWTDSFKILAY | |
| | LCQKFLDLHKLTLILNAVSEVELLNLARTFKGK | |
| | VRRSSHGTNICRIRVPSLGPTFISEGWAYFKK | |
| | LDILMDRNFLLMVKDVIIGRMQTVLSMVCRID | |
| | NLFSEQDIFSLLNIYRIGDKIVERQGNFSYDLIK | |
| | MVEPICNLKLMKLARESRPLVPQFPHFENHIK | |
| | TSVDEGAKIDRGIRFLHDQIMSVKTVDLTLVIY | |
| | GSFRHWGHPFIDYYTGLEKLHSQVTMKKDID | |
| | VSYAKALASDLARIVLFQQFNDHKKWFVNGD | |
| | LLPHDHPFKSHVKENTWPTAAQVQDFGDKW | |
| | HELPLIKCFEIPDLLDPSIIYSDKSHSMNRSEVL | |
| | KHVRMNPNTPIPSKKVLQTMLDTKATNWKEF | |
| | LKEIDEKGLDDDDLIIGLKGKERELKLAGRFFS | |
| | LMSWKLREYFVITEYLIKTHFVPMFKGLTMAD | |
| | DLTAVIKKMLDSSSGQGLKSYEAICIANHIDYE | |
| | KWNNHQRKLSNGPVFRVMGQFLGYPSLIER | |
| | THEFFEKSLIYYNGRPDLMRVHNNTLINSTSQ | |
| | RVCWQGQEGGLEGLRQKGWSILNLLVIQRE | |
| | AKIRNTAVKVLAQGDNQVICTQYKTKKSRNVV | |
| | ELQGALNQMVSNNEKIMTAIKIGTGKLGLLIND | |
| | DETMQSADYLNYGKIPIFRGVIRGLETKRWSR | |
| | VTCVTNDQIPTCANIMSSVSTNALTVAHFAEN | |
| | PINAMIQYNYFGTFARLLLMMHDPALRQSLYE | |
| | VQDKIPGLHSSTFKYAMLYLDPSIGGVSGMSL | |
| | SRFLIRAFPDPVTESLSFWRFIHVHARSEHLK | |
| | EMSAVFGNPEIAKFRITHIDKLVEDPTSLNIAM | |
| | GMSPANLLKTEVKKCLIESRQTIRNQVIKDATI | |
| | YLYHEEDRLRSFLWSINPLFPRFLSEFKSGTF | |
| | LGVADGLISLFQNSRTIRNSFKKKYHRELDDLI | |
| | VRSEVSSLTHLGKLHLRRGSCKMWTCSATH | |
| | ADTLRYKSWGRTVIGTTVPHPLEMLGPQHRK | |
| | ETPCAPCNTSGFNYVSVHCPDGIHDVFSSRG | |
| | PLPAYLGSKTSESTSILQPWERESKVPLIKRA | |
| | TRLRDAISWFVEPDSKLAMTILSNIHSLTGEE | |
| | WTKRQHGFKRTGSALHRFSTSRMSHGGFAS | |
| | QSTAALTRLMATTDTMRDLGDQNFDFLFQAT | |
| | LLYAQITTTVARDGWITSCTDHYHIACKSCLR | |
| | PIEEITLDSSMDYTPPDVSHVLKTWRNGEGS | |
| | WGQEIKQIYPLEGNWKNLAPAEQSYQVGRCI | |
| | GFLYGDLAYRKSTHAEDSSLFPLSIQGRIRGR | |
| | GFLKGLLDGLMRASCCQVIHRRSLAHLKRPA | |
| | NAVYGGLIYLIDKLSVSPPFLSLTRSGPIRDEL | |
| | ETIPHKIPTSYPTSNRDMGVIVRNYFKYQCRLI | |
| | EKGKYRSHYSQLWLFSDVLSIDFIGPFSISTTL | |
| | LQILYKPFLSGKDKNELRELANLSSLLRSGEG | |
| | WEDIHVKFFTKDILLCPEEIRHACKFGIAKDNN | |
| | KDMSYPPWGRESRGTITTIPVYYTTTPYPKML | |
| | EMPPRIQNPLLSGIRLGQLPTGAHYKIRSILHG | |
| | MGIHYRDFLSCGDGSGGMTAALLRENVHSR | |
| | GIFNSLLELSGSVMRGASPEPPSALETLGGD | |
| | KSRCVNGETCWEYPSDLCDPRTWDYFLRLK | |
| | AGLGLQIDLIVMDMEVRDSSTSLKIETNVRNY | |
| | VHRILDEQGVLIYKTYGTYICESEKNAVTILGP | |
| | MFKTVDLVQTEFSSSQTSEVYMVCKGLKKLI | |
| | DEPNPDWSSINESWKNLYAFQSSEQEFARA | |
| | KKVSTYFTLTGIPSQFIPDPFVNIETMLQIFGV | |
| | PTGVSHAAALKSSDRPADLLTISLFYMAIISYY | |
| | NINHIRVGPIPPNPPSDGIAQNVGIAITGISFWL | |
| | SLMEKDIPLYQQCLAVIQQSFPIRWEAVSVKG | |
| | GYKQKWSTRGDGLPKDTRISDSLAPIGNWIR | |
| | SLELVRNQVRLNPFNEILFNQLCRTVDNHLK | |
| | WSNLRRNTGMIEWINRRISKEDRSILMLKSDL | |
| | HEENSWRD | |
| SEQ ID NO: 58 | MGQIVTMFEALPHIIDEVINIVIIVLIIITSIKAVYN | glycoprotein (GP) of |
| | FATCGILALVSFLFLAGRSCGMYGLNGPDIYK | rVSV |
| | GVYQFKSVEFDMSHLNLTMPNACSANNSHH | |
| | YISMGSSGLELTFTNDSILNHNFCNLTSAFNK | |
| | KTFDHTLMSIVSSLHLSIRGNSNHKAVSCDFN | |
| | NGITIQYNLSFSDPQSAISQCRTFRGRVLDMF | |
| | RTAFGGKYMRSGWGWAGSDGKTTWCSQTS | |
| | YQYLIIQNRTWENHCRYAGPFGMSRILFAQE | |
| | KTKFLTRRLAGTFTWTLSDSSGVENPGGYCL | |
| | TKWMILAAELKCFGNTAVAKCNVNHDEEFCD | |
| | MLRLIDYNKAALSKFKQDVESALHVFKTTVNS | |
| | LISDQLLMRNHLRDLMGVPYCNYSKFWYLEH | |
| | AKTGETSVPKCWLVTNGSYLNETHFSDQIEQ | |
| | EADNMITEMLRKDYIKRQGSTPLALMDLLMFS | |
| | TSAYLISIFLHLVKIPTHRHIKGGSCPKPHRLTN | |
| | KGICSCGAFKVPGVKTIWKRR | |
| SEQ ID NO: 59 | MANRTLTLFNVTRNDARAYVSGIQNSVSANR | pVSV-GP128-Mad- |
| | SDPVTLDVLPDSSYLSGANLNLSCHSASPQY | domain |
| | SWRINGIPQQHTQVLFIAKITPNNNGTYACFV | |
| | SNLATGRNNSIVKSITVSASGTSPGLSAAPTL | |
| | PPAWQPFLKDHRISTFKNWPFLEGSAVKKQF | |
| | EELTLGEFLKLDRERAAVARRNERERNRVKL | |
| | VNLGFQALRQHVPHGGASKKLSKVETLRSAV | |
| | EYIRALQRLLAEHDAVRNALAGGLRPQAVRP | |
| | SAPRGPSEGALSPAERELLDFSSWLGGY | |
| SEQ ID NO: 60 | KRYKNRVASRKSRAKFKQLLQHYREVAAAKS | Complex of the first |
| | SENDRLRLLLKNRTLTLFNVTRNDARAYVSGI | component of the |
| | QNSVSANRSDPVTLDVLPDSSYLSGANLNLS | vaccine (“ATP128”) |
| | CHSASPQYSWRINGIPQQHTQVLFIAKITPNN | |
| | NGTYACFVSNLATGRNNSIVKSITVSASGTSP | |
| | GLSAAPTLPPAWQPFLKDHRISTFKNWPFLE | |
| | GSAVKKQFEELTLGEFLKLDRERAAVARRNE | |
| | RERNRVKLVNLGFQALRQHVPHGGASKKLS | |
| | KVETLRSAVEYIRALQRLLAEHDAVRNALAGG | |
| | LRPQAVRPSAPRGPSEGALSPAERELLDFSS | |
| | WLGGYSTVHEILSKLSLEGDHSTPPSAYGSV | |
| | KPYTNFDAE | |
| SEQ ID NO: 61 | EVMLVESGGGLVQPGGSLRLSCTASGFTFSA | HC of PD1-1 |
| | SAMSWVRQAPGKGLEWVAYISGGGGDTYYS | |
| | SSVKGRFTISRDNAKNSLYLQMNSLRAEDTA | |
| | VYYCARHSNVNYYAMDYWGQGTLVTVSSAS | |
| | TKGPSVFPLAPCSRSTSESTAALGCLVKDYFP | |
| | EPVTVSWNSGALTSGVHTFPAVLQSSGLYSL | |
| | SSVVTVPSSSLGTKTYTCNVDHKPSNTKVDK | |
| | RVESKYGPPCPPCPAPEFLGGPSVFLFPPKP | |
| | KDTLMISRTPEVTCVVVDVSQEDPEVQFNWY | |
| | VDGVEVHNAKTKPREEQFNSTYRVVSVLTVL | |
| | HQDWLNGKEYKCKVSNKGLPSSIEKTISKAK | |
| | GQPREPQVYTLPPSQEEMTKNQVSLTCLVKG | |
| | FYPSDIAVEWESNGQPENNYKTTPPVLDSDG | |
| | SFFLYSRLTVDKSRWQEGNVFSCSVMHEALH | |
| | NHYTQKSLSLSLG | |
| SEQ ID NO: 62 | EIVLTQSPATLSLSPGERATMSCRASENIDTS | LC of PD1-1 |
| | GISFMNWYQQKPGQAPKLLIYVASNQGSGIP | |
| | ARFSGSGSGTDFTLTISRLEPEDFAVYYCQQ | |
| | SKEVPWTFGQGTKLEIKRTVAAPSVFIFPPSD | |
| | EQLKSGTASVVCLLNNFYPREAKVQWKVDN | |
| | ALQSGNSQESVTEQDSKDSTYSLSSTLTLSK | |
| | ADYEKHKVYACEVTHQGLSSPVTKSFNRGEC | |
| SEQ ID NO: 63 | EVMLVESGGGLVQPGGSLRLSCTASGFTFSA | HC of PD1-2 |
| | SAMSWVRQAPGKGLEWVAYISGGGGDTYYS | |
| | SSVKGRFTISRDNAKNSLYLQMNSLRAEDTA | |
| | VYYCARHSNPNYYAMDYWGQGTLVTVSSAS | |
| | TKGPSVFPLAPCSRSTSESTAALGCLVKDYF | |
| | PEPVTVSWNSGALTSGVHTFPAVLQSSGLYS | |
| | LSSVVTVPSSSLGTKTYTCNVDHKPSNTKVD | |
| | KRVESKYGPPCPPCPAPEFLGGPSVFLFPPK | |
| | PKDTLMISRTPEVTCVVVDVSQEDPEVQFNW | |
| | YVDGVEVHNAKTKPREEQFNSTYRVVSVLTV | |
| | LHQDWLNGKEYKCKVSNKGLPSSIEKTISKAK | |
| | GQPREPQVYTLPPSQEEMTKNQVSLTCLVK | |
| | GFYPSDIAVEWESNGQPENNYKTTPPVLDSD | |
| | GSFFLYSRLTVDKSRWQEGNVFSCSVMHEA | |
| | LHNHYTQKSLSLSLG | |
| SEQ ID NO: 64 | EIVLTQSPATLSLSPGERATMSCRASENIDTS | LC of PD1-2 |
| | GISFMNWYQQKPGQAPKLLIYVASNQGSGIP | |
| | ARFSGSGSGTDFTLTISRLEPEDFAVYYCQQ | |
| | SKEVPWTFGQGTKLEIKRTVAAPSVFIFPPSD | |
| | EQLKSGTASVVCLLNNFYPREAKVQWKVDN | |
| | ALQSGNSQESVTEQDSKDSTYSLSSTLTLSK | |
| | ADYEKHKVYACEVTHQGLSSPVTKSFNRGEC | |
| SEQ ID NO: 65 | EVMLVESGGGLVQPGGSLRLSCTASGFTFSK | HC of PD1-3 |
| | SAMSWVRQAPGKGLEWVAYISGGGGDTYYS | |
| | SSVKGRFTISRDNAKNSLYLQMNSLRAEDTA | |
| | VYYCARHSNVNYYAMDYWGQGTLVTVSSAS | |
| | TKGPSVFPLAPCSRSTSESTAALGCLVKDYF | |
| | PEPVTVSWNSGALTSGVHTFPAVLQSSGLYS | |
| | LSSVVTVPSSSLGTKTYTCNVDHKPSNTKVD | |
| | KRVESKYGPPCPPCPAPEFLGGPSVFLFPPK | |
| | PKDTLMISRTPEVTCVVVDVSQEDPEVQFNW | |
| | YVDGVEVHNAKTKPREEQFNSTYRWSVLTV | |
| | LHQDWLNGKEYKCKVSNKGLPSSIEKTISKAK | |
| | GQPREPQVYTLPPSQEEMTKNQVSLTCLVK | |
| | GFYPSDIAVEWESNGQPENNYKTTPPVLDSD | |
| | GSFFLYSRLTVDKSRWQEGNVFSCSVMHEA | |
| | LHNHYTQKSLSLSLG | |
| SEQ ID NO: 66 | EIVLTQSPATLSLSPGERATMSCRASENIDVS | LC of PD1-3 |
| | GISFMNWYQQKPGQAPKLLIYVASNQGSGIP | |
| | ARFSGSGSGTDFTLTISRLEPEDFAVYYCQQ | |
| | SKEVPWTFGQGTKLEIKRTVAAPSVFIFPPSD | |
| | EQLKSGTASVVCLLNNFYPREAKVQWKVDN | |
| | ALQSGNSQESVTEQDSKDSTYSLSSTLTLSK | |
| | ADYEKHKVYACEVTHQGLSSPVTKSFNRGEC | |
| SEQ ID NO: 67 | EVMLVESGGGLVQPGGSLRLSCTASGFTFSK | HC of PD1-4 |
| | SAMSWVRQAPGKGLEWVAYISGGGGDTYYS | |
| | SSVKGRFTISRDNAKNSLYLQMNSLRAEDTA | |
| | VYYCARHSNVNYYAMDYWGQGTLVTVSSAS | |
| | TKGPSVFPLAPCSRSTSESTAALGCLVKDYF | |
| | PEPVTVSWNSGALTSGVHTFPAVLQSSGLYS | |
| | LSSVVTVPSSSLGTKTYTCNVDHKPSNTKVD | |
| | KRVESKYGPPCPPCPAPEFLGGPSVFLFPPK | |
| | PKDTLMISRTPEVTCVVVDVSQEDPEVQFNW | |
| | YVDGVEVHNAKTKPREEQFNSTYRWSVLTV | |
| | LHQDWLNGKEYKCKVSNKGLPSSIEKTISKAK | |
| | GQPREPQVYTLPPSQEEMTKNQVSLTCLVK | |
| | GFYPSDIAVEWESNGQPENNYKTTPPVLDSD | |
| | GSFFLYSRLTVDKSRWQEGNVFSCSVMHEA | |
| | LHNHYTQKSLSLSLG | |
| SEQ ID NO: 68 | EIVLTQSPATLSLSPGERATMSCRASENIDVS | LC of PD1-4 |
| | GISFMNWYQQKPGQAPKLLIYVASNQGSGIP | |
| | ARFSGSGSGTDFTLTISRLEPEDFAVYYCQQ | |
| | SKEVPWTFGQGTKLEIKRTVAAPSVFIFPPSD | |
| | EQLKSGTASVVCLLNNFYPREAKVQWKVDN | |
| | ALQSGNSQESVTEQDSKDSTYSLSSTLTLSK | |
| | ADYEKHKVYACEVTHQGLSSPVTKSFNRGEC | |
| SEQ ID NO: 69 | EVMLVESGGGLVQPGGSLRLSCTASGFTFSK | HC of PD1-5 |
| | SAMSWVRQAPGKGLEWVAYISGGGGDTYYS | |
| | SSVKGRFTISRDNAKNSLYLQMNSLRAEDTA | |
| | VYYCARHSNVNYYAMDYWGQGTLVTVSSAS | |
| | TKGPSVFPLAPCSRSTSESTAALGCLVKDYF | |
| | PEPVTVSWNSGALTSGVHTFPAVLQSSGLYS | |
| | LSSVVTVPSSSLGTKTYTCNVDHKPSNTKVD | |
| | KRVESKYGPPCPPCPAPEFLGGPSVFLFPPK | |
| | PKDTLMISRTPEVTCVVVDVSQEDPEVQFNW | |
| | YVDGVEVHNAKTKPREEQFNSTYRVVSVLTV | |
| | LHQDWLNGKEYKCKVSNKGLPSSIEKTISKAK | |
| | GQPREPQVYTLPPSQEEMTKNQVSLTCLVK | |
| | GFYPSDIAVEWESNGQPENNYKTTPPVLDSD | |
| | GSFFLYSRLTVDKSRWQEGNVFSCSVMHEA | |
| | LHNHYTQKSLSLSLG | |
| SEQ ID NO: 70 | EIVLTQSPATLSLSPGERATMSCRASENIDVS | LC of PD1-5 |
| | GISFMNWYQQKPGQAPKLLIYVASNQGSGIP | |
| | ARFSGSGSGTDFTLTISRLEPEDFAVYYCQQ | |
| | SKEVPWTFGQGTKLEIKRTVAAPSVFIFPPSD | |
| | EQLKSGTASVVCLLNNFYPREAKVQWKVDN | |
| | ALQSGNSQESVTEQDSKDSTYSLSSTLTLSK | |
| | ADYEKHKVYACEVTHQGLSSPVTKSFNRGEC | |
| SEQ ID NO: 71 | NRTLTLFNVTRNDARAYVSGIQNSVSANRSD | Mad128-Anaxa |
| | PVTLDVLPDSSYLSGANLNLSCHSASPQYSW | |
| | RINGIPQQHTQVLFIAKITPNNNGTYACFVSNL | |
| | ATGRNNSIVKSITVSASGTSPGLSAAPTLPPA | |
| | WQPFLKDHRISTFKNWPFLEGSAVKKQFEEL | |
| | TLGEFLKLDRERAAVARRNERERNRVKLVNL | |
| | GFQALRQHVPHGGASKKLSKVETLRSAVEYI | |
| | RALQRLLAEHDAVRNALAGGLRPQAVRPSAP | |
| | RGPSEGALSPAERELLDFSSWLGGYSTVHEI | |
| | LSKLSLEGDHSTPPSAYGSVKPYTNFDAE | |
| SEQ ID NO: 72 | VMNIVLIALSILAVLKGLYNIATCGLIGLVTFFLL | LASV GP, matured |
| | CGRSCSSNLYKGVYELQSLDLNMETLNMTM | UniProtID D6NLU4 |
| | PLSCTKNNSHHYIRVGNETGLELTLTNTSLLD | |
| | HKFCNLSDAHKKNLYDHALMSIISTFHLSIPNF | |
| | NQYEAMSCDFNGGKITVQYNLSHSYAGDAA | |
| | RHCGTIANGVLQTFMRMAWGGSYIALDSGH | |
| | GNWDCIMTSYQYLIIQNTTWEDHCQFSRPSPI | |
| | GYLSLLSQRTRDIYISRRLLGTFTWTLSDSEG | |
| | NAT | |
| SEQ ID NO: 73 | DPNAPKRPPSAFFLFCSEKRYKNRVASRKSR | Hp-91 |
| | AKFKQLLQHYREVAAAKSSENDRLRLLLKESL | |
| | KISQAVHAAHAEINEAGREVVGVGALKVPRN | |
| | QDWLGVPRFAKFASFEAQGALANIAVDKANL | |
| | DVEQLESIINFEKLTEWTGS | |
| SEQ ID NO: 74 | ESLKISQAVHAAHAEINEAGREVVGVGALKVP | Mad5 |
| | RNQDWLGVPRFAKFASFEAQGALANIAVDKA | |
| | NLDVEQLESIINFEKLTEWTGS | |
| SEQ ID NO: 75 | QAEPDRAHYNIVTFSSKS | Mad25 |
| SEQ ID NO: 76 | NGRVLELFRAAQLANDVVLQIMELSGATRPT | Mad24 |
| | GIPVHLELASMTNMELMSSIVHQGNNV | |
| SEQ ID NO: 77 | ESLKISQAVHAAHAEINEAGREVVGVGALKVP | Mad39 |
| | RNQDWLGVPRFAKFASFEAQGALANIAVDKA | |
| | NLDVEQLESIINFEKLTEWTGS | |
| SEQ ID NO: 78 | QAEPDRAHYNIVTFCCKC | Mad10 |
| SEQ ID NO: 79 | LFRAAQLANDVVLQIMEHLELASMTNMELMS | Mad12 |
| | SIVVISASIIVFNLLELEG | |

What is claimed is:

1. A vaccine comprising a first component (K) and a second component (V), wherein the first component (K) comprises a complex, said complex consisting of or comprising:
(i) a cell penetrating peptide;
(ii) an antigenic domain, comprising at least one antigen or antigenic epitope; and
(iii) at least one TLR peptide agonist,
wherein the components i)-iii) are covalently linked, and
wherein the second component (V) comprises an oncolytic rhabdovirus.

2. The vaccine according to claim 1, wherein the complex of the first component (K) is a recombinant peptide, polypeptide or protein.

3. The vaccine according to claim 1, wherein the cell penetrating peptide of the first component (K) comprises an amino acid sequence according to any one of SEQ ID NO: 2 (Z13), SEQ ID NO: 3 (Z14), SEQ ID NO: 4 (Z15), or SEQ ID NO: 5 (Z18).

4. The vaccine according to claim 1, wherein the TLR peptide agonist comprises or consists of an amino acid sequence according to SEQ ID NO: 6 and/or SEQ ID NO: 7, or a functional sequence variant of SEQ ID NO: 6 and/or SEQ ID NO: 7.

5. The vaccine according to claim 1, wherein the antigenic domain of said first component (K) comprises more than one antigen or antigenic epitope, in particular 2, 3, 4, 5, 6, 7, 8, 9, 10 or more antigens or antigenic epitopes, wherein the more than one antigen or antigenic epitope, is positioned consecutively in the antigenic domain of the first component.

6. The vaccine according to claim 1, wherein the at least one tumor epitope of said first component (K) is selected from a tumor associated antigen, tumor-specific antigen, or tumor neoantigen.

7. The vaccine according to claim 1, wherein at least one tumor or cancer epitope of the antigenic domain of said first component (K) is selected from the group of tumors or cancers consisting of: gastrointestinal tumors comprising anal cancer, appendix cancer, cholangiocarcinoma, carcinoid tumor, gastrointestinal colon cancer, extrahepatic bile duct cancer, gallbladder cancer, gastric (stomach) cancer, gastrointestinal carcinoid tumor, gastrointestinal stromal tumor (GIST), hepatocellular cancer, pancreatic cancer, rectal cancer, colorectal cancer, and metastatic colorectal cancer.

8. The vaccine according to claim 1, wherein at least one tumor or cancer epitope of the antigenic domain of said first component (K) is an epitope of an antigen selected from the group consisting of EpCAM, HER-2, MUC-1, TOMM34, RNF 43, KOC1, VEGFR, βhCG, survivin, CEA, TGFβR2, p53, KRas, OGT, CASP5, COA-1, MAGE, SART, IL13Ralpha2, ASCL2, NY-ESO-1, MAGE-A3, PRAME, and WT1.

9. The vaccine according to claim 1, wherein the antigenic domain of said first component (K) comprises a peptide consisting of the amino acid sequence according to SEQ ID NO: 22.

10. The vaccine according to claim 1, wherein the antigenic domain of said first component (K) comprises a peptide having an amino acid sequence according to SEQ ID NO: 26 and/or a peptide having an amino acid sequence according to SEQ ID NO: 27.

11. The vaccine according to claim 1, wherein the antigenic domain comprises a peptide consisting of an amino acid sequence according to SEQ ID NO: 16 and/or a peptide having an amino acid sequence according to SEQ ID NO: 17.

12. The vaccine according to claim 1, wherein the antigenic domain comprises in N- to C-terminal direction:
a peptide having an amino acid sequence according to SEQ ID NO: 24, or a fragment thereof having a length of at least 10 amino acids, or a functional sequence variant thereof having at least 70% sequence identity;
a peptide having an amino acid sequence according to SEQ ID NO: 12, or a fragment thereof having a length of at least 10 amino acids, or a functional sequence variant thereof having at least 70% sequence identity; and
a peptide having an amino acid sequence according to SEQ ID NO: 15, or a fragment thereof having a length of at least 10 amino acids, or a functional sequence variant thereof having at least 70% sequence identity.

13. The vaccine according to claim 1, wherein the complex of the first component (K) comprises or consists of an amino acid sequence according to SEQ ID NO: 60, or a functional sequence variant thereof having at least 70%, 75%, 80%, 85%, 90% or 95% sequence identity.

14. The vaccine according to claim 1, wherein the oncolytic rhabdovirus of the second component (V) is an oncolytic recombinant vesicular stomatitis virus, which is preferably one of Vesicular stomatitis Indiana virus (VSIV) or Vesicular stomatitis New Jersey virus (VSNJV).

15. The vaccine according to claim 13, wherein the oncolytic rhabdovirus is an oncolytic recombinant vesicular stomatitis virus that is replication-competent.

16. The vaccine according to claim 13, wherein the oncolytic recombinant vesicular stomatitis virus
(i) lacks a functional gene coding for glycoprotein G, and/or
(ii) lacks a functional glycoprotein G, wherein
(i) the gene coding for the glycoprotein G is replaced by the gene coding for the glycoprotein GP of Lymphocyte choriomeningitis virus (LCMV), and/or
(ii) the glycoprotein G is replaced by the glycoprotein GP of LCMV.

17. The vaccine according to claim 13, wherein the oncolytic recombinant vesicular stomatitis virus of the second component (V) encodes in its genome a second antigenic domain consisting of the amino acid sequence of the antigenic domain of the first component (K) as defined in claim 8.

18. The vaccine according to claim 17, wherein the complex of the first component (K) consists of the amino acid sequence according to SEQ ID NO: 60 and wherein the oncolytic recombinant vesicular stomatitis virus of the second component (V) encodes in its genome
a phosphoprotein (P) comprising the amino acid consisting of SEQ ID NO: 54,
a nucleoprotein (N) comprising the amino acid sequence consisting of SEQ ID NO: 55,
a matrix protein (M) comprising the amino acid sequence consisting of SEQ ID NO: 56,
a large protein (L) comprising the amino acid sequence consisting of SEQ ID NO: 57,
a glycoprotein (GP) comprising the amino acid sequence consisting of SEQ ID NO: 58, and
an antigenic domain which comprises the amino acid sequence consisting of SEQ ID NO: 59.

19. A method of treating a patient in need thereof afflicted with a tumor, wherein the method comprises administering to said patient, an effective amount of the vaccine according to claim 1.

20. The method according to claim 19, wherein the tumor is selected from endocrine tumors, gastrointestinal tumors, genitourinary and gynecologic tumors, breast cancer, head and neck tumors, hematopoietic tumors, skin tumors, thoracic and respiratory tumors, preferably colorectal cancer or metastatic colorectal cancer.

21. The method according to claim 20, wherein the tumor is selected from the group of gastrointestinal tumors comprising anal cancer, appendix cancer, cholangiocarcinoma, carcinoid tumor, gastrointestinal colon cancer, extrahepatic bile duct cancer, gallbladder cancer, gastric (stomach) cancer, gastrointestinal carcinoid tumor, gastrointestinal stromal tumor (GIST), hepatocellular cancer, pancreatic cancer, rectal cancer, colorectal cancer, or metastatic colorectal cancer.

22. The method according to claim 19, wherein the vaccine is co-administered with one or more of an immunotherapeutic agent, such as an immune checkpoint inhibitor, a chemotherapeutic agent, or a targeted drug.

23. The method according to claim 22, wherein the checkpoint modulator is administered concomitantly, sequentially, alternately or following the administration of the vaccine.

24. The method according to claim 19, wherein the first component (K) and the second component (V) of the vaccine are administered by different routes of administration.

25. The method according to claim 19, wherein the first component (K) and the second component (V) of the vaccine are administered in the order first component (K), followed by the second component (V), preferably in the order K-V-K, K-V-K-K.

26. A method of increasing the infiltration of a tumor with tumor antigen-specific T-cells in a patient, the method comprising administering to a patient afflicted with a tumor or cancer the vaccine according to claim 1.

27. A vesicular stomatitis virus, wherein the RNA genome of the vesicular stomatitis virus comprises or consists of an RNA sequence identical or at least 90%, 91%, 92%, 93%, 94%, 95%, 96%, 97%, 98%, or 99% identical to SEQ ID NO: 80.

28. The vaccine according to claim 1, wherein second component (V) encodes at least one antigen or an antigenic epitope thereof of the complex of the first component (K).
